(19) United States
(12) Patent Application Publication
(10) Pub. No.: US 2023/0149535 A1
(43) Pub. Date: May 18, 2023

(54) VACCINES FOR CORONAVIRUS AND METHODS OF USING THE SAME

(71) Applicant: THE WISTAR INSTITUTE OF ANATOMY & BIOLOGY, Philadelphia, PA (US)

(72) Inventors: Daniel W. KULP, Moorestown, NJ (US); David B. WEINER, Merion, PA (US); Yuanhan WU, Philadelphia, PA (US)

(21) Appl. No.: 17/918,321

(22) PCT Filed: Apr. 12, 2021
(86) PCT No.: PCT/US21/26864

§ 371 Date: Oct. 11, 2022

Related U.S. Application Data
(60) Provisional application No. 63/008,733, filed on Apr. 11, 2020.

(51) Int. Cl. A61K 39/215; C07K 14/005; A61P 37/04; C07K 14/195

(52) U.S. Cl. CPC A61K 39/215; C07K 14/005; A61P 37/04; C07K 14/195; C12N 2770/20022; C12N 2770/20034; A61K 2039/53

(57) ABSTRACT

Disclosed are compositions comprising an expressible nucleic acid sequence comprising a first nucleic acid sequence comprising a sequence that encodes a self-assembling polypeptide or a pharmaceutically acceptable salt thereof and a second nucleic acid sequence comprising a sequence that encodes an antigen from a virus from the family of Coronaviridae. In some embodiments, the expressible nucleic acid sequence further comprises a nucleic acid sequence encoding at least one viral antigen or a pharmaceutically acceptable salt thereof. In some embodiments, the expressible nucleic acid sequence further comprises at least one nucleic acid sequence encoding a linker.

### CROSS-REFERENCE TO RELATED APPLICATIONS

This application claims priority to U.S. Provisional Application No. 63/008,733 filed Apr. 11, 2020, the entire content of which is hereby incorporated by reference in its entirety into the present application.

### SUBMISSION OF SEQUENCE LISTING

The Sequence Listing associated with this application is filed in electronic format as a text file and hereby incorporated by reference into the specification in its entirety. The name of the text file containing the Sequence Listing is 37925_0008_SL_ST25 and the size of the text file is 675 KB.

### BACKGROUND

Vaccination is an extremely important public health measure that has demonstrated prophylactic and therapeutic utility against many infectious diseases^{[1-3]}, and impacted some forms of cancer^{[4]}. In the past decade, advances in material engineering has allowed for the development and study of a new generation of vaccines, such as nanoparticle vaccines^{[5-7]}. Hepatitis B and human papillomavirus (HPV) vaccines are examples of such self-assembling virus-like particles which have impacted millions of people^{[8, 9]}. Nanoparticles may come in several shapes and forms. Inorganic materials^{[10, 11]}, nontoxic phospholipids^{[12]}, virus-like particles (VLPs) or self-assembling protein nanoparticles (SAPN)^{[13-16]} can all scaffold and present antigens in repetitive multimeric manners to robustly stimulate immunity in animal models^{[16-18]}. An exemplary disclosure on compositions comprising such self-assembling vaccines and methods of preparing and using the same was provided in PCT application No. PCT/US2019/68444 filed on Dec. 23, 2019 based on U.S. Provisional Application No. 62/784,318 filed Dec. 21, 2018, each of which is incorporated by reference in its entirety.

Another example of such new generation of vaccines are recombinant native-like viral trimer vaccines^{[70-72]}. An exemplary disclosure on compositions comprising nucleic acids encoding such structural trimers and methods of preparing and using the same was provided in PCT application No. PCT/US2020/26948 filed on Apr. 6, 2020 based on U.S. Provisional Application No. 62/829,629 filed on Apr. 4, 2019, each of which is incorporated by reference in its entirety.

Coronaviruses are a group of viruses that cause diseases in mammals and birds. Coronaviruses were first discovered in the 1960s. The earliest virus from the family of Coronaviridae discovered were infectious bronchitis virus in chickens and two viruses from the nasal cavities of human patients with the common cold that were subsequently named human coronavirus 229E (HCoV-229E) and human coronavirus OC43 (HCoV-OC43). Other members of this family have since been identified, including SARS-CoV in 2003, HCoV NL63 in 2004, HKU1 in 2005, MERS-CoV in 2012, and SARS-CoV-2 (formerly known as 2019-nCoV or novel coronavirus 2019, which caused the global COVID-19 pandemic). Most of these have involved serious respiratory tract infections.

Symptoms of coronavirus infection vary in species. In chicken, for instance, they cause an upper respiratory tract disease, while in cows and pigs they cause diarrhea. In humans, coronaviruses cause respiratory tract infections that are typically mild, such as some cases of the common cold (among other possible causes, predominantly rhinoviruses), though rarer forms can be lethal, such as SARS, MERS, and COVID-19.

Coronaviruses vary significantly in risk factor. Some can kill more than 30% of those infected (such as MERS-CoV), and some are relatively harmless, such as the common cold. Coronaviruses cause flu-like symptoms, such as fever and sore throat from swollen adenoids, primarily in the winter and early spring seasons. Coronaviruses can cause pneumonia—either direct viral pneumonia or a secondary bacterial pneumonia—and may cause bronchitis—either direct viral bronchitis or a secondary bacterial bronchitis. The much publicized human coronavirus discovered in 2003, SARS-CoV, which causes severe acute respiratory syndrome (SARS), has a unique pathogenesis because it causes both upper and lower respiratory tract infections. The novel human coronavirus discovered in 2019, SARS-CoV-2, causes mild symptoms, most often fever, dry cough, and shortness of breath, to complications including pneumonia and acute respiratory distress syndrome.

### SUMMARY OF EMBODIMENTS

Since it was emerged in late 2019, SARS-CoV-2 was rapidly characterized as a new member of the betacoronavirus genus, closely related to several bat coronaviruses and to severe acute respiratory syndrome coronavirus (SARS-CoV). Compared to SARS-CoV, however, SARS-CoV-2 appears to be more readily transmitted from human to human.

SARS-CoV-2 makes use of a densely glycosylated Spike (S) protein to gain entry into host cells. The S protein is a trimeric class I fusion protein that exists in a metastable prefusion conformation that undergoes a substantial structural rearrangement to fuse the viral membrane with the host cell membrane^{[73, 74]}. This process is triggered when the S1 subunit of the S protein binds to a host cell receptor. Receptor binding destabilizes the prefusion trimer, resulting in shedding of the S1 subunit and transition of the S2 subunit to a stable postfusion conformation^{[75]}. To engage a host cell receptor, the receptor-binding domain (RBD) of S1 undergoes hinge-like conformational movements that transiently hide or expose the determinants of receptor binding. These two states are referred to as the “down” conformation and the “up” conformation, where down corresponds to the receptor-inaccessible state and up corresponds to the receptor-accessible state, which is thought to be less stabler^{[76-79]}. Because of the indispensable function of the S protein, it represents a target for vaccine design and development. See Wrapp et al., Science, 2020, 367, 1260-1263.

In one aspect, the disclosure relates to a composition comprising an expressible nucleic acid sequence. In some embodiments, the expressible nucleic acid sequence comprises a first nucleic acid sequence encoding a scaffold domain comprising a self-assembling polypeptide and a second nucleic acid sequence encoding a viral antigen from a virus of the family Coronaviridae. In some embodiments, the expressible nucleic acid sequence further comprises a third nucleic acid sequence encoding a leader sequence. In some embodiments, the expressible nucleic acid sequence further comprises a nucleic acid sequence encoding a linker, said nucleic acid sequence positioned between the first nucleic acid sequence and the second nucleic acid sequence in the 5′ to 3′ orientation.

In other embodiments, the expressible nucleic acid sequence comprises a first nucleic acid sequence encoding a leader sequence and a second nucleic acid sequence encoding a soluble viral trimer or a soluble monomer thereof, wherein the soluble viral trimer or the soluble monomer thereof is from a virus of the family Coronaviridae. In some embodiments, the expressible nucleic acid sequence further comprises a third nucleic acid sequence encoding a linker. In some embodiments, the soluble viral trimer or the soluble monomer thereof comprises at least a portion of SARS-CoV-2 spike protein. In some embodiments, the expressible nucleic acid sequence further comprises a nucleic acid sequence encoding a self-assembling polypeptide or a functional fragment or variant thereof.

In some embodiments, the disclosure relates to a vaccine comprising a polypeptide. In some embodiments, the polypeptide comprises a scaffold domain comprising a self-assembling polypeptide and an antigen domain comprising a viral antigen from a virus of the family Coronaviridae. In some embodiments, the polypeptide further comprises a leader sequence. In some embodiments, the polypeptide further comprises a linker domain comprising a linker peptide located between the scaffold domain and the antigen domain. In some embodiments, the polypeptide comprises at least 70% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88 or SEQ ID NO: 91, or a functional fragment or variant thereof.

In some embodiments, the polypeptide comprises a leader sequence and an antigen domain comprising a soluble viral trimer or a soluble monomer thereof, wherein the soluble viral trimer or the soluble monomer thereof is from a virus of the family Coronaviridae. In some embodiments, the polypeptide further comprises one or a plurality of linker. In some embodiments, the soluble viral trimer or the soluble monomer thereof comprises at least a portion of SARS-CoV-2 spike protein. In some embodiments, the polypeptide further comprises a self-assembling polypeptide or a functional fragment or variant thereof. In some embodiments, the polypeptide comprises at least 70% sequence identity to SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

In a further aspect, the disclosure relates to a cell comprising an expressible nucleic acid sequence. In some embodiments, the expressible nucleic acid sequence comprises a first nucleic acid sequence encoding a scaffold domain comprising a self-assembling polypeptide and a second nucleic acid sequence encoding a viral antigen from a virus of the family Coronaviridae. In some embodiments, the expressible nucleic acid sequence further comprises a third nucleic acid sequence encoding a leader sequence. In some embodiments, the expressible nucleic acid sequence further comprises a nucleic acid sequence encoding a linker, said nucleic acid sequence positioned between the first nucleic acid sequence and the second nucleic acid sequence in the 5′ to 3′ orientation. In some embodiments, the expressible nucleic acid sequence comprises a nucleic acid sequence encoding a leader sequence, a nucleic acid sequence encoding a scaffold domain, a nucleic acid sequence encoding a linker, and a nucleic acid sequence encoding a viral antigen from Coronaviridae, in the 5′ to 3′ orientation.

In some embodiments, the expressible nucleic acid sequence comprises a first nucleic acid sequence encoding a leader sequence and a second nucleic acid sequence encoding a soluble viral trimer or a soluble monomer thereof, wherein the soluble viral trimer or the soluble monomer thereof is from a virus of the family Coronaviridae. In some embodiments, the expressible nucleic acid sequence further comprises a third nucleic acid sequence encoding a linker. In some embodiments, the soluble viral trimer or the soluble monomer thereof comprises at least a portion of SARS-CoV-2 spike protein. In some embodiments, the expressible nucleic acid sequence further comprises a nucleic acid sequence encoding a self-assembling polypeptide or a functional fragment or variant thereof.

In some embodiments, the self-assembling polypeptide of the disclosure is from Aquifex aeolicus, Helicobacter pylori, Pyrococcus furiosus or Thermotoga maritima. In some embodiments, the self-assembling polypeptide of the disclosure comprises at least 70% sequence identity to SEQ ID NO: 8, SEQ ID NO: 10, SEQ ID NO: 12, SEQ ID NO: 14, SEQ ID NO: 16, SEQ ID NO: 18 or SEQ ID NO: 20, or a functional fragment or variant thereof.

In some embodiments, the viral antigen of the disclosure is an antigen from a coronavirus. In some embodiments, the viral antigen is an antigen from SARS-CoV-2. In some embodiments, the viral antigen comprises at least 70% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof.

In some embodiments, the leader sequence of the disclosure comprises at least 70% sequence identity to SEQ ID NO: 1 or SEQ ID NO: 5, or a functional fragment or variant thereof.

In some embodiments, the linker of the disclosure comprises at least 70% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof.

In some embodiments, the expressible nucleic acid sequence of the disclosure comprises at least 70% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89 or SEQ ID NO: 90, or a functional fragment or variant thereof. In some embodiments, the expressible nucleic acid sequence of the disclosure encodes a polypeptide comprising at least 70% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88 or SEQ ID NO: 91, or a functional fragment or variant thereof. In some embodiments, the expressible nucleic acid sequence of the disclosure comprises at least 70% sequence identity to SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the expressible nucleic acid sequence of the disclosure encodes a polypeptide comprising at least 70% sequence identity to SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

In some embodiments, the expressible nucleic acid sequence of the disclosure is operably linked to one or a plurality of regulatory sequences. In some embodiments, the expressible nucleic acid sequence of the disclosure is comprised in a nucleic acid molecule. In some embodiments, the expressible nucleic acid sequence of the disclosure is comprised in a nucleic acid molecule which is a plasmid.

In some embodiments, the disclosure relates to a pharmaceutical composition comprising a pharmaceutically effective amount of any of the composition disclosed herein and a pharmaceutically acceptable carrier. In some embodiments, the pharmaceutical composition comprises any of the disclosed composition from about 1 to about 100 micrograms. In some embodiments, the pharmaceutical composition comprises any of the disclosed composition from about 1 to about 20 micrograms.

In some embodiments, the disclosure relates to a method of vaccinating a subject comprising administering a therapeutically effective amount of any of the disclosed pharmaceutical composition to the subject.

In other aspects, the disclosure relates to a method of inducing an immune response in a subject comprising administering to the subject any of the disclosed pharmaceutical composition. In some embodiments, the immune response is an antigen-specific immune response against SARS-CoV-2. In some embodiments, the subject is diagnosed with or suspected of having a SARS-CoV-2 infection. In some embodiments, the immune response is an antigen-specific immune response against a SARS-CoV-2 antigen. In some embodiments, the immune response is an antigen-specific immune response against a SARS-CoV-2 spike antigen.

In some embodiments, the disclosure relates to a method of neutralizing one or a plurality of viruses in a subject comprising administering to the subject any of the disclosed pharmaceutical composition.

In some embodiments, the disclosure relates to a method of stimulating a therapeutically effective antigen-specific immune response against a virus in a mammal infected with a virus comprising administering a therapeutically effective amount of any of the disclosed pharmaceutical composition. In some embodiments, the subject is infected with SARS-CoV-2.

In some embodiments, the disclosure relates to a method of inducing expression of a self-assembling vaccine in a subject comprising administering any of the disclosed pharmaceutical composition. In some embodiments, the method is free of administering any polypeptide directly to the subject.

In some embodiments, the administering in any of the disclosed methods is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or a combination thereof. In some embodiments, the therapeutically effective amount used in any of the disclosed methods is from about 1 to about 2000 micrograms of the expressible nucleic acid sequence. In some embodiments, the therapeutically effective amount used in any of the disclosed methods is from about 1 to about 30 micrograms of the expressible nucleic acid sequence. In some embodiments, the disclosed method is free of activating any mannose-binding lectin or complement process. In some embodiments, the subject is a human. In some embodiments, the therapeutically effective dose used in any of the disclosed method is from about 0.3 microgram of the composition per kilogram of the subject to about 30 micrograms per kilogram of the subject. In some embodiments, the therapeutically effective dose used in any of the disclosed method is from about 0.001 microgram of composition per kilogram of the subject to about 0.05 microgram per kilogram of the subject.

The disclosure further relates to a DNA vaccine comprising an expressible nucleic acid sequence encoding a polypeptide comprising at least about 70% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88 or SEQ ID NO: 91, or a functional fragment or variant thereof. In some embodiments, the expressible nucleic acid sequence of the disclosed DNA vaccine comprises at least about 70% sequence identity to SEQ ID NO: 68, SEQ ID NO: 71, SEQ ID NO: 74, SEQ ID NO: 77, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155 or SEQ ID NO: 158, or a functional fragment or variant thereof. In some embodiments, the disclosed DNA vaccine further comprises a pharmaceutically acceptable excipient. In some embodiments, the pharmaceutically acceptable excipient further comprises an adjuvant.

The disclosure also relates to a RNA vaccine comprising an expressible nucleic acid sequence encoding a polypeptide comprising at least about 70% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88 or SEQ ID NO: 91, or a functional fragment or variant thereof. In some embodiments, the expressible nucleic acid sequence of the disclosed RNA vaccine comprises at least about 70% sequence identity to SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, SEQ ID NO: 156 or SEQ ID NO: 159, or a functional fragment or variant thereof. In some embodiments, the disclosed RNA vaccine further comprises a pharmaceutically acceptable excipient. In some embodiments, the pharmaceutically acceptable excipient further comprises an adjuvant.

The disclosure relates to a viral particle or a self-assemblying particle comprising either a pharmaceutically effective amount of antigen from Coronaviridae in a trimer configuration and/or a viral particle comprising a pharmaceutically effective amount of any nuclec acid sequence disclosed herein. In some embodiments, the viral particle is an adeno-associated vector (AAV) or lentiviral vector.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A-1B show the structure of SARS-CoV-2 in the prefusion conformation. FIG. 1A is a schematic of SARS-CoV-2 S primary structure grayscaled by domain. Domains that could not be visualized in the final map are colored white. SS: signal sequence; S2′: S2′ protease cleavage site; FP: fusion peptide; HR1: heptad repeat 1; CH: central helix; CD: connector domain; HR2: heptad repeat 2; TM: transmembrane domain; CT: cytoplasmic tail. Arrows denote protease cleavage sites. FIG. 1B shows the side and top views of the prefusion structure of the SARS-CoV-2 protein with a single RBD in the up conformation. The two RBD down protomers are shown as cryo-EM density in either white or gray and the RBD up protomer is shown in ribbons grayscaled corresponding to the schematic in (A). Source: Wrapp et al., Science, 2020, 367, 1260-1263.

FIG. 2 shows that the fusion peptide from SARS-CoV2 was scaffolded onto a modified version of Lumazine Synthase (60mer) with an expression domain as a genetic fusion. SARS-CoV-2 fusion peptide nanoparticle shown on the left is decorated by sixty copies of the fusion peptide.

FIG. 3A-3B depict the expression of 2 SARS-CoV-2 fusion peptide nanoparticle designs. FIG. 3A depicts the expression of construct WuhanS_FP_L9GT60_pVax. FIG. 3B depicts the expression of construct WuhanS_FP12_L9GT60_pVax. For SARS-Cov2 fusion peptide purification, DNA was maxiprepped and transfected into Expi293 cells with PEI. Constructs were harvested on day 6. Constructs were purified by lectin chromatography and run on a size-exclusion column (GE Superdex S6 increase). Control nanoparticles elute at ˜12.5 ml using the same protocols.

FIG. 4A-4E show the results of SARS-CoV-2 fusion peptide binding ELISA. FIG. 4A: Week 1 post vaccination; FIG. 4B: Week 2 post vaccination; FIG. 4C: Week 3 post vaccination; FIG. 4D: Week 4 post vaccination; FIG. 4E: Comparison of endpoint titers to WuFuse1 between WuhanS_FP12_L9GT60_pVax (“S_FP12_L9GT60”) and WuhanS_FP_L9GT60_pVax (“S_FP_L9GT60”). ELISA plates were coated with 2 μg/mL streptavidin at room temperature for 8 hours and then blocked overnight at 4° C. The next day, the plates were incubated with fusion peptide at 2 μg/mL at room temp for 2 hours and then with sera at 37° C. for 2 hours, followed by addition of anti-mouse IgG (H+L)-HRP to the plates. The plates were developed with TMB substrates for 5 minutes, absorbances at 450 nm and 570 nm were recorded.

FIG. 5A-5E show the results of SARS-CoV-2 full-length spike protein binding ELISA. FIG. 5A: Week 1 post vaccination; FIG. 5B: Week 2 post vaccination; FIG. 5C: Week 3 post vaccination; FIG. 5D: Week 4 post vaccination; FIG. 5E: Comparison of endpoint titers to SARS-CoV-2 full-length spike protein (“FL SARS2 Spike”) between WuhanS_FP12_L9GT60_pVax (“S_FP12_L9GT60”) and WuhanS_FP_L9GT60_pVax (“S_FP_L9GT60”). ELISA plates were coated with 1 μg/mL anti-histine antibody at room temp for 8 hours and then blocked overnight at 4° C. The next day, the plates were incubated with His-tagged SARS-CoV-2 spike protein at 2 μg/mL at room temperature for 2 hours and then with sera at 37° C. for 2 hours, followed by addition of anti-mouse IgG (H+L)-HRP to the plates. The plates were developed with TMB substrates for 5 minutes, absorbances at 450 nm and 570 nm were recorded.

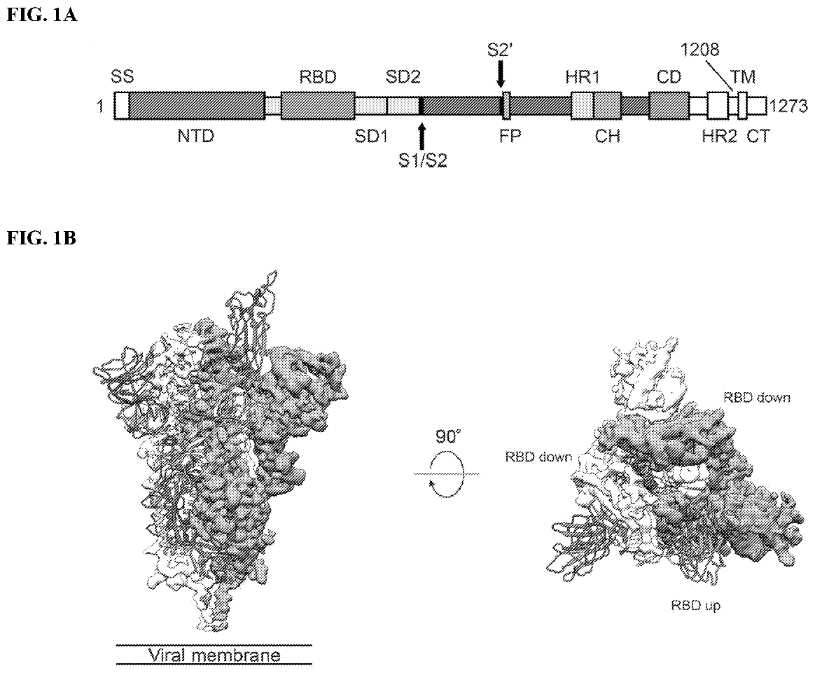

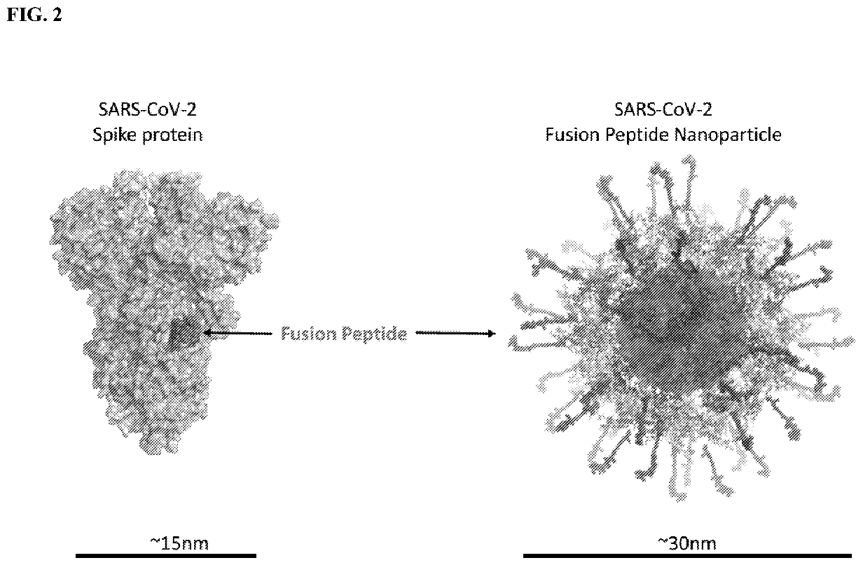

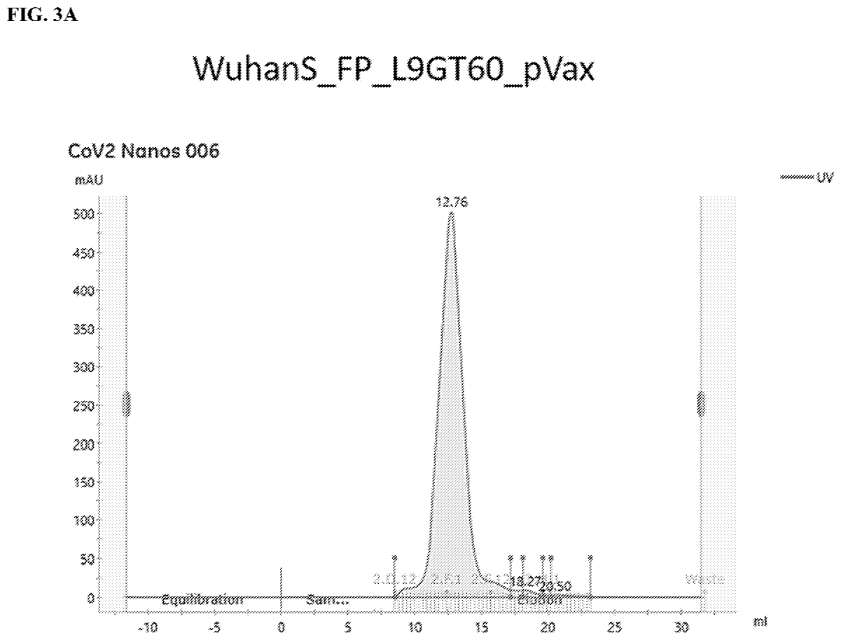

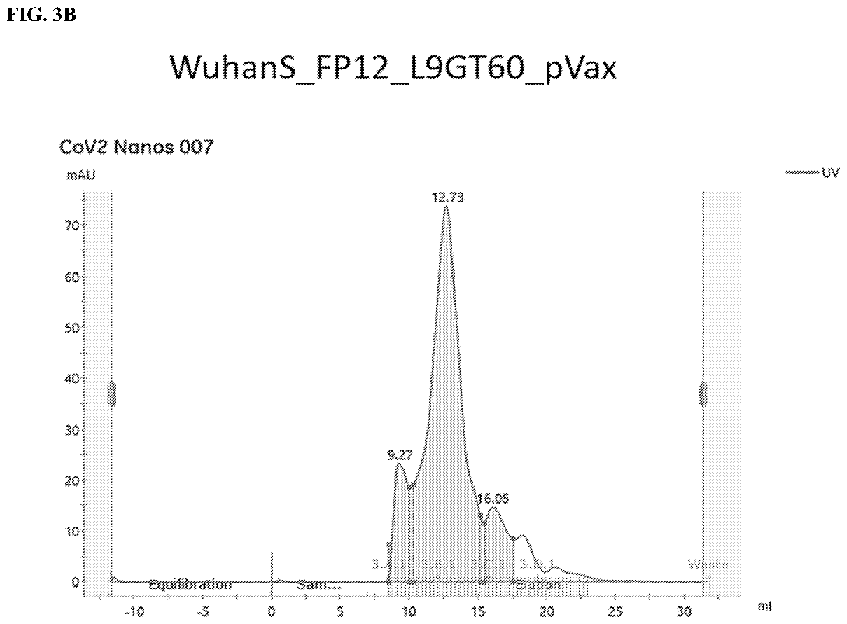

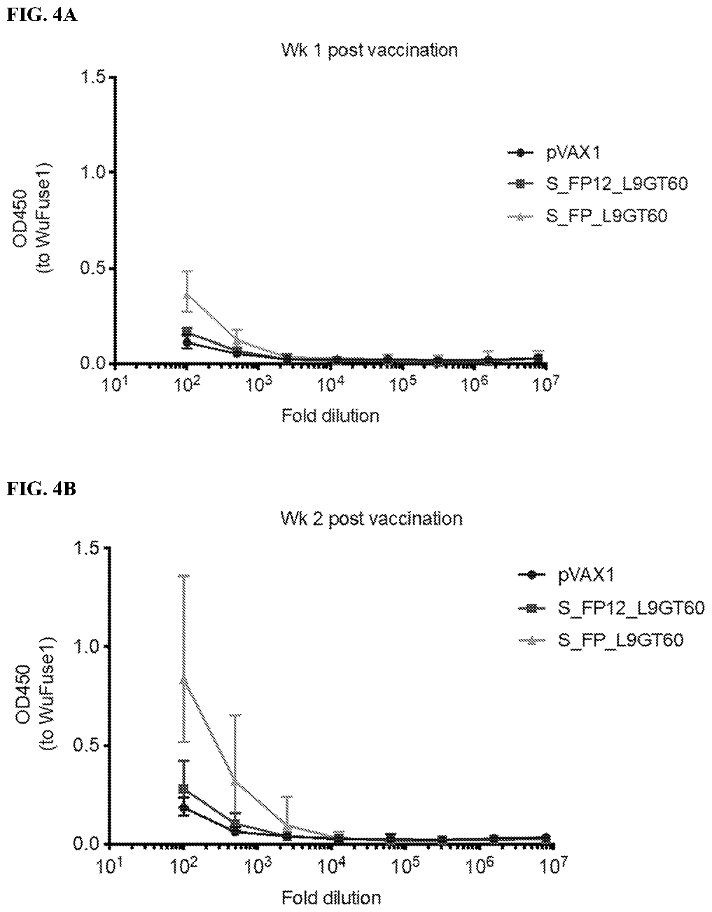

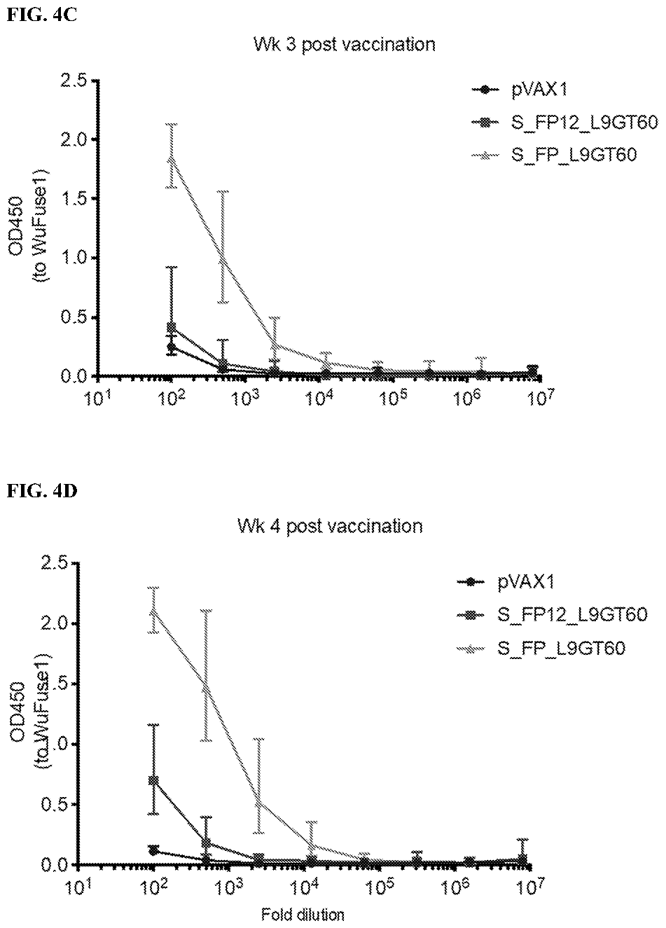

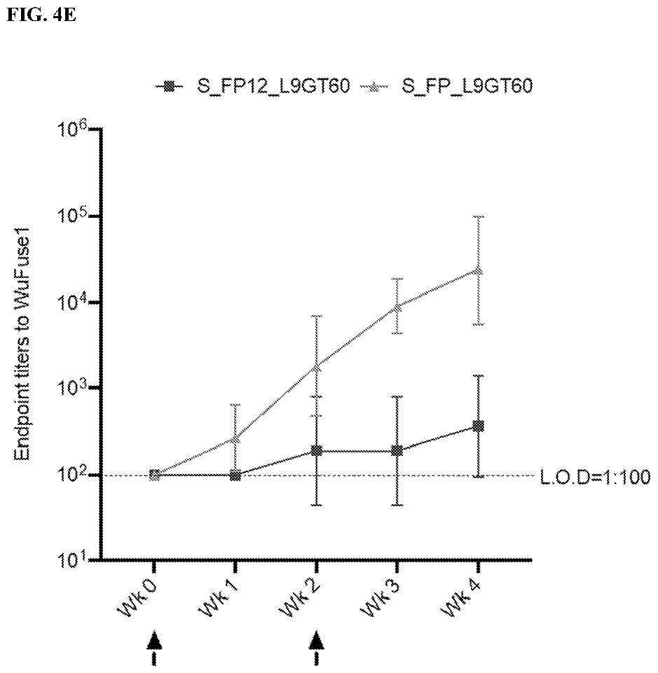

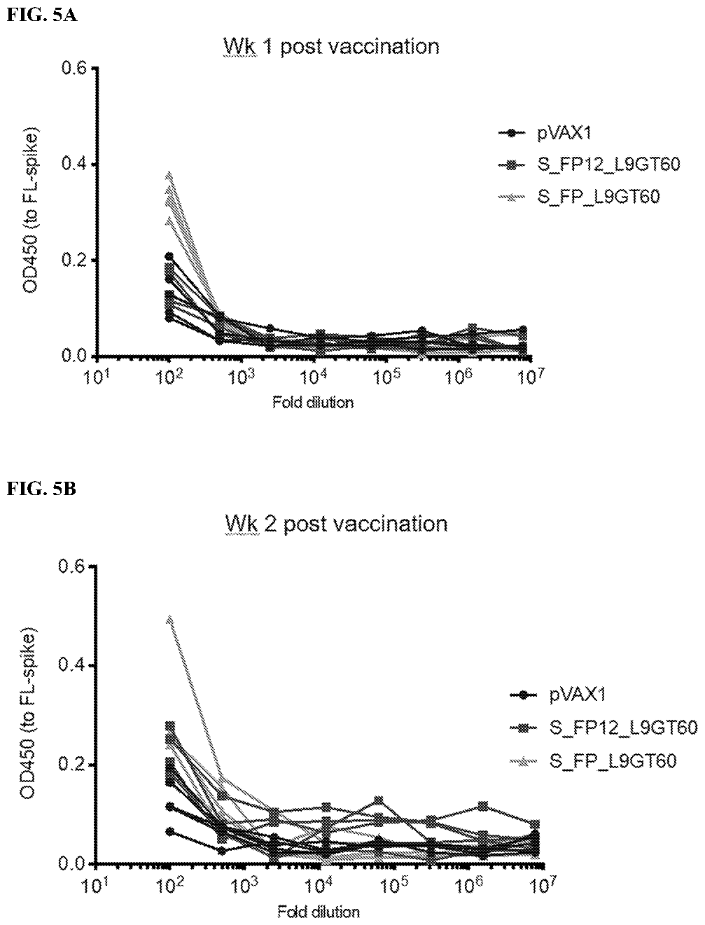

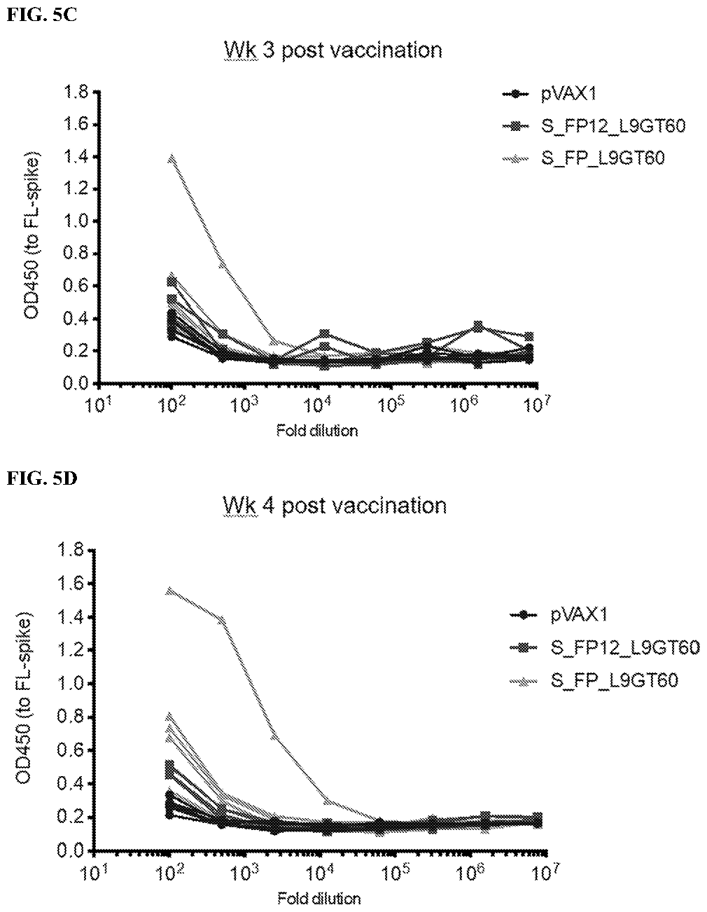

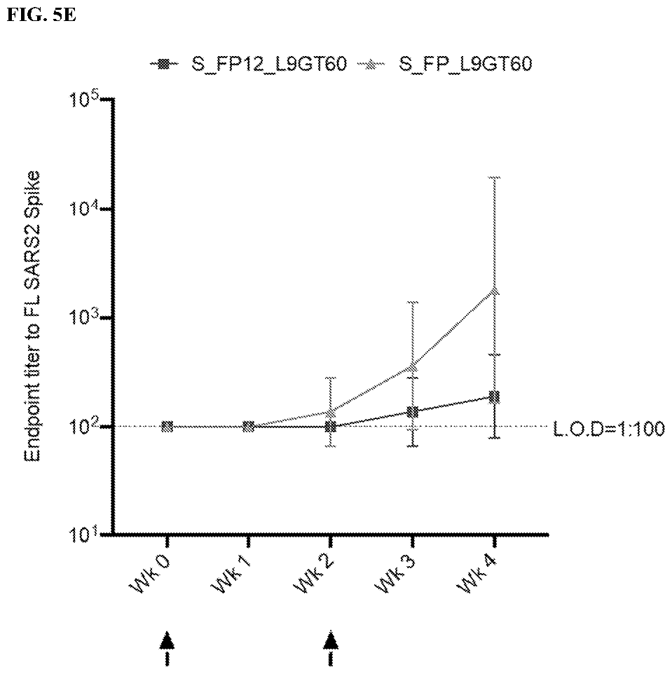

### DETAILED DESCRIPTION

The disclosed method and compositions may be understood more readily by reference to the following detailed description of particular embodiments and the examples included therein and to the figures and their previous and following description. It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only, and is not intended to limit the scope of the present disclosure which will be limited only by the appended claims.

### Definitions

Unless defined otherwise, all technical and scientific terms used herein have the same meaning as commonly understood by one of ordinary skill in the art to which the disclosure pertains.

It must be noted that as used herein and in the appended claims, the singular forms “a”, “an,” and “the” include plural reference unless the context clearly dictates otherwise. Thus, for example, reference to “a nucleic acid sequence” includes a plurality of nucleotides that are formed, reference to “the nucleic acid sequence” is a reference to one or more nucleic acid sequences and equivalents thereof known to those skilled in the art, and so forth.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, indirect or direct ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

The term “combination therapy” as used herein is meant to refer to administration of one or more therapeutic agents in a sequential manner, that is, wherein each therapeutic agent is administered at a different time; as well as administration of these therapeutic agents, or at least two of the therapeutic agents, in a substantially simultaneous manner. In some embodiments, the therapeutic agents are administered within 1, 2, 3, 4, 5, 6, 7, 8, 9, or 10 minutes within each other. Substantially simultaneous administration can be accomplished, for example, by administering to the subject a single dose having a fixed ratio of each therapeutic agent or in multiple, individual doses for each of the therapeutic agents. For example, one combination of the present disclosure may comprise a pooled sample of one or more nucleic acid molecules comprising one or a plurality of expressible nucleic acid sequences and an adjuvant and/or an anti-viral agent administered at the same or different times. In some embodiments, the pharmaceutical composition of the disclosure can be formulated as a single, co-formulated pharmaceutical composition comprising one or more nucleic acid molecules comprising one or a plurality of expressible nucleic acid sequences and one or more adjuvants and/or one or more anti-viral agents. As another example, a combination of the present disclosure (e.g., DNA or RNA vaccines and anti-viral agent) may be formulated as separate pharmaceutical compositions that can be administered at the same or different time. As used herein, the term “simultaneously” is meant to refer to administration of one or more agents at the same time. For example, in certain embodiments, antiviral vaccine or immunogenic composition and antiviral agents are administered simultaneously). Simultaneously includes administration contemporaneously or immediately sequentially, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered intramuscularly only. The components may be administered in any therapeutically effective sequence. A “combination” embraces groups of compounds or non-small chemical compound therapies useful as part of a combination therapy. In some embodiments, the therapeutic agent is an anti-retroviral therapy, (such as one or a combination of efavirenz, lamivudine and tenofovir disoproxil fumarate) or anti-flu therapy (such as TamiFlu®). In some embodiments, the therapeutic agent is one or a combiantion of: abacavir/dolutegravir/lamivudine (Triumeq), dolutegravir/rilpivirine (Juluca), elvitegravir/cobicistat/emtricitabine/tenofovir disoproxil fumarate (Stribild), elvitegravir/cobicistat/emtricitabine/tenofovir alafenami de (Genvoya), efavirenz/emtricitabine/tenofovir disoproxil fumarate (Atripla), emtricitabine/rilpivirine/tenofovir disoproxil fumarate (Complera), emtricitabine/rilpivirine/tenofovir alafenamide (Odefsey), bictegravir, emtricitabine, and tenofovir alafenamide (Biktarvy). In some embodiments, the therapeutic agent is one or a combination of a reverse transcrioptase inhibitor of a retrovirus such as efavirenz (Sustiva), etravirine (Intelence), nevirapine (Viramune), nevirapine extended-release (Viramune XR), rilpivirine (Edurant), delavirdine mesylate (Rescriptor). In some embodiments, the therapeutic agent is one or a combination of a protease inhibitor of a retrovirus, such as: atazanavir/cobicistat (Evotaz), darunavir/cobicistat (Prezcobix), lopinavir/ritonavir (Kaletra), ritonavir (Norvir), atazanavir (Reyataz), darunavir (Prezista), fosamprenavir (Lexiva), tipranavir (Aptivus).

As used herein, “expression” refers to the process by which a polynucleotide is transcribed from a DNA template (such as into and mRNA or other RNA transcript) and/or the process by which a transcribed mRNA (or administered mRNA) is translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. In some embodiments, the at least one expressible nucleic acid sequence comprises only DNA nucleotides, RNA nucleotides or comprises both RNA and DNA nucleotides. In some embodiments, the at least one expressible nucleic acid consist of RNA. In some embodiments, the at least one expressible nucleic acid consist of DNA.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based (such wild-type or full length sequences “reference sequences” or each individually a “reference sequence”). In some embodiments, the functional fragment is derived from the sequence of an organism, such as a human. In such embodiments, the functional fragment may retain about 99%, 98%, 97%, 96%, 95%, 94%, 93%, 92%, 91%, or 90% sequence identity to the wild-type human sequence upon which the sequence is derived. In some embodiments, the functional fragment may retain about 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, 100, 200, 300, 400, 500, 600, 700, 800, 900, 1000 or more nucleotides or amino acids.

“Optional” or “optionally” means that the subsequently described event, circumstance, or material may or may not occur or be present, and that the description includes instances where the event, circumstance, or material occurs or is present and instances where it does not occur or is not present.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiments, to B without A (optionally including elements other than A); in yet another embodiments, to both A and B (optionally including other elements); etc.

As used herein in the specification and in the claims, “or” should he understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein an “antigen” is meant to refer to any substance that elicits an immune response.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other. In some of the disclosed methods of treatment or prevention, the method comprises a step of electroporation of a subject's tissue for a sufficient time and with a sufficient electrical field capable of inducing uptake of the pharmaceutical compositions disclosed herein into the antigen-presenting cells. In some embodiments, the cells are antigen presenting cells.

The term “pharmaceutically acceptable excipient,” “pharmaceutically acceptable carrier” or “pharmaceutically acceptable diluent” as used herein is meant to refer to an excipient, carrier or diluent that can be administered to a subject, together with an agent or the pharmaceutical compositions disclosed herein, and which is inert or fails to eliminate the pharmacological activity of the active agent of the pharmaceutical composition. In some embodiments, the pharmaceutically acceptable carrier does fails to destroy or is incapable of eliminating the pharmacological activity of an active agent/vaccine and and is nontoxic when administered in doses sufficient to deliver a therapeutic amount of the active agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the present disclosure. That is, e.g., a purified polypeptide of the present disclosure is a polypeptide that is at least from about 70 to 100% pure, i.e., the polypeptide is present in a composition wherein the polypeptide constitutes from about 70 to about 100% by weight of the total composition. In some embodiments, the purified polypeptide of the present disclosure is from about 75% to about 99% by weight pure, from about 80% to about 99% by weight pure, from about 90 to about 99% by weight pure, or from about 95% to about 99% by weight pure.

The terms “subject,” “individual,” and “patient” are used interchangeably herein to refer to a vertebrate, preferably a mammal, more preferably a human. Mammals include, but are not limited to, murine, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the subject is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal. The term “mammal” encompasses both humans and non-humans and includes but is not limited to humans, non-human primates, canines, felines, murine, bovines, equines, caprine, and porcines.

The term “therapeutic effect” as used herein is meant to refer to some extent of relief of one or more of the symptoms of a disorder (e.g., SARS-CoV-2 infection) or its associated pathology. A “therapeutically effective amount” as used herein is meant to refer to an amount of an agent which is effective, upon single or multiple dose administration (such as a first, second and/or third booster) to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the present disclosure employed in a pharmaceutical composition at levels lower than that required in order to achieve the desired therapeutic effect and gradually increase the dosage until the desired effect is achieved.

The terms “treat,” “treated,” “treating,” “treatment,” and the like as used herein are meant to refer to reducing or ameliorating a disorder and/or symptoms associated therewith (e.g., a viral infection). “Treating” can refer to administration of the DNA and/or RNA vaccines described herein to a subject after the onset, or suspected onset, of a viral infection. “Treating” includes the concepts of “alleviating,” which refers to lessening the frequency of occurrence or recurrence, or the severity, of any symptoms or other ill effects related to a virus and/or the side effects associated with viral therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

For any therapeutic agent described herein the therapeutically effective amount may be initially determined from preliminary in vitro studies and/or animal models. A therapeutically effective dose may also be determined from human data. The applied dose can be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or 0-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties.

Nucleic acids containing one or more non-naturally occurring or modified nucleotides are also included within one definition of nucleic acids. The modified nucleotide analog may he located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR, NH₂, NHR, N₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Pat. No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

As used herein, the term “nucleic acid molecule” is a molecule that comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of viral antigens. In some embodiments, the disclosure relates to a pharmaceutical composition comprising a first, second, third or more nucleic acid molecule, each of which encoding one or a plurality of viral antigens and at least one of each plasmid comprising one or more of the compositions disclosed herein.

The terms “polypeptide,” “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The term “hybridization” or “hybridizes” as used herein refers to the formation of a duplex between nucleotide sequences that are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary.” Usually two sequences are sufficiently complementary when at least about 90% (preferably at least about 95%) of the nucleotides share base pair organization over a defined length of the molecule.

By “substantially identical” is meant nucleic acid molecule (or polypeptide) exhibiting at least about 50% identity to a reference amino acid sequence (for example, any one of the amino acid sequences described herein) or nucleic acid sequence (for example, any one of the nucleic acid sequences described herein). In some embodiments, such a sequence is at least about 60%, 70%, 80% or 85%, 90%, 95% or even 99% identical at the amino acid level or nucleic acid to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), comprising additional, exogenous DNA, RNA or hybrid DNA or RNA molecules that can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein. In some embodiments, the expression vector includes from about 30 to about 100,000 nucleotides (e.g., from about 30 to about 50, from about 30 to about 100, from about 30 to about 250, from about 30 to about 500, from about 30 to about 1,000, from about 30 to about 1,500, from about 30 to about 3,000, from about 30 to about 5,000, from about 30 to about 7,000, from about 30 to about 10,000, from about 30 to about 25,000, from about 30 to about 50,000, from about 30 to about 70,000, from about 100 to about 250, from about 100 to about 500, from about 100 to about 1,000, from about 100 to about 1,500, from about 100 to about 3,000, from about 100 to about 5,000, from about 100 to about 7,000, from about 100 to about 10,000, from about 100 to about 25,000, from about 100 to about 50,000, from about 100 to about 70,000, from about 100 to about 100,000, from about 500 to about 1,000, from about 500 to about 1,500, from about 500 to about 2,000, from about 500 to about 3,000, from about 500 to about 5,000, from about 500 to about 7,000, from about 500 to about 10,000, from about 500 to about 25,000, from about 500 to about 50,000, from about 500 to about 70,000, from about 500 to about 100,000, from about 1,000 to about 1,500, from about 1,000 to about 2,000, from about 1,000 to about 3,000, from about 1,000 to about 5,000, from about 1,000 to about 7,000, from about 1,000 to about 10,000, from about 1,000 to about 25,000, from about 1,000 to about 50,000, from about 1,000 to about 70,000, from about 1,000 to about 100,000, from about 1,500 to about 3,000, from about 1,500 to about 5,000, from about 1,500 to about 7,000, from about 1,500 to about 10,000, from about 1,500 to about 25,000, from about 1,500 to about 50,000, from about 1,500 to about 70,000, from about 1,500 to about 100,000, from about 2,000 to about 3,000, from about 2,000 to about 5,000, from about 2,000 to about 7,000, from about 2,000 to about 10,000, from about 2,000 to about 25,000, from about 2,000 to about 50,000, from about 2,000 to about 70,000, and from about 2,000 to about 100,000 nucleotides).

The term “vaccine” as used herein is meant to refer to a composition capable of generating immunity for the prophylaxis and/or treatment of diseases (e.g., viral infections). In some embodiments, the vaccine is a composition capable of generating therapeutically effective immunity for the prophylaxis or treatment of Coronaviridae infection or propagation in a subject. Accordingly, vaccines are medicaments which comprise antigens in protein and/or nucleic acid forms and are in animals for generating specific defense and protective substance by vaccination. A “vaccine composition” can include a pharmaceutically acceptable excipient, carrier or diluent. A “vaccine composition” or “nucleic acid vaccine composition” as used herein can comprise a DNA vaccine, a RNA vaccine or a combination thereof.

“Variants” are intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring or endogenous nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native or wild-type protein upon which the variant structure is based. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly. In some embodiments, the nucleic acid molecules or the nucleic acid sequences comprise conservative mutations of 1, 2, 3, 4, 5, 6, 7, 8, 9, 10 or more nucleotides.

As used herein, “SARS-CoV-2” refers to severe acute respiratory syndrome-related coronavirus-2 or a subtype or variant thereof. Non-limiting examples of the SARS-CoV-2 virus, variants and subtypes thereof are described, for example, in Morais et al. (Sci. Rep., 2020, 10(1):18289), Zhao et al. (PLoS Comput Biol., 2020, 16(9):e1008269), Forster et al. (PNAS, 2020, 117(17):9241-9243), and Tang et al. (National Science Review, 2020, 7(6):1012-1023), all of which are incorporated by reference herein. In some embodiments, the SARS-CoV-2 is one of the 6 subtypes and 10 tentative subtypes identified in Morais et al. (Sci. Rep., 2020, 10(1):18289). In some embodiments, the SARS-CoV-2 is one of the 3 SARS-CoV-2 variants identified in Foster et al. (PNAS, 2020, 117(17):9241-9243). In some embodiments, the SARS-CoV-2 is one of the two SARS-CoV-2 subtypes identified in Tang et al. (National Science Review, 2020, 7(6):1012-1023). In some embodiments, the SARS-CoV-2 is SARS-CoV-2 hCoV-19/Australia/VIC01/2020 or a variant thereof. In some embodiments, SARS-COV-2 comprises the sequences as described in NCBI Reference Sequence: NC 045512.2 or a variant thereof. In some embodiments, SARS-CoV-2 comprises the sequence as described in GenBank: MN908947.3 or a variant thereof. In some embodiments, the SARS-Cov-2 variant is the B.1.1.7 varian, also referred to as lineage B.1.1.7, VOC 202012/01 or 20I/501Y.V1. In some embodiments, the SARS-Cov-2 variant is the B.1.351 variant, also referred to as B.1.351 lineage. In some embodiments, the SARS-Cov-2 variant is the B.1.1.28 subclade (renamed “P.1”). In some embodiments, the SARS-Cov-2 variant is the B.1.1.7 variant, also referred to as B.1.1.7 lineage or 20I/501Y.V1.

As used herein, a “scaffold domain” refers to a region of a polypeptide that structurally facilitates, or acts as a scaffold for, presentation of one or a plurality of viral antigens. In some embodiments, a scaffold domain of the disclosure comprises a self-assembling polypeptide which, when expressed, forms an inner scaffold or core upon which one or a plurality of viral antigens are displayed. In some embodiments, the self-assemblying peptide forms a three dimensional shape with identical single polypeptide surfaces. In some embodiments, the scaffold doman forms a secondary and/or tertiary structure that is a polyhedron. In some embodiments, the scaffold domain encodes a monomer or series of monomers that form a 7, 14, 20, 28 or 60 faces of a polyhedron upon which antigens are encoded or displayed.

As used herein, the term “trimer” refers to a protein complex formed by three individual monomers. In some embodiments, the monomer subunits of the trimer are bound together to form a trimer by non-covalent protein-protein interactions.

Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior disclosure. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### A. Nucleic Acid Compositions

Disclosed are compositions and pharmaceutical compositions comprising one or plurality of expressible nucleic acid sequences. In some embodiments, the expressible nucleic acid sequence is a DNA. In other embodiments, the expressible nucleic acid sequence is a RNA. In some embodiments, the expressible nucleic acid is operably linked to one or a plurality of regulatory sequences. In some embodiments, the expressible nucleic acid sequence is comprised and forms a part of a nucleic acid molecule, such as a vector or plasmid.

In one aspect, the expressible nucleic acid sequence of the disclosure comprises a first nucleic acid sequence encoding a scaffold domain comprising a self-assembling polypeptide or a pharmaceutically acceptable salt thereof, and a second nucleic acid sequence encoding an antigen domain comprising a viral antigen or a pharmaceutically acceptable salt thereof. The self-assembling polypeptide is a self-assembling peptide that is expressed to facilitate presentation of the viral antigen. Transformed or transfected cells exposed to such expressible nucleic acid sequences can produce the self-assembling peptide which is enveloped by the viral antigens, thereby stimulating the viral antigen-specific immune response against the antigen. In some embodiments, the antigen-specific immune response is a therapeutically effective immune response against the virus from which the antigen amino acid sequence is obtained. In some embodiments, the viral antigen encoded by the expressible nucleic acid of the disclosure comprises a coronaviral antigen. In some embodiments, the expressible nucleic acid sequence further comprises a third nucleic acid sequence encoding a leader sequence or a pharmaceutically acceptable salt thereof. In some embodiments, the leader sequence is an IgE or IgG leader sequence. In some embodiments, the expressible nucleic acid sequence further comprises a fourth nucleic acid sequence encoding a linker peptide or a pharmaceutically acceptable salt thereof, wherein the fourth nucleic acid sequence is positioned between the first nucleic acid sequence and the second nucleic acid sequence in the 5′ to 3′ orientation. In some embodiments, the expressible nucleic acid sequence comprises a first nucleic acid sequence encoding a scaffold domain comprising a self-assembling polypeptide or a pharmaceutically acceptable salt thereof, a second nucleic acid sequence encoding an antigen domain comprising a viral antigen or a pharmaceutically acceptable salt thereof, and a third nucleic acid sequence encoding a leader sequence or a pharmaceutically acceptable salt thereof. In some embodiments, the expressible nucleic acid sequence comprises a first nucleic acid sequence encoding a scaffold domain comprising a self-assembling polypeptide or a pharmaceutically acceptable salt thereof, a second nucleic acid sequence encoding an antigen domain comprising a viral antigen or a pharmaceutically acceptable salt thereof, a third nucleic acid sequence encoding a leader sequence or a pharmaceutically acceptable salt thereof, and a fourth nucleic acid sequence encoding a linker peptide or a pharmaceutically acceptable salt thereof, wherein the fourth nucleic acid sequence is positioned between the first nucleic acid sequence and the second nucleic acid sequence in the 5′ to 3′ orientation.

In some embodiments, the expressible nucleic acid sequence of the disclosure comprises a nucleic acid sequence encoding a viral trimer polypeptide, a functional fragment thereof or a pharmaceutically acceptable salt thereof. In some embodiments, the expressible nucleic acid sequence comprises, in a 5′ to 3′ orientation, a first nucleic acid sequence encoding a leader sequence or a pharmaceutically acceptable salt thereof, and a second nucleic acid sequence encoding a viral trimer polypeptide, a functional fragment or variant thereof or a pharmaceutically acceptable salt thereof. In some embodiments, the leader sequence is an IgE or IgG leader sequence. In some embodiments, the expressible nucleic acid sequence comprises, in a 5′ to 3′ orientation, a first nucleic acid sequence encoding a leader sequence or a pharmaceutically acceptable salt thereof, and a second nucleic acid sequence encoding a viral polypeptide that is a component of a viral trimer, a functional fragment or variant thereof or a pharmaceutically acceptable salt thereof. In some embodiments, the viral polypeptide that is a component of a viral trimer is a monomer of a viral trimer, such that, upon expression, the monomers spontaneously aggregate to form a trimeric viral polypeptide. In some embodiments, the viral trimer encoded by the expressible nucleic acid of the disclosure comprises a coronaviral trimer. In some embodiments, the viral trimer comprises the spike protein of SARS-CoV-2, a functional fragment or variant thereof or a pharmaceutically acceptable salt thereof.

In some embodiments, the nucleic acid sequences encoding the viral antigens or viral trimers comprised in the expressible nucleic acid of the disclosure comprise one or a plurality of mutations so to tailor the vaccine induced responses. Such mutations result in creating glycan sites in the encoded polypeptide so that glycosylation events can be obtained. In some embodiments, such glycan modifications or mutations decrease the bottom reactivity. In some embodiments, such glycan modifications or mutations increase antigen activity.

1. Leader Sequence

The expressible nucleic acid sequence of the present disclosure optionally comprises a nucleic acid sequence encoding a leader sequence, a functional fragment or variant thereof or a pharmaceutically acceptable salt thereof. A “leader sequence” may from time to time refer to a “signal peptide” and thus, the terms “leader sequence” and “signal peptide” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences, when present, are linked at the N terminus of the protein. The presence of a leader sequence may be required for proper secretion of the viral antigen or trimer encoded by the expressible nucleic acid sequence of the disclosure.

A non-limiting example of the leader sequence is the IgE leader sequence comprising the amino acid sequence of MDWTWILFLVAAATRVHS (SEQ ID NO: 1; also named “MD39”) encoded by one of the following nucleic acid sequences:

(SEQ ID NO: 2; “MD39”)
atggactggacatggattctgttcctggtcgctgccgctacaagagtgca
ttcc;
(SEQ ID NO: 3; “CPG9.2”)
atggattggacttggattctgttcctggtcgcagcagccacacgagtgca
tagc;
and
(SEQ ID NO: 4)
atggactggacctggattctgttcctggtggccgccgccacaagggtgca
cagc.

Another non-limiting example of the leader sequence is the amino acid sequence of MDWTWRILFLVAAATGTHA (SEQ ID NO: 5) encoded by the nucleic acid sequence of atggactggacctggagaatcctgttcctggtggccgccgccaccggcacacacgccgatacacacttccccatctgcatcttttgctg tggctgttgccataggtccaagtgtgggatgtgctgcaaaact (SEQ ID NO: 6).

Thus, in some embodiments when the leader sequence is present, the leader sequence comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 1 or SEQ ID NO: 5, or a functional fragment or variant thereof. In some embodiments, the leader sequence comprises the amino acid sequence of SEQ ID NO: 1 or SEQ ID NO: 5, or a functional fragment or variant thereof. In some embodiments, the leader sequence is encoded by a nucleic acid sequence comprising at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 2, SEQ ID NO: 3, SEQ ID NO: 4 or SEQ ID NO: 6, or a functional fragment or variant thereof. In some embodiments, the leader sequence is encoded by the nucleic acid sequence of SEQ ID NO: 2, SEQ ID NO: 3, SEQ ID NO: 4 or SEQ ID NO: 6, or a functional fragment or variant thereof. In other embodiments, the leader sequence is encoded by a nucleic acid sequence comprising at least about 60%, 65%, 70%, 75%, 80%, 85%, 86%, 87%, 88%, 89%, 90%, 91%, 92%, 93%, 94%, 95%, 96%, 97%, 98% or 99% sequence identity to a nucleic acid sequence that is complementary to SEQ ID NO: 2, SEQ ID NO: 3, SEQ ID NO: 4 or SEQ ID NO: 6, or a functional fragment or variant thereof. In some embodiments, the leader sequence is encoded by a nucleic acid sequence that is complementary to the nucleic acid sequence of SEQ ID NO: 2, SEQ ID NO: 3, SEQ ID NO: 4 or SEQ ID NO: 6, or a functional fragment or variant thereof

2. Self-Assembling Polypeptide

The disclosure relates to an expressible nucleic acid sequence comprising at least one nucleic acid sequence encoding a scaffold domain comprising a self-assembling polypeptide, a functional fragment or variant thereof or a pharmaceutically acceptable salt thereof. Self-assembling polypeptide are polypeptides capable of undergoing spontaneous assembling into ordered nanostructures. Effectively self-assembling polypeptides can act as building blocks to form the scaffold domain of the present disclosure. In some embodiments, the self-assembling polypeptides encoded by the expressible nucleic acid sequence of the disclosure are monomeric forms of viral trimers or variants thereof. In some embodiments, the self-assembling polypeptides are monomers of nanoparticle structural proteins that self-assemble into nanoparticles upon expression. Any self-assembling polypeptide can be used. In some embodiments, the self assembling polypeptide is from Aquifex aeolicus, Helicobacter pylori, Pyrococcus furiosus or Thermotoga maritima.

A non-limiting example of a self-assembling polypeptide is the lumazine synthase of hyperthermophilic bacterium Aquifex aeolicus having the amino acid sequence of SEQ ID NO: 8 (LS scaffold) encoded by the nucleic acid sequence of SEQ ID NO: 7.

(SEQ ID NO: 7)
atgcagatctacgaaggaaaactgaccgctgagggactgaggttcggaat
tgtcgcaagccgcgcgaatcacgcactggtggataggctggtggaaggcg
ctatcgacgcaattgtccggcacggcgggagagaggaagacatcacactg
gtgagagtctgcggcagctgggagattcccgtggcagctggagaactggc
tcgaaaggaggacatcgatgccgtgatcgctattggggtcctgtgccgag
gagcaactcccagcttcgactacatcgcctcagaagtgagcaaggggctg
gctgatctgtccctggagctgaggaaacctatcacttttggcgtgattac
tgccgacaccctggaacaggcaatcgaggcggccggcacctgccatggaa
acaaaggctgggaagcagccctgtgcgctattgagatggcaaatctgttc
aaatctctgcga
(SEQ ID NO: 8)
MQIYEGKLTAEGLRFGIVASRANHALVDRLVEGAIDAIVRHGGREEDITL
VRVCGSWEIPVAAGELARKEDIDAVIAIGVLCRGATPSFDYIASEVSKGL
ADLSLELRKPITFGVITADTLEQAIEAAGTCHGNKGWEAALCAIEMANLF
KSLR

Another non-limiting example of a self-assembling polypeptide is ferritin from Helicobacter pylori having the amino acid sequence of SEQ ID NO: 10 (3BVE scaffold) encoded by the nucleic acid sequence of SEQ ID NO: 9.

(SEQ ID NO: 9)
gggctgagtaaggacattatcaagctgctgaacgaacaggtgaacaaaga
gatgcagtctagcaacctgtacatgtccatgagctcctggtgctataccc
actctctggacggagcaggcctgttcctgtttgatcacgccgccgaggag
tacgagcacgccaagaagctgatcatcttcctgaatgagaacaatgtgcc
cgtgcagctgacctctatcagcgcccctgagcacaagttcgagggcctga
cacagatctttcagaaggcctacgagcacgagcagcacatctccgagtct
atcaacaatatcgtggaccacgccatcaagtccaaggatcacgccacatt
caactttctgcagtggtacgtggccgagcagcacgaggaggaggtgctgt
ttaaggacatcctggataagatcgagctgatcggcaatgagaaccacggg
ctgtacctggcagatcagtatgtcaagggcatcgctaagtcaaggaaaag
c
(SEQ ID NO: 10)
GLSKDIIKLLNEQVNKEMQSSNLYMSMSSWCYTHSLDGAGLFLFDHAAEE
YEHAKKLIIFLNENNVPVQLTSISAPEHKFEGLTQIFQKAYEHEQHISES
INNIVDHAIKSKDHATFNFLQWYVAEQHEEEVLFKDILDKIELIGNENHG
LYLADQYVKGIAKSRKS

A yet another non-limiting example of a self-assembling polypeptide is PfV viral cage from Pyrococcus furiosus (2e0z) having the amino acid sequence of SEQ ID NO: 12 (RBE scaffold) encoded by the nucleic acid sequence of SEQ ID NO: 11.

(SEQ ID NO: 11)
ctgagcattgcccccacactgattaaccgggacaaaccctacaccaaaga
ggaactgatggagattctgagactggctattatcgctgagctggacgcca
tcaacctgtacgagcagatggcccggtattctgaggacgagaatgtgcgc
aagatcctgctggatgtggccagggaggagaaggcacacgtgggagagtt
catggccctgctgctgaacctggaccccgagcaggtgaccgagctgaagg
gcggctttgaggaggtgaaggagctgacaggcatcgaggcccacatcaac
gacaataagaaggaggagagcaacgtggagtatttcgagaagctgagatc
cgccctgctggatggcgtgaataagggcaggagcctgctgaagcacctgc
ctgtgaccaggatcgagggccagagcttcagagtggacatcatcaagttt
gaggatggcgtgcgcgtggtgaagcaggagtacaagcccatccctctgct
gaagaagaagttctacgtgggcatcagggagctgaacgacggcacctacg
atgtgagcatcgccacaaaggccggcgagctgctggtgaaggacgaggag
tccctggtcatccgcgagatcctgtctacagagggcatcaagaagatgaa
gctgagctcctgggacaatccagaggaggccctgaacgatctgatgaatg
ccctgcaggaggcatctaacgcaagcgccggaccattcggcctgatcatc
aatcccaagagatacgccaagctgctgaagatctatgagaagtccggcaa
gatgctggtggaggtgctgaaggagatcttccggggcggcatcatcgtga
ccctgaacatcgatgagaacaaagtgatcatctttgccaacacccctgcc
gtgctggacgtggtggtgggacaggatgtgacactgcaggagctgggacc
agagggcgacgatgtggcctttctggtgtccgaggccatcggcatcagga
tcaagaatccagaggcaatcgtggtgctggag
(SEQ ID NO: 12)
LSIAPTLINRDKPYTKEELMEILRLAIIAELDAINLYEQMARYSEDENVR
KILLDVAREEKAHVGEFMALLLNLDPEQVTELKGGFEEVKELTGIEAHIN
DNKKEESNVEYFEKLRSALLDGVNKGRSLLKHLPVTRIEGQSFRVDIIKF
EDGVRVVKQEYKPIPLLKKKFYVGIRELNDGTYDVSIATKAGELLVKDEE
SLVIREILSTEGIKKMKLSSWDNPEEALNDLMNALQEASNASAGPFGLII
NPKRYAKLLKIYEKSGKMLVEVLKEIFRGGIIVTLNIDENKVIIFANTPA
VLDVVVGQDVTLQELGPEGDDVAFLVSEAIGIRIKNPEAIVVLE

A further non-limiting example of a self-assembling polypeptide is the self-assembling polypeptide from Thermotoga maritima having the amino acid sequence of SEQ ID NO: 14 (13 scaffold) encoded by the nucleic acid sequence of SEQ ID NO: 13.

(SEQ ID NO: 13)
atgaagatggaagaactgttcaagaagcacaagatcgtggccgtgctgag
ggccaactccgtggaggaggccaagaagaaggccctggccgtgttcctgg
gcggcgtgcacctgatcgagatcacctttacagtgcccgacgccgatacc
gtgatcaaggagctgtctttcctgaaggagatgggagcaatcatcggagc
aggaaccgtgacaagcgtggagcagtgcagaaaggccgtggagagcggcg
ccgagtttatcgtgtcccctcacctggacgaggagatctctcagttctgt
aaggagaagggcgtgttttacatgccaggcgtgatgacccccacagagct
ggtgaaggccatgaagctgggccacacaatcctgaagctgttccctggcg
aggtggtgggcccacagtttgtgaaggccatgaagggccccttccctaat
gtgaagtttgtgcccaccggcggcgtgaacctggataacgtgtgcgagtg
gttcaaggcaggcgtgctggcagtgggcgtgggcagcgccctggtgaagg
gcacacccgtggaagtcgctgagaaggcaaaggcattcgtggaaaagatt
agggggtgtactgag
(SEQ ID NO: 14)
MKMEELFKKHKIVAVLRANSVEEAKKKALAVFLGGVHLIEITFTVPDADT
VIKELSFLKEMGAIIGAGTVTSVEQCRKAVESGAEFIVSPHLDEEISQFC
KEKGVFYMPGVMTPTELVKAMKLGHTILKLFPGEVVGPQFVKAMKGPFPN
VKFVPTGGVNLDNVCEWFKAGVLAVGVGSALVKGTPVEVAEKAKAFVEKI
RGCTE

Additional non-limiting examples of self-assembling polypeptides include: QB scaffold (SEQ ID NO: 16) encoded by the nucleic acid sequence of SEQ ID NO: 15;

(SEQ ID NO: 15)
gcaaagctggagacagtgacactgggcaacatcggcaaggacggcaagca
gacactggtgctgaatcccaggggcgtgaaccctaccaatggagtggcat
ctctgagccaggcaggagcagtgcctgccctggagaagagagtgaccgtg
tccgtgtctcagcccagcaggaacagaaagaattataaggtgcaggtgaa
gatccagaacccaaccgcctgcacagccaatggcagctgtgacccatccg
tgacaaggcaggcatacgcagatgtgaccttctcttttacacagtatagc
accgatgaggagagggccttcgtgcgcaccgagctggccgccctgctggc
atcccctctgctgattgacgctattgaccagctgaaccctgcttac
(SEQ ID NO: 16)
AKLETVTLGNIGKDGKQTLVLNPRGVNPTNGVASLSQAGAVPALEKRVTV
SVSQPSRNRKNYKVQVKIQNPTACTANGSCDPSVTRQAYADVTFSFTQYS
TDEERAFVRTELAALLASPLLIDAIDQLNPAY
ICI scaffold (SEQ ID NO: 18) encoded by the
nucleic acid sequence of SEQ ID NO: 17;
(SEQ ID NO: 17)
gaccctgagtttaccaaaaatgctctgaatgtcgtcaaaaatgatctgat
tgctaaggtggaccagctgagcggagagcaggaggtgctgaggggcgagc
tggaggccgccaagcaggcaaaggtgaaactggaaaaccgaatcaaggaa
ctggaagaagaactgaaaagagtc
(SEQ ID NO: 18)
DPEFTKNALNVVKNDLIAKVDQLSGEQEVLRGELEAAKQAKVKLENRIKE
LEEELKRV
and IC2 scaffold (SEQ ID NO: 20) encoded by the
nucleic acid sequence of SEQ ID NO: 19.
(SEQ ID NO: 19)
gccgaccccaagaaggtgctggataaagccaaagatcaggcagaaaatag
agtcagggaactgaagcagaagctggaggagctgtacaaggaggcccgga
agctggacctgacccaggagatgaggagaaagctggagctgcgctacatc
gccgccatgctgatggccatcggcgacatctataacgccatcaggcaggc
caagcaggaggccgataagctgaagaaggccggcctggtgaatagccagc
agctggacgagctgaagcggcgcctggaggagctgaaggaggaggccagc
aggaaggccagagattacggcagggagttccagctgaagctggagtatgg
cggcggcagcggctccggctctggcggcaagatcgagcagatcctgcaga
agatcgaaaagatcctgcagaagattgagtggattctgcagaagattgaa
cagatcctgcagggg
(SEQ ID NO: 20)
ADPKKVLDKAKDQAENRVRELKQKLEELYKEARKLDLTQEMRRKLELRYI
AAMLMAIGDIYNAIRQAKQEADKLKKAGLVNSQQLDELKRRLEELKEEAS
RKARDYGREFQLKLEYGGGSGSGSGGKIEQILQKIEKILQKIEWILQKIE
QILQG

Accordingly, in some embodiments, the self-assembling polypeptide encoded by the expressible nucleic acid sequence of the present disclosure comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 8, SEQ ID NO: 10, SEQ ID NO: 12, SEQ ID NO: 14, SEQ ID NO: 16, SEQ ID NO: 18 or SEQ ID NO: 20, or a functional fragment or variant thereof. In some embodiments, the self-assembling polypeptide comprises the amino acid sequence of SEQ ID NO: 8, SEQ ID NO: 10, SEQ ID NO: 12, SEQ ID NO: 14, SEQ ID NO: 16, SEQ ID NO: 18 or SEQ ID NO: 20, or a functional fragment or variant thereof. In some embodiments, the nucleic acid sequence encoding the self-assembling polypeptide comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment or variant thereof. In some embodiments, the nucleic acid sequence encoding the self-assembling polypeptide comprises the nucleotide sequence of SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment or variant thereof. In other embodiments, the nucleic acid sequence encoding the self-assembling polypeptide comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to a nucleic acid sequence that is complementary to SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment thereof. In some embodiments, the nucleic acid sequence encoding the self-assembling polypeptide comprises a nucleic acid sequence that is complementary to SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment thereof.

In some embodiments, the composition or pharmaceutical composition of the disclosure comprises a vector or a nucleic acid molecule comprising an expressible RNA sequence that encodes a self-assembling polypeptide that is optionally in sequence with one or more additional expressible RNA sequences that encode a viral antigen. In some embodiments, the expressible DNA or RNA sequence that encodes a self-assembling polypeptide is from about 300 to about 500 nucleotides in length. In some embodiments, the expressible DNA or RNA sequence that encodes a self-assembling polypeptide is from about 350 to about 480 nucleotides in length. In some embodiments, the expressible DNA or RNA sequence that encodes a self-assembling polypeptide is from about 350 to about 460 nucleotides in length. In some embodiments, the expressible DNA or RNA sequence that encodes a self-assembling polypeptide is from about 300 to about 500 nucleotides in length. In some embodiments, the expressible DNA or RNA sequence that encodes a self-assembling polypeptide is from about 400 to about 500 nucleotides in length. In some embodiments, the expressible DNA or RNA sequence that encodes a self-assembling polypeptide is from about 390 to about 410 nucleotides in length. In some embodiments, the expressible DNA or RNA sequence that encodes a self-assembling polypeptide is from about 300 to about 410 nucleotides in length. In some embodiments, the expressible DNA or RNA sequence that encodes a self-assembling polypeptide is from about 300 to about 500 nucleotides in length.

3. Linker

The expressible nucleic acid sequence of the present disclosure optionally comprises a nucleic acid sequence encoding a linker peptide, a functional fragment or variant thereof or a pharmaceutically acceptable salt thereof. Any type of linker or linker peptide can be used. The term “linker” or “linker peptide” is used interchangeable herein.

In some embodiments, each linker or linker peptide is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length.

In some embodiments, each linker or linker peptide is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, each linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, each linker or linker peptide is about 21 natural or non-natural amino acids in length.

In some embodiments, the length of each linker or linker peptide is different. For example, in some embodiments, the length of a first linker or linker peptide is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length, and the length of a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length, where the length of the first linker is different from the length of the second linker. Various configurations can be envisioned by the present disclosure, where the linker domain comprises 1, 2, 3, 4, 5, 6, 7, 8, 9, 10 or more linkers or linker peptides wherein the linkers or linker peptides are of similar or different lengths.

In some embodiments, two linkers or linker peptides can be used together. Accordingly, in some embodiments, the first linker or linker peptide is independently selectable from about 0 to about 25 natural or non-natural amino acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length. In some embodiments, the second linker or linker peptide is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length. In some embodiments, the first linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, the second linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length.

A non-limiting example of a linker peptide may comprise the amino acid sequence of GGSGGSGGSGGSGGG (SEQ ID NO: 22) encoded by the nucleic acid sequence of ggaggctccggaggatctggagggagtggaggctcaggaggaggc (SEQ ID NO: 21).

A linker or linker peptide can be either flexible or rigid or a combination thereof. An example of a flexible linker is a GGS repeat. In some embodiments, the GGS can be repeated about 1, 2, 3, 4, 5, 6, 7, 8, 9, or 10 times. Non-limiting examples of such linker peptides may comprise the amino acid sequence of GGSGGSGGS (SEQ ID NO: 23), GGSGGSGGSGGS (SEQ ID NO: 24), or GGSGGSGGSGGSGGGGSGGGSGGG (SEQ ID NO: 25). An example of a rigid linker is 4QTL-115 Angstroms, single chain 3-helix bundle represented by the sequence:

(SEQ ID NO: 26)
NEDDMKKLYKQMVQELEKARDRMEKLYKEMVELIQKAIELMRKIFQEVKQEVEKA
IEEMKKLYDEAKKKIEQMIQQIKQGGDKQKMEELLKRAKEEMKKVKDKMEKLLEK
LKQIMQEAKQKMEKLLKQLKEEMKKMKEKMEKLLKEMKQRMEEVKKKMDGDDE
LLEKIKKNIDDLKKIAEDLIKKAEENIKEAKKIAEQLVKRAKQLIEKAKQVAEELIKKI
LQLIEKAKEIAEKVLKGLE.
Other non-limiting examples of linker peptides may be encoded by the nucleic
acid sequence of
(SEQ ID NO: 27)
ggcggctctggcggaagtggcggaagtgggggaagtggaggcggcggaagcgggggaggcagcgggggaggg,
(SEQ ID NO: 28)
ggcggaagcggcggaagcggcgggtct,
(SEQ ID NO: 29)
ggcggcagcggcggcagcggcgggagcggaggaagt,
or
(SEQ ID NO: 30)
ggcggctctggcggaagtggcggaagtgggggaagtggaggcggcggaagcgggggaggcagcgggggaggg.
Additional non-limiting examples of linker peptides include Link 14 linker (SEQ
ID NO: 32) encoded by the nucleic acid sequence of SEQ ID NO: 31;
(SEQ ID NO: 31)
tctcacagcggctccggcggctctggcagcggcggccacgcc
(SEQ ID NO: 32)
SHSGSGGSGSGGHA
CPG9.2 linker 1 (SEQ ID NO: 34) encoded by the nucleic acid sequence of SEQ ID NO: 33;
(SEQ ID NO: 33)
gggggaaatagtagcggc
(SEQ ID NO: 34)
GGNSSG
CPG9.2 linker 2 (SEQ ID NO: 36) encoded by the nucleic acid sequence of SEQ ID NO: 35;
(SEQ ID NO: 35)
ggcggcaacggcagcggcggcggcagcggctccggcggcaacggctctagcggc
(SEQ ID NO: 36)
GGNGSGGGSGSGGNGSSG
PDGFR linker (between tri mer or TSI and PDGFR; SEQ ID NO: 38) encoded by the nucleic
acid sequence of SEQ ID NO: 37;
(SEQ ID NO: 37)
ggaggaggaagcgggggaagcgggggaagcggaggaagcgggggaagcgggggaagc
(SEQ ID NO: 38)
GGGSGGSGGSGGSGGSGGS
Foldon PDGFR linker 1 (SEQ ID NO: 40) encoded by the nucleic acid sequence of SEQ ID
NO: 39;
(SEQ ID NO: 39)
ggaggaggaagcgggggaagcggcggcggc
(SEQ ID NO: 40)
GGGSGGSGGG
Foldon PDGFR linker 2 (SEQ ID NO: 42) encoded by the nucleic acid sequence of SEQ ID
NO: 41;
(SEQ ID NO: 41)
gggggaagcggaggaagcgggggaagcgggggaagc
(SEQ ID NO: 42)
GGSGGSGGSGGS
3BVE linker (SEQ ID NO: 44) encoded by the nucleic acid sequence of SEQ ID NO: 43;
(SEQ ID NO: 43)
ggaagcggc
(SEQ ID NO: 44)
GSG
13_1 linker (SEQ ID NO: 46) encoded by the nucleic acid sequence of SEQ ID NO: 45;
(SEQ ID NO: 45)
ggcggcagcggcagcggcgggagcggagga
(SEQ ID NO: 46)
GGSGSGGSGG
13_2 linker (SEQ ID NO: 48) encoded by the nucleic acid sequence of SEQ ID NO: 47;
(SEQ ID NO: 47)
ggagggagcgatatgagaaaggacgccgagagacggtttgataagttcgtggaggctgctaagaataagtttgacaagtttaaggct
gccctgcggaagggcgacatcaaggaggagaggagaaaggatatgaagaagctggcaaggaaggaggcagagcaggcaagga
gggccgtgaggaacagactgagcgagctgctgtccaagatcaacgacatgcccatcaccaatgatcagaagaagctgatgtctaatg
acgtgctgaagttcgccgcagaagccgaaaagaagattgaagccctggcagcagacgccgaaggaggaagcgggagc
(SEQ ID NO: 48)
GGSDMRKDAERRFDKFVEAAKNKFDKFKAALRKGDIKEERRKDMKKLARKEAEQA
RRAVRNRLSELLSKINDMPITNDQKKLMSNDVLKFAAEAEKKIEALAADAEGGSGS
LS_1 linker (SEQ ID NO: 50) encoded by the nucleic acid sequence of SEQ ID NO: 49;
(SEQ ID NO: 49)
gggggctctagcgggaaaagtctggtggataccgtctatgctctgaaagatgaggtgcaggaactgaggcaggacaacaaaaagat
gaagaagagcctggaggaggagcagagggccagaaaggacctggaaaaactggtgcggaaagtgctgaaaaacatgaatgacg
gagggagtagcggg
(SEQ ID NO: 50)
GGSSGKSLVDTVYALKDEVQELRQDNKKMKKSLEEEQRARKDLEKLVRKVLKNMN
DGGSSG
LS_2 linker (SEQ ID NO: 52) encoded by the nucleic acid sequence of SEQ ID NO: 51;
(SEQ ID NO: 51)
gggggctctagcggggcagacccaaagaaagtgctggataaggcaaaggatcaggcagagaatagagtgagagaactgaaacag
aaactggaggaactgtataaggaggcccggaagctggacctgacccaggagatgaggagaaagctggagctgcgctacatcgccg
ccatgctgatggccatcggcgacatctataacgccatcaggcaggccaagcaggaggccgataagctgaagaaggccggcctggt
gaatagccagcagctggacgagctgaagcggcgcctggaggagctgaaggaggaggcctccaggaaggccagagattatgggc
gggaatttcagctgaaactggagtatggcggcggaagcggaagcgggagcggg
(SEQ ID NO: 52)
GGSSGADPKKVLDKAKDQAENRVRELKQKLEELYKEARKLDLTQEMRRKLELRYIA
AMLMAIGDIYNAIRQAKQEADKLKKAGLVNSQQLDELKRRLEELKEEASRKARDYG
REFQLKLEYGGGSGSGSG
QB_1 linker (SEQ ID NO: 54) encoded by the nucleic acid sequence of SEQ ID NO: 53;
(SEQ ID NO: 53)
ggaggctcttcaggcggcacagacgtgggggcaatcgctggaaaggctaacgaggctggacagggggcttatgatgctcaggtca
aaaacgacgagcaggatgtggagctggccgaccacgaggccaggatcaagcagctgagaatcgatgtggacgatcacgagtctcg
gatcaccgccaacacaaaggccatcacagccctgaatgtgcgcgtgaccacagcagagggagagatcgcatccctgcagaccaac
gtgagcgccctggacggaagggtgaccacagcagagaacaatatctccgccctgcaggcagattacgtgagcggcggcagctccg
gctccgga
(SEQ ID NO: 54)
GGSSGGTDVGAIAGKANEAGQGAYDAQVKNDEQDVELADHEARIKQLRIDVDDHE
SRITANTKAITALNVRVTTAEGEIASLQTNVSALDGRVTTAENNISALQADYVSGGSS
GSG
QB 2 linker (SEQ ID NO: 56) encoded by the nucleic acid sequence of SEQ ID NO: 55;
and
(SEQ ID NO: 55)
ggaggctctggaagcgggggaagtagcggacctcacatgattgctccaggacatcgggacgagtttgaccctaagctgccaacagg
cgagaaagaagaggtgccaggcaagcccggcatcaagaaccctgagacaggcgacgtggtgaggccccctgtggattctgtgaca
aagtacggcccagtgaagggcgacagcatcgtggagaaggaggagatccccttcgagaaggagaggaagtttaaccctgatctgg
ccccaggcaccgagaaggtgacaagagagggccagaagggcgagaagaccatcaccacacccacactgaagaatcctctgaccg
gcgagatcatcagcaagggcgagtccaaggaggagatcacaaaggaccccatcaacgaactgaccgaatggggaccagagacag
gaggaagcggcagcggcggaagcagc
(SEQ ID NO: 56)
GGSGSGGSSGPHMIAPGHRDEFDPKLPTGEKEEVPGKPGIKNPETGDVVRPPVDSVT
KYGPVKGDSIVEKEEIPFEKERKFNPDLAPGTEKVTREGQKGEKTITTPTLKNPLTGEI
ISKGESKEEITKDPINELTEWGPETGGSGSGGSS
IC1/IC2 linker (SEQ ID NO: 58) encoded by the nucleic acid sequence of SEQ ID NO: 57
(SEQ ID NO: 57)
ggaggcagcggcagcggcagcggg
(SEQ ID NO: 58)
GGSGSGSG

Accordingly, in some embodiments, the linker peptide encoded by the expressible nucleic acid sequence of the present disclosure comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the linker peptide comprises the amino acid sequence of SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the nucleic acid sequence encoding the linker peptide comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof. In some embodiments, the nucleic acid sequence encoding the linker peptide comprises the nucleotide sequence of SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof. In other embodiments, the nucleic acid sequence encoding the linker peptide comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to a nucleic acid sequence that is complementary to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof. In some embodiments, the nucleic acid sequence encoding the linker peptide comprises a nucleic acid sequence that is complementary to the nucleotide sequence of SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof

4. Viral Antigens

The expressible nucleic acid sequence of the present disclosure comprises a nucleic acid sequence encoding an antigen domain comprising a viral antigen, a functional fragment or variant thereof or a pharmaceutically acceptable salt thereof. In some embodiments, the viral antigen comprises an antigen from a virus from the family of Coronaviridae. In some embodiments, the viral antigen comprises an antigen from a coronavirus. In some embodiments, the viral antigen comprises an antigen from SARS-CoV. In some embodiments, the viral antigen comprises an antigen from SARS-CoV-2. In some embodiments, the viral antigen comprises the spike protein of SARS-CoV-2, a functional fragment or variant thereof or a pharmaceutically acceptable salt thereof. In some embodiments, the viral antigen comprises a viral trimer polypeptide, a functional fragment or variant thereof or a pharmaceutically acceptable salt thereof. In some embodiments, the viral trimer comprises a trimer from a virus from the family of Coronaviridae. In some embodiments, the viral trimer comprises a trimer from a coronavirus. In some embodiments, the viral trimer comprises a trimer from SARS-CoV. In some embodiments, the viral trimer comprises a trimer from SARS-CoV-2. In some embodiments, the viral trimer comprises the spike protein of SARS-CoV-2, a functional fragment or variant thereof or a pharmaceutically acceptable salt thereof.

A non-limiting example of a viral antigen is a fragment of the surface glycoprotein (or spike protein or S protein) of SARS-CoV-2 having the amino acid sequence of SEQ ID NO: 60 encoded by the nucleic acid sequence of SEQ ID NO: 59 (GenBank Accession No. QHD43416).

(SEQ ID NO: 59)
atgtttgtttttcttgttttattgccactagtctctagtcagtgtgttaatcttacaaccagaactcaattaccccctgcatacactaattctttcac
acgtggtgtttattaccctgacaaagttttcagatcctcagttttacattcaactcaggacttgttcttacctttcttttccaatgttacttggttcc
atgctatacatgtctctgggaccaatggtactaagaggtttgataaccctgtcctaccatttaatgatggtgtttattttgcttccactgagaa
gtctaacataataagaggctggatttttggtactactttagattcgaagacccagtccctacttattgttaataacgctactaatgttgttatta
aagtctgtgaatttcaattttgtaatgatccatttttgggtgtttattaccacaaaaacaacaaaagttggatggaaagtgagttcagagtttat
tctagtgcgaataattgcacttttgaatatgtctctcagccttttcttatggaccttgaaggaaaacagggtaatttcaaaaatcttagggaat
ttgtgtttaagaatattgatggttattttaaaatatattctaagcacacgcctattaatttagtgcgtgatctccctcagggtttttcggctttaga
accattggtagatttgccaataggtattaacatcactaggtttcaaactttacttgctttacatagaagttatttgactcctggtgattcttcttca
ggttggacagctggtgctgcagcttattatgtgggttatcttcaacctaggacttttctattaaaatataatgaaaatggaaccattacagat
gctgtagactgtgcacttgaccctctctcagaaacaaagtgtacgttgaaatccttcactgtagaaaaaggaatctatcaaacttctaactt
tagagtccaaccaacagaatctattgttagatttcctaatattacaaacttgtgcccttttggtgaagtttttaacgccaccagatttgcatctg
tttatgcttggaacaggaagagaatcagcaactgtgttgctgattattctgtcctatataattccgcatcattttccacttttaagtgttatgga
gtgtctcctactaaattaaatgatctctgctttactaatgtctatgcagattcatttgtaattagaggtgatgaagtcagacaaatcgctccag
ggcaaactggaaagattgctgattataattataaattaccagatgattttacaggctgcgttatagcttggaattctaacaatcttgattctaa
ggttggtggtaattataattacctgtatagattgtttaggaagtctaatctcaaaccttttgagagagatatttcaactgaaatctatcaggcc
ggtagcacaccttgtaatggtgttgaaggttttaattgttactttcctttacaatcatatggtttccaacccactaatggtgttggttaccaacc
atacagagtagtagtactttcttttgaacttctacatgcaccagcaactgtttgtggacctaaaaagtctactaatttggttaaaaacaaatgt
gtcaatttcaacttcaatggtttaacaggcacaggtgttcttactgagtctaacaaaaagtttctgcctttccaacaatttggcagagacatt
gctgacactactgatgctgtccgtgatccacagacacttgagattcttgacattacaccatgttcttttggtggtgtcagtgttataacacca
ggaacaaatacttctaaccaggttgctgttctttatcaggatgttaactgcacagaagtccctgttgctattcatgcagatcaacttactcct
acttggcgtgtttattctacaggttctaatgtttttcaaacacgtgcaggctgtttaataggggctgaacatgtcaacaactcatatgagtgt
gacatacccattggtgcaggtatatgcgctagttatcagactcagactaattctcctcggcgggcacgtagtgtagctagtcaatccatca
ttgcctacactatgtcacttggtgcagaaaattcagttgcttactctaataactctattgccatacccacaaattttactattagtgttaccaca
gaaattctaccagtgtctatgaccaagacatcagtagattgtacaatgtacatttgtggtgattcaactgaatgcagcaatcttttgttgcaat
atggcagtttttgtacacaattaaaccgtgctttaactggaatagctgttgaacaagacaaaaacacccaagaagtttttgcacaagtcaa
acaaatttacaaaacaccaccaattaaagattttggtggttttaatttttcacaaatattaccagatccatcaaaaccaagcaagaggtcatt
tattgaagatctacttttcaacaaagtgacacttgcagatgctggcttcatcaaacaatatggtgattgccttggtgatattgctgctagaga
cctcatttgtgcacaaaagtttaacggccttactgttttgccacctttgctcacagatgaaatgattgctcaatacacttctgcactgttagcg
ggtacaatcacttctggttggacctttggtgcaggtgctgcattacaaataccatttgctatgcaaatggcttataggtttaatggtattgga
gttacacagaatgttctctatgagaaccaaaaattgattgccaaccaatttaatagtgctattggcaaaattcaagactcactttcttccaca
gcaagtgcacttggaaaacttcaagatgtggtcaaccaaaatgcacaagctttaaacacgcttgttaaacaacttagctccaattttggtg
caatttcaagtgttttaaatgatatcctttcacgtcttgacaaagttgaggctgaagtgcaaattgataggttgatcacaggcagacttcaaa
gtttgcagacatatgtgactcaacaattaattagagctgcagaaatcagagcttctgctaatcttgctgctactaaaatgtcagagtgtgta
cttggacaatcaaaaagagttgatttttgtggaaagggctatcatcttatgtccttccctcagtcagcacctcatggtgtagtcttcttgcatg
tgacttatgtccctgcacaagaaaagaacttcacaactgctcctgccatttgtcatgatggaaaagcacactttcctcgtgaaggtgtcttt
gtttcaaatggcacacactggtttgtaacacaaaggaatttttatgaaccacaaatcattactacagacaacacatttgtgtctggtaactgt
gatgttgtaataggaattgtcaacaacacagtttatgatcctttgcaacctgaattagactcattcaaggaggagttagataaatattttaag
aatcatacatcaccagatgttgatttaggtgacatctctggcattaatgcttcagttgtaaacattcaaaaagaaattgaccgcctcaatga
ggttgccaagaatttaaatgaatctctcatcgatctccaagaacttggaaagtatgagcagtatataaaatggccatggtacatttggcta
ggttttatagctggcttgattgccatagtaatggtgacaattatgctttgctgtatgaccagttgctgtagttgtctcaagggctgttgttcttgt
ggatcctgctgcaaatttgatgaagacgactctgagccagtgctcaaaggagtcaaattacattacacataa
(SEQ ID NO: 60)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
WPWYIWLGFIAGLIAIVMVTIMLCCMTSCCSCLKGCCSCGSCCKFDEDDSEPVLKGV
KLHYT

Non-limiting examples of fragments of the S protein of SARS-CoV-2 comprises the following sequences:

(SEQ ID NO: 171)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
WPW
(SEQ ID NO: 172)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
WP
(SEQ ID NO: 173)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQ
(SEQ ID NO: 174)
SQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQGN
FKNLREFVFKNIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAG
STPCNGVEGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGSNVFQTRA
GCLIGAEHVNNSYECDIPIGAGICASYQTQTNSPRRARSVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSFIEDLLFN
KVTLADAGFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGKIQDSLS
STASALGKLQDVVNQNAQALNTLVKQLSSNFGAISSVLNDILSRLDKVEAEVQIDRLI
TGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNF
YEPQIITTDNTFVSGNCDVVIGIVNNTVYDPLQPELDSFKEELDKYFKNHTSPDVDLG
DISGINASVVNIQKEIDRLNEVAKNLNESLIDLQELGKYEQ
(SEQ ID NO: 175)
NLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGG
NYNYLYRLFRKSNLKPFERDIST
(SEQ ID NO: 176)
SFIEDLLFNKVTLADAGFIKQYGDCLGDIAARDLICAQKF
(SEQ ID NO: 177)
PSKRSFIEDLLFNKV

A further non-limiting example of a viral antigen is a fragment of the envelop protein (or E protein) of SARS-CoV-2 having the amino acid sequence of SEQ ID NO: 62 encoded by the nucleic acid sequence of SEQ ID NO: 61 (GenBank Accession No. QHD43418).

(SEQ ID NO: 61)
atgtactcattcgtttcggaagagacaggtacgttaatagttaatagcgt
acttctttttcttgctttcgtggtattcttgctagttacactagccatcc
ttactgcgcttcgattgtgtgcgtactgctgcaatattgttaacgtgagt
cttgtaaaaccttctttttacgtttactctcgtgttaaaaatctgaattc
ttctagagttcctgatcttctggtctaa
(SEQ ID NO: 62)
MYSFVSEETGTLIVNSVLLFLAFVVFLLVTLAILTALRLCAYCCNIVNVS
LVKPSFYVYSRVKNLNSSRVPDLLV

Another non-limiting example of a viral antigen is a fragment of the membrane glycoprotein (or M protein) of SARS-CoV-2 having the amino acid sequence of SEQ ID NO: 64 encoded by the nucleic acid sequence of SEQ ID NO: 63 (GenBank Accession No. QHD43419).

(SEQ ID NO: 63)
ccatggcagattccaacggtactattaccgttgaagagcttaaaaagctc
cttgaacaatggaacctagtaataggtttcctattccttacatggatttg
tcttctacaatttgcctatgccaacaggaataggtttttgtatataatta
agttaattttcctctggctgttatggccagtaactttagcttgttttgtg
cttgctgctgtttacagaataaattggatcaccggtggaattgctatcgc
aatggcttgtcttgtaggcttgatgtggctcagctacttcattgcttctt
tcagactgtttgcgcgtacgcgttccatgtggtcattcaatccagaaact
aacattcttctcaacgtgccactccatggcactattctgaccagaccgct
tctagaaagtgaactcgtaatcggagctgtgatccttcgtggacatcttc
gtattgctggacaccatctaggacgctgtgacatcaaggacctgcctaaa
gaaatcactgttgctacatcacgaacgctttcttattacaaattgggagc
ttcgcagcgtgtagcaggtgactcaggttttgctgcatacagtcgctaca
ggattggcaactataaattaaacacagaccattccagtagcagtgacaat
attgctttgcttgtacagtaa
(SEQ ID NO: 64)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ

A yet another non-limiting example of a viral antigen is a fragment of the nucleocapsid phosphoprotein (or N protein) of SARS-CoV-2 having the amino acid sequence of SEQ ID NO: 66 encoded by the nucleic acid sequence of SEQ ID NO: 65 (GenBank Accession No. QHD43423).

(SEQ ID NO: 65)
atgtctgataatggaccccaaaatcagcgaaatgcaccccgcattacgtt
tggtggaccctcagattcaactggcagtaaccagaatggagaacgcagtg
gggcgcgatcaaaacaacgtcggccccaaggtttacccaataatactgcg
tcttggttcaccgctctcactcaacatggcaaggaagaccttaaattccc
tcgaggacaaggcgttccaattaacaccaatagcagtccagatgaccaaa
ttggctactaccgaagagctaccagacgaattcgtggtggtgacggtaaa
atgaaagatctcagtccaagatggtatttctactacctaggaactgggcc
agaagctggacttccctatggtgctaacaaagacggcatcatatgggttg
caactgagggagccttgaatacaccaaaagatcacattggcacccgcaat
cctgctaacaatgctgcaatcgtgctacaacttcctcaaggaacaacatt
gccaaaaggcttctacgcagaagggagcagaggcggcagtcaagcctctt
ctcgttcctcatcacgtagtcgcaacagttcaagaaattcaactccaggc
agcagtaggggaacttctcctgctagaatggctggcaatggcggtgatgc
tgctcttgctttgctgctgcttgacagattgaaccagcttgagagcaaaa
tgtctggtaaaggccaacaacaacaaggccaaactgtcactaagaaatct
gctgctgaggcttctaagaagcctcggcaaaaacgtactgccactaaagc
atacaatgtaacacaagctttcggcagacgtggtccagaacaaacccaag
gaaattttggggaccaggaactaatcagacaaggaactgattacaaacat
tggccgcaaattgcacaatttgcccccagcgcttcagcgttcttcggaat
gtcgcgcattggcatggaagtcacaccttcgggaacgtggttgacctaca
caggtgccatcaaattggatgacaaagatccaaatttcaaagatcaagtc
attttgctgaataagcatattgacgcatacaaaacattcccaccaacaga
gcctaaaaaggacaaaaagaagaaggctgatgaaactcaagccttaccgc
agagacagaagaaacagcaaactgtgactcttcttcctgctgcagatttg
gatgatttctccaaacaattgcaacaatccatgagcagtgctgactcaac
tcaggcctaa
(SEQ ID NO: 66)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA

Accordingly, in some embodiments, the viral antigen encoded by the expressible nucleic acid sequence of the present disclosure comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof or a pharmaceutically acceptable salt thereof. In some embodiments, the viral antigen comprises the amino acid sequence of SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof or a pharmaceutically acceptable salt thereof. In some embodiments, the nucleic acid sequence encoding the viral antigen comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to SEQ ID NO: 59, SEQ ID NO: 61, SEQ ID NO: 63 or SEQ ID NO: 65, or a functional fragment or variant thereof or a pharmaceutically acceptable salt thereof. In some embodiments, the nucleic acid sequence encoding the viral antigen comprises the nucleotide sequence of SEQ ID NO: 59, SEQ ID NO: 61, SEQ ID NO: 63 or SEQ ID NO: 65, or a functional fragment or variant thereof or a pharmaceutically acceptable salt thereof. In some embodiments, the nucleic acid sequence encoding the viral antigen comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to a nucleic acid sequence that is complementary to SEQ ID NO: 59, SEQ ID NO: 61, SEQ ID NO: 63 or SEQ ID NO: 65, or a functional fragment or variant thereof or a pharmaceutically acceptable salt thereof. In some embodiments, the nucleic acid sequence encoding the viral antigen comprises a nucleic acid sequence that is complementary to the nucleotide sequence of SEQ ID NO: 59, SEQ ID NO: 61, SEQ ID NO: 63 or SEQ ID NO: 65, or a functional fragment or variant thereof or a pharmaceutically acceptable salt thereof.

In some embodiments, the composition or pharmaceutical composition of the disclosure comprises an expressible DNA or RNA sequence that encodes a viral antigen and is from about 1100 to about 1300 nucletides in length. In some embodiments, the composition or pharmaceutical composition of the disclosure comprises an expressible DNA or RNA sequence that encodes a viral antigen and is from about 1100 to about 1200 nucletides in length. In some embodiments, the composition or pharmaceutical composition of the disclosure comprises an expressible DNA or RNA sequence that encodes a viral antigen and is from about 1100 to about 1300 nucletides in length. In some embodiments, the composition or pharmaceutical composition of the disclosure comprises an expressible DNA or RNA sequence that encodes a viral antigen and is from about 1200 to about 1210 nucletides in length. In some embodiments, the composition or pharmaceutical composition of the disclosure comprises an expressible DNA or RNA sequence that encodes a viral antigen and is from about 1180 to about 1220 nucletides in length. In some embodiments, the composition or pharmaceutical composition of the disclosure comprises an expressible DNA or RNA sequence that encodes a viral antigen and is from about 1180 to about 1215 nucletides in length. In some embodiments, the composition or pharmaceutical composition of the disclosure comprises an expressible DNA or RNA sequence that encodes a viral antigen and is from about 1180 to about 1210 nucletides in length. In some embodiments, the composition or pharmaceutical composition of the disclosure comprises an expressible DNA or RNA sequence that encodes a viral antigen and is from about 1180 to about 1200 nucletides in length. In some embodiments, the composition or pharmaceutical composition of the disclosure comprises an expressible DNA or RNA sequence that encodes a viral antigen and is from about 1220 to about 1230 nucletides in length. In some embodiments, the composition or pharmaceutical composition of the disclosure comprises an expressible DNA or RNA sequence that encodes a viral antigen and is from about 1100 to about 1300 nucletides in length. In some embodiments, the composition or pharmaceutical composition of the disclosure comprises an expressible DNA or RNA sequence that encodes a viral antigen and is from about 1100 to about 1300 nucletides in length.

In some embodiments, the expressible nucleic acid sequence encodes a fusion protein comprising one or a plurality of coronaviral envelope polypeptides or functional fragments thereof. In some embodiments, the fusion protein comprise a furin cleavage site. In some embodiments, the expressible nucleic acid sequence comprises a first nucleic acid sequence encoding, in a 5′ to 3′ orientation, at least three monomers of coronaviral envelope proteins. In some embodiments, the at least three monomers of coronaviral envelope proteins are separated by a furin cleavage site. In some embodiments, the furin cleavage site comprises at least about 60%, 65%, 70%, 75%, 80%, 85%, 86%, 87%, 88%, 89%, 90%, 91%, 92%, 93%, 94%, 95%, 96%, 97%, 98% or 99% sequence identity to RRRRRR (SEQ ID NO: 67), or a functional fragment or variant thereof or a pharmaceutically acceptable salt thereof. In some embodiments, the furin cleavage site comprises the amino acid sequence of SEQ ID NO: 67, or a functional fragment or variant thereof or a pharmaceutically acceptable salt thereof. In some embodiments, the expressible nucleic acid sequence encodes a polypeptide free of carbohydrate proximate to at least 30 amino acids from the carboxy end of the polypeptide. In some embodiments, the expressible nucleic acid sequence encodes a polypeptide free of carbohydrate proximate to at least 20 amino acids from the carboxy end of the polypeptide. In some embodiments, the expressible nucleic acid sequence encodes a polypeptide free of carbohydrate proximate to at least 10 amino acids from the carboxy end of the polypeptide. In some embodiments, the expressible nucleic acid sequence encodes a polypeptide free of carbohydrate proximate to at least 50 amino acids from the carboxy end of the polypeptide.

In some embodiments, the expressible nucleic acid sequence of the disclosure comprises at least a first nucleic acid sequence encoding a first, a second and/or a third polypeptides, each first, second or third polypeptide comprising a viral antigen. In some embodiments, the expressible nucleic acid sequence encodes one or a plurality of fusion proteins, each fusion protein comprising at least a first, a second, and/or a third polypeptide contiguously linked by a linker sequence. In some embodiments, the expressible nucleic acid sequence of the disclosure comprises at least a first nucleic acid sequence encoding at least one self-assembling polypeptide. In some embodiments, the self-assembling polypeptide is at least one self-assembling component of a nanoparticle or at least one coronaviral monomer, the coronaviral monomer capable of assembling into a coronaviral trimer upon expression in a cell. In some embodiments, the expressible nucleic acid sequence comprises a nucleic acid sequence encoding a coronaviral antigen, but free of a nucleic acid sequence encoding a self-assembling polypeptide. In some embodiments, the expressible nucleic acid sequence of the disclosure comprises a nucleic acid sequence operably linked to a regulatory sequence and encodes a fusion peptide comprising one or a plurality of self-assembling polypeptides, wherein at least one of the self-assembling polypeptides is a self-assembling coronaviral antigen.

In some embodiments, upon administration to a subject a composition comprising the expressible nucleic acid sequence of the disclosure, the expressible nucleic acid sequence is transfected or transduced into an antigen presenting cell. After a plurality of expressible nucleic acid sequences are expressed, the self-assembling polypeptides assemble with into a non-native form of a viral antigen. In some embodiments, the non-native form of a viral antigen comprises a coronaviral trimer exposing an amino acid sequence that is not naturally exposed or free of carbohydrate as compared to its corresponding native form or variants thereof. Expression and presentation of the one or plurality of self-assembling polypeptides elicits an immune response against an epitope. In some embodiments, the epitope comprises a non-native secondary structure of the one or plurality of self-assembling polypeptides.

In some embodiments, the comopsitions comprise a nucleic acid seqeunce encoding any combination of nucleic acid sequences disclosed herein or vairants thereof. In some embodiments, the comopsitions comprise a viral particle that comprises an expressible nucleic acid seqeunce encoding any combination of nucleic acid sequences disclosed herein or variants thereof. The component of the self-assemblying peptide can be any monomer that, upon expression, self-assembles into a particle comprising 7, 14, 27 or 60 peptides sided particle, each peptide side fused to at least one antigen from the Coronoviridae family. In some embodiments, the composition comprises a particle comprising 7, 14, 27 or 60 peptides sided particle, each peptide side is fused to at least one antigen from the Coronoviridae family, wherein the antigen is positioned in an energetically stable state as compared to the unassociated energy state. In some emboidments, the disclosure relates to a cell comprising a particle comprising 7, 14, 27 or 60 peptides sided particle, each peptide side is fused to at least one antigen from the Coronoviridae family, wherein the antigen is positioned in an energetically stable state as compared to the unassociated energy state. In some embodiments, the particle is able to display the at least one viral antigen in a conformationally stable state that is more immunologically active, such activity elicting a more therapeutically effective immune response as compared to eh immune response elicited from administration of the DNA or RNA. In some embodiments, the energetically stable state is identified by association of the peptide to an antibody through surface plasmon resonance (SPR). In some embodiments, the energetically stable state is measured by absorbance units when either a ligand for the antigen or the antigen is immobilized to a surface, and the other binding partner is then passed over the surface as analyte. In some embodiments, the association can be measured through SPR on a BIACORE® system.

A detailed discussion of the technical aspects of the BIACORE® instruments and the phenomenon of SPR may be found in U.S. Pat. No. 5,313,264 (the full disclosure of which is incorporated by reference herein in its entirety). In the BIACORE® system, the SPR response values are expressed in resonance units (RU). One RU represents a change of 0.0001° in the angle of minimum reflected light intensity. For an SPR based sensor system like the BIACORE® system, a difference in refractive index between the two guiding fluids of, say, about 100 RU may be convenient, and the fluid interface position may be determined by means of per se conventional sensorgrams.

In some embodiments, it may be preferred to keep the total flow rate constant when introducing the sample flow. In such a case, the flow rates of the two guiding fluids are reduced while maintaining the flow rate ratio between them. Assume, for example, that the flow rate of one guiding fluid is 70 μl/min and the flow rate of the other guiding fluid is 30 μl/min, the total flow rate being 100 μl/min, and that a sample fluid flow of 20 μl/min is introduced between the guiding fluids. To maintain the total fluid flow rate at 100 μl/min, the flow rates of the guiding fluids will have to be reduced to 60 and 20 μl/min, respectively. The position of a sample fluid flow on a surface may be presented in various ways. A non-limiting example of a experiment indicating the relative responses obtained at different detector rows as the sample flow is guided laterally across the sensing surface of a flow cell by two guiding buffers in a BIACORE® system equipped with a ψ-cell (BIACORE® S51 is a SPR-based biosensor instrument, normally equipped with two Y-type flow cells, each allowing a dual flow over the a sensor surface for hydrodynamic addressing; Biacore AB, Uppsala, Sweden). Total buffer flow can be set to 100 μl/min, and the flow rates of the two buffer flows can be changed in steps of 2 μl/min, starting with 2 μl/min for one buffer and 98 μl/min for the other. Sample fluid flow can be 20 μl/min all the time. Relative responses>0.1 (i.e. 10% coverage of the detector row) are represented are measured as absorbance over time. This approach thus permits convenient visual monitoring of the sample fluid flow.

In some embodiments, the stability of an antigen secondary structure with an elevated stability as compared to a native antigen or antigen not fused to self-assemblying peptide is from about 10 to about 10,000 RU more than the RU from a control as measured by SPR. In some embodiments, the stability of an antigen secondary structure with an elevated stability as compared to a native antigen or antigen not fused to self-assemblying peptide is from about 5 to about 1,000 RU more than the RU from a control as measured by SPR. In some embodiments, the stability of an antigen secondary structure with an elevated stability as compared to a native antigen or antigen not fused to self-assemblying peptide is from about 100 to about 10,000 RU more than the RU from a control as measured by SPR. In some embodiments, the stability of an antigen secondary structure with an elevated stability as compared to a native antigen or antigen not fused to self-assemblying peptide is from about 100 to about 500 RU more than the RU from a control as measured by SPR. In some embodiments, the stability of an antigen secondary structure with an elevated stability as compared to a native antigen or antigen not fused to self-assemblying peptide is from about 100 to about 200 RU more than the RU from a control as measured by SPR.

5. Regulatory Sequences

In some embodiments, the expressible nucleic acid sequence can be operably linked to one or a plurality of regulatory sequences. The term “regulatory sequence” as used herein refer to DNA sequences which are necessary to effect expression of sequences to which they are ligated. The term “regulatory sequence” is intended to include, as a minimum, all components necessary for expression and optionally additional advantageous components. Examples of regulatory sequences include, but not limited to, promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. In some embodiments, the regulatory sequence is a promoter sequence. As used herein, a “promoter” means a region of DNA upstream from the transcription start and which is involved in binding RNA polymerase and other proteins to start transcription. Reference herein to a “promoter” is to be taken in its broadest context and includes the transcriptional regulatory sequences derived from a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. Consequently, a repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions. The term “promoter” also includes the transcriptional regulatory sequences of a classical prokaryotic gene, in which case it may include a −35 box sequence and/or a −10 box transcriptional regulatory sequences. The term “promoter” is also used to describe a synthetic or fusion molecule, or derivative which confers, activates or enhances expression of a nucleic acid molecule in a cell, tissue or organ.

6. Expressible Nucleic Acid Sequences

The expressible nucleic acid sequence comprised in the composition of the present disclosure can be in form of a DNA molecule, a RNA molecule or transcript, or a DNA/RNA hybrid. In some embodiments, the expressible nucleic acid sequence is in form of a DNA molecule. In some embodiments, the expressible nucleic acid sequence is in form of a RNA molecule or transcript. In some embodiments, the expressible nucleic acid sequence is in form of a DNA/RNA hybrid.

In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 8, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 10, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 12, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 14, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 16, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 18, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 20, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof.

In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 8, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 175. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 8, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 176. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 8, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 177. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 12, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 175. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 12, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 176. In some embodiments, the expressible nucleic acid sequence comprises a first nucleic acid sequence encoding a self-assembling polypeptide comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 12, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 177.

In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof.

In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 171. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 172. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 173. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 174. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 175. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 176. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 177. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 171. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 172. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 173. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 174. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 175. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 176. In some embodiments, the expressible nucleic acid sequence comprises a first nucleic acid sequence encoding a leader sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 5, or a functional fragment or variant thereof, and a second nucleic acid sequence encoding a viral antigen comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 177.

Exemplary expressible nucleic acid sequences include, but not limited to those provided in TABLE 1.

TABLE 1
Exemplary Expressible Nucleic Acid Sequences (DNA and RNA) of the
Disclosure and the corresponding coding polypeptide sequences (underlined amino acid
residues are glycan sites).
I. CoV2 Nanoparticle Constructs
WuhanS_FP12_L9GT60_pVax
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcgacaccatcacactgccatgccgccctgcac
cacctccacattgtagctccaacatcaccggcctgattctgacaagacaggggggatatagtaacgataataccgtgattttcaggccct
caggaggggactggagggacatcgcacgatgccagattgctggaacagtggtctctactcagctgtttctgaacggcagtctggctga
ggaagaggtggtcatccgatctgaagactggcgggataatgcaaagtcaatttgtgtgcagctgaacacaagcgtcgagatcaattgc
actggcgcagggcactgtaacatttctcgggccaaatgggataataccctgaagcagatcgccagtaaactgagagagcagtacggc
aataagacaatcatcttcaagccttctagtggaggcgacccagagttcgtgaaccatagctttaattgcgggggagagttcttttattgtg
attccacacagctgttcgatagcacttggtttgattccaccggtgggagcggaagtggcggttccggatcattcattgaagaccttctcttt
aacaaggtgaccctcgccgatgcaggtttcattaagcaatatggtgattgcctgggagacatcgcggctcgtgatcttatttgtgcgcag
aaattttaatga (SEQ ID NO: 68)
ggauccgccaccauggacuggaccuggauucuguuccugguggccgccgccacaagggugcacagcaugcagaucuacg
aaggaaaacugaccgcugagggacugagguucggaauugucgcaagccgcgcgaaucacgcacugguggauaggcuggu
ggaaggcgcuaucgacgcaauuguccggcacggcgggagagaggaagacaucacacuggugagagucugcggcagcugg
gagauucccguggcagcuggagaacuggcucgaaaggaggacaucgaugccgugaucgcuauugggguccugugccgag
gagcaacucccagcuucgacuacaucgccucagaagugagcaaggggcuggcugaucugucccuggagcugaggaaacc
uaucacuuuuggcgugauuacugccgacacccuggaacaggcaaucgaggcggccggcaccugccauggaaacaaaggcu
gggaagcagcccugugcgcuauugagauggcaaaucuguucaaaucucugcgaggaggcuccggaggaucuggagggag
uggaggcucaggaggaggcgacaccaucacacugccaugccgcccugcaccaccuccacauuguagcuccaacaucaccg
gccugauucugacaagacaggggggauauaguaacgauaauaccgugauuuucaggcccucaggaggggacuggaggga
caucgcacgaugccagauugcuggaacaguggucucuacucagcuguuucugaacggcagucuggcugaggaagaggug
gucauccgaucugaagacuggcgggauaaugcaaagucaauuugugugcagcugaacacaagcgucgagaucaauugca
cuggcgcagggcacuguaacauuucucgggccaaaugggauaauacccugaagcagaucgccaguaaacugagagagcag
uacggcaauaagacaaucaucuucaagccuucuaguggaggcgacccagaguucgugaaccauagcuuuaauugcgggg
gagaguucuuuuauugugauuccacacagcuguucgauagcacuugguuugauuccaccggugggagcggaaguggcg
guuccggaucauucauugaagaccuucucuuuaacaaggugacccucgccgaugcagguuucauuaagcaauaugguga
uugccugggagacaucgcggcucgugaucuuauuugugcgcagaaauuuuaauga (SEQ ID NO: 69)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGDTITLPCRPAPPPHCSSNITGLILTRQGGYSNDNTVIFRPSGGD
WRDIARCQIAGTVVSTQLFLNGSLAEEEVVIRSEDWRDNAKSICVQLNTSVEINCTGA
GHCNISRAKW [symbol] LKQIASKLREQYGNKTIIFKPSSGGDPEFVNHSFNCGGEFFYCDS
TQLF [symbol] WF [symbol] GGSGSGGSGSFIEDLLFNKVTLADAGFIKQYGDCLGDIAARDLICA
QKF** (SEQ ID NO: 70)
WuhanS_FP_L9GT60_pVax
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcgacaccatcacactgccatgccgccctgcac
cacctccacattgtagctccaacatcaccggcctgattctgacaagacaggggggatatagtaacgataataccgtgattttcaggccct
caggaggggactggagggacatcgcacgatgccagattgctggaacagtggtctctactcagctgtttctgaacggcagtctggctga
ggaagaggtggtcatccgatctgaagactggcgggataatgcaaagtcaatttgtgtgcagctgaacacaagcgtcgagatcaattgc
actggcgcagggcactgtaacatttctcgggccaaatgggataataccctgaagcagatcgccagtaaactgagagagcagtacggc
aataagacaatcatcttcaagccttctagtggaggcgacccagagttcgtgaaccatagctttaattgcgggggagagttcttttattgtg
attccacacagctgttcgatagcacttggtttgattccaccggtgggagcggaagtggcggttccggaccttcaaagagatctttcattg
aagacctgcttttcaacaaggtctaatga (SEQ ID NO: 71)
ggauccgccaccauggacuggaccuggauucuguuccugguggccgccgccacaagggugcacagcaugcagaucuacg
aaggaaaacugaccgcugagggacugagguucggaauugucgcaagccgcgcgaaucacgcacugguggauaggcuggu
ggaaggcgcuaucgacgcaauuguccggcacggcgggagagaggaagacaucacacuggugagagucugcggcagcugg
gagauucccguggcagcuggagaacuggcucgaaaggaggacaucgaugccgugaucgcuauugggguccugugccgag
gagcaacucccagcuucgacuacaucgccucagaagugagcaaggggcuggcugaucugucccuggagcugaggaaacc
uaucacuuuuggcgugauuacugccgacacccuggaacaggcaaucgaggcggccggcaccugccauggaaacaaaggcu
gggaagcagcccugugcgcuauugagauggcaaaucuguucaaaucucugcgaggaggcuccggaggaucuggagggag
uggaggcucaggaggaggcgacaccaucacacugccaugccgcccugcaccaccuccacauuguagcuccaacaucaccg
gccugauucugacaagacaggggggauauaguaacgauaauaccgugauuuucaggcccucaggaggggacuggaggga
caucgcacgaugccagauugcuggaacaguggucucuacucagcuguuucugaacggcagucuggcugaggaagaggug
gucauccgaucugaagacuggcgggauaaugcaaagucaauuugugugcagcugaacacaagcgucgagaucaauugca
cuggcgcagggcacuguaacauuucucgggccaaaugggauaauacccugaagcagaucgccaguaaacugagagagcag
uacggcaauaagacaaucaucuucaagccuucuaguggaggcgacccagaguucgugaaccauagcuuuaauugcgggg
gagaguucuuuuauugugauuccacacagcuguucgauagcacuugguuugauuccaccggugggagcggaaguggcg
guuccggaccuucaaagagaucuuucauugaagaccugcuuuucaacaaggucuaauga (SEQ ID NO: 72)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGDTITLPCRPAPPPHCSSNITGLILTRQGGYSNDNTVIFRPSGGD
WRDIARCQIAGTVVSTQLFLNGSLAEEEVVIRSEDWRDNAKSICVQLNTSVEINCTGA
GHCNISRAKW [symbol] LKQIASKLREQYGNKTIIFKPSSGGDPEFVNHSFNCGGEFFYCDS
TQLF [symbol] WF [symbol] GGSGSGGSGPSKRSFIEDLLFNKV** (SEQ ID NO: 73)
WuhanS_RBD_gmax_180_pVax
ggatccgccaccatggactggacctggattctgttcctggtggccgccgccacaagggtgcacagcctctcaattgccccaacgttga
tcaaccgggacaagccatacacgaaagaggaacttatggagatattgcggttggccattatagctgaactcgatgcaattaatctctatg
aacaaatggcccgctatagcgaagacgaaaatgtgagaaagatcttgttggacgtcgctagggaagagaaagcacacgtaggagag
ttcatggctttgttgcttaacctcgaccctgagcaagtcacagagctgaagggcgggttcgaggaagttaaagaattgaccggtataga
agctcacattaatgacaacaagaaagaggaaagtaatgtagagtatttcgagaagctcagatctgccttgttggatggagtcaacaagg
gtcgcagcttgctcaaacatctgcccgttacaagaatagaagggcagtcttttcgagtagacatcatcaaatttgaggacggcgtccga
gtggttaaacaagagtataagcctataccccttcttaagaagaagttctacgtcggcattcgagaactgaatgacgggacatatgatgtc
agcattgctactaaagccggtgagctgctggttaaagacgaagaaagtcttgtgatccgggaaattttgtcaacggaaggcatcaagaa
aatgaaattgtcatcctgggacaatccagaagaagccctgaatgatttgatgaatgcgctccaagaagctagcaatgctagtgctggcc
ccttcggccttattatcaatccaaagcggtacgccaaactgctgaagatctatgaaaagtcaggtaagatgctcgtagaagtactcaagg
aaatcttccggggtggaataatcgtaactcttaatatcgacgaaaacaaagtgattatcttcgctaatacgcccgccgttctggacgtggt
ggtgggtcaagacgttacgctccaggagcttggtccggaaggggatgatgtcgcattcctggtcagtgaagccattggtataagaatc
aagaacccggaagctatagttgttctcgaaggcgggtctggtgggagcggtggtagtggtggttctggtggtggtgggtcaggtggc
ggctcaggcggcggcaatctgtgccctttcggtgaggtctttaatgcaacaagatttgcaagtgtttacgcctggaaccgtaagcgcatt
agcaactgcactgccgattactctgtgctgtacaacagcacaagcttttccacatttaaatgttacggggtttcccctaccaacctcagcg
acctctgctttactaatgtttacgcagattccttcgttatccgaggcgatgaagtccggcagatagctcccggacagaccggcaaaatcg
ctgactacaactataaactgccgaacgacagcacagggtgtgtaattgcttggaacagcaataacctcgattcaaaggttggcggaaat
tacaattatctttaccgtctgttccggaaaagcaatctgaaaccctttgagagagacatcagcacggaaatttatcaagccggttcaacac
catgtaacggagttgaaggctttaattgctattttcccctgcaatcttacggatttcaacctacgaacggggtcggttaccaaccttaccgg
gtggtcgtgctgagcttcgaattgcttcatgccccagccaccgtgtgtgggccataatga (SEQ ID NO: 74)
ggauccgccaccauggacuggaccuggauucuguuccugguggccgccgccacaagggugcacagccucucaauugccc
caacguugaucaaccgggacaagccauacacgaaagaggaacuuauggagauauugcgguuggccauuauagcugaacuc
gaugcaauuaaucucuaugaacaaauggcccgcuauagcgaagacgaaaaugugagaaagaucuuguuggacgucgcua
gggaagagaaagcacacguaggagaguucauggcuuuguugcuuaaccucgacccugagcaagucacagagcugaaggg
cggguucgaggaaguuaaagaauugaccgguauagaagcucacauuaaugacaacaagaaagaggaaaguaauguagag
uauuucgagaagcucagaucugccuuguuggauggagucaacaagggucgcagcuugcucaaacaucugcccguuacaa
gaauagaagggcagucuuuucgaguagacaucaucaaauuugaggacggcguccgagugguuaaacaagaguauaagcc
uauaccccuucuuaagaagaaguucuacgucggcauucgagaacugaaugacgggacauaugaugucagcauugcuacu
aaagccggugagcugcugguuaaagacgaagaaagucuugugauccgggaaauuuugucaacggaaggcaucaagaaaa
ugaaauugucauccugggacaauccagaagaagcccugaaugauuugaugaaugcgcuccaagaagcuagcaaugcuag
ugcuggccccuucggccuuauuaucaauccaaagcgguacgccaaacugcugaagaucuaugaaaagucagguaagaugc
ucguagaaguacucaaggaaaucuuccgggguggaauaaucguaacucuuaauaucgacgaaaacaaagugauuaucuu
cgcuaauacgcccgccguucuggacgugguggugggucaagacguuacgcuccaggagcuugguccggaaggggauga
ugucgcauuccuggucagugaagccauugguauaagaaucaagaacccggaagcuauaguuguucucgaaggcgggucu
ggugggagcggugguaguggugguucuggugguggugggucagguggcggcucaggcggcggcaaucugugcccuuu
cggugaggucuuuaaugcaacaagauuugcaaguguuuacgccuggaaccguaagcgcauuagcaacugcacugccgau
uacucugugcuguacaacagcacaagcuuuuccacauuuaaauguuacgggguuuccccuaccaaccucagcgaccucug
cuuuacuaauguuuacgcagauuccuucguuauccgaggcgaugaaguccggcagauagcucccggacagaccggcaaa
aucgcugacuacaacuauaaacugccgaacgacagcacaggguguguaauugcuuggaacagcaauaaccucgauucaaa
gguuggcggaaauuacaauuaucuuuaccgucuguuccggaaaagcaaucugaaacccuuugagagagacaucagcacg
gaaauuuaucaagccgguucaacaccauguaacggaguugaaggcuuuaauugcuauuuuccccugcaaucuuacggau
uucaaccuacgaacggggucgguuaccaaccuuaccggguggucgugcugagcuucgaauugcuucaugccccagccac
cgugugugggccauaauga (SEQ ID NO: 75)
MDWTWILFLVAAATRVHSLSIAPTLINRDKPYTKEELMEILRLAIIAELDAINLYEQM
ARYSEDENVRKILLDVAREEKAHVGEFMALLLNLDPEQVTELKGGFEEVKELTGIEA
HINDNKKEESNVEYFEKLRSALLDGVNKGRSLLKHLPVTRIEGQSFRVDIIKFEDGVR
VVKQEYKPIPLLKKKFYVGIRELNDGTYDVSIATKAGELLVKDEESLVIREILSTEGIK
KMKLSSWDNPEEALNDLMNALQEASNASAGPFGLIINPKRYAKLLKIYEKSGKMLV
EVLKEIFRGGIIVTLNIDENKVIIFANTPAVLDVVVGQDVTLQELGPEGDDVAFLVSEA
IGIRIKNPEAIVVLEGGSGGSGGSGGSGGGGSGGGSGGGNLCPFGEVFNATRFASVYA
WNRKRISNCTADYSVLYNSTSFSTFKCYGVSPTNLSDLCFTNVYADSFVIRGDEVRQI
APGQTGKIADYNYKLPNDSTGCVIAWNSNNLDSKVGGNYNYLYRLFRKSNLKPFER
DISTEIYQAGSTPCNGVEGFNCYFPLQSYGFQPTNGVGYQPYRVVVLSFELLHAPATV
CGP** (SEQ ID NO: 76)
WuhanS_RBD_gmax_LS_pVax
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcaatctgtgccctttcggtgaggtctttaatgcaa
caagatttgcaagtgtttacgcctggaaccgtaagcgcattagcaactgcactgccgattactctgtgctgtacaacagcacaagcttttc
cacatttaaatgttacggggtttcccctaccaacctcagcgacctctgctttactaatgtttacgcagattccttcgttatccgaggcgatga
agtccggcagatagctcccggacagaccggcaaaatcgctgactacaactataaactgccgaacgacagcacagggtgtgtaattgc
ttggaacagcaataacctcgattcaaaggttggcggaaattacaattatctttaccgtctgttccggaaaagcaatctgaaaccctttgag
agagacatcagcacggaaatttatcaagccggttcaacaccatgtaacggagttgaaggctttaattgctattttcccctgcaatcttacg
gatttcaacctacgaacggggtcggttaccaaccttaccgggtggtcgtgctgagcttcgaattgcttcatgccccagccaccgtgtgtg
ggccataatga (SEQ ID NO: 77)
ggauccgccaccauggacuggaccuggauucuguuccugguggccgccgccacaagggugcacagcaugcagaucuacg
aaggaaaacugaccgcugagggacugagguucggaauugucgcaagccgcgcgaaucacgcacugguggauaggcuggu
ggaaggcgcuaucgacgcaauuguccggcacggcgggagagaggaagacaucacacuggugagagucugcggcagcugg
gagauucccguggcagcuggagaacuggcucgaaaggaggacaucgaugccgugaucgcuauugggguccugugccgag
gagcaacucccagcuucgacuacaucgccucagaagugagcaaggggcuggcugaucugucccuggagcugaggaaacc
uaucacuuuuggcgugauuacugccgacacccuggaacaggcaaucgaggcggccggcaccugccauggaaacaaaggcu
gggaagcagcccugugcgcuauugagauggcaaaucuguucaaaucucugcgaggaggcuccggaggaucuggagggag
uggaggcucaggaggaggcaaucugugcccuuucggugaggucuuuaaugcaacaagauuugcaaguguuuacgccug
gaaccguaagcgcauuagcaacugcacugccgauuacucugugcuguacaacagcacaagcuuuuccacauuuaaauguu
acgggguuuccccuaccaaccucagcgaccucugcuuuacuaauguuuacgcagauuccuucguuauccgaggcgauga
aguccggcagauagcucccggacagaccggcaaaaucgcugacuacaacuauaaacugccgaacgacagcacagggugug
uaauugcuuggaacagcaauaaccucgauucaaagguuggcggaaauuacaauuaucuuuaccgucuguuccggaaaag
caaucugaaacccuuugagagagacaucagcacggaaauuuaucaagccgguucaacaccauguaacggaguugaaggcu
uuaauugcuauuuuccccugcaaucuuacggauuucaaccuacgaacggggucgguuaccaaccuuaccggguggucgu
gcugagcuucgaauugcuucaugccccagccaccgugugugggccauaauga (SEQ ID NO: 78)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGNLCPFGEVFNATRFASVYAWNRKRISNCTADYSVLYNSTSFS
TFKCYGVSPTNLSDLCFTNVYADSFVIRGDEVRQIAPGQTGKIADYNYKLPNDSTGC
VIAWNSNNLDSKVGGNYNYLYRLFRKSNLKPFERDISTEIYQAGSTPCNGVEGFNCY
FPLQSYGFQPTNGVGYQPYRVVVLSFELLHAPATVCGP** (SEQ ID NO: 79)
CoV2-RBD_7 mer_pVax
ggatccgccaccatggactggacctggattctgttcctggtggccgccgccacaagggtgcacagcaatctctgtccattcggagagg
ttttcaacgcgacgagattcgcctcagtttatgcctggaaccgtaaacggatatcaaactgcgtggctgactactctgttttatacaactcc
gcctctttcagtaccttcaagtgttacggtgtcagccctaccaaattgaatgatctctgctttacaaatgtttacgcagattcttttgtcataag
gggcgatgaggttcggcaaatcgcccccgggcagacaggcaaaattgcggactataattataagttgccagacgatttcacgggctg
cgtcatcgcctggaacagtaataatctcgattcaaaagtgggtgggaactacaattatctctacaggttattccggaagtcaaatctgaag
cccttcgaacgcgacatcagtacggagatttaccaggctggaagcactccgtgcaacggggtggaggggttcaactgttattttcctct
gcagtcttatgggtttcagcccactaatggtgtgggataccagccgtacagagtcgtggtgctgtccttcgaacttctccacgctcccgc
caccgtctgtggtcccgggggatctggcggatcagggggtagtggaggtagcggcggcgggaagaaacagggagacgctgacgt
ctgtggggaagtggcttacatccagagcgtggtgtctgattgccatgtaccaaccgcggagctcaggactcttttagagattcggaaac
tgtttctggagatccaaaagctgaaggtcgaactccagggcctgtcaaaagaatgataa (SEQ ID NO: 80)
ggauccgccaccauggacuggaccuggauucuguuccugguggccgccgccacaagggugcacagcaaucucuguccau
ucggagagguuuucaacgcgacgagauucgccucaguuuaugccuggaaccguaaacggauaucaaacugcguggcuga
cuacucuguuuuauacaacuccgccucuuucaguaccuucaaguguuacggugucagcccuaccaaauugaaugaucuc
ugcuuuacaaauguuuacgcagauucuuuugucauaaggggcgaugagguucggcaaaucgcccccgggcagacaggca
aaauugcggacuauaauuauaaguugccagacgauuucacgggcugcgucaucgccuggaacaguaauaaucucgauuc
aaaagugggugggaacuacaauuaucucuacagguuauuccggaagucaaaucugaagcccuucgaacgcgacaucagua
cggagauuuaccaggcuggaagcacuccgugcaacgggguggagggguucaacuguuauuuuccucugcagucuuaug
gguuucagcccacuaauggugugggauaccagccguacagagucguggugcuguccuucgaacuucuccacgcucccgc
caccgucuguggucccgggggaucuggcggaucaggggguaguggagguagcggcggcgggaagaaacagggagacgc
ugacgucuguggggaaguggcuuacauccagagcguggugucugauugccauguaccaaccgcggagcucaggacucuu
uuagagauucggaaacuguuucuggagauccaaaagcugaaggucgaacuccagggccugucaaaagaaugauaa
(SEQ ID NO: 81)
NLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGKKQGDADVCGEVAYIQSVV
SDCHVPTAELRTLLEIRKLFLEIQKLKVELQGLSKE** (SEQ ID NO: 82)
CoV2-RBD_gmax_14 mer_pVax
ggatccgccaccatggactggacctggattctgttcctggtggccgccgccacaagggtgcacagcaatctgtgtcccttcggggag
gttttcaatgctaccagatttgccagtgtgtatgcttggaatcggaagagaatctccaattgcacagcagattattcagttctctacaactct
acatcttttagtacctttaagtgttacggggtgagtcccactaacctttcagatttatgtttcaccaatgtctacgctgactccttcgtgatccg
gggggatgaggtgagacagattgcacctggacaaactggcaaaatcgccgactacaattacaaacttccaaacgactctacagggtg
tgtaatcgcttggaacagcaataatctggatagcaaagtaggcggcaattataattacctctacagactgtttaggaagtccaacctgaa
accatttgagagggacatcagcactgaaatctaccaggcggggagcaccccttgtaatggagtcgagggtttcaactgttacttcccac
tgcagagctacgggttccagcctaccaatggtgtcggttaccagccctatcgagttgtggtgttgtcattcgaactgttacatgcacctgc
aacggtctgtggacccgggggttcagggggtagtggggggtccggtgggagcggtgggggcaagaaacaggggatgaatccgct
catcgccgccgcctctgtgatagctgctggcctggccgtgggcctggcatcaatcgggcccggggtgggccaaggcaccgccgcc
ggccaggccgtcgagggtattgcaaggcagccggaggcagaaggcaaaattagagggaccctgttgttgtctttagcgttcatggaa
gccctcactatttacggactggttgtggccttagcccttctgtttgccaatcctttcgtgtaatga (SEQ ID NO: 83)
ggauccgccaccauggacuggaccuggauucuguuccugguggccgccgccacaagggugcacagcaaucugugucccu
ucggggagguuuucaaugcuaccagauuugccaguguguaugcuuggaaucggaagagaaucuccaauugcacagcaga
uuauucaguucucuacaacucuacaucuuuuaguaccuuuaaguguuacggggugagucccacuaaccuuucagauuua
uguuucaccaaugucuacgcugacuccuucgugauccggggggaugaggugagacagauugcaccuggacaaacuggca
aaaucgccgacuacaauuacaaacuuccaaacgacucuacaggguguguaaucgcuuggaacagcaauaaucuggauagc
aaaguaggcggcaauuauaauuaccucuacagacuguuuaggaaguccaaccugaaaccauuugagagggacaucagcac
ugaaaucuaccaggcggggagcaccccuuguaauggagucgaggguuucaacuguuacuucccacugcagagcuacggg
uuccagccuaccaauggugucgguuaccagcccuaucgaguugugguguugucauucgaacuguuacaugcaccugcaa
cggucuguggacccggggguucaggggguagugggggguccggugggagcggugggggcaagaaacaggggaugaauc
cgcucaucgccgccgccucugugauagcugcuggccuggccgugggccuggcaucaaucgggcccggggugggccaagg
caccgccgccggccaggccgucgaggguauugcaaggcagccggaggcagaaggcaaaauuagagggacccuguuguug
ucuuuagcguucauggaagcccucacuauuuacggacugguuguggccuuagcccuucuguuugccaauccuuucgug
uaauga (SEQ ID NO: 84)
NLCPFGEVFNATRFASVYAWNRKRISNCTADYSVLYNSTSFSTFKCYGVSPTNLSDL
CFTNVYADSFVIRGDEVRQIAPGQTGKIADYNYKLPNDSTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGKKQGMNPLIAAASVIAAGL
AVGLASIGPGVGQGTAAGQAVEGIARQPEAEGKIRGTLLLSLAFMEALTIYGLVVAL
ALLFANPFV** (SEQ ID NO: 85)
CoV2-RBD_gmax_24 mer_pVax
ggatccgccaccatggactggacctggattctgttcctggtggccgccgccacaagggtgcacagcaacctgtgccccttcggggaa
gtttttaatgccactcggtttgcctccgtttacgcctggaacaggaagcgcatttccaactgcaccgccgactatagcgtcctttataacag
cacatccttttcaactttcaagtgttacggggtttcccctacaaatctctctgacctgtgttttacaaatgtgtatgcagactctttcgtgattag
gggagatgaggtgcgccagatcgcccctggacagaccggaaaaatcgccgattataattataagcttcccaacgactccacaggctg
tgtaattgcctggaattctaataacctggactctaaagtgggcggtaactacaattatctgtatagactcttcagaaagtctaacctcaaac
catttgaacgggacatctcaaccgagatctaccaagccgggtccaccccctgtaacggcgtggaaggcttcaactgttatttccccctc
cagtcctatggcttccaacccacaaatggagtcggctaccagccttacagggtggttgtgctgtcatttgagctcctccacgctcctgcc
actgtatgtgggccaggcgggtccggaggttcaggcggtagcggcggctcaggtggaggaggactgtctaaagatattataaaactg
ctgaacgaacaagtgaacaaggagatgcagagcagcaacctttacatgtctatgagcagttggtgttacactcactctctcgacggcgc
cggcctgttcctgtttgatcacgccgcggaggagtatgaacatgctaaaaagcttatcatcttcctcaacgaaaataacgtgccagtgca
gttgacctctatttccgctcccgaacataagttcgaaggcctcacacagatctttcagaaggcttacgagcatgaacaacacatttcaga
gagcatcaacaacatcgtggaccatgcgatcaagtctaaggaccacgcgacttttaacttcctccagtggtatgtcgccgaacagcatg
aggaggaagtgttgttcaaagacatcctggacaagattgaacttattggcaacgaaaaccacggcctctacctggccgatcagtacgt
gaaaggtatcgcgaagtcacgaaagagttaatga (SEQ ID NO: 86)
ggauccgccaccauggacuggaccuggauucuguuccugguggccgccgccacaagggugcacagcaaccugugccccu
ucggggaaguuuuuaaugccacucgguuugccuccguuuacgccuggaacaggaagcgcauuuccaacugcaccgccga
cuauagcguccuuuauaacagcacauccuuuucaacuuucaaguguuacgggguuuccccuacaaaucucucugaccug
uguuuuacaaauguguaugcagacucuuucgugauuaggggagaugaggugcgccagaucgccccuggacagaccggaa
aaaucgccgauuauaauuauaagcuucccaacgacuccacaggcuguguaauugccuggaauucuaauaaccuggacucu
aaagugggcgguaacuacaauuaucuguauagacucuucagaaagucuaaccucaaaccauuugaacgggacaucucaac
cgagaucuaccaagccggguccacccccuguaacggcguggaaggcuucaacuguuauuucccccuccaguccuauggcu
uccaacccacaaauggagucggcuaccagccuuacagggugguugugcugucauuugagcuccuccacgcuccugccac
uguaugugggccaggcggguccggagguucaggcgguagcggcggcucagguggaggaggacugucuaaagauauuau
aaaacugcugaacgaacaagugaacaaggagaugcagagcagcaaccuuuacaugucuaugagcaguugguguuacacuc
acucucucgacggcgccggccuguuccuguuugaucacgccgcggaggaguaugaacaugcuaaaaagcuuaucaucuu
ccucaacgaaaauaacgugccagugcaguugaccucuauuuccgcucccgaacauaaguucgaaggccucacacagaucu
uucagaaggcuuacgagcaugaacaacacauuucagagagcaucaacaacaucguggaccaugcgaucaagucuaaggac
cacgcgacuuuuaacuuccuccagugguaugucgccgaacagcaugaggaggaaguguuguucaaagacauccuggaca
agauugaacuuauuggcaacgaaaaccacggccucuaccuggccgaucaguacgugaaagguaucgcgaagucacgaaag
aguuaauga (SEQ ID NO: 87)
NLCPFGEVFNATRFASVYAWNRKRISNCTADYSVLYNSTSFSTFKCYGVSPTNLSDL
CFTNVYADSFVIRGDEVRQIAPGQTGKIADYNYKLPNDSTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGGLSKDIIKLLNEQVNKEMQS
SNLYMSMSSWCYTHSLDGAGLFLFDHAAEEYEHAKKLIIFLNENNVPVQLTSISAPE
HKFEGLTQIFQKAYEHEQHISESINNIVDHAIKSKDHATFNFLQWYVAEQHEEEVLFK
DILDKIELIGNENHGLYLADQYVKGIAKSRKS** (SEQ ID NO: 88)
CoV2-RBD_gmax_7 mer_pVax
ggatccgccaccatggactggacctggattctgttcctggtggccgccgccacaagggtgcacagcaatctttgtccattcggggaag
tgtttaacgccactaggttcgctagtgtgtacgcctggaatcggaagcggatttcaaattgtaccgccgattattctgtcctttacaacagt
accagcttttccacttttaaatgctacggagtatctcctacaaacttgagtgacctgtgttttacgaacgtctacgctgactctttcgttattag
gggagacgaagttagacaaatcgctccaggccagactggcaaaatagccgactataactataaactcccaaacgattccacaggctg
cgttattgcctggaacagcaataacctggactctaaagtcggaggtaactataactacttgtacaggctcttccgcaagagcaaccttaa
gccatttgagcgagatatctccaccgagatttatcaggcagggagcaccccatgcaacggagtggaggggtttaattgctattttccact
gcagtcctatggctttcaaccaacaaacggagtaggctaccaaccgtatcgcgttgtcgtcctgagtttcgaactgttgcacgcccctgc
gaccgtatgtggccccggcggctcaggggggagtggtgggagcgggggctctgggggggggaaaaaacagggggacgccgat
gtttgcggcgaggtggcctatatacagtcagtggtctccgactgtcatgtaccaactgccgaactcaggactcttctggagataaggaa
gttgttcctggagatacagaagctcaaggtcgagttacagggtctctcaaaggaatgatga (SEQ ID NO: 89)
ggauccgccaccauggacuggaccuggauucuguuccugguggccgccgccacaagggugcacagcaaucuuuguccau
ucggggaaguguuuaacgccacuagguucgcuaguguguacgccuggaaucggaagcggauuucaaauuguaccgccga
uuauucuguccuuuacaacaguaccagcuuuuccacuuuuaaaugcuacggaguaucuccuacaaacuugagugaccug
uguuuuacgaacgucuacgcugacucuuucguuauuaggggagacgaaguuagacaaaucgcuccaggccagacuggca
aaauagccgacuauaacuauaaacucccaaacgauuccacaggcugcguuauugccuggaacagcaauaaccuggacucu
aaagucggagguaacuauaacuacuuguacaggcucuuccgcaagagcaaccuuaagccauuugagcgagauaucuccac
cgagauuuaucaggcagggagcaccccaugcaacggaguggagggguuuaauugcuauuuuccacugcaguccuauggc
uuucaaccaacaaacggaguaggcuaccaaccguaucgcguugucguccugaguuucgaacuguugcacgccccugcgac
cguauguggccccggcggcucaggggggaguggugggagcgggggcucugggggggggaaaaaacagggggacgccga
uguuugcggcgagguggccuauauacagucaguggucuccgacugucauguaccaacugccgaacucaggacucuucug
gagauaaggaaguuguuccuggagauacagaagcucaaggucgaguuacagggucucucaaaggaaugauga (SEQ
ID NO: 90)
NLCPFGEVFNATRFASVYAWNRKRISNCTADYSVLYNSTSFSTFKCYGVSPTNLSDL
CFTNVYADSFVIRGDEVRQIAPGQTGKIADYNYKLPNDSTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGKKQGDADVCGEVAYIQSVV
SDCHVPTAELRTLLEIRKLFLEIQKLKVELQGLSKE** (SEQ ID NO: 91)
II. CoV2 Trimer Constructs
WuhanS_SolTri_FS1_pVax
ggatccgccaccatggactggacctggattctgttcctggtggccgccgccacaagggtgcacagcatgtttgttttcctcgtcctcttg
cccctcgtctctagtcaatgtgttaatttgaccacacgaacccaactgccacctgcctacaccaacagttttaccagaggagtttattaccc
cgacaaagtattcaggtcatcagtgctgcatagtacccaagacttgtttctccccttctttagtaacgttacatggttccacgccattcacgt
gagtgggacaaatggaacaaaacgcttcgacaaccctgtgctccccttcaacgatggtgtatactttgctagtaccgagaagagcgga
attatccgcgggtggatctttggaacaacactggacagcaaaacccaaagcctgcttatcgttaacaatgctactaacgttgtgatcaaa
gtgtgtgaattccaattttgtaatgatccgtttctcggagtttactaccacaagaacaacaaaagttggatggaaagcgaattccgggtgta
ctcctcagcaaataattgtacctttgagtacgtgagtcaaccctttctcatggacctggaaggaaaacaaggcaatttcaagaacctgcg
ggagtttgtgttcaagaatattgatggctattttaaaatttattctaagcatactccaatcaacctggtaagggacctgccccaaggcttttca
gccctcgaaccgcttgtagatttgcctatcgggataaacattacgcgatttcaaacgctgttggcgctccaccggagctacttgactcctg
gcgatagcagctccggttggaccgctggagcggccgcttattacgtcggctatctgcaacccaggacgttcctgctcaagtataatga
gaacgggacgattacagatgcagtggattgtgcgcttgatcctctctctgaaaccaagtgcactctcaagtctttcacggtggagaaag
gcatttatcaaactagtaactttcgagtacagcctactgagagtatcgttaggttcccaaacattacgaacctctgtccctttggagaagtat
tcaatgctactcgctttgcaagcgtttatgcctggaatcgcaaacgcatcagcaattgcgtcgccgattattctgtcctttataatagcgcat
cattttcaacatttaagtgttatggggtgagtccgactaagctcaatgatttgtgcttcacaaacgtctacgcggacagctttgtgataagg
ggcgacgaagttcgccaaatcgctcccggccaaactgggaaaatcgcggattacaactataaattgcccgatgacttcaccggctgtg
tcattgcctggaactctaataacctcgatagcaaggtgggcgggaactataattatttgtaccgcctgtttcgaaagtccaatctcaaacc
ctttgagcgggacatttccactgagatctatcaggcagggagtacaccttgtaacggcgtggaaggctttaactgttattttcccctgcaa
agttacggttttcaacctaccaacggagttggctatcaaccttatcgagtcgtcgtgctgagttttgagttgctgcatgccccagccaccgt
ctgtggacctaagaaatccaccaacctcgtgaagaacaagtgcgtcaattttaattttaacggcctgactgggaccggtgtcctcaccga
atctaataagaagttcctgccatttcaacaattcggacgggacatcgctggaacgacagatgctgtccgtgatcctcagacactggaga
ttctggacatcactccttgcagctttggcggagtctctgttattactcccggaactaacacttctaaccaagttgctgtcctctatcaggacg
tgaactgcactgaagtgcccgtggcaatccatgcaggccaactgacccccacttggagagtctacagcacggggagcaatgtcttcc
aaacaagggccggatgccttattggagcggagcacgttaataactcatacgagtgtgatataccaattggagcaggaatttgtgcttcct
accagacccaaactaacagtcccaggcgggctaggagtgtcgctagccagagcatcatcgcgtacacaatgtctctcggcgcagaa
aactcagtcgcctatagcaacaactcaattgccattcccaccaacttcacaatttccgtaaccactgaaattctgcctgttagcatgacaaa
gacatcagttgattgtacaatgtacatatgtggagacagcaccgaatgcagcaaccttttgcttcaatatggctccttttgtacccaactca
acagggcactcactgggatagcagtcgaacaagataagaacacccaagaggtgtttgcacaagtcaaacaaatctataaaacgccgc
ccataaaagactttggcggattcaatttcagccagatcttgcctgacccatccaagccttcaaagaggagctttattgaggatcttctcttc
aataaagtgacactggcggacgccggttttatcaagcaatatggtgattgtctcggtgacatagcagctagagatctgatttgcgctcag
aaatttaatggccttactgtgcttcccccactgctgaccgatgaaatgattgcacaatatacaagcgcccttttggccgggactattacttc
cgggtggaccttcggcgccggcgccgctctgcaaattcctttcgcaatgcagatggcctaccggttcaatggcataggtgtcactcag
aacgttctttatgagaatcagaaactcatcgcgaaccagtttaattcagcgatcggcaagattcaggactccttgtcctcaactgcgtcag
ctttgggaaaacttcaagacgtcgtgaaccagaatgctcaggcgctcaataccctggtgaaacaacttagcagtaactttggggctattt
ctagcggtccaaacgatatactgtcccgactcccgaaagtcgaggccgaagtccaaattgatcgtcttattacagggagactccaatct
cttcaaacatatgtcactcaacagctcattagggctgcggagatccgggcttccgcaaatcttgccgcgacaaagatgagtgaatgcgt
cttgggacaatctaagagggtggacttttgtggaaaaggttaccatctcatgtccttccctcagtcagcgccccacggagtcgttttcctg
cacgtaacgtatgtcccggctcaagagaagaacttcactactgcaccagcgatttgccatgacggtaaagcccattttccccgcgagg
gcgtatttgtgtccaacggtacccactggttcgtaacccaacggaatttctatgagccccaaatcattacaacagataatacagatgtttcc
gggaattgcgacgttgttattggcatcgttaacaacaccgtttacgatcccttgcaaccggaactggactcctttaaagaagaactcgac
aagtattttaagaaccacacatcaccagatgtcgatcttggcgacatttccggcattaacgcttcagttgtaaatattcagaaagagatag
atcgcctgaatgaggtggctaagaacctgaacgaatctctcattgatctccaagagctgggaaagtacgaacaatacatcaaatggcct
tctgggcgtcgccgaagacgacgagggtccggcggctcagggagcggctatatccctgaggcgcctcgggacggacaagcttatg
tgaggaaagatggagaatgggtattgctgtcaaccttcctgggataatga (SEQ ID NO: 92)
ggauccgccaccauggacuggaccuggauucuguuccugguggccgccgccacaagggugcacagcauguuuguuuucc
ucguccucuugccccucgucucuagucaauguguuaauuugaccacacgaacccaacugccaccugccuacaccaacagu
uuuaccagaggaguuuauuaccccgacaaaguauucaggucaucagugcugcauaguacccaagacuuguuucuccccu
ucuuuaguaacguuacaugguuccacgccauucacgugagugggacaaauggaacaaaacgcuucgacaacccugugcuc
cccuucaacgaugguguauacuuugcuaguaccgagaagagcggaauuauccgcggguggaucuuuggaacaacacugg
acagcaaaacccaaagccugcuuaucguuaacaaugcuacuaacguugugaucaaagugugugaauuccaauuuuguaau
gauccguuucucggaguuuacuaccacaagaacaacaaaaguuggauggaaagcgaauuccggguguacuccucagcaaa
uaauuguaccuuugaguacgugagucaacccuuucucauggaccuggaaggaaaacaaggcaauuucaagaaccugcgg
gaguuuguguucaagaauauugauggcuauuuuaaaauuuauucuaagcauacuccaaucaaccugguaagggaccugc
cccaaggcuuuucagcccucgaaccgcuuguagauuugccuaucgggauaaacauuacgcgauuucaaacgcuguuggc
gcuccaccggagcuacuugacuccuggcgauagcagcuccgguuggaccgcuggagcggccgcuuauuacgucggcuau
cugcaacccaggacguuccugcucaaguauaaugagaacgggacgauuacagaugcaguggauugugcgcuugauccuc
ucucugaaaccaagugcacucucaagucuuucacgguggagaaaggcauuuaucaaacuaguaacuuucgaguacagccu
acugagaguaucguuagguucccaaacauuacgaaccucugucccuuuggagaaguauucaaugcuacucgcuuugcaa
gcguuuaugccuggaaucgcaaacgcaucagcaauugcgucgccgauuauucuguccuuuauaauagcgcaucauuuuc
aacauuuaaguguuauggggugaguccgacuaagcucaaugauuugugcuucacaaacgucuacgcggacagcuuugug
auaaggggcgacgaaguucgccaaaucgcucccggccaaacugggaaaaucgcggauuacaacuauaaauugcccgauga
cuucaccggcugugucauugccuggaacucuaauaaccucgauagcaaggugggcgggaacuauaauuauuuguaccgc
cuguuucgaaaguccaaucucaaacccuuugagcgggacauuuccacugagaucuaucaggcagggaguacaccuugua
acggcguggaaggcuuuaacuguuauuuuccccugcaaaguuacgguuuucaaccuaccaacggaguuggcuaucaacc
uuaucgagucgucgugcugaguuuugaguugcugcaugccccagccaccgucuguggaccuaagaaauccaccaaccuc
gugaagaacaagugcgucaauuuuaauuuuaacggccugacugggaccgguguccucaccgaaucuaauaagaaguucc
ugccauuucaacaauucggacgggacaucgcuggaacgacagaugcuguccgugauccucagacacuggagauucugga
caucacuccuugcagcuuuggcggagucucuguuauuacucccggaacuaacacuucuaaccaaguugcuguccucuau
caggacgugaacugcacugaagugcccguggcaauccaugcaggccaacugacccccacuuggagagucuacagcacggg
gagcaaugucuuccaaacaagggccggaugccuuauuggagcggagcacguuaauaacucauacgagugugauauacca
auuggagcaggaauuugugcuuccuaccagacccaaacuaacagucccaggcgggcuaggagugucgcuagccagagca
ucaucgcguacacaaugucucucggcgcagaaaacucagucgccuauagcaacaacucaauugccauucccaccaacuuca
caauuuccguaaccacugaaauucugccuguuagcaugacaaagacaucaguugauuguacaauguacauauguggaga
cagcaccgaaugcagcaaccuuuugcuucaauauggcuccuuuuguacccaacucaacagggcacucacugggauagcag
ucgaacaagauaagaacacccaagagguguuugcacaagucaaacaaaucuauaaaacgccgcccauaaaagacuuuggcg
gauucaauuucagccagaucuugccugacccauccaagccuucaaagaggagcuuuauugaggaucuucucuucaauaaa
gugacacuggcggacgccgguuuuaucaagcaauauggugauugucucggugacauagcagcuagagaucugauuugc
gcucagaaauuuaauggccuuacugugcuucccccacugcugaccgaugaaaugauugcacaauauacaagcgcccuuuu
ggccgggacuauuacuuccggguggaccuucggcgccggcgccgcucugcaaauuccuuucgcaaugcagauggccuac
cgguucaauggcauaggugucacucagaacguucuuuaugagaaucagaaacucaucgcgaaccaguuuaauucagcga
ucggcaagauucaggacuccuuguccucaacugcgucagcuuugggaaaacuucaagacgucgugaaccagaaugcuca
ggcgcucaauacccuggugaaacaacuuagcaguaacuuuggggcuauuucuagcgguccaaacgauauacugucccga
cucccgaaagucgaggccgaaguccaaauugaucgucuuauuacagggagacuccaaucucuucaaacauaugucacuca
acagcucauuagggcugcggagauccgggcuuccgcaaaucuugccgcgacaaagaugagugaaugcgucuugggacaa
ucuaagaggguggacuuuuguggaaaagguuaccaucucauguccuucccucagucagcgccccacggagucguuuucc
ugcacguaacguaugucccggcucaagagaagaacuucacuacugcaccagcgauuugccaugacgguaaagcccauuuu
ccccgcgagggcguauuuguguccaacgguacccacugguucguaacccaacggaauuucuaugagccccaaaucauuac
aacagauaauacagauguuuccgggaauugcgacguuguuauuggcaucguuaacaacaccguuuacgaucccuugcaa
ccggaacuggacuccuuuaaagaagaacucgacaaguauuuuaagaaccacacaucaccagaugucgaucuuggcgacau
uuccggcauuaacgcuucaguuguaaauauucagaaagagauagaucgccugaaugagguggcuaagaaccugaacgaa
ucucucauugaucuccaagagcugggaaaguacgaacaauacaucaaauggccuucugggcgucgccgaagacgacgagg
guccggcggcucagggagcggcuauaucccugaggcgccucgggacggacaagcuuaugugaggaaagauggagaaugg
guauugcugucaaccuuccugggauaauga (SEQ ID NO: 93)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSGIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIAGTTDAVRDPQTLEILDITPCSFGGVSVITPGTNTSNQVAVLYQDV
NCTEVPVAIHAGQLTPTWRVYSTGSNVFQTRAGCLIGAEHVNNSYECDIPIGAGICAS
YQTQTNSPRRARSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTDVSGNCDVVIGIVNNTV
YDPLQPELDSFKEELDKYFKNHTSPDVDLGDISGINASVVNIQKEIDRLNEVAKNLNE
SLIDLQELGKYEQYIKWPSGRRRRRRGSGGSGSGYIPEAPRDGQAYVRKDGEWVLLS
TFLG** (SEQ ID NO: 94)
WuS_IgE_StrepHis_pVax
gccaccatggactggacctggatactctttctcgtagcagcagccacacgagtgcattcaagccaatgcgtgaatctgactacaagga
ctcagctgccccctgcatacacgaacagtttcacccgcggtgtatattatccggacaaagtattcaggtctagtgtgctgcactcaaccc
aggatttgtttctgcccttcttctctaacgtgacatggttccacgccatccatgtgtcaggtacgaacggtaccaagagatttgataacccg
gtactgccatttaatgacggcgtctattttgcttccactgagaagagcaacatcataagaggctggatctttggaactactctggacagca
aaacccagagcttgctgatcgtgaacaacgcgacaaatgtagtgatcaaagtatgtgagtttcaattctgtaacgatcccttccttggggt
ttattaccataagaataataagagttggatggagtccgaatttagagtttactcctcagctaataactgtacgttcgagtatgtctcccaacc
ttttcttatggatctcgaagggaaacagggtaactttaagaatcttcgagaatttgtgttcaagaacatcgacggttattttaagatctacagt
aagcatactcccataaatctggttagagatctcccgcaaggattttccgcactggagccccttgtagaccttcccattggaataaacataa
cacgtttccagacactcctcgctctgcataggtcatatctcaccccgggcgattcttccagcggatggaccgctggagctgctgcttact
acgtaggatacctgcaaccccggacatttctgctcaagtataacgaaaatgggactattacggacgctgtggactgtgctcttgaccca
cttagcgagacaaaatgcacgctgaaaagttttaccgtggagaaggggatctatcaaacgagcaattttagggttcagcctaccgaatc
aatcgtcagatttcccaatatcactaacctgtgccctttcggggaagttttcaacgcaacccggtttgcgagcgtatacgcttggaatcgc
aaaaggataagcaattgcgttgccgattactccgttctttacaattcagcatcattttctacttttaaatgctacggcgtgtctcccacaaaac
tgaatgacctgtgttttacgaacgtgtatgcagacagctttgtgattaggggtgatgaggttagacaaatcgcaccaggtcagaccggta
agatcgctgattacaactacaaactgcccgatgacttcacaggatgcgtgattgcctggaattccaacaatctggattctaaggttggcg
gcaattacaattacctgtataggttgtttcggaagtcaaacctgaaacccttcgaaagagacatttctaccgagatttatcaagcgggttca
actccttgtaatggagttgaaggcttcaattgttactttccccttcaatcatacggattccaaccaaccaatggggtcggataccagccata
tagggttgttgtcctgtcattcgaacttctccacgcaccagccaccgtatgtggacccaagaagtctactaatctggtgaagaacaaatg
cgtcaatttcaactttaatgggttgaccggcactggggtgctgactgaatccaacaagaagtttctgccgttccaacaattcggacgcga
tatcgctgatacaaccgatgccgttagagatccccaaacattggagattctggatattacgccttgttcattcggtggtgtttccgtgattac
ccctggcaccaatacgagtaaccaagtggcggtgctgtatcaagatgtgaactgtactgaagtgccggtggctatacatgccgaccaa
ctcacaccaacatggagagtatatagcacgggttccaatgtgtttcaaactagggctggctgtttgattggcgctgaacatgttaataattc
ctatgaatgcgatattcccatcggtgccgggatttgcgcaagttatcaaacgcaaactaactcccccgggtcagcatcctctgtcgcttc
ccaatcaatcatcgcctataccatgagtcttggggcagaaaattccgttgcttattctaacaattccattgcaattcctacgaacttcaccat
ctcagttactacagaaatacttcccgtgtcaatgacgaagacatccgtagattgcacaatgtatatatgtggggactcaactgaatgctca
aacctgctcctgcaatacggatcattttgcacccaactgaacagagcattgaccggtatagccgtggagcaagataagaacactcaag
aagtattcgcccaggtcaaacaaatctataaaactccgcctataaaagattttggcggctttaacttttcccaaatactgcctgacccaagt
aagccctcaaaacgtagctttatagaggacctcttgtttaataaggtgacactcgctgacgctggattcattaagcaatatggtgactgctt
gggagatattgccgcccgcgatctcatttgtgcacaaaagttcaacggcctcacagtcctgccccctctgctgacggatgaaatgatcg
ctcaatacacctcagctctcctggcaggcaccataacaagcgggtggacatttggtgccggggcagcactgcaaatcccattcgcaat
gcaaatggcttataggttcaatgggatcggcgtaactcaaaatgtcctctacgagaaccagaaactcatagctaaccaattcaattctgc
aatcgggaaaatccaggactccctgagctcaacggccagcgcactgggcaagctccaagatgtggtcaaccaaaacgcacaagca
ctgaatactcttgtgaaacaactcagctccaatttcggggcaatatcaagtgtcctcaatgatattcttagcaggcttgatccacccgaag
ccgaggtgcagatcgacaggctcataacaggcaggctccagtcccttcaaacgtatgtaactcagcaactgattcgggctgccgagat
tcgagcttcagctaatttggcagctacgaagatgagcgaatgcgtcctgggacagtctaaaagagtagacttttgcggcaaagggtatc
atctgatgagcttcccacaaagtgctccacatggcgtggttttcctgcatgtcacttatgttcccgcacaagagaagaacttcactaccgc
accagcgatctgtcacgatggtaaagcacatttcccgcgggaaggcgtattcgtatctaacggcacccactggttcgttactcaacgca
acttttatgaaccacaaatcattacaaccgataacacttttgtttcaggcaattgcgatgttgtcatcggcattgtgaataacactgtgtacg
atccacttcaaccagaattggacagctttaaagaggagcttgataagtatttcaagaatcatacctctcccgacgtggacctcggggaca
tctctggaataaatgctagcgtcgttaatatacagaaagagattgatcgtctgaacgaagtggctaagaatctgaatgaaagccttatcga
tctgcaagaactggggaagtacgaacagggatacataccggaagccccacgcgacggtcaggcttatgttaggaaggatggagaat
gggttttgctctccacgtttctcgggcttgaagttttgttccaaggaccctggtcacacccccaatttgagaaacaccatcaccaccatca
ccaccactgataa (SEQ ID NO: 95)
gccaccauggacuggaccuggauacucuuucucguagcagcagccacacgagugcauucaagccaaugcgugaaucugac
uacaaggacucagcugcccccugcauacacgaacaguuucacccgcgguguauauuauccggacaaaguauucaggucua
gugugcugcacucaacccaggauuuguuucugcccuucuucucuaacgugacaugguuccacgccauccaugugucagg
uacgaacgguaccaagagauuugauaacccgguacugccauuuaaugacggcgucuauuuugcuuccacugagaagagc
aacaucauaagaggcuggaucuuuggaacuacucuggacagcaaaacccagagcuugcugaucgugaacaacgcgacaaa
uguagugaucaaaguaugugaguuucaauucuguaacgaucccuuccuugggguuuauuaccauaagaauaauaagagu
uggauggaguccgaauuuagaguuuacuccucagcuaauaacuguacguucgaguaugucucccaaccuuuucuuaugg
aucucgaagggaaacaggguaacuuuaagaaucuucgagaauuuguguucaagaacaucgacgguuauuuuaagaucua
caguaagcauacucccauaaaucugguuagagaucucccgcaaggauuuuccgcacuggagccccuuguagaccuuccca
uuggaauaaacauaacacguuuccagacacuccucgcucugcauaggucauaucucaccccgggcgauucuuccagcgga
uggaccgcuggagcugcugcuuacuacguaggauaccugcaaccccggacauuucugcucaaguauaacgaaaauggga
cuauuacggacgcuguggacugugcucuugacccacuuagcgagacaaaaugcacgcugaaaaguuuuaccguggagaa
ggggaucuaucaaacgagcaauuuuaggguucagccuaccgaaucaaucgucagauuucccaauaucacuaaccugugcc
cuuucggggaaguuuucaacgcaacccgguuugcgagcguauacgcuuggaaucgcaaaaggauaagcaauugcguugc
cgauuacuccguucuuuacaauucagcaucauuuucuacuuuuaaaugcuacggcgugucucccacaaaacugaaugacc
uguguuuuacgaacguguaugcagacagcuuugugauuaggggugaugagguuagacaaaucgcaccaggucagaccg
guaagaucgcugauuacaacuacaaacugcccgaugacuucacaggaugcgugauugccuggaauuccaacaaucuggau
ucuaagguuggcggcaauuacaauuaccuguauagguuguuucggaagucaaaccugaaacccuucgaaagagacauuu
cuaccgagauuuaucaagcggguucaacuccuuguaauggaguugaaggcuucaauuguuacuuuccccuucaaucaua
cggauuccaaccaaccaauggggucggauaccagccauauaggguuguuguccugucauucgaacuucuccacgcaccag
ccaccguauguggacccaagaagucuacuaaucuggugaagaacaaaugcgucaauuucaacuuuaauggguugaccgg
cacuggggugcugacugaauccaacaagaaguuucugccguuccaacaauucggacgcgauaucgcugauacaaccgaug
ccguuagagauccccaaacauuggagauucuggauauuacgccuuguucauucggugguguuuccgugauuaccccugg
caccaauacgaguaaccaaguggcggugcuguaucaagaugugaacuguacugaagugccgguggcuauacaugccgac
caacucacaccaacauggagaguauauagcacggguuccaauguguuucaaacuagggcuggcuguuugauuggcgcug
aacauguuaauaauuccuaugaaugcgauauucccaucggugccgggauuugcgcaaguuaucaaacgcaaacuaacucc
cccgggucagcauccucugucgcuucccaaucaaucaucgccuauaccaugagucuuggggcagaaaauuccguugcuu
auucuaacaauuccauugcaauuccuacgaacuucaccaucucaguuacuacagaaauacuucccgugucaaugacgaag
acauccguagauugcacaauguauauauguggggacucaacugaaugcucaaaccugcuccugcaauacggaucauuuu
gcacccaacugaacagagcauugaccgguauagccguggagcaagauaagaacacucaagaaguauucgcccaggucaaa
caaaucuauaaaacuccgccuauaaaagauuuuggcggcuuuaacuuuucccaaauacugccugacccaaguaagcccuc
aaaacguagcuuuauagaggaccucuuguuuaauaaggugacacucgcugacgcuggauucauuaagcaauauggugac
ugcuugggagauauugccgcccgcgaucucauuugugcacaaaaguucaacggccucacaguccugcccccucugcuga
cggaugaaaugaucgcucaauacaccucagcucuccuggcaggcaccauaacaagcggguggacauuuggugccggggc
agcacugcaaaucccauucgcaaugcaaauggcuuauagguucaaugggaucggcguaacucaaaauguccucuacgaga
accagaaacucauagcuaaccaauucaauucugcaaucgggaaaauccaggacucccugagcucaacggccagcgcacug
ggcaagcuccaagauguggucaaccaaaacgcacaagcacugaauacucuugugaaacaacucagcuccaauuucggggc
aauaucaaguguccucaaugauauucuuagcaggcuugauccacccgaagccgaggugcagaucgacaggcucauaacag
gcaggcuccagucccuucaaacguauguaacucagcaacugauucgggcugccgagauucgagcuucagcuaauuuggc
agcuacgaagaugagcgaaugcguccugggacagucuaaaagaguagacuuuugcggcaaaggguaucaucugaugagc
uucccacaaagugcuccacauggcgugguuuuccugcaugucacuuauguucccgcacaagagaagaacuucacuaccgc
accagcgaucugucacgaugguaaagcacauuucccgcgggaaggcguauucguaucuaacggcacccacugguucguu
acucaacgcaacuuuuaugaaccacaaaucauuacaaccgauaacacuuuuguuucaggcaauugcgauguugucaucgg
cauugugaauaacacuguguacgauccacuucaaccagaauuggacagcuuuaaagaggagcuugauaaguauuucaag
aaucauaccucucccgacguggaccucggggacaucucuggaauaaaugcuagcgucguuaauauacagaaagagauuga
ucgucugaacgaaguggcuaagaaucugaaugaaagccuuaucgaucugcaagaacuggggaaguacgaacagggauac
auaccggaagccccacgcgacggucaggcuuauguuaggaaggauggagaauggguuuugcucuccacguuucucgggc
uugaaguuuuguuccaaggacccuggucacacccccaauuugagaaacaccaucaccaccaucaccaccacugauaa
(SEQ ID NO: 96)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT
TLDSKTQSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVRDLPQGFSA
LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYL
YRLFRKSNLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS
VLNDILSRLD [symbol] EAEVQIDRLITGRLQSLQTYVTQQLIRAAEIRASANLAATKMSECVL
GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQGYIPEAPRDGQAYVRKDGEWVLLSTFLGLEVLFQGPWSHPQFEKHHHHHHHH**
(SEQ ID NO: 97)
WuS_IgE_DownDS2_2P_pVax
gccaccatggactggacctggatactctttctcgtagcagcagccacacgagtgcattcatcacagtgcgttaatctgaccacccgtac
acaactcccacccgcatacacaaatagctttacacgcggagtgtattaccccgataaagtctttcggagctcagtgctccattctactcaa
gatcttttcctgccgttctttagtaacgttacttggtttcatgcaatacatgtgtctggcacaaacggaaccaaacgttttgataatccggtgtt
gccatttaatgatggtgtatattttgcttccacggaaaagtcaaacatcatccgtgggtggatctttggcaccactcttgatagcaaatgtca
aagccttctgattgttaataacgctacaaacgtcgtaattaaagtgtgtgaattccagttctgtaatgaccccttcctcggagtatattacca
caagaataacaaatcttggatggagagcgaatttagagtttacagttcagccaataactgtacatttgaatatgtcagtcagcctttcctcat
ggacctcgaaggtaaacaaggtaattttaagaacttgagagagttcgtgtttaagaacatcgatggctatttcaaaatttactctaagcaca
caccaatcaacctggttcgagacctgccccagggtttctcagctttggaaccattggtggacctgccaatcggcattaacattaccagat
ttcaaactttgttggcactccaccggtcatatcttacccccggagacagttcctcaggctggacggcaggcgccgccgcgtactatgttg
ggtatctccaaccccgaaccttccttctcaaatacaatgaaaacgggacgattacagatgcagtcgattgcgccctggaccccttgtcc
gaaactaaatgcactctgaagagtttcacggtagagaagggaatctatcaaacgagcaattttcgagtccaaccaacggaatctattgtg
cggtttcccaatatcacaaacctctgtccattcggagaagtctttaatgctaccaggtttgcgtctgtatatgcatggaaccgaaagagga
tttccaattgcgtagcggactacagtgtcctttataacagcgcttcattttccacgtttaagtgttatggtgtttctccaacgaaactcaacga
cctctgttttactaacgtttacgctgacagctttgttatacgtggggacgaagtcaggcaaattgctcctggacagactggaaagatcgct
gattataattataaacttcctgacgatttcaccggctgcgttattgcatggaactccaacaatctggattcaaaagtgggtggaaattataat
tatctgtataggttgtttcggaagagcaatcttaagccctttgagcgggacatatgtaccgaaatttaccaagcaggctccaccccatgca
atggagtagaagggttcaattgctattttcctctgcaaagttatggctttcaacccaccaacggagttgggtatcaaccttacagggttgtc
gtgctgagtttcgaattgctccacgcacccgctacagtatgtggccccaagaagtccactaatcttgttaagaataaatgcgtgaacttca
acttcaatggacttacaggtactggagtactcacggaatcaaacaagaaatttctcccatttcaacagtttggccgagatatagctgacac
cacagatgctgttcgcgacccccagacgttggaaatacttgatatcactccctgcagcttcggcggcgtgagcgtgatcactccaggta
ctaatacgagcaatcaagttgccgttctgtaccaagatgtgaactgcaccgaggttccagtggcaattcacgccgaccaacttactccc
acctggcgggtctattccaccggatcaaacgtcttccaaactcgcgctggttgccttatcggtgcagagcacgttaataattcctatgaat
gtgacattcccataggagcaggcatctgtgcatcttatcaaacccagactaattcccctggttccgcttcctctgttgcatcccagtccata
attgcctacactatgagtctcggggctgaaaattccgtggcctattctaataattcaatcgccatcccaaccaattttaccatatccgtaacg
actgaaatacttcctgtcagtatgaccaagacctcagtggactgcaccatgtacatctgcggcgattctactgaatgttccaatctgctttt
gcaatatggttcattctgcacccaactcaacagggctcttacagggatcgccgtcgaacaggataagaatacccaggaagtgttcgcc
caagttaagcaaatttacaagacaccacccatcaaggacttcggcgggttcaacttcagccaaattctgcccgacccgtctaagccttct
aagcgctctttcattgaggatcttttgttcaataaggttacgcttgccgatgcagggtttatcaaacagtatggcgactgtcttggggatatc
gcagctagggatcttatttgtgcacagaaatttaatggcctgactgttcttccccctttgctcactgacgagatgattgcccagtacacttca
gctctcctggccgggactataacttctggttggaccttcggagctggcgccgccctgcaaattccatttgcaatgcagatggcttatcgct
tcaacggaattggggtgacccaaaatgttctctacgagaaccagaaactcattgcaaaccagttcaattctgcgatcgggaagatccag
gattccctgtctagtacggctagtgccctcggtaagctccaagacgtcgtcaaccaaaacgcccaggccttgaacacccttgtcaaaca
actgagctccaattttggggctattagcagtgtgctgaatgatatcctgtcccgccttgacccaccggaagcggaagtccaaattgatcg
actgatcactgggcgtctccaatcccttcaaacttacgtgacccaacaactcatccgagcagctgagattagggctagcgctaaccttg
ctgctactaagatgtcagagtgtgtcctcggccagtctaagagagtggacttttgtgggaaagggtaccacttgatgtcattcccacaaa
gcgccccacacggcgtggtgtttctccacgtcacttacgttccagctcaggaaaagaactttaccaccgcccccgctatatgtcatgatg
ggaaggcccactttcctcgtgaaggtgtctttgtcagcaatggcacacactggtttgtgacccaacggaatttctatgagcctcagattatt
accacggataacactttcgtatcagggaattgtgatgtggttatcggcatcgttaataatacagtgtatgacccactgcagccagagcttg
acagcttcaaagaagagctcgataagtactttaagaatcatacaagtcctgacgttgatcttggggatattagtgggattaacgccagcg
tcgtcaatattcagaaagagattgacaggttgaacgaagtagctaagaatcttaatgaaagcctgatagatttgcaagaacttggtaagta
tgagcaggggtacatacccgaggctcctcgggatgggcaggcctatgtacgcaaagacggtgaatgggtattgctcagcacttttctc
ggctgataa (SEQ ID NO: 98)
gccaccauggacuggaccuggauacucuuucucguagcagcagccacacgagugcauucaucacagugcguuaaucugac
cacccguacacaacucccacccgcauacacaaauagcuuuacacgcggaguguauuaccccgauaaagucuuucggagcu
cagugcuccauucuacucaagaucuuuuccugccguucuuuaguaacguuacuugguuucaugcaauacaugugucugg
cacaaacggaaccaaacguuuugauaauccgguguugccauuuaaugaugguguauauuuugcuuccacggaaaaguca
aacaucauccguggguggaucuuuggcaccacucuugauagcaaaugucaaagccuucugauuguuaauaacgcuacaa
acgucguaauuaaagugugugaauuccaguucuguaaugaccccuuccucggaguauauuaccacaagaauaacaaaucu
uggauggagagcgaauuuagaguuuacaguucagccaauaacuguacauuugaauaugucagucagccuuuccucaugg
accucgaagguaaacaagguaauuuuaagaacuugagagaguucguguuuaagaacaucgauggcuauuucaaaauuua
cucuaagcacacaccaaucaaccugguucgagaccugccccaggguuucucagcuuuggaaccauugguggaccugccaa
ucggcauuaacauuaccagauuucaaacuuuguuggcacuccaccggucauaucuuacccccggagacaguuccucaggc
uggacggcaggcgccgccgcguacuauguuggguaucuccaaccccgaaccuuccuucucaaauacaaugaaaacgggac
gauuacagaugcagucgauugcgcccuggaccccuuguccgaaacuaaaugcacucugaagaguuucacgguagagaag
ggaaucuaucaaacgagcaauuuucgaguccaaccaacggaaucuauugugcgguuucccaauaucacaaaccucugucc
auucggagaagucuuuaaugcuaccagguuugcgucuguauaugcauggaaccgaaagaggauuuccaauugcguagcg
gacuacaguguccuuuauaacagcgcuucauuuuccacguuuaaguguuaugguguuucuccaacgaaacucaacgacc
ucuguuuuacuaacguuuacgcugacagcuuuguuauacguggggacgaagucaggcaaauugcuccuggacagacugg
aaagaucgcugauuauaauuauaaacuuccugacgauuucaccggcugcguuauugcauggaacuccaacaaucuggau
ucaaaaguggguggaaauuauaauuaucuguauagguuguuucggaagagcaaucuuaagcccuuugagcgggacauau
guaccgaaauuuaccaagcaggcuccaccccaugcaauggaguagaaggguucaauugcuauuuuccucugcaaaguua
uggcuuucaacccaccaacggaguuggguaucaaccuuacaggguugucgugcugaguuucgaauugcuccacgcaccc
gcuacaguauguggccccaagaaguccacuaaucuuguuaagaauaaaugcgugaacuucaacuucaauggacuuacagg
uacuggaguacucacggaaucaaacaagaaauuucucccauuucaacaguuuggccgagauauagcugacaccacagaug
cuguucgcgacccccagacguuggaaauacuugauaucacucccugcagcuucggcggcgugagcgugaucacuccagg
uacuaauacgagcaaucaaguugccguucuguaccaagaugugaacugcaccgagguuccaguggcaauucacgccgacc
aacuuacucccaccuggcgggucuauuccaccggaucaaacgucuuccaaacucgcgcugguugccuuaucggugcaga
gcacguuaauaauuccuaugaaugugacauucccauaggagcaggcaucugugcaucuuaucaaacccagacuaauuccc
cugguuccgcuuccucuguugcaucccaguccauaauugccuacacuaugagucucggggcugaaaauuccguggccua
uucuaauaauucaaucgccaucccaaccaauuuuaccauauccguaacgacugaaauacuuccugucaguaugaccaaga
ccucaguggacugcaccauguacaucugcggcgauucuacugaauguuccaaucugcuuuugcaauaugguucauucug
cacccaacucaacagggcucuuacagggaucgccgucgaacaggauaagaauacccaggaaguguucgcccaaguuaagc
aaauuuacaagacaccacccaucaaggacuucggcggguucaacuucagccaaauucugcccgacccgucuaagccuucu
aagcgcucuuucauugaggaucuuuuguucaauaagguuacgcuugccgaugcaggguuuaucaaacaguauggcgacu
gucuuggggauaucgcagcuagggaucuuauuugugcacagaaauuuaauggccugacuguucuucccccuuugcucac
ugacgagaugauugcccaguacacuucagcucuccuggccgggacuauaacuucugguuggaccuucggagcuggcgcc
gcccugcaaauuccauuugcaaugcagauggcuuaucgcuucaacggaauuggggugacccaaaauguucucuacgaga
accagaaacucauugcaaaccaguucaauucugcgaucgggaagauccaggauucccugucuaguacggcuagugcccuc
gguaagcuccaagacgucgucaaccaaaacgcccaggccuugaacacccuugucaaacaacugagcuccaauuuuggggc
uauuagcagugugcugaaugauauccugucccgccuugacccaccggaagcggaaguccaaauugaucgacugaucacu
gggcgucuccaaucccuucaaacuuacgugacccaacaacucauccgagcagcugagauuagggcuagcgcuaaccuugc
ugcuacuaagaugucagaguguguccucggccagucuaagagaguggacuuuugugggaaaggguaccacuugauguc
auucccacaaagcgccccacacggcgugguguuucuccacgucacuuacguuccagcucaggaaaagaacuuuaccaccg
cccccgcuauaugucaugaugggaaggcccacuuuccucgugaaggugucuuugucagcaauggcacacacugguuugu
gacccaacggaauuucuaugagccucagauuauuaccacggauaacacuuucguaucagggaauugugaugugguuauc
ggcaucguuaauaauacaguguaugacccacugcagccagagcuugacagcuucaaagaagagcucgauaaguacuuuaa
gaaucauacaaguccugacguugaucuuggggauauuagugggauuaacgccagcgucgucaauauucagaaagagauu
gacagguugaacgaaguagcuaagaaucuuaaugaaagccugauagauuugcaagaacuugguaaguaugagcaggggu
acauacccgaggcuccucgggaugggcaggccuauguacgcaaagacggugaauggguauugcucagcacuuuucucgg
cugauaa (SEQ ID NO: 99)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT
TLDSK [symbol] QSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVRDLPQGFSA
LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYL
YRLFRKSNLKPFERDI [symbol] TEIYQAGSTPCNGVEGFNCYFPLQSYGFQPTNGVGYQPYRV
VVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS
VLNDILSRLD [symbol] EAEVQIDRLITGRLQSLQTYVTQQLIRAAEIRASANLAATKMSECVL
GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQGYIPEAPRDGQAYVRKDGEWVLLSTFLG** (SEQ ID NO: 100)
WuS_IgE_DownDS1 _2P_pVax
gccaccatggactggacctggatactctttctcgtagcagcagccacacgagtgcattcatcccagtgcgtgaacctgaccacccgaa
ctcaactcccaccagcatacaccaactcatttacaagaggagtttattacccggacaaggtatttcgaagttcagttcttcacagcaccca
agacctgtttctgccattcttcagtaatgtcacttggtttcacgcgatacatgtcagcggtacaaacgggacaaagcgattcgataaccca
gtactcccattcaacgacggagtgtattttgcatctacagagaaatccaacattatacgcgggtggatctttggaactactctggactcca
agacacagagcctgctcattgtgaacaatgcaacgaatgtcgtcataaaagtctgtgaatttcaattttgcaacgatcctttcctcggagtc
tattaccataagaacaataagagttggatggagagtgagtttcgcgtctattcttccgcgaacaattgtacatttgaatatgtatcacaacc
ctttcttatggatttggaaggcaaacaaggtaacttcaagaacttgcgcgagttcgtgttcaagaacatagactgttattttaagatctatag
taagcatacgccaatcaatctggtgcgagatttgcctcagggcttttctgctcttgaacccttggttgatctgcccatcgggatcaacataa
ccagatttcaaacgttgctcgcactccaccgcagctatctcactcctggcgattcctcatctgggtggaccgccggagctgctgcttatta
cgtcggctatctccagccgcgtactttcctgctcaagtataatgagaatggcaccattaccgatgctgtggattgtgctcttgatccactct
ctgaaaccaaatgcactctcaagtcttttaccgtggaaaagggtatttatcagacatctaattttcgggtgcaacctactgagtcaattgtac
ggtttcctaacataactaacctttgtccatttggggaagtcttcaatgccacgcggttcgcatcagtctatgcatggaacagaaaacgtatc
tccaactgcgtcgccgattattccgtcctttacaatagcgctagcttttccacattcaaatgttatggcgtatcaccaaccaaacttaacgat
ctctgctttactaatgtctacgctgactctttcgttattcgaggtgacgaggtgcgccaaattgcgcctggtcaaaccggaaagattgccg
attataactacaagctccccgacgactttacgggttgtgtgatcgcctggaatagcaataacctcgattctaaagttggcggtaattataac
tatctgtacagactctttaggaaaagtaatctcaagcccttttgcagggatatctcaaccgaaatctaccaagccggcagcactccttgca
atggtgtcgaggggtttaattgttatttcccactgcaatcttacggctttcaaccgactaatggagtcggttatcaaccctatagggtggtg
gtactctcctttgaacttttgcacgctccggcaacagtttgtggaccaaagaaaagtacgaaccttgttaagaataagtgtgttaatttcaat
tttaacggcctcactggaacaggtgtcctcacagaaagcaacaagaagtttctccctttccaacagtttggacgggatatcgccgacact
actgacgccgtcagagatcctcaaactctcgaaatcttggatatcacaccatgttctttcggtggtgtctccgtcataacaccaggaacta
acacctctaatcaagtggccgtgctctatcaggacgtcaattgcacagaagtgcctgtcgcaatccatgctgatcagctcactcccacct
ggcgtgtgtattccactggctctaatgtctttcagacacgggcaggttgccttattggggcagagcatgtgaacaattcctacgaatgcg
atatacccattggggcaggcatttgcgccagctaccaaacccaaactaacagccccgggagtgccagcagcgtggcatctcagtcca
ttattgcctatacgatgagcctgggtgctgaaaatagcgtggcttatagtaataactctatcgccatacccacaaacttcaccatttcagtg
accaccgaaatccttcctgtttctatgaccaaaacgtccgtcgattgtacaatgtacatttgcggcgatagcactgaatgttcaaacctgct
cctgcaatacggctctttctgtactcagctcaaccgggcactcaccggcatagccgtcgaacaagacaagaatacccaggaagtctttg
cgcaggtgaaacaaatctataagaccccaccaataaaagatttcggcggttttaatttcagccaaatcttgcctgatcccagcaagccat
ctaaacggtctttcattgaagatctcctgttcaacaaggttacgctggctgacgccgggtttattaagcaatatggcgattgccttgggga
cattgccgcacgagacctcatttgtgcccagaaattcaacgggctcaccgtattgcccccgctcctcacagacgaaatgatcgcccaat
atacaagcgccctgcttgcgggaaccattacaagcggttggacctttggtgccggcgcagctctgcaaatacccttcgcaatgcaaat
ggcatatcggtttaatggaattggcgtaacccaaaacgtgctgtatgaaaaccagaaactgatcgcaaatcaattcaatagtgctatagg
aaagatccaagacagtctgtcttccactgctagcgcgctggggaagctccaagacgttgtgaaccaaaacgcgcaggccctgaatac
cctggtgaagcaactttcaagcaatttcggtgctatatcttctgtcctcaatgacattctctctcggctcgatcccccggaagccgaagttc
agatagaccgtttgatcacaggccgcttgcaatccctgcaaacctacgttacacaacaactgattcgcgccgccgaaattcgggcatcc
gccaatctggccgcaaccaaaatgtccgagtgtgttctcggtcaatccaaacgcgtggatttctgcggaaaaggataccatttgatgtca
tttccacaatcagctccacacggtgttgtattcctgcacgtgacctacgtgccagcccaggagaagaattttactactgcgcccgccattt
gtcatgacgggaaggctcattttcctcgggaaggggttttcgtctcaaacggtacccattggttcgtgactcagaggaacttttatgaacc
tcaaatcataacgaccgataacacgtttgtaagtggcaattgcgacgtggtcatcgggattgtaaacaatactgtctatgaccctctccaa
ccagagcttgacagctttaaagaagagcttgataaatactttaagaaccatacctcaccagacgtcgatttgggagatatcagtggcatt
aatgcctctgtcgtcaatatccagaaagagattgaccgcttgaacgaagttgccaagaatcttaatgagtctctgattgacttgcaagaatt
gggaaaatatgaacaaggatatattccagaagcccctcgcgatgggcaagcatatgttcgaaaggatggggaatgggtgctgctcag
cacctttctcggttgataa (SEQ ID NO: 101)
gccaccauggacuggaccuggauacucuuucucguagcagcagccacacgagugcauucaucccagugcgugaaccugac
cacccgaacucaacucccaccagcauacaccaacucauuuacaagaggaguuuauuacccggacaagguauuucgaaguu
caguucuucacagcacccaagaccuguuucugccauucuucaguaaugucacuugguuucacgcgauacaugucagcgg
uacaaacgggacaaagcgauucgauaacccaguacucccauucaacgacggaguguauuuugcaucuacagagaaaucca
acauuauacgcggguggaucuuuggaacuacucuggacuccaagacacagagccugcucauugugaacaaugcaacgaau
gucgucauaaaagucugugaauuucaauuuugcaacgauccuuuccucggagucuauuaccauaagaacaauaagaguu
ggauggagagugaguuucgcgucuauucuuccgcgaacaauuguacauuugaauauguaucacaacccuuucuuaugga
uuuggaaggcaaacaagguaacuucaagaacuugcgcgaguucguguucaagaacauagacuguuauuuuaagaucuau
aguaagcauacgccaaucaaucuggugcgagauuugccucagggcuuuucugcucuugaacccuugguugaucugccca
ucgggaucaacauaaccagauuucaaacguugcucgcacuccaccgcagcuaucucacuccuggcgauuccucaucuggg
uggaccgccggagcugcugcuuauuacgucggcuaucuccagccgcguacuuuccugcucaaguauaaugagaauggca
ccauuaccgaugcuguggauugugcucuugauccacucucugaaaccaaaugcacucucaagucuuuuaccguggaaaa
ggguauuuaucagacaucuaauuuucgggugcaaccuacugagucaauuguacgguuuccuaacauaacuaaccuuugu
ccauuuggggaagucuucaaugccacgcgguucgcaucagucuaugcauggaacagaaaacguaucuccaacugcgucg
ccgauuauuccguccuuuacaauagcgcuagcuuuuccacauucaaauguuauggcguaucaccaaccaaacuuaacgau
cucugcuuuacuaaugucuacgcugacucuuucguuauucgaggugacgaggugcgccaaauugcgccuggucaaaccg
gaaagauugccgauuauaacuacaagcuccccgacgacuuuacggguugugugaucgccuggaauagcaauaaccucga
uucuaaaguuggcgguaauuauaacuaucuguacagacucuuuaggaaaaguaaucucaagcccuuuugcagggauauc
ucaaccgaaaucuaccaagccggcagcacuccuugcaauggugucgagggguuuaauuguuauuucccacugcaaucuu
acggcuuucaaccgacuaauggagucgguuaucaacccuauaggguggugguacucuccuuugaacuuuugcacgcucc
ggcaacaguuuguggaccaaagaaaaguacgaaccuuguuaagaauaaguguguuaauuucaauuuuaacggccucacu
ggaacagguguccucacagaaagcaacaagaaguuucucccuuuccaacaguuuggacgggauaucgccgacacuacuga
cgccgucagagauccucaaacucucgaaaucuuggauaucacaccauguucuuucgguggugucuccgucauaacaccag
gaacuaacaccucuaaucaaguggccgugcucuaucaggacgucaauugcacagaagugccugucgcaauccaugcugau
cagcucacucccaccuggcguguguauuccacuggcucuaaugucuuucagacacgggcagguugccuuauuggggcag
agcaugugaacaauuccuacgaaugcgauauacccauuggggcaggcauuugcgccagcuaccaaacccaaacuaacagc
cccgggagugccagcagcguggcaucucaguccauuauugccuauacgaugagccugggugcugaaaauagcguggcuu
auaguaauaacucuaucgccauacccacaaacuucaccauuucagugaccaccgaaauccuuccuguuucuaugaccaaaa
cguccgucgauuguacaauguacauuugcggcgauagcacugaauguucaaaccugcuccugcaauacggcucuuucug
uacucagcucaaccgggcacucaccggcauagccgucgaacaagacaagaauacccaggaagucuuugcgcaggugaaac
aaaucuauaagaccccaccaauaaaagauuucggcgguuuuaauuucagccaaaucuugccugaucccagcaagccaucu
aaacggucuuucauugaagaucuccuguucaacaagguuacgcuggcugacgccggguuuauuaagcaauauggcgauu
gccuuggggacauugccgcacgagaccucauuugugcccagaaauucaacgggcucaccguauugcccccgcuccucaca
gacgaaaugaucgcccaauauacaagcgcccugcuugcgggaaccauuacaagcgguuggaccuuuggugccggcgcag
cucugcaaauacccuucgcaaugcaaauggcauaucgguuuaauggaauuggcguaacccaaaacgugcuguaugaaaac
cagaaacugaucgcaaaucaauucaauagugcuauaggaaagauccaagacagucugucuuccacugcuagcgcgcuggg
gaagcuccaagacguugugaaccaaaacgcgcaggcccugaauacccuggugaagcaacuuucaagcaauuucggugcua
uaucuucuguccucaaugacauucucucucggcucgaucccccggaagccgaaguucagauagaccguuugaucacagg
ccgcuugcaaucccugcaaaccuacguuacacaacaacugauucgcgccgccgaaauucgggcauccgccaaucuggccg
caaccaaaauguccgaguguguucucggucaauccaaacgcguggauuucugcggaaaaggauaccauuugaugucauu
uccacaaucagcuccacacgguguuguauuccugcacgugaccuacgugccagcccaggagaagaauuuuacuacugcgc
ccgccauuugucaugacgggaaggcucauuuuccucgggaagggguuuucgucucaaacgguacccauugguucgugac
ucagaggaacuuuuaugaaccucaaaucauaacgaccgauaacacguuuguaaguggcaauugcgacguggucaucggg
auuguaaacaauacugucuaugacccucuccaaccagagcuugacagcuuuaaagaagagcuugauaaauacuuuaagaa
ccauaccucaccagacgucgauuugggagauaucaguggcauuaaugccucugucgucaauauccagaaagagauugacc
gcuugaacgaaguugccaagaaucuuaaugagucucugauugacuugcaagaauugggaaaauaugaacaaggauauau
uccagaagccccucgcgaugggcaagcauauguucgaaaggauggggaaugggugcugcucagcaccuuucucgguuga
uaa (SEQ ID NO: 102)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT
TLDSKTQSLLIVNNATNVVIKVCEFQFCNDPFLGVYYHKNNKSWMESEFRVYSSAN
NCTFEYVSQPFLMDLEGKQGNFKNLREFVFKNID [symbol] YFKIYSKHTPINLVRDLPQGFSA
LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYL
YRLFRKSNLKPFCRDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS
VLNDILSRLDPPEAEVQIDRLITGRLQSLQTYVTQQLIRAAEIRASANLAATKMSECVL
GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQGYIPEAPRDGQAYVRKDGEWVLLSTFLG** (SEQ ID NO: 103)
WuS_IgE_2P_UpGly_pVax
gccaccatggattggacctggatacttttcctcgtggccgcagcaacaagagtccactcctctcagtgcgttaacctgactactagaacc
caattgcccccggcatacacaaactctttcacccggggtgtctactatcccgacaaagtgtttagaagtagcgtgctgcacagcaccca
agatctctttctgccattcttctcaaacgtcacctggtttcacgccatccatgtaagcgggaccaacggcacaaagcgttttgataaccct
gttttgccattcaatgatggcgtgtattttgcttccactgagaaaagcaacatcattagagggtggatatttggcacaacgcttgactccaa
gacgcagagtcttttgatagtaaacaacgcaactaatgtggtcattaaagtctgtgaatttcaattttgcaatgaccccttccttggagtctat
taccacaagaacaacaaaagctggatggaaagcgaatttagggtctacagctctgccaataactgcacattcgaatacgtcagccaac
cattcttgatggacctggaaggcaagcaaggaaactttaagaatctgagggaatttgtgtttaagaatatcgacggatattttaagatctat
tccaagcatactcccattaatctcgttcgtgaccttcctcagggtttctctgcattggaacccctcgtagatttgcccattgggattaatatca
ctagattccagacgctgcttgcactccatcgatcttatctgacccctggtgactcctcttccgggtggacggcgggtgctgcagcctact
acgttggctatttgcaacctaggacctttctgttgaagtataatgagaatgggactattactgatgccgttgattgcgccctcgatccgctgt
cagaaacaaagtgcaccctgaagagcttcacagtagaaaagggaatctatcaaacctcaaatttccgcgttcaaccaactgaatcaatc
gtgcgttttcctaacatcacaaatctgtgtccgtttggagaagtatttaatgcgacgcgtttcgcaagcgtctacgcgtggaatcgcaaac
gtatctctaattgcgtagcagattattctgtgctgtacaatagcgcatctttctcaacgtttaagtgctacggcgttaatgggaccaagctga
atgatctctgtttcactaatgtgtacgcagacagttttgtaattagaggagacgaggttaggcaaatagcaccgggtcaaactggcaaaa
tcgccgactataactacaagctccctgatgacttcacgggctgcgtaattgcttggaactctaataacctggactctaaagtcggcggga
attataattatctctatcggttgtttcgaaaatccaatctcaaaccctttgagcgggacatcaatactacaatttatcaagctggtagtactcct
tgcaatggggtagaaggcttcaattgttatttcccccttcaatcttacggatttcaacccacgaacggcgtagggtaccagccctatcgag
tggtggtactgtcattcgaacttaatcacgccccagcaacagtctgcgggcctaagaaaagcacgaatcttgtcaagaataagtgtgta
aatttcaacttcaatggtcttacaggcacgggagtgctcactgagtctaataagaaatttcttcctttccaacaattcggtcgtgatattgcc
gatactactgatgcagtccgagatccacaaactctcgaaatcctcgatattactccttgtagttttggcggcgtctccgtgatcaccccag
ggaccaacactagtaaccaagtggcggtgctctaccaagatgttaactgcacagaagtcccggtagcgatccatgccgaccagctca
ctcccacatggcgtgtttacagcacagggtcaaacgttttccagacccgtgccggatgtcttataggagccgaacacgtaaataacagt
tatgaatgcgatatcccaattggtgcaggtatctgtgcgtcatatcaaacccaaactaattctccggggtccgcctcaagcgttgcctcac
aatcaataatcgcctacacaatgtccctcggtgccgaaaattcagtcgcttactctaacaatagcattgctatccctaccaacttcactattt
ctgttaccacggaaattttgcctgtatccatgaccaaaacatctgttgattgcacgatgtacatctgcggggattctaccgaatgttctaac
ctgcttctgcaatacggctccttctgcacccaattgaaccgcgcactgactgggattgctgtggaacaagacaagaatactcaagaagt
atttgcccaggtcaaacagatttacaaaactcccccaattaaagatttcggcggtttcaattttagtcaaattctgccagatccaagtaagc
catccaaacgctcatttattgaggacctgctctttaataaagtcacgctggccgacgccggcttcataaaacagtatggcgattgtcttgg
agacatcgccgcccgcgacctcatttgcgcacaaaagttcaatgggctcaccgtgttgccaccactgctcacagatgagatgatcgca
cagtacacgagcgcccttcttgccggcactatcacgtctggttggacgttcggtgccggagccgctctgcaaattccctttgcaatgcaa
atggcctatagatttaatggaattggcgtaacacagaacgtgttgtacgagaaccagaagctcattgccaaccagttcaattccgctattg
gcaaaatacaagactctctcagctcaactgctagcgcactgggaaaattgcaagacgtagtcaatcaaaatgcccaagccctcaatact
ctcgtcaaacagttgtcttccaactttggggctatcagtagtgtactcaatgacattctttcaagactggacccgcccgaggcggaagtcc
aaattgatcgtctgataactggaaggttgcaaagccttcagacctacgttacgcaacaacttattagggctgccgaaataagggcatcc
gctaatctggcagctacaaagatgtctgaatgtgttttgggacagagcaaacgggttgacttctgcggtaaaggttaccatctcatgtcttt
tccacaaagcgcaccgcacggagtcgtcttcctgcatgtaacatacgtcccagcccaagaaaagaattttaccacagccccagccatc
tgccacgacggcaaggcgcatttcccaagggaaggcgtgtttgtatccaacgggacgcattggtttgtcactcaaaggaacttttacga
accccaaattattaccactgataacaccttcgtttctgggaactgtgatgtcgtgattgggatagtaaacaacacggtatatgatccactgc
aaccagaactggattccttcaaagaagagctggacaaatacttcaagaatcatactagtcctgacgtcgacctgggcgatatcagtgga
atcaacgctagcgtcgtaaacattcaaaaggagatcgatagacttaacgaggtcgccaagaatctcaatgaaagcctcatcgatttgca
agaactcggaaaatatgagcaaagcggatcagggtacattccggaagcccccagggacggacaggcatatgtccgcaaggacgga
gaatgggttcttcttagcacttttctggggtaatga (SEQ ID NO: 104)
gccaccauggauuggaccuggauacuuuuccucguggccgcagcaacaagaguccacuccucucagugcguuaaccugac
uacuagaacccaauugcccccggcauacacaaacucuuucacccggggugucuacuaucccgacaaaguguuuagaagua
gcgugcugcacagcacccaagaucucuuucugccauucuucucaaacgucaccugguuucacgccauccauguaagcggg
accaacggcacaaagcguuuugauaacccuguuuugccauucaaugauggcguguauuuugcuuccacugagaaaagca
acaucauuagaggguggauauuuggcacaacgcuugacuccaagacgcagagucuuuugauaguaaacaacgcaacuaau
guggucauuaaagucugugaauuucaauuuugcaaugaccccuuccuuggagucuauuaccacaagaacaacaaaagcu
ggauggaaagcgaauuuagggucuacagcucugccaauaacugcacauucgaauacgucagccaaccauucuugauggac
cuggaaggcaagcaaggaaacuuuaagaaucugagggaauuuguguuuaagaauaucgacggauauuuuaagaucuauu
ccaagcauacucccauuaaucucguucgugaccuuccucaggguuucucugcauuggaaccccucguagauuugcccau
ugggauuaauaucacuagauuccagacgcugcuugcacuccaucgaucuuaucugaccccuggugacuccucuuccggg
uggacggcgggugcugcagccuacuacguuggcuauuugcaaccuaggaccuuucuguugaaguauaaugagaauggg
acuauuacugaugccguugauugcgcccucgauccgcugucagaaacaaagugcacccugaagagcuucacaguagaaaa
gggaaucuaucaaaccucaaauuuccgcguucaaccaacugaaucaaucgugcguuuuccuaacaucacaaaucuguguc
cguuuggagaaguauuuaaugcgacgcguuucgcaagcgucuacgcguggaaucgcaaacguaucucuaauugcguagc
agauuauucugugcuguacaauagcgcaucuuucucaacguuuaagugcuacggcguuaaugggaccaagcugaaugau
cucuguuucacuaauguguacgcagacaguuuuguaauuagaggagacgagguuaggcaaauagcaccgggucaaacug
gcaaaaucgccgacuauaacuacaagcucccugaugacuucacgggcugcguaauugcuuggaacucuaauaaccuggac
ucuaaagucggcgggaauuauaauuaucucuaucgguuguuucgaaaauccaaucucaaacccuuugagcgggacauca
auacuacaauuuaucaagcugguaguacuccuugcaaugggguagaaggcuucaauuguuauuucccccuucaaucuua
cggauuucaacccacgaacggcguaggguaccagcccuaucgaguggugguacugucauucgaacuuaaucacgccccag
caacagucugcgggccuaagaaaagcacgaaucuugucaagaauaaguguguaaauuucaacuucaauggucuuacaggc
acgggagugcucacugagucuaauaagaaauuucuuccuuuccaacaauucggucgugauauugccgauacuacugaug
caguccgagauccacaaacucucgaaauccucgauauuacuccuuguaguuuuggcggcgucuccgugaucaccccagg
gaccaacacuaguaaccaaguggcggugcucuaccaagauguuaacugcacagaagucccgguagcgauccaugccgacc
agcucacucccacauggcguguuuacagcacagggucaaacguuuuccagacccgugccggaugucuuauaggagccga
acacguaaauaacaguuaugaaugcgauaucccaauuggugcagguaucugugcgucauaucaaacccaaacuaauucuc
cgggguccgccucaagcguugccucacaaucaauaaucgccuacacaaugucccucggugccgaaaauucagucgcuuac
ucuaacaauagcauugcuaucccuaccaacuucacuauuucuguuaccacggaaauuuugccuguauccaugaccaaaac
aucuguugauugcacgauguacaucugcggggauucuaccgaauguucuaaccugcuucugcaauacggcuccuucugc
acccaauugaaccgcgcacugacugggauugcuguggaacaagacaagaauacucaagaaguauuugcccaggucaaaca
gauuuacaaaacucccccaauuaaagauuucggcgguuucaauuuuagucaaauucugccagauccaaguaagccaucca
aacgcucauuuauugaggaccugcucuuuaauaaagucacgcuggccgacgccggcuucauaaaacaguauggcgauug
ucuuggagacaucgccgcccgcgaccucauuugcgcacaaaaguucaaugggcucaccguguugccaccacugcucacag
augagaugaucgcacaguacacgagcgcccuucuugccggcacuaucacgucugguuggacguucggugccggagccgc
ucugcaaauucccuuugcaaugcaaauggccuauagauuuaauggaauuggcguaacacagaacguguuguacgagaac
cagaagcucauugccaaccaguucaauuccgcuauuggcaaaauacaagacucucucagcucaacugcuagcgcacuggg
aaaauugcaagacguagucaaucaaaaugcccaagcccucaauacucucgucaaacaguugucuuccaacuuuggggcua
ucaguaguguacucaaugacauucuuucaagacuggacccgcccgaggcggaaguccaaauugaucgucugauaacugg
aagguugcaaagccuucagaccuacguuacgcaacaacuuauuagggcugccgaaauaagggcauccgcuaaucuggcag
cuacaaagaugucugaauguguuuugggacagagcaaacggguugacuucugcgguaaagguuaccaucucaugucuuu
uccacaaagcgcaccgcacggagucgucuuccugcauguaacauacgucccagcccaagaaaagaauuuuaccacagcccc
agccaucugccacgacggcaaggcgcauuucccaagggaaggcguguuuguauccaacgggacgcauugguuugucacu
caaaggaacuuuuacgaaccccaaauuauuaccacugauaacaccuucguuucugggaacugugaugucgugauuggga
uaguaaacaacacgguauaugauccacugcaaccagaacuggauuccuucaaagaagagcuggacaaauacuucaagaau
cauacuaguccugacgucgaccugggcgauaucaguggaaucaacgcuagcgucguaaacauucaaaaggagaucgauag
acuuaacgaggucgccaagaaucucaaugaaagccucaucgauuugcaagaacucggaaaauaugagcaaagcggaucag
gguacauuccggaagcccccagggacggacaggcauauguccgcaaggacggagaauggguucuucuuagcacuuuucu
gggguaauga (SEQ ID NO: 105)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT
TLDSKTQSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVRDLPQGFSA
LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGV [symbol] TKLNDLCFTNVY
ADSFVIRGDEVRQIAPGQTGKIADYNYKLPDDFTGCVIAWNSNNLDSKVGGNYNYL
YRLFRKSNLKPFERDI [symbol] T [symbol] IYQAGSTPCNGVEGFNCYFPLQSYGFQPTNGVGYQPYRV
VVLSFEL [symbol] HAPATVCGPKKSTNLVKNKCVNFNFNGLTGTGVLTESNKKFLPFQQFGR
DIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS
VLNDILSRLDPPEAEVQIDRLITGRLQSLQTYVTQQLIRAAEIRASANLAATKMSECVL
GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQSGSGYIPEAPRDGQAYVRKDGEWVLLSTFLG** (SEQ ID NO: 106)
WuS_DownDS3_D2P_Furin_pVax
gccaccatggactggacctggatactctttctcgtagcagcagccacacgagtgcattcaatgttcgttttcctcgtgctcttgcctttggtt
tcttctcagtgcgtaaacctcacgactcgaacccaactgcccccagcttatacaaattcctttacgcggggcgtctattacccggataag
gttttcagatccagcgtgctgcatagtacacaagatctctttcttcctttcttctcaaatgtaacctggtttcacgctattcatgtatccggcac
caatggaactaaaagatttgataacccggtgttgcccttcaatgatggtgtgtatttcgcttccacggaaaagtcaaacatcatcagaggg
tggatattcggcacaacattggattccaagtgccagtcactcctcatagtgaacaatgctactaacgtggttataaaggtctgcgaatttca
attttgtaatgatcctttcctcggtgtttactatcacaagaacaataagtcctggatggaatcagaattccgtgtatacagttctgcgaacaat
tgcacattcgaatatgtgtcccaaccctttctcatggatctggaagggaagcagggtaactttaagaatctgagagaattcgtgttcaaga
acattgactgctattttaaaatctatagcaaacacacccctataaacttggtacgggatttgcctcaaggattctcagcactcgaacccttg
gtcgatttgccaatcggcatcaatatcacccggtttcagacactcctggctcttcaccgctcctacttgacacctggtgattcctcatctggt
tggaccgccggcgcagcggcatactatgtcggctatcttcaaccaagaaccttcttgctgaaatataatgagaacggaactataactgat
gccgttgattgtgcccttgatccacttagcgaaacaaagtgcactctgaagtccttcacagttgaaaaggggatctaccaaacatccaac
ttccgggtacaacctactgagtccatagtgcgatttcctaacattaccaatctgtgcccatttggagaagtattcaacgcaactaggttcgc
gtccgtttacgcgtggaacaggaaaaggatttccaattgcgtcgccgactatagcgttctctataacagcgcctcatttagcacgtttaag
tgttacggggttagtccgaccaaactcaatgacttgtgttttaccaatgtctatgcagactcctttgttattagaggcgacgaggtcagaca
aattgcccccggacagacaggtaagattgcagattataattataaactgccggacgacttcacggggtgtgttattgcatggaactccaa
taacctggactctaaagtaggcgggaactataactatctgtatcgcctgtttcgcaaatctaacctgaaacccttctgcagggacatatgt
actgaaatatatcaagctggcagcacaccttgtaatggcgtcgagggattcaattgttacttcccacttcaatcttacggttttcagcctact
aacggcgtagggtatcaaccctatagagttgtagtgctctctttcgaattgctccatgcccccgcgactgtttgtggacctaagaagtcca
cgaacctggtaaagaacaagtgtgttaattttaattttaatggactgaccgggactggagtgctgactgaaagtaacaagaaatttctgcc
tttccaacaatttggccgcgatatcgctgataccaccgacgccgtcagagatccgcagactctcgaaatcctggacatcacgccctgct
cattcggcggggttagcgttattactccaggcactaacactagcaatcaagttgcagttctgtaccaggatgtgaactgtaccgaagtcc
ccgtcgccattcatgccgatcagctgaccccgacttggcgggtatattcaaccggcagcaatgtctttcaaacaagggcgggttgtctc
atcggagcggagcatgtaaataatagttatgaatgcgacatccccattggcgcggggatctgtgcttcatatcaaactcaaaccaattcc
ccacggcggagacgatcagtagccagtcaatcaataattgcgtatacgatgagtcttggggcagaaaatagcgtggcttattctaataat
agcatcgctatacctacaaattttacaatcagtgtaactaccgaaatccttcctgtcagcatgaccaaaactagcgtagattgcacgatgt
atatttgcggagactcaactgagtgcagtaacctgttgttgcaatacggaagtttctgtacccagctgaaccgcgctcttacgggcattgc
agtagaacaagataagaatacccaagaagtgtttgcccaggtgaaacaaatctacaagactcccccgattaaagactttggcgggttca
acttcagccagatattgcccgacccgtctcgtcgtagacggtcctttattgaagacctgctcttcaacaaggtcacactggctgatgcag
gttttattaagcaatacggcgactgtcttggcgacatcgccgctagggaccttatatgtgctcagaaattcaatggtctgacagttctgcca
cccttgctcactgacgaaatgatcgctcaatatacaagcgccttgctggctgggactattacttccggatggacattcggggcgggtgc
cgccttgcaaattccttttgcaatgcaaatggcataccgtttcaacggaatcggcgtaacccagaatgtgctctatgaaaaccagaaattg
atagcaaatcaatttaactcagccataggaaagattcaagactctctcagctcaaccgcgagtgctctcggcaagctccaagacgtagt
aaatcaaaatgcacaagctttgaacactttggtaaagcaattgtcttccaacttcggggcgatctcatctggccctaacgacatcctgtcc
cggttgcccaaagtggaagccgaggtgcagatcgaccgcctcatcaccggccgacttcaatcactccaaacctacgtgactcaacaa
ctgatccgggcagccgagataagggcgagtgcaaacttggcagctacgaaaatgtcagaatgtgttctcggccagagtaaacgggta
gacttttgtgggaaaggttatcacttgatgtctttccctcaaagcgctcctcacggcgtcgtcttcttgcatgtgacttacgtgccagctcaa
gaaaagaacttcaccaccgcccctgctatatgccatgacggtaaagctcacttcccccgagagggcgtgttcgttagtaatggaaccc
attggtttgtgactcaacgaaacttttatgaacctcaaataattaccacggataacacttttgttagtggtaattgtgacgtggtgatcggcat
tgtgaataacacagtctacgatcctctgcaaccagaactggacagctttaaagaggaacttgacaaatatttcaagaaccatacaagcc
ccgacgtcgacctgggcgacatcagtggaatcaatgcgtccgtagtcaatatccagaaggagattgatcggcttaatgaagtcgctaa
gaatttgaatgaaagtcttatagatctgcaagaactcgggaagtacgagcaatatattaaatggccttggtccggacgtagaaggcgca
ggcggggctcaggcggttcagggtcagggtatattcccgaggcgccacgcgatgggcaagcgtacgtgcgtaaagatggcgaatg
ggtgttgctttccacattcttggggtgataa (SEQ ID NO: 107)
gccaccauggacuggaccuggauacucuuucucguagcagcagccacacgagugcauucaauguucguuuuccucgugc
ucuugccuuugguuucuucucagugcguaaaccucacgacucgaacccaacugcccccagcuuauacaaauuccuuuacg
cggggcgucuauuacccggauaagguuuucagauccagcgugcugcauaguacacaagaucucuuucuuccuuucuucu
caaauguaaccugguuucacgcuauucauguauccggcaccaauggaacuaaaagauuugauaacccgguguugcccuu
caaugaugguguguauuucgcuuccacggaaaagucaaacaucaucagaggguggauauucggcacaacauuggauucc
aagugccagucacuccucauagugaacaaugcuacuaacgugguuauaaaggucugcgaauuucaauuuuguaaugauc
cuuuccucgguguuuacuaucacaagaacaauaaguccuggauggaaucagaauuccguguauacaguucugcgaacaa
uugcacauucgaauaugugucccaacccuuucucauggaucuggaagggaagcaggguaacuuuaagaaucugagagaa
uucguguucaagaacauugacugcuauuuuaaaaucuauagcaaacacaccccuauaaacuugguacgggauuugccuca
aggauucucagcacucgaacccuuggucgauuugccaaucggcaucaauaucacccgguuucagacacuccuggcucuuc
accgcuccuacuugacaccuggugauuccucaucugguuggaccgccggcgcagcggcauacuaugucggcuaucuuca
accaagaaccuucuugcugaaauauaaugagaacggaacuauaacugaugccguugauugugcccuugauccacuuagc
gaaacaaagugcacucugaaguccuucacaguugaaaaggggaucuaccaaacauccaacuuccggguacaaccuacuga
guccauagugcgauuuccuaacauuaccaaucugugcccauuuggagaaguauucaacgcaacuagguucgcguccguu
uacgcguggaacaggaaaaggauuuccaauugcgucgccgacuauagcguucucuauaacagcgccucauuuagcacgu
uuaaguguuacgggguuaguccgaccaaacucaaugacuuguguuuuaccaaugucuaugcagacuccuuuguuauuag
aggcgacgaggucagacaaauugcccccggacagacagguaagauugcagauuauaauuauaaacugccggacgacuuca
cgggguguguuauugcauggaacuccaauaaccuggacucuaaaguaggcgggaacuauaacuaucuguaucgccuguu
ucgcaaaucuaaccugaaacccuucugcagggacauauguacugaaauauaucaagcuggcagcacaccuuguaauggcg
ucgagggauucaauuguuacuucccacuucaaucuuacgguuuucagccuacuaacggcguaggguaucaacccuauag
aguuguagugcucucuuucgaauugcuccaugcccccgcgacuguuuguggaccuaagaaguccacgaaccugguaaag
aacaaguguguuaauuuuaauuuuaauggacugaccgggacuggagugcugacugaaaguaacaagaaauuucugccuu
uccaacaauuuggccgcgauaucgcugauaccaccgacgccgucagagauccgcagacucucgaaauccuggacaucacg
cccugcucauucggcgggguuagcguuauuacuccaggcacuaacacuagcaaucaaguugcaguucuguaccaggaug
ugaacuguaccgaaguccccgucgccauucaugccgaucagcugaccccgacuuggcggguauauucaaccggcagcaau
gucuuucaaacaagggcggguugucucaucggagcggagcauguaaauaauaguuaugaaugcgacauccccauuggcg
cggggaucugugcuucauaucaaacucaaaccaauuccccacggcggagacgaucaguagccagucaaucaauaauugcg
uauacgaugagucuuggggcagaaaauagcguggcuuauucuaauaauagcaucgcuauaccuacaaauuuuacaauca
guguaacuaccgaaauccuuccugucagcaugaccaaaacuagcguagauugcacgauguauauuugcggagacucaacu
gagugcaguaaccuguuguugcaauacggaaguuucuguacccagcugaaccgcgcucuuacgggcauugcaguagaac
aagauaagaauacccaagaaguguuugcccaggugaaacaaaucuacaagacucccccgauuaaagacuuuggcggguuc
aacuucagccagauauugcccgacccgucucgucguagacgguccuuuauugaagaccugcucuucaacaaggucacacu
ggcugaugcagguuuuauuaagcaauacggcgacugucuuggcgacaucgccgcuagggaccuuauaugugcucagaaa
uucaauggucugacaguucugccacccuugcucacugacgaaaugaucgcucaauauacaagcgccuugcuggcuggga
cuauuacuuccggauggacauucggggcgggugccgccuugcaaauuccuuuugcaaugcaaauggcauaccguuucaa
cggaaucggcguaacccagaaugugcucuaugaaaaccagaaauugauagcaaaucaauuuaacucagccauaggaaaga
uucaagacucucucagcucaaccgcgagugcucucggcaagcuccaagacguaguaaaucaaaaugcacaagcuuugaac
acuuugguaaagcaauugucuuccaacuucggggcgaucucaucuggcccuaacgacauccugucccgguugcccaaag
uggaagccgaggugcagaucgaccgccucaucaccggccgacuucaaucacuccaaaccuacgugacucaacaacugauc
cgggcagccgagauaagggcgagugcaaacuuggcagcuacgaaaaugucagaauguguucucggccagaguaaacggg
uagacuuuugugggaaagguuaucacuugaugucuuucccucaaagcgcuccucacggcgucgucuucuugcaugugac
uuacgugccagcucaagaaaagaacuucaccaccgccccugcuauaugccaugacgguaaagcucacuucccccgagagg
gcguguucguuaguaauggaacccauugguuugugacucaacgaaacuuuuaugaaccucaaauaauuaccacggauaa
cacuuuuguuagugguaauugugacguggugaucggcauugugaauaacacagucuacgauccucugcaaccagaacug
gacagcuuuaaagaggaacuugacaaauauuucaagaaccauacaagccccgacgucgaccugggcgacaucaguggaau
caaugcguccguagucaauauccagaaggagauugaucggcuuaaugaagucgcuaagaauuugaaugaaagucuuaua
gaucugcaagaacucgggaaguacgagcaauauauuaaauggccuugguccggacguagaaggcgcaggcggggcucag
gcgguucagggucaggguauauucccgaggcgccacgcgaugggcaagcguacgugcguaaagauggcgaaugggugu
ugcuuuccacauucuuggggugauaa (SEQ ID NO: 108)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKCQSLLIVNNATNVVIKVCEFQFCNDPFLGVYYHKNNKSWM
ESEFRVYSSANNCTFEYVSQPFLMDLEGKQGNFKNLREFVFKNID [symbol] YFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPF [symbol] RDI [symbol] TEIYQAGSTPCNGVEGFNCYFPLQSYGFQPT
NGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRR [symbol] RSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIY
KTPPIKDFGGFNFSQILPDPS [symbol] RSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISS [symbol] NDILSRL [symbol] KVEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 109)
WuS_DownDS3_D2P_F_NoTriCle_pVax
gccaccatggactggacctggatactctttctcgtagcagcagccacacgagtgcattcaatgttcgtgttcctcgtgctcctgcctctcg
ttagcagccaatgtgttaatctcaccaccagaacacagctcccacccgcgtatactaactcttttacgaggggagtttattatcccgataa
ggttttccggtctagcgtactccactccacccaagatctgttcctgcctttctttagcaacgtgacgtggtttcatgcaatccacgtgagtg
gcaccaatggaaccaagcggttcgataatcctgtgttgccgtttaacgatggcgtgtattttgcctcaactgaaaagtctaacataatacg
cggctggatcttcgggaccacattggatagtaagtgtcaatctctgcttatcgtgaacaacgctactaacgtcgttataaaggtctgtgag
ttccaattctgcaacgacccattcctgggtgtgtattaccacaagaataataaatcttggatggagtctgagtttcgcgtatactcttctgcta
acaactgcacctttgaatatgtaagtcaaccattcctcatggatctggaaggaaaacaaggcaactttaagaacttgcgggaatttgtctt
caagaacatcgactgttattttaaaatttactcaaaacacaccccgattaatctggtccgcgatttgccccaagggttctctgcattggaac
cccttgtggacttgcctataggaattaatatcacccgctttcaaactcttctggcgctgcaccgtagctacctgacaccaggagatagctc
tagtggctggactgctggagccgcggcatattatgtggggtatctgcagccacgtacatttctcctcaaatataatgaaaatggtacaata
acggatgcagtcgactgcgcattggaccctctgagtgaaacaaaatgcactctcaagagcttcactgttgaaaagggcatataccaaa
catctaattttagagtccaacccactgaatccattgtccgatttcctaatattacaaacctctgcccatttggagaagtgttcaacgccacta
ggtttgcatccgtgtacgcatggaacagaaaacgaatttctaattgtgtggcagactatagcgtgctgtataactcagcaagctttagcac
atttaagtgttatggagttagcccaaccaaattgaatgatctttgtttcacgaacgtgtacgccgatagcttcgttattcgaggggacgagg
tgaggcaaatcgctccaggtcaaaccggtaaaatcgccgattacaattataaacttcctgatgacttcactggctgtgtcatagcatgga
actctaataatctcgacagcaaggtcggtgggaactataactatctttatcgactctttagaaagagtaatctcaaaccattttgcagagac
atttgtacagagatttatcaggcagggagcacaccatgtaatggggtcgagggcttcaactgttacttccccctgcaatcttatgggttcc
agccgaccaatggagtgggctaccaaccttatcgcgtggtggtcctgtcttttgaactgcttcatgctccagccaccgtatgcggcccta
agaagtctacaaatttggtcaagaacaagtgcgtcaattttaacttcaatggtctgactggaaccggtgtcctcacagaatctaacaagaa
atttctgccatttcaacaatttggaagagatatcgcggatactacggatgctgttagggacccccaaacacttgaaattctcgacattaca
ccctgttcctttggcggggtcagtgtcattaccccgggtacaaatactagtaaccaagtcgcagtactgtatcaagatgttaattgtaccg
aagtgccggtagcaatacacgctgatcaacttacaccaacatggcgagtgtattctacggggagtaatgtcttccaaacgcgggccgg
gtgtctgattggcgcggaacacgtaaacaactcctacgaatgtgatattccaataggcgcaggcatatgtgcgagctatcaaacacaaa
ctaactcccctagacggcgtcggagtgtggctagtcaatcaatcattgcctatacaatgtctctgggagcagaaaacagcgtggcatatt
ccaataattccatcgctatacctaccaactttaccatcagcgtcactactgagattcttcccgtctccatgacgaaaacttccgttgattgta
ctatgtacatctgcggagacagcaccgaatgcagtaaccttctcttgcaatatggcagcttttgtactcagctcaacagagctctcacag
gtattgccgtcgaacaagataagaacacccaagaggtgttcgcccaggtgaaacagatatataagaccccacccatcaaggatttcgg
cgggtttaattttagtcaaatcctgcccgatccctcacggcgtcgcaggtcctttattgaagatcttctgttcaataaggtcacactcgctga
cgcaggctttatcaagcagtatggagattgtctgggcgatatagctgcgagggacttgatctgcgcacaaaagttcaacggccttacag
tgctgcccccgttgctgacagatgagatgattgcgcaatacacttccgcgcttctcgcagggaccatcacgagcggctggacgttcgg
cgctggcgccgctctgcaaatcccgtttgcaatgcaaatggcctataggtttaatggtatcggtgtaacgcaaaacgtactttatgaaaac
cagaaactgatcgctaaccaattcaattccgctattggcaaaattcaagacagcctcagcagcacggctagtgcactgggtaaactcca
agacgtggtgaaccaaaatgcccaagcattgaatacacttgtcaagcaacttagttccaacttcggtgcaatttcaagtggtccaaatga
catacttagcaggctgcctaaagtagaagccgaagtgcaaatcgatagacttatcaccggccgcctgcaatcccttcaaacatacgtga
ctcagcagcttatcagggctgctgagattcgagcaagtgcgaacctggccgccaccaaaatgagtgagtgcgtccttgggcaatcca
agcgcgttgacttttgtggtaaggggtatcatctcatgagcttcccccaatccgcccctcacggagtagtgtttctccatgtgacgtatgtt
cctgcacaagagaagaacttcacaacggctccggctatatgtcatgacggaaaagcgcactttcctcgcgaaggagtgtttgtgtcaaa
tggaacgcactggttcgtgacgcaaaggaatttctacgagcctcaaatcatcactacagataatacttttgtctctgggaattgcgacgtg
gtcattggaatcgtcaacaatacggtttacgatcccctgcaaccagaactggattcattcaaagaagaactcgacaagtacttcaagaat
cataccagtcctgatgtggatctgggcgatatcagtgggatcaatgcaagcgttgtcaacattcaaaaggaaatagaccgcctcaacga
agtcgcaaagaatctcaatgaaagccttattgatcttcaagagctcggaaaatatgagcaatatattaagtggccttggtccggcggctc
aggcggaagtggctcaggatatattcctgaggctccccgagatggacaagcatacgtgagaaaagatggggagtgggtgttgctga
gtacgttccttggatgataa (SEQ ID NO: 110)
gccaccauggacuggaccuggauacucuuucucguagcagcagccacacgagugcauucaauguucguguuccucgugc
uccugccucucguuagcagccaauguguuaaucucaccaccagaacacagcucccacccgcguauacuaacucuuuuacg
aggggaguuuauuaucccgauaagguuuuccggucuagcguacuccacuccacccaagaucuguuccugccuuucuuua
gcaacgugacgugguuucaugcaauccacgugaguggcaccaauggaaccaagcgguucgauaauccuguguugccguu
uaacgauggcguguauuuugccucaacugaaaagucuaacauaauacgcggcuggaucuucgggaccacauuggauagu
aagugucaaucucugcuuaucgugaacaacgcuacuaacgucguuauaaaggucugugaguuccaauucugcaacgacc
cauuccuggguguguauuaccacaagaauaauaaaucuuggauggagucugaguuucgcguauacucuucugcuaacaa
cugcaccuuugaauauguaagucaaccauuccucauggaucuggaaggaaaacaaggcaacuuuaagaacuugcgggaau
uugucuucaagaacaucgacuguuauuuuaaaauuuacucaaaacacaccccgauuaaucugguccgcgauuugccccaa
ggguucucugcauuggaaccccuuguggacuugccuauaggaauuaauaucacccgcuuucaaacucuucuggcgcugc
accguagcuaccugacaccaggagauagcucuaguggcuggacugcuggagccgcggcauauuaugugggguaucugca
gccacguacauuucuccucaaauauaaugaaaaugguacaauaacggaugcagucgacugcgcauuggacccucugagug
aaacaaaaugcacucucaagagcuucacuguugaaaagggcauauaccaaacaucuaauuuuagaguccaacccacugaa
uccauuguccgauuuccuaauauuacaaaccucugcccauuuggagaaguguucaacgccacuagguuugcauccgugu
acgcauggaacagaaaacgaauuucuaauuguguggcagacuauagcgugcuguauaacucagcaagcuuuagcacauu
uaaguguuauggaguuagcccaaccaaauugaaugaucuuuguuucacgaacguguacgccgauagcuucguuauucga
ggggacgaggugaggcaaaucgcuccaggucaaaccgguaaaaucgccgauuacaauuauaaacuuccugaugacuucac
uggcugugucauagcauggaacucuaauaaucucgacagcaaggucggugggaacuauaacuaucuuuaucgacucuuu
agaaagaguaaucucaaaccauuuugcagagacauuuguacagagauuuaucaggcagggagcacaccauguaaugggg
ucgagggcuucaacuguuacuucccccugcaaucuuauggguuccagccgaccaauggagugggcuaccaaccuuaucg
cguggugguccugucuuuugaacugcuucaugcuccagccaccguaugcggcccuaagaagucuacaaauuuggucaag
aacaagugcgucaauuuuaacuucaauggucugacuggaaccgguguccucacagaaucuaacaagaaauuucugccauu
ucaacaauuuggaagagauaucgcggauacuacggaugcuguuagggacccccaaacacuugaaauucucgacauuacac
ccuguuccuuuggcggggucagugucauuaccccggguacaaauacuaguaaccaagucgcaguacuguaucaagaugu
uaauuguaccgaagugccgguagcaauacacgcugaucaacuuacaccaacauggcgaguguauucuacggggaguaau
gucuuccaaacgcgggccgggugucugauuggcgcggaacacguaaacaacuccuacgaaugugauauuccaauaggcg
caggcauaugugcgagcuaucaaacacaaacuaacuccccuagacggcgucggaguguggcuagucaaucaaucauugcc
uauacaaugucucugggagcagaaaacagcguggcauauuccaauaauuccaucgcuauaccuaccaacuuuaccaucag
cgucacuacugagauucuucccgucuccaugacgaaaacuuccguugauuguacuauguacaucugcggagacagcacc
gaaugcaguaaccuucucuugcaauauggcagcuuuuguacucagcucaacagagcucucacagguauugccgucgaac
aagauaagaacacccaagagguguucgcccaggugaaacagauauauaagaccccacccaucaaggauuucggcggguuu
aauuuuagucaaauccugcccgaucccucacggcgucgcagguccuuuauugaagaucuucuguucaauaaggucacac
ucgcugacgcaggcuuuaucaagcaguauggagauugucugggcgauauagcugcgagggacuugaucugcgcacaaaa
guucaacggccuuacagugcugcccccguugcugacagaugagaugauugcgcaauacacuuccgcgcuucucgcaggg
accaucacgagcggcuggacguucggcgcuggcgccgcucugcaaaucccguuugcaaugcaaauggccuauagguuua
augguaucgguguaacgcaaaacguacuuuaugaaaaccagaaacugaucgcuaaccaauucaauuccgcuauuggcaaa
auucaagacagccucagcagcacggcuagugcacuggguaaacuccaagacguggugaaccaaaaugcccaagcauugaa
uacacuugucaagcaacuuaguuccaacuucggugcaauuucaagugguccaaaugacauacuuagcaggcugccuaaag
uagaagccgaagugcaaaucgauagacuuaucaccggccgccugcaaucccuucaaacauacgugacucagcagcuuauc
agggcugcugagauucgagcaagugcgaaccuggccgccaccaaaaugagugagugcguccuugggcaauccaagcgcg
uugacuuuugugguaagggguaucaucucaugagcuucccccaauccgccccucacggaguaguguuucuccaugugac
guauguuccugcacaagagaagaacuucacaacggcuccggcuauaugucaugacggaaaagcgcacuuuccucgcgaag
gaguguuugugucaaauggaacgcacugguucgugacgcaaaggaauuucuacgagccucaaaucaucacuacagauaa
uacuuuugucucugggaauugcgacguggucauuggaaucgucaacaauacgguuuacgauccccugcaaccagaacug
gauucauucaaagaagaacucgacaaguacuucaagaaucauaccaguccugauguggaucugggcgauaucaguggga
ucaaugcaagcguugucaacauucaaaaggaaauagaccgccucaacgaagucgcaaagaaucucaaugaaagccuuauu
gaucuucaagagcucggaaaauaugagcaauauauuaaguggccuugguccggcggcucaggcggaaguggcucaggau
auauuccugaggcuccccgagauggacaagcauacgugagaaaagauggggaguggguguugcugaguacguuccuug
gaugauaa (SEQ ID NO: 111)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSK [symbol] QSLLIVNNATNVVIKVCEFQFCNDPFLGVYYHKNNKSWM
ESEFRVYSSANNCTFEYVSQPFLMDLEGKQGNFKNLREFVFKNID [symbol] YFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPF [symbol] RDI [symbol] TEIYQAGSTPCNGVEGFNCYFPLQSYGFQPT
NGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRR [symbol] RSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIY
KTPPIKDFGGFNFSQILPDPS [symbol] RSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISS [symbol] NDILSRL [symbol] KVEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGGSGGSGSGYIPEAPRDGQAYVRKDGEWVLLSTFLG**
(SEQ ID NO: 112)
WuS_DownDS2_2P_pVax
gccaccatggactggacctggatactctttctcgtagcagcagccacacgagtgcattcaatgttcgtgttcttggtgctgctgcctcttgt
ctcatcacagtgcgttaatctgaccacccgtacacaactcccacccgcatacacaaatagctttacacgcggagtgtattaccccgataa
agtctttcggagctcagtgctccattctactcaagatcttttcctgccgttctttagtaacgttacttggtttcatgcaatacatgtgtctggca
caaacggaaccaaacgttttgataatccggtgttgccatttaatgatggtgtatattttgcttccacggaaaagtcaaacatcatccgtggg
tggatctttggcaccactcttgatagcaaatgtcaaagccttctgattgttaataacgctacaaacgtcgtaattaaagtgtgtgaattccag
ttctgtaatgaccccttcctcggagtatattaccacaagaataacaaatcttggatggagagcgaatttagagtttacagttcagccaataa
ctgtacatttgaatatgtcagtcagcctttcctcatggacctcgaaggtaaacaaggtaattttaagaacttgagagagttcgtgtttaagaa
catcgatggctatttcaaaatttactctaagcacacaccaatcaacctggttcgagacctgccccagggtttctcagctttggaaccattg
gtggacctgccaatcggcattaacattaccagatttcaaactttgttggcactccaccggtcatatcttacccccggagacagttcctcag
gctggacggcaggcgccgccgcgtactatgttgggtatctccaaccccgaaccttccttctcaaatacaatgaaaacgggacgattac
agatgcagtcgattgcgccctggaccccttgtccgaaactaaatgcactctgaagagtttcacggtagagaagggaatctatcaaacga
gcaattttcgagtccaaccaacggaatctattgtgcggtttcccaatatcacaaacctctgtccattcggagaagtctttaatgctaccagg
tttgcgtctgtatatgcatggaaccgaaagaggatttccaattgcgtagcggactacagtgtcctttataacagcgcttcattttccacgttt
aagtgttatggtgtttctccaacgaaactcaacgacctctgttttactaacgtttacgctgacagctttgttatacgtggggacgaagtcag
gcaaattgctcctggacagactggaaagatcgctgattataattataaacttcctgacgatttcaccggctgcgttattgcatggaactcca
acaatctggattcaaaagtgggtggaaattataattatctgtataggttgtttcggaagagcaatcttaagccctttgagcgggacatatgt
accgaaatttaccaagcaggctccaccccatgcaatggagtagaagggttcaattgctattttcctctgcaaagttatggctttcaaccca
ccaacggagttgggtatcaaccttacagggttgtcgtgctgagtttcgaattgctccacgcacccgctacagtatgtggccccaagaag
tccactaatcttgttaagaataaatgcgtgaacttcaacttcaatggacttacaggtactggagtactcacggaatcaaacaagaaatttct
cccatttcaacagtttggccgagatatagctgacaccacagatgctgttcgcgacccccagacgttggaaatacttgatatcactccctg
cagcttcggcggcgtgagcgtgatcactccaggtactaatacgagcaatcaagttgccgttctgtaccaagatgtgaactgcaccgag
gttccagtggcaattcacgccgaccaacttactcccacctggcgggtctattccaccggatcaaacgtcttccaaactcgcgctggttgc
cttatcggtgcagagcacgttaataattcctatgaatgtgacattcccataggagcaggcatctgtgcatcttatcaaacccagactaattc
ccctggttccgcttcctctgttgcatcccagtccataattgcctacactatgagtctcggggctgaaaattccgtggcctattctaataattc
aatcgccatcccaaccaattttaccatatccgtaacgactgaaatacttcctgtcagtatgaccaagacctcagtggactgcaccatgtac
atctgcggcgattctactgaatgttccaatctgcttttgcaatatggttcattctgcacccaactcaacagggctcttacagggatcgccgt
cgaacaggataagaatacccaggaagtgttcgcccaagttaagcaaatttacaagacaccacccatcaaggacttcggcgggttcaa
cttcagccaaattctgcccgacccgtctaagccttctaagcgctctttcattgaggatcttttgttcaataaggttacgcttgccgatgcagg
gtttatcaaacagtatggcgactgtcttggggatatcgcagctagggatcttatttgtgcacagaaatttaatggcctgactgttcttccccc
tttgctcactgacgagatgattgcccagtacacttcagctctcctggccgggactataacttctggttggaccttcggagctggcgccgc
cctgcaaattccatttgcaatgcagatggcttatcgcttcaacggaattggggtgacccaaaatgttctctacgagaaccagaaactcatt
gcaaaccagttcaattctgcgatcgggaagatccaggattccctgtctagtacggctagtgccctcggtaagctccaagacgtcgtcaa
ccaaaacgcccaggccttgaacacccttgtcaaacaactgagctccaattttggggctattagcagtgtgctgaatgatatcctgtcccg
ccttgacccaccggaagcggaagtccaaattgatcgactgatcactgggcgtctccaatcccttcaaacttacgtgacccaacaactca
tccgagcagctgagattagggctagcgctaaccttgctgctactaagatgtcagagtgtgtcctcggccagtctaagagagtggactttt
gtgggaaagggtaccacttgatgtcattcccacaaagcgccccacacggcgtggtgtttctccacgtcacttacgttccagctcaggaa
aagaactttaccaccgcccccgctatatgtcatgatgggaaggcccactttcctcgtgaaggtgtctttgtcagcaatggcacacactgg
tttgtgacccaacggaatttctatgagcctcagattattaccacggataacactttcgtatcagggaattgtgatgtggttatcggcatcgtt
aataatacagtgtatgacccactgcagccagagcttgacagcttcaaagaagagctcgataagtactttaagaatcatacaagtcctgac
gttgatcttggggatattagtgggattaacgccagcgtcgtcaatattcagaaagagattgacaggttgaacgaagtagctaagaatctt
aatgaaagcctgatagatttgcaagaacttggtaagtatgagcaggggtacatacccgaggctcctcgggatgggcaggcctatgtac
gcaaagacggtgaatgggtattgctcagcacttttctcggctgataa (SEQ ID NO: 113)
gccaccauggacuggaccuggauacucuuucucguagcagcagccacacgagugcauucaauguucguguucuuggugc
ugcugccucuugucucaucacagugcguuaaucugaccacccguacacaacucccacccgcauacacaaauagcuuuaca
cgcggaguguauuaccccgauaaagucuuucggagcucagugcuccauucuacucaagaucuuuuccugccguucuuua
guaacguuacuugguuucaugcaauacaugugucuggcacaaacggaaccaaacguuuugauaauccgguguugccauu
uaaugaugguguauauuuugcuuccacggaaaagucaaacaucauccguggguggaucuuuggcaccacucuugauagc
aaaugucaaagccuucugauuguuaauaacgcuacaaacgucguaauuaaagugugugaauuccaguucuguaaugacc
ccuuccucggaguauauuaccacaagaauaacaaaucuuggauggagagcgaauuuagaguuuacaguucagccaauaac
uguacauuugaauaugucagucagccuuuccucauggaccucgaagguaaacaagguaauuuuaagaacuugagagagu
ucguguuuaagaacaucgauggcuauuucaaaauuuacucuaagcacacaccaaucaaccugguucgagaccugccccag
gguuucucagcuuuggaaccauugguggaccugccaaucggcauuaacauuaccagauuucaaacuuuguuggcacucc
accggucauaucuuacccccggagacaguuccucaggcuggacggcaggcgccgccgcguacuauguuggguaucucca
accccgaaccuuccuucucaaauacaaugaaaacgggacgauuacagaugcagucgauugcgcccuggaccccuuguccg
aaacuaaaugcacucugaagaguuucacgguagagaagggaaucuaucaaacgagcaauuuucgaguccaaccaacggaa
ucuauugugcgguuucccaauaucacaaaccucuguccauucggagaagucuuuaaugcuaccagguuugcgucuguau
augcauggaaccgaaagaggauuuccaauugcguagcggacuacaguguccuuuauaacagcgcuucauuuuccacguu
uaaguguuaugguguuucuccaacgaaacucaacgaccucuguuuuacuaacguuuacgcugacagcuuuguuauacgu
ggggacgaagucaggcaaauugcuccuggacagacuggaaagaucgcugauuauaauuauaaacuuccugacgauuuca
ccggcugcguuauugcauggaacuccaacaaucuggauucaaaaguggguggaaauuauaauuaucuguauagguuguu
ucggaagagcaaucuuaagcccuuugagcgggacauauguaccgaaauuuaccaagcaggcuccaccccaugcaauggag
uagaaggguucaauugcuauuuuccucugcaaaguuauggcuuucaacccaccaacggaguuggguaucaaccuuacag
gguugucgugcugaguuucgaauugcuccacgcacccgcuacaguauguggccccaagaaguccacuaaucuuguuaag
aauaaaugcgugaacuucaacuucaauggacuuacagguacuggaguacucacggaaucaaacaagaaauuucucccauu
ucaacaguuuggccgagauauagcugacaccacagaugcuguucgcgacccccagacguuggaaauacuugauaucacuc
ccugcagcuucggcggcgugagcgugaucacuccagguacuaauacgagcaaucaaguugccguucuguaccaagaugu
gaacugcaccgagguuccaguggcaauucacgccgaccaacuuacucccaccuggcgggucuauuccaccggaucaaacg
ucuuccaaacucgcgcugguugccuuaucggugcagagcacguuaauaauuccuaugaaugugacauucccauaggagc
aggcaucugugcaucuuaucaaacccagacuaauuccccugguuccgcuuccucuguugcaucccaguccauaauugccu
acacuaugagucucggggcugaaaauuccguggccuauucuaauaauucaaucgccaucccaaccaauuuuaccauaucc
guaacgacugaaauacuuccugucaguaugaccaagaccucaguggacugcaccauguacaucugcggcgauucuacuga
auguuccaaucugcuuuugcaauaugguucauucugcacccaacucaacagggcucuuacagggaucgccgucgaacag
gauaagaauacccaggaaguguucgcccaaguuaagcaaauuuacaagacaccacccaucaaggacuucggcggguucaa
cuucagccaaauucugcccgacccgucuaagccuucuaagcgcucuuucauugaggaucuuuuguucaauaagguuacg
cuugccgaugcaggguuuaucaaacaguauggcgacugucuuggggauaucgcagcuagggaucuuauuugugcacag
aaauuuaauggccugacuguucuucccccuuugcucacugacgagaugauugcccaguacacuucagcucuccuggccg
ggacuauaacuucugguuggaccuucggagcuggcgccgcccugcaaauuccauuugcaaugcagauggcuuaucgcuu
caacggaauuggggugacccaaaauguucucuacgagaaccagaaacucauugcaaaccaguucaauucugcgaucggga
agauccaggauucccugucuaguacggcuagugcccucgguaagcuccaagacgucgucaaccaaaacgcccaggccuug
aacacccuugucaaacaacugagcuccaauuuuggggcuauuagcagugugcugaaugauauccugucccgccuugacc
caccggaagcggaaguccaaauugaucgacugaucacugggcgucuccaaucccuucaaacuuacgugacccaacaacuc
auccgagcagcugagauuagggcuagcgcuaaccuugcugcuacuaagaugucagaguguguccucggccagucuaaga
gaguggacuuuugugggaaaggguaccacuugaugucauucccacaaagcgccccacacggcgugguguuucuccacgu
cacuuacguuccagcucaggaaaagaacuuuaccaccgcccccgcuauaugucaugaugggaaggcccacuuuccucgug
aaggugucuuugucagcaauggcacacacugguuugugacccaacggaauuucuaugagccucagauuauuaccacgga
uaacacuuucguaucagggaauugugaugugguuaucggcaucguuaauaauacaguguaugacccacugcagccagag
cuugacagcuucaaagaagagcucgauaaguacuuuaagaaucauacaaguccugacguugaucuuggggauauuagug
ggauuaacgccagcgucgucaauauucagaaagagauugacagguugaacgaaguagcuaagaaucuuaaugaaagccu
gauagauuugcaagaacuugguaaguaugagcagggguacauacccgaggcuccucgggaugggcaggccuauguacgc
aaagacggugaauggguauugcucagcacuuuucucggcugauaa (SEQ ID NO: 114)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSK [symbol] QSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDI [symbol] TEIYQAGSTPCNGVEGFNCYFPLQSYGFQPT
NGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSP [symbol] A [symbol] SVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIY
KTPPIKDFGGFNFSQILPDPSKPSKRSFIEDLLFNKVTLADAGFIKQYGDCLGDIAARD
LICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTL
VKQLSSNFGAISSVLNDILSRLD [symbol] EAEVQIDRLITGRLQSLQTYVTQQLIRAAEIRASA
NLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKNL
NESLIDLQELGKYEQGYIPEAPRDGQAYVRKDGEWVLLSTFLG** (SEQ ID NO: 115)
WuSDownDS1_2P_pVax
gccaccatggactggacctggatactctttctcgtagcagcagccacacgagtgcattcaatgtttgttttccttgttctgctcccgcttgtg
tcttcccagtgcgtgaacctgaccacccgaactcaactcccaccagcatacaccaactcatttacaagaggagtttattacccggacaa
ggtatttcgaagttcagttcttcacagcacccaagacctgtttctgccattcttcagtaatgtcacttggtttcacgcgatacatgtcagcgg
tacaaacgggacaaagcgattcgataacccagtactcccattcaacgacggagtgtattttgcatctacagagaaatccaacattatacg
cgggtggatctttggaactactctggactccaagacacagagcctgctcattgtgaacaatgcaacgaatgtcgtcataaaagtctgtga
atttcaattttgcaacgatcctttcctcggagtctattaccataagaacaataagagttggatggagagtgagtttcgcgtctattcttccgc
gaacaattgtacatttgaatatgtatcacaaccctttcttatggatttggaaggcaaacaaggtaacttcaagaacttgcgcgagttcgtgtt
caagaacatagactgttattttaagatctatagtaagcatacgccaatcaatctggtgcgagatttgcctcagggcttttctgctcttgaacc
cttggttgatctgcccatcgggatcaacataaccagatttcaaacgttgctcgcactccaccgcagctatctcactcctggcgattcctcat
ctgggtggaccgccggagctgctgcttattacgtcggctatctccagccgcgtactttcctgctcaagtataatgagaatggcaccatta
ccgatgctgtggattgtgctcttgatccactctctgaaaccaaatgcactctcaagtcttttaccgtggaaaagggtatttatcagacatcta
attttcgggtgcaacctactgagtcaattgtacggtttcctaacataactaacctttgtccatttggggaagtcttcaatgccacgcggttcg
catcagtctatgcatggaacagaaaacgtatctccaactgcgtcgccgattattccgtcctttacaatagcgctagcttttccacattcaaat
gttatggcgtatcaccaaccaaacttaacgatctctgctttactaatgtctacgctgactctttcgttattcgaggtgacgaggtgcgccaa
attgcgcctggtcaaaccggaaagattgccgattataactacaagctccccgacgactttacgggttgtgtgatcgcctggaatagcaat
aacctcgattctaaagttggcggtaattataactatctgtacagactctttaggaaaagtaatctcaagcccttttgcagggatatctcaacc
gaaatctaccaagccggcagcactccttgcaatggtgtcgaggggtttaattgttatttcccactgcaatcttacggctttcaaccgactaa
tggagtcggttatcaaccctatagggtggtggtactctcctttgaacttttgcacgctccggcaacagtttgtggaccaaagaaaagtacg
aaccttgttaagaataagtgtgttaatttcaattttaacggcctcactggaacaggtgtcctcacagaaagcaacaagaagtttctccctttc
caacagtttggacgggatatcgccgacactactgacgccgtcagagatcctcaaactctcgaaatcttggatatcacaccatgttctttcg
gtggtgtctccgtcataacaccaggaactaacacctctaatcaagtggccgtgctctatcaggacgtcaattgcacagaagtgcctgtc
gcaatccatgctgatcagctcactcccacctggcgtgtgtattccactggctctaatgtctttcagacacgggcaggttgccttattgggg
cagagcatgtgaacaattcctacgaatgcgatatacccattggggcaggcatttgcgccagctaccaaacccaaactaacagccccg
ggagtgccagcagcgtggcatctcagtccattattgcctatacgatgagcctgggtgctgaaaatagcgtggcttatagtaataactctat
cgccatacccacaaacttcaccatttcagtgaccaccgaaatccttcctgtttctatgaccaaaacgtccgtcgattgtacaatgtacattt
gcggcgatagcactgaatgttcaaacctgctcctgcaatacggctctttctgtactcagctcaaccgggcactcaccggcatagccgtc
gaacaagacaagaatacccaggaagtctttgcgcaggtgaaacaaatctataagaccccaccaataaaagatttcggcggttttaatttc
agccaaatcttgcctgatcccagcaagccatctaaacggtctttcattgaagatctcctgttcaacaaggttacgctggctgacgccggg
tttattaagcaatatggcgattgccttggggacattgccgcacgagacctcatttgtgcccagaaattcaacgggctcaccgtattgcccc
cgctcctcacagacgaaatgatcgcccaatatacaagcgccctgcttgcgggaaccattacaagcggttggacctttggtgccggcgc
agctctgcaaatacccttcgcaatgcaaatggcatatcggtttaatggaattggcgtaacccaaaacgtgctgtatgaaaaccagaaact
gatcgcaaatcaattcaatagtgctataggaaagatccaagacagtctgtcttccactgctagcgcgctggggaagctccaagacgttg
tgaaccaaaacgcgcaggccctgaataccctggtgaagcaactttcaagcaatttcggtgctatatcttctgtcctcaatgacattctctct
cggctcgatcccccggaagccgaagttcagatagaccgtttgatcacaggccgcttgcaatccctgcaaacctacgttacacaacaac
tgattcgcgccgccgaaattcgggcatccgccaatctggccgcaaccaaaatgtccgagtgtgttctcggtcaatccaaacgcgtgga
tttctgcggaaaaggataccatttgatgtcatttccacaatcagctccacacggtgttgtattcctgcacgtgacctacgtgccagcccag
gagaagaattttactactgcgcccgccatttgtcatgacgggaaggctcattttcctcgggaaggggttttcgtctcaaacggtacccatt
ggttcgtgactcagaggaacttttatgaacctcaaatcataacgaccgataacacgtttgtaagtggcaattgcgacgtggtcatcggga
ttgtaaacaatactgtctatgaccctctccaaccagagcttgacagctttaaagaagagcttgataaatactttaagaaccatacctcacca
gacgtcgatttgggagatatcagtggcattaatgcctctgtcgtcaatatccagaaagagattgaccgcttgaacgaagttgccaagaat
cttaatgagtctctgattgacttgcaagaattgggaaaatatgaacaaggatatattccagaagcccctcgcgatgggcaagcatatgttc
gaaaggatggggaatgggtgctgctcagcacctttctcggttgataa (SEQ ID NO: 116)
gccaccauggacuggaccuggauacucuuucucguagcagcagccacacgagugcauucaauguuuguuuuccuuguuc
ugcucccgcuugugucuucccagugcgugaaccugaccacccgaacucaacucccaccagcauacaccaacucauuuacaa
gaggaguuuauuacccggacaagguauuucgaaguucaguucuucacagcacccaagaccuguuucugccauucuucag
uaaugucacuugguuucacgcgauacaugucagcgguacaaacgggacaaagcgauucgauaacccaguacucccauuca
acgacggaguguauuuugcaucuacagagaaauccaacauuauacgcggguggaucuuuggaacuacucuggacuccaa
gacacagagccugcucauugugaacaaugcaacgaaugucgucauaaaagucugugaauuucaauuuugcaacgauccu
uuccucggagucuauuaccauaagaacaauaagaguuggauggagagugaguuucgcgucuauucuuccgcgaacaauu
guacauuugaauauguaucacaacccuuucuuauggauuuggaaggcaaacaagguaacuucaagaacuugcgcgaguu
cguguucaagaacauagacuguuauuuuaagaucuauaguaagcauacgccaaucaaucuggugcgagauuugccucag
ggcuuuucugcucuugaacccuugguugaucugcccaucgggaucaacauaaccagauuucaaacguugcucgcacucc
accgcagcuaucucacuccuggcgauuccucaucuggguggaccgccggagcugcugcuuauuacgucggcuaucucca
gccgcguacuuuccugcucaaguauaaugagaauggcaccauuaccgaugcuguggauugugcucuugauccacucucu
gaaaccaaaugcacucucaagucuuuuaccguggaaaaggguauuuaucagacaucuaauuuucgggugcaaccuacug
agucaauuguacgguuuccuaacauaacuaaccuuuguccauuuggggaagucuucaaugccacgcgguucgcaucagu
cuaugcauggaacagaaaacguaucuccaacugcgucgccgauuauuccguccuuuacaauagcgcuagcuuuuccacau
ucaaauguuauggcguaucaccaaccaaacuuaacgaucucugcuuuacuaaugucuacgcugacucuuucguuauucg
aggugacgaggugcgccaaauugcgccuggucaaaccggaaagauugccgauuauaacuacaagcuccccgacgacuuua
cggguugugugaucgccuggaauagcaauaaccucgauucuaaaguuggcgguaauuauaacuaucuguacagacucuu
uaggaaaaguaaucucaagcccuuuugcagggauaucucaaccgaaaucuaccaagccggcagcacuccuugcaauggug
ucgagggguuuaauuguuauuucccacugcaaucuuacggcuuucaaccgacuaauggagucgguuaucaacccuauag
gguggugguacucuccuuugaacuuuugcacgcuccggcaacaguuuguggaccaaagaaaaguacgaaccuuguuaag
aauaaguguguuaauuucaauuuuaacggccucacuggaacagguguccucacagaaagcaacaagaaguuucucccuu
uccaacaguuuggacgggauaucgccgacacuacugacgccgucagagauccucaaacucucgaaaucuuggauaucaca
ccauguucuuucgguggugucuccgucauaacaccaggaacuaacaccucuaaucaaguggccgugcucuaucaggacg
ucaauugcacagaagugccugucgcaauccaugcugaucagcucacucccaccuggcguguguauuccacuggcucuaa
ugucuuucagacacgggcagguugccuuauuggggcagagcaugugaacaauuccuacgaaugcgauauacccauuggg
gcaggcauuugcgccagcuaccaaacccaaacuaacagccccgggagugccagcagcguggcaucucaguccauuauugc
cuauacgaugagccugggugcugaaaauagcguggcuuauaguaauaacucuaucgccauacccacaaacuucaccauuu
cagugaccaccgaaauccuuccuguuucuaugaccaaaacguccgucgauuguacaauguacauuugcggcgauagcacu
gaauguucaaaccugcuccugcaauacggcucuuucuguacucagcucaaccgggcacucaccggcauagccgucgaaca
agacaagaauacccaggaagucuuugcgcaggugaaacaaaucuauaagaccccaccaauaaaagauuucggcgguuuua
auuucagccaaaucuugccugaucccagcaagccaucuaaacggucuuucauugaagaucuccuguucaacaagguuacg
cuggcugacgccggguuuauuaagcaauauggcgauugccuuggggacauugccgcacgagaccucauuugugcccaga
aauucaacgggcucaccguauugcccccgcuccucacagacgaaaugaucgcccaauauacaagcgcccugcuugcggga
accauuacaagcgguuggaccuuuggugccggcgcagcucugcaaauacccuucgcaaugcaaauggcauaucgguuua
auggaauuggcguaacccaaaacgugcuguaugaaaaccagaaacugaucgcaaaucaauucaauagugcuauaggaaag
auccaagacagucugucuuccacugcuagcgcgcuggggaagcuccaagacguugugaaccaaaacgcgcaggcccugaa
uacccuggugaagcaacuuucaagcaauuucggugcuauaucuucuguccucaaugacauucucucucggcucgauccc
ccggaagccgaaguucagauagaccguuugaucacaggccgcuugcaaucccugcaaaccuacguuacacaacaacugau
ucgcgccgccgaaauucgggcauccgccaaucuggccgcaaccaaaauguccgaguguguucucggucaauccaaacgcg
uggauuucugcggaaaaggauaccauuugaugucauuuccacaaucagcuccacacgguguuguauuccugcacgugac
cuacgugccagcccaggagaagaauuuuacuacugcgcccgccauuugucaugacgggaaggcucauuuuccucgggaa
gggguuuucgucucaaacgguacccauugguucgugacucagaggaacuuuuaugaaccucaaaucauaacgaccgaua
acacguuuguaaguggcaauugcgacguggucaucgggauuguaaacaauacugucuaugacccucuccaaccagagcu
ugacagcuuuaaagaagagcuugauaaauacuuuaagaaccauaccucaccagacgucgauuugggagauaucaguggca
uuaaugccucugucgucaauauccagaaagagauugaccgcuugaacgaaguugccaagaaucuuaaugagucucugau
ugacuugcaagaauugggaaaauaugaacaaggauauauuccagaagccccucgcgaugggcaagcauauguucgaaag
gauggggaaugggugcugcucagcaccuuucucgguugauaa(SEQ ID NO: 117)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNID [symbol] YFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPF [symbol] RDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSP [symbol] A [symbol] SVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSVLNDILSRLD [symbol] EAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQGYIPEAPRDGQAYVRKDGEWVLLSTFLG** (SEQ ID NO: 118)
WuS_2P_pVax
gccaccatggactggacctggatactctttctcgtagcagcagccacacgagtgcattcaatgtttgtgtttcttgtcctgttgccattggtg
agctcccaatgtgtcaatctgaccacccggacacaattgccccctgcatatacaaattcattcacgagaggagtatactatcccgacaaa
gttttccggtcctcagtccttcattccactcaagatcttttccttccattcttttctaacgtaacctggttccatgcaattcatgtcagtgggacc
aacggcacgaaacggtttgataatccagttcttccattcaatgacggagtatattttgcatcaactgagaaatctaatatcattagagggtg
gattttcggaacaactcttgactccaagacccaatccttgctcatcgttaacaatgctacaaatgtggttattaaggtctgtgagtttcaattc
tgtaacgacccctttctcggcgtatactaccataagaataataagtcttggatggagtctgaatttcgtgtctactcatcagcgaacaattgt
acatttgaatatgtgtcccaaccattcctgatggatctcgaaggaaagcagggcaattttaagaaccttcgggagttcgtctttaagaatat
cgatggatactttaaaatatatagtaaacacacaccaatcaatctggtccgagatctcccccagggttttagtgctctggagccgctggtg
gatttgcccatcggtatcaatattacgcgcttccaaacattgctcgccctgcatcggagttaccttacgcctggcgacagtagcagcgga
tggaccgctggagccgccgcctactatgtcgggtaccttcaaccacgcacttttctcctgaaatacaacgaaaatgggacaattacaga
cgctgttgattgcgcactcgatcccctgtcagaaacaaaatgtacacttaaatcttttacggtcgagaaagggatttaccagacatctaac
ttccgagtacaaccaaccgaatctatagtgcggttccccaacattacgaacctgtgcccgttcggcgaagtgttcaacgcaacacgattt
gcttctgtttacgcttggaaccggaaacgcatctccaattgcgtcgccgattacagcgttctttataattctgcatctttctccaccttcaaat
gctatggtgtctctcccacaaaactcaatgacctctgttttaccaatgtgtatgcggactccttcgtcatacgcggcgacgaggtgagaca
aatcgcaccagggcagactggcaagatcgctgattataattacaaactgcctgatgattttaccggatgcgttattgcttggaattctaata
acctcgattccaaagttggcgggaactacaattacctctaccgattgtttcgcaaatctaaccttaagccgtttgagagagatatcagcac
agagatttatcaagctggctctaccccttgcaatggagtagaaggctttaactgctattttcctcttcagtcttatggatttcaacctaccaac
ggggtagggtaccaaccctatagagtcgtcgtgctctcatttgaactccttcacgcccccgctaccgtgtgtgggcctaagaaatccact
aatctcgttaagaataagtgtgtgaattttaatttcaatggcctgacagggaccggggttctgactgaatctaacaagaaatttctgccgtt
ccaacaattcgggcgcgatattgcagacacgaccgacgcggtgcgcgatcctcaaacactcgaaatccttgatatcactccttgttcttt
cggcggtgtaagcgtcattactcctggcaccaatacctctaaccaagtggcagtactctatcaagatgtgaactgcactgaggtcccgg
ttgcaatacatgcggatcaactcaccccaacatggcgagtgtattccacagggagcaatgtgtttcaaacgagggccggctgtctcatt
ggggccgaacacgttaataatagttatgagtgcgatattcccattggagcgggcatttgtgccagctatcagacccaaactaactcccc
cgggtccgcctcatcagtcgctagccaatctattattgcgtacacaatgtccctgggagctgaaaacagcgtggcctactcaaataaca
gcattgcaatacccacaaattttacgatttcagtaaccactgaaatcctgcccgtctccatgaccaaaacctctgtcgactgcactatgtac
atatgcggcgactccaccgagtgttccaatctccttctccaatatggaagtttctgcacgcagttgaacagggcacttacagggattgca
gtcgagcaagacaagaacacccaagaagtattcgcacaagtaaaacagatctacaagacacccccaatcaaagattttggtggcttca
acttctcccaaatacttccagatccgtcaaagccatccaaacgctcattcatcgaagaccttctgttcaataaggtcacattggcggatgc
tggatttatcaagcaatatggggattgtttgggagatattgcagcgcgggacctgatatgcgcgcaaaagttcaatgggttgacggtgct
gccccctctcctcactgacgagatgatagctcagtatacgagcgctctcctcgcgggcactatcacctcaggttggaccttcggggctg
gcgcggcacttcaaataccatttgctatgcaaatggcctatcgttttaatggcatcggggtgacccaaaacgtgctctatgaaaaccaga
aactgatagctaatcaattcaatagtgccatcggcaaaatccaggattcattgtccagcaccgcctcagctctcgggaaattgcaagac
gtcgtcaaccaaaatgctcaagcgctcaacaccctcgttaaacaactctcaagtaatttcggcgcgattagtagcgtgctgaacgatatc
ttgagtcgtcttgatccacctgaagcagaagtccaaatcgacaggcttattaccggacgtctgcaaagcctgcaaacctacgttacaca
acaacttataagggcagccgaaataagggcttctgcaaatctggctgccacgaagatgagcgagtgtgtcctcggacaaagcaaaag
agttgacttttgcggcaaagggtaccaccttatgagtttccctcagtctgcgccccatggagtagtgtttctccacgtgacttatgtaccgg
cacaagaaaagaactttaccacagccccagcaatatgtcacgatggaaaagcacactttccacgggaaggggttttcgtgtccaacgg
gacccattggtttgttactcaacgcaacttttatgaaccccaaatcataaccactgataatacatttgtctctgggaactgtgatgtcgtgat
cggaatagtcaacaacacagtgtatgatccgttgcaaccagagctggattccttcaaagaagaactcgacaagtattttaagaatcacac
atcaccggacgtggatcttggagacatatcaggcataaacgctagtgtggtgaatatccaaaaggagatcgacaggcttaacgaagtt
gcaaagaacctcaatgaatctcttatcgatttgcaagaattgggcaaatacgagcaaggctacattcctgaagcaccacgggacgggc
aagcttacgtgcggaaagatggcgaatgggtgctcttgagtacctttctgggttgataa (SEQ ID NO: 119)
gccaccauggacuggaccuggauacucuuucucguagcagcagccacacgagugcauucaauguuuguguuucuugucc
uguugccauuggugagcucccaaugugucaaucugaccacccggacacaauugcccccugcauauacaaauucauucacg
agaggaguauacuaucccgacaaaguuuuccgguccucaguccuucauuccacucaagaucuuuuccuuccauucuuuu
cuaacguaaccugguuccaugcaauucaugucagugggaccaacggcacgaaacgguuugauaauccaguucuuccauu
caaugacggaguauauuuugcaucaacugagaaaucuaauaucauuagaggguggauuuucggaacaacucuugacucc
aagacccaauccuugcucaucguuaacaaugcuacaaaugugguuauuaaggucugugaguuucaauucuguaacgacc
ccuuucucggcguauacuaccauaagaauaauaagucuuggauggagucugaauuucgugucuacucaucagcgaacaa
uuguacauuugaauaugugucccaaccauuccugauggaucucgaaggaaagcagggcaauuuuaagaaccuucgggag
uucgucuuuaagaauaucgauggauacuuuaaaauauauaguaaacacacaccaaucaaucugguccgagaucuccccca
ggguuuuagugcucuggagccgcugguggauuugcccaucgguaucaauauuacgcgcuuccaaacauugcucgcccug
caucggaguuaccuuacgccuggcgacaguagcagcggauggaccgcuggagccgccgccuacuaugucggguaccuuc
aaccacgcacuuuucuccugaaauacaacgaaaaugggacaauuacagacgcuguugauugcgcacucgauccccuguca
gaaacaaaauguacacuuaaaucuuuuacggucgagaaagggauuuaccagacaucuaacuuccgaguacaaccaaccga
aucuauagugcgguuccccaacauuacgaaccugugcccguucggcgaaguguucaacgcaacacgauuugcuucuguu
uacgcuuggaaccggaaacgcaucuccaauugcgucgccgauuacagcguucuuuauaauucugcaucuuucuccaccu
ucaaaugcuauggugucucucccacaaaacucaaugaccucuguuuuaccaauguguaugcggacuccuucgucauacg
cggcgacgaggugagacaaaucgcaccagggcagacuggcaagaucgcugauuauaauuacaaacugccugaugauuuu
accggaugcguuauugcuuggaauucuaauaaccucgauuccaaaguuggcgggaacuacaauuaccucuaccgauugu
uucgcaaaucuaaccuuaagccguuugagagagauaucagcacagagauuuaucaagcuggcucuaccccuugcaaugga
guagaaggcuuuaacugcuauuuuccucuucagucuuauggauuucaaccuaccaacgggguaggguaccaacccuaua
gagucgucgugcucucauuugaacuccuucacgcccccgcuaccgugugugggccuaagaaauccacuaaucucguuaa
gaauaagugugugaauuuuaauuucaauggccugacagggaccgggguucugacugaaucuaacaagaaauuucugccg
uuccaacaauucgggcgcgauauugcagacacgaccgacgcggugcgcgauccucaaacacucgaaauccuugauaucac
uccuuguucuuucggcgguguaagcgucauuacuccuggcaccaauaccucuaaccaaguggcaguacucuaucaagau
gugaacugcacugaggucccgguugcaauacaugcggaucaacucaccccaacauggcgaguguauuccacagggagcaa
uguguuucaaacgagggccggcugucucauuggggccgaacacguuaauaauaguuaugagugcgauauucccauugga
gcgggcauuugugccagcuaucagacccaaacuaacucccccggguccgccucaucagucgcuagccaaucuauuauugc
guacacaaugucccugggagcugaaaacagcguggccuacucaaauaacagcauugcaauacccacaaauuuuacgauuu
caguaaccacugaaauccugcccgucuccaugaccaaaaccucugucgacugcacuauguacauaugcggcgacuccacc
gaguguuccaaucuccuucuccaauauggaaguuucugcacgcaguugaacagggcacuuacagggauugcagucgagc
aagacaagaacacccaagaaguauucgcacaaguaaaacagaucuacaagacacccccaaucaaagauuuugguggcuuca
acuucucccaaauacuuccagauccgucaaagccauccaaacgcucauucaucgaagaccuucuguucaauaaggucaca
uuggcggaugcuggauuuaucaagcaauauggggauuguuugggagauauugcagcgcgggaccugauaugcgcgcaa
aaguucaauggguugacggugcugcccccucuccucacugacgagaugauagcucaguauacgagcgcucuccucgcgg
gcacuaucaccucagguuggaccuucggggcuggcgcggcacuucaaauaccauuugcuaugcaaauggccuaucguuu
uaauggcaucggggugacccaaaacgugcucuaugaaaaccagaaacugauagcuaaucaauucaauagugccaucggca
aaauccaggauucauuguccagcaccgccucagcucucgggaaauugcaagacgucgucaaccaaaaugcucaagcgcuc
aacacccucguuaaacaacucucaaguaauuucggcgcgauuaguagcgugcugaacgauaucuugagucgucuugauc
caccugaagcagaaguccaaaucgacaggcuuauuaccggacgucugcaaagccugcaaaccuacguuacacaacaacuua
uaagggcagccgaaauaagggcuucugcaaaucuggcugccacgaagaugagcgaguguguccucggacaaagcaaaaga
guugacuuuugcggcaaaggguaccaccuuaugaguuucccucagucugcgccccauggaguaguguuucuccacguga
cuuauguaccggcacaagaaaagaacuuuaccacagccccagcaauaugucacgauggaaaagcacacuuuccacgggaa
gggguuuucguguccaacgggacccauugguuuguuacucaacgcaacuuuuaugaaccccaaaucauaaccacugaua
auacauuugucucugggaacugugaugucgugaucggaauagucaacaacacaguguaugauccguugcaaccagagcu
ggauuccuucaaagaagaacucgacaaguauuuuaagaaucacacaucaccggacguggaucuuggagacauaucaggca
uaaacgcuaguguggugaauauccaaaaggagaucgacaggcuuaacgaaguugcaaagaaccucaaugaaucucuuauc
gauuugcaagaauugggcaaauacgagcaaggcuacauuccugaagcaccacgggacgggcaagcuuacgugcggaaaga
uggcgaaugggugcucuugaguaccuuucuggguugauaa (SEQ ID NO: 120)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSP [symbol] A [symbol] SVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQGYIPEAPRDGQAYVRKDGEWVLLSTFLG** (SEQ ID NO: 121)
WuDivS_3F_D2P_Gly_pVax
ggatccgccaccatggattggacatggatattgttcttggttgcagcagctacccgggtacattccatgttcgtcttcctcgtactgctccc
acttgtcagtagtcaatgtgtgaacttgactacccggacgcagttgcccccggcctacactaatagcataacgcgtggagtctattaccc
cgacaaggtgttcaggtcatccgtcctgtatagcactcaagatctcttcttgcccttctttagtaacgtcacttggttccatgcaatccacgt
aagtggcactaatggcaccaagcgattcgacaatcccgtactcccttttaacgatggggtgtatttcgcgagcacagagaagtccaaca
tcatccgtggttggatcttcggcaccacactggattctaaaacccaaagcctgcttatagtaaataatgcaacaaacgtggtcattaaagt
ttgcgaatttcagttttgtaacgaccccttccttggcgtatattaccacaagaacaataaatcctggatggagagcgaatttagggtttaca
gttcagccaataattgtacattcgaatacgtaagccaacccttcttgatggacctggaaggaaagcaaggaaatttcaagaatctccgtg
aattcgtgttcaagaacatagacggctattttaaaatatattcaaaacacacaccgattaacctggtacgagatcttccgcaaggattctct
gcactggaaccgctggtcgatcttcctatcggcattaatatcactcggtttcaaacattgcttgctttgcatcggcgatatcttacacccgg
ggatagctcaagtggatggactgccggggctgctgcctattacgtaggctatctccaaccacggacattcctgctgaaatataacgaga
atgggacaatcacagatgctgttgactgcgctttggaccctttgagcgaaacaaagtgcacactcaaatccttcaccgtggaaaaggga
atctaccaaacgtctaattttcgcgtccaaccaaccgagagcatcgtcagattcccaaacattactaatctttgcccctttggcgaagtctt
caatgctacgcgatttgcgtccgtctacgcgtggaatcggaagcgcattagcaattgcgtcgcagactattcctttctctataactctgcat
ccttttctacctttaaatgttatggagtcaacgggacaaagctcaatgacctttgctttacaaatgtctatgcagactcttttgtcatccgtggt
gatgaggtacgacagatcgcgccaggacaaaccgggaagatcgccgattacaactacaaactgcccgacgatttcaccgggtgcgt
tattgcttggaactccaataatcttgatagtaaagttggcggcaactacaactacctgtatcgacttttccgtaaaagtaatctcaagccattt
gaaagagacatcaacacaacaatttatcaggctggatctaccccatgcaacggcgttgaaggatttaactgctacttccctctccaaagt
tacggtttccaaccaacaaacggcgttggctatcaaccttatagagtcgttgtcctctcttttgagcttaaccatgccccagcgacagtgtg
tgggccgaagaaaagcactaatttggttaagaataaatgtgttaactttaattttaatggattgacggggacaggggttctgacagagtct
aacaagaaatttctgccgttccaacagtttgggcgagatattgcagataccacggacgccgttcgagacccccaaacacttgaaattct
cgatataactccctgcagctttggcggtgtatccgttatcacgcccgggacaaataccagtaaccaagtcgcagtcctgtatcaaggcg
taaattgtacggaagtgcccgttgctatacacgctgaccaactgactcccacatggagagtctatagtactggttctaatgtgttccaaac
acgagccggttgcctgatcggagccgaacatgttaacaactcatacgaatgtgacataccgattggcgccggcatttgcgccagctat
caaacgcagaccaactcaccaagaaggcgtcgcagtgtagcaagtcaatctattatagcgtataccatgtctttgggagcagaaaactc
cgttgcttactctaataattctattgctatcccaaccaattttacaatctcagttactaccgaaatactgccggtaagcatgactaagacatcc
gtggattgcactatgtacatctgtggggactcaacagagtgtagtaatttgctgcttcaatatggctccttctgcactcaactgaatcgtgct
ctcacgggaattgctgttgagcaagataagaatacccaggaagtgtttgcccaagtcaaacaaatttataagacaccaccaattaaagat
tttggtggatttaatttcagccaaatacttccagatccctcacgcagacgacggtctttcatcgaggaccttctgttcaacaaagttactctg
gctgatgcaggcttcattaagcagtacggtgattgtcttggagacatcgctgcgcgcgacctcatatgcgcccagaaatttaatgggctg
accgtacttccccctttgctgactgatgagatgattgcacaatacacttccgcactccttgcgggtactatcacatccgggtggacttttgg
agctggcgccgctcttcaaattcccttcgccatgcaaatggcgtacaggtttaatggcatcggtgtgacacagaatgtgctctatgagaa
ccagaaacttatcgcaaaccagttcaattcagccatcgggaaaatccaagatagtctcagtagtactgcctcagctctcggcaagctcc
aggatgtagtgaatcagaatgcacaagccttgaacactctcgttaaacaactttcttccaactttggtgccatcagcagtgggcctaacg
atatattgagccgcttgcccaaagtggaagcggaagtccaaatagatagacttattaccggccggctgcaatctctgcaaacctatgtg
actcaacaattgatccgagctgccgaaatccgtgccagtgcaaatctcgccgcgaccaagatgagcgaatgtgtcttgggacagagc
aaaagagtcgatttctgcggaaaaggctaccacctgatgtctttccctcaatctgccccgcacggagtggtctttctccatgtgacttatgt
gccagcccaagaaaagaactttacaaccgcaccggcaatttgccatgacggaaaggcgcatttcccccgtgagggagtctttgtgag
caacgggacccattggttcgtgacacaacgcaatttctatgagcctcagatcattaccacggacaatactttcgtgtctggcaactgtga
cgtgctgataggcatcgtgaataataccgtctacgatcccttgcaacttgaactggactcattcaaagaagagctggataagtattttaag
aaccatacaagccctgatgtcgatcttggggatatatcaggcataaacgcatctgttgtgaatatccaaaaggaaattgatagattgaac
gaagttgccaagaacctcaatgaaagtcttatcgacctgcaagaactgggaaaatatgagcaatatataaaatggccatggagcgggc
gccggagacggagaaggggtagcggcggtagtggtagcgggtacatcccagaggcacccagagatggacaagcttacgtaagga
aggacggggaatgggtgctgctcagtacatttcttggatgataa (SEQ ID NO: 122)
ggauccgccaccauggauuggacauggauauuguucuugguugcagcagcuacccggguacauuccauguucgucuucc
ucguacugcucccacuugucaguagucaaugugugaacuugacuacccggacgcaguugcccccggccuacacuaauagc
auaacgcguggagucuauuaccccgacaagguguucaggucauccguccuguauagcacucaagaucucuucuugcccu
ucuuuaguaacgucacuugguuccaugcaauccacguaaguggcacuaauggcaccaagcgauucgacaaucccguacuc
ccuuuuaacgaugggguguauuucgcgagcacagagaaguccaacaucauccgugguuggaucuucggcaccacacugg
auucuaaaacccaaagccugcuuauaguaaauaaugcaacaaacguggucauuaaaguuugcgaauuucaguuuuguaac
gaccccuuccuuggcguauauuaccacaagaacaauaaauccuggauggagagcgaauuuaggguuuacaguucagccaa
uaauuguacauucgaauacguaagccaacccuucuugauggaccuggaaggaaagcaaggaaauuucaagaaucuccgug
aauucguguucaagaacauagacggcuauuuuaaaauauauucaaaacacacaccgauuaaccugguacgagaucuuccg
caaggauucucugcacuggaaccgcuggucgaucuuccuaucggcauuaauaucacucgguuucaaacauugcuugcuu
ugcaucggcgauaucuuacacccggggauagcucaaguggauggacugccggggcugcugccuauuacguaggcuaucu
ccaaccacggacauuccugcugaaauauaacgagaaugggacaaucacagaugcuguugacugcgcuuuggacccuuuga
gcgaaacaaagugcacacucaaauccuucaccguggaaaagggaaucuaccaaacgucuaauuuucgcguccaaccaaccg
agagcaucgucagauucccaaacauuacuaaucuuugccccuuuggcgaagucuucaaugcuacgcgauuugcguccgu
cuacgcguggaaucggaagcgcauuagcaauugcgucgcagacuauuccuuucucuauaacucugcauccuuuucuacc
uuuaaauguuauggagucaacgggacaaagcucaaugaccuuugcuuuacaaaugucuaugcagacucuuuugucaucc
guggugaugagguacgacagaucgcgccaggacaaaccgggaagaucgccgauuacaacuacaaacugcccgacgauuuc
accgggugcguuauugcuuggaacuccaauaaucuugauaguaaaguuggcggcaacuacaacuaccuguaucgacuuu
uccguaaaaguaaucucaagccauuugaaagagacaucaacacaacaauuuaucaggcuggaucuaccccaugcaacggc
guugaaggauuuaacugcuacuucccucuccaaaguuacgguuuccaaccaacaaacggcguuggcuaucaaccuuauag
agucguuguccucucuuuugagcuuaaccaugccccagcgacagugugugggccgaagaaaagcacuaauuugguuaag
aauaaauguguuaacuuuaauuuuaauggauugacggggacagggguucugacagagucuaacaagaaauuucugccgu
uccaacaguuugggcgagauauugcagauaccacggacgccguucgagacccccaaacacuugaaauucucgauauaacu
cccugcagcuuuggcgguguauccguuaucacgcccgggacaaauaccaguaaccaagucgcaguccuguaucaaggcg
uaaauuguacggaagugcccguugcuauacacgcugaccaacugacucccacauggagagucuauaguacugguucuaa
uguguuccaaacacgagccgguugccugaucggagccgaacauguuaacaacucauacgaaugugacauaccgauuggc
gccggcauuugcgccagcuaucaaacgcagaccaacucaccaagaaggcgucgcaguguagcaagucaaucuauuauagc
guauaccaugucuuugggagcagaaaacuccguugcuuacucuaauaauucuauugcuaucccaaccaauuuuacaauc
ucaguuacuaccgaaauacugccgguaagcaugacuaagacauccguggauugcacuauguacaucuguggggacucaa
cagaguguaguaauuugcugcuucaauauggcuccuucugcacucaacugaaucgugcucucacgggaauugcuguuga
gcaagauaagaauacccaggaaguguuugcccaagucaaacaaauuuauaagacaccaccaauuaaagauuuugguggau
uuaauuucagccaaauacuuccagaucccucacgcagacgacggucuuucaucgaggaccuucuguucaacaaaguuacu
cuggcugaugcaggcuucauuaagcaguacggugauugucuuggagacaucgcugcgcgcgaccucauaugcgcccaga
aauuuaaugggcugaccguacuucccccuuugcugacugaugagaugauugcacaauacacuuccgcacuccuugcggg
uacuaucacauccggguggacuuuuggagcuggcgccgcucuucaaauucccuucgccaugcaaauggcguacagguuu
aauggcaucggugugacacagaaugugcucuaugagaaccagaaacuuaucgcaaaccaguucaauucagccaucgggaa
aauccaagauagucucaguaguacugccucagcucucggcaagcuccaggauguagugaaucagaaugcacaagccuuga
acacucucguuaaacaacuuucuuccaacuuuggugccaucagcagugggccuaacgauauauugagccgcuugcccaaa
guggaagcggaaguccaaauagauagacuuauuaccggccggcugcaaucucugcaaaccuaugugacucaacaauugau
ccgagcugccgaaauccgugccagugcaaaucucgccgcgaccaagaugagcgaaugugucuugggacagagcaaaagag
ucgauuucugcggaaaaggcuaccaccugaugucuuucccucaaucugccccgcacggaguggucuuucuccaugugac
uuaugugccagcccaagaaaagaacuuuacaaccgcaccggcaauuugccaugacggaaaggcgcauuucccccgugagg
gagucuuugugagcaacgggacccauugguucgugacacaacgcaauuucuaugagccucagaucauuaccacggacaa
uacuuucgugucuggcaacugugacgugcugauaggcaucgugaauaauaccgucuacgaucccuugcaacuugaacug
gacucauucaaagaagagcuggauaaguauuuuaagaaccauacaagcccugaugucgaucuuggggauauaucaggca
uaaacgcaucuguugugaauauccaaaaggaaauugauagauugaacgaaguugccaagaaccucaaugaaagucuuauc
gaccugcaagaacugggaaaauaugagcaauauauaaaauggccauggagcgggcgccggagacggagaagggguagcg
gcgguagugguagcggguacaucccagaggcacccagagauggacaagcuuacguaaggaaggacggggaaugggugcu
gcucaguacauuucuuggaugauaa (SEQ ID NO: 123)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGV [symbol] T
KLNDLCFTNVYADSFVIRGDEVRQIAPGQTGKIADYNYKLPDDFTGCVIAWNSNNLD
SKVGGNYNYLYRLFRKSNLKPFERDI [symbol] T [symbol] IYQAGSTPCNGVEGFNCYFPLQSYGFQPT
NGVGYQPYRVVVLSFEL [symbol] HAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIY
KTPPIKDFGGFNFSQILPDPS [symbol] RSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISS [symbol] NDILSRL [symbol] KVEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 124)
WuS_3F_2P_NoTri_pVax
ggatccgccaccatggactggacgtggattttgtttcttgtcgctgcagctacccgggttcactccatgtttgttttcctggtgctccttccc
cttgtaagctcacaatgcgtcaatttgaccacgcgtacacaactgcccccagcatatactaattctttcacacgcggggtctactatcccg
ataaagtctttagaagtagcgtacttcatagtacccaagatttgtttctgcccttcttcagcaatgtcacgtggtttcatgcgatccatgtatc
cggcacgaacgggacaaaacgatttgataaccccgtgctccccttcaacgacggggtttatttcgccagcaccgagaaatcaaatatta
tcaggggctggattttcgggacaacacttgattccaagacacaatctcttcttatcgtgaataatgcaactaatgtggtgatcaaggtttgc
gagttccaattttgtaatgacccttttcttggcgtgtactatcataagaataacaagagttggatggaatcagagttccgggtctacagcag
tgctaacaattgtacgtttgaatacgtttctcagccttttctgatggaccttgaaggtaagcaaggcaatttcaagaacttgcgggaatttgt
cttcaagaacatagatggctattttaagatatatagcaaacacactcccataaatctcgtcagagatcttccacagggctttagcgccctg
gaaccattggttgatttgccaattggaataaacataactcgattccaaaccttgctcgcactccatcggagctacctgacgcctggagatt
cctcttccggctggactgccggagcagcagcttattatgtaggctacttgcaaccccgcacgttcctgctcaaatataatgaaaatggca
ctataaccgatgcggtagactgcgctcttgatcccctgagtgaaactaaatgtacgttgaaaagctttactgtagagaaaggcatctatca
gactagtaactttagggtgcaacccacggagtccattgtacggttcccaaacattaccaacctctgtccattcggagaagtgtttaatgcc
acaagattcgcttcagtgtatgcctggaaccggaaacgcatctcaaattgcgttgccgattattcagtactttacaactcagccagtttctct
acttttaagtgctatggcgtttccccgacgaagctcaatgatctgtgctttactaacgtttacgcagactctttcgtcatcagaggcgatgaa
gtcaggcaaatagctcctggtcaaaccggcaagatcgccgactacaactataaactgcccgatgatttcactgggtgtgtgatcgcgtg
gaattccaataatttggactctaaggtaggtggcaactataactacctctatcgactcttccgaaaatccaaccttaagccgtttgaacgcg
atattagtaccgaaatataccaagccgggtctacaccctgtaacggcgttgaaggtttcaattgttactttccactgcagagttatggctttc
aacccaccaacggggttggctatcagccctatagggttgtggtcctcagttttgagcttctgcatgcaccagcaaccgtgtgcggacct
aagaagtcaacaaatctcgtgaagaacaagtgtgttaatttcaatttcaatggccttacagggaccggagtgcttacagaaagcaataag
aagttcttgccctttcaacagttcggcagggacatagcggacacgacagatgcagttcgagacccgcaaactctcgaaattctggatat
cacaccttgcagttttggtggcgtgtctgttatcacaccaggcaccaacacttccaaccaggtggcagttttgtaccaggatgttaattgta
cagaggtcccagtggcaatacacgctgaccaactgactccaacttggagagtctactctacaggctcaaacgtcttccaaacacgggc
ggggtgtctgatcggagcagaacacgttaataacagttacgagtgtgatatcccgataggagctggtatttgcgcttcataccagacgc
aaacgaactcaccacgaagacgccggtcagttgcatcacaatccattattgcatacaccatgtcactcggagcggagaattctgtagca
tacagtaacaatagtatcgcaatacctacgaactttaccatttccgtcacaactgaaatcttgcccgtctcaatgacaaagacaagcgtag
attgtacaatgtatatttgcggagattcaacagagtgctccaacctgctgctccagtacggtagtttctgtacccagctcaatagggccct
caccggaattgcagttgaacaagacaagaacacccaagaagtgtttgcacaagtcaaacaaatctataaaacacccccaatcaaagat
ttcggtggcttcaacttttcacaaattctccctgatcctagccgccgccgcagatcattcatcgaagacttgctcttcaataaggttaccctg
gcagacgccggttttattaaacaatacggagattgcctcggtgacatcgccgctagagaccttatctgtgcccaaaagttcaacggact
caccgtgctgcccccattgctgaccgatgaaatgattgctcaatatacatctgcgctcctcgcagggaccattacttcagggtggactttt
ggggctggcgccgcattgcagattcccttcgccatgcagatggcatataggtttaacggcattggagttacccaaaatgtactctacga
gaaccaaaagctgattgcaaatcagttcaacagtgcaataggcaaaatacaagactctctgtcttcaaccgccagcgctcttggaaagc
tccaagatgttgttaatcaaaatgcccaagcgttgaataccctcgtgaagcaactctccagcaattttggtgccatctctagcgtgctgaa
cgacattctgtcacggctcgatcccccggaagccgaggtacaaattgaccgattgataaccgggcgactccaaagccttcagacctac
gttacacaacagctcattcgcgctgcagaaattagagcctctgcaaatcttgcagctacaaagatgtcagagtgcgttctcggtcaaagc
aaaagagtggatttctgcggaaaggggtaccacctcatgagtttcccacagagtgcccctcatggcgtagtctttcttcatgttacttatgt
accagcccaagaaaagaatttcactacagcacccgcgatttgtcatgatggcaaagcgcacttccctcgggaaggcgtgttcgtgtcta
atggaacacattggttcgtgacgcaacggaatttctacgagccccaaattatcactactgataacaccttcgtctccggaaactgcgatgt
tgttattggcattgtcaacaataccgtttacgacccgctccaacctgagctggattcatttaaagaggaattggacaaatattttaagaatca
tacctctccagacgtggatttgggtgacattagcggaataaatgcatctgtggtcaatatccaaaaggaaattgataggctgaacgaggt
cgccaagaatttgaacgaatctttgattgatcttcaagaacttggcaagtatgaacaatacataaaatggccctggtgatag (SEQ ID
NO: 125)
ggauccgccaccauggacuggacguggauuuuguuucuugucgcugcagcuacccggguucacuccauguuuguuuuc
cuggugcuccuuccccuuguaagcucacaaugcgucaauuugaccacgcguacacaacugcccccagcauauacuaauuc
uuucacacgcggggucuacuaucccgauaaagucuuuagaaguagcguacuucauaguacccaagauuuguuucugccc
uucuucagcaaugucacgugguuucaugcgauccauguauccggcacgaacgggacaaaacgauuugauaaccccgugc
uccccuucaacgacgggguuuauuucgccagcaccgagaaaucaaauauuaucaggggcuggauuuucgggacaacacu
ugauuccaagacacaaucucuucuuaucgugaauaaugcaacuaauguggugaucaagguuugcgaguuccaauuuugu
aaugacccuuuucuuggcguguacuaucauaagaauaacaagaguuggauggaaucagaguuccgggucuacagcagug
cuaacaauuguacguuugaauacguuucucagccuuuucugauggaccuugaagguaagcaaggcaauuucaagaacuu
gcgggaauuugucuucaagaacauagauggcuauuuuaagauauauagcaaacacacucccauaaaucucgucagagauc
uuccacagggcuuuagcgcccuggaaccauugguugauuugccaauuggaauaaacauaacucgauuccaaaccuugcu
cgcacuccaucggagcuaccugacgccuggagauuccucuuccggcuggacugccggagcagcagcuuauuauguaggc
uacuugcaaccccgcacguuccugcucaaauauaaugaaaauggcacuauaaccgaugcgguagacugcgcucuugaucc
ccugagugaaacuaaauguacguugaaaagcuuuacuguagagaaaggcaucuaucagacuaguaacuuuagggugcaa
cccacggaguccauuguacgguucccaaacauuaccaaccucuguccauucggagaaguguuuaaugccacaagauucgc
uucaguguaugccuggaaccggaaacgcaucucaaauugcguugccgauuauucaguacuuuacaacucagccaguuuc
ucuacuuuuaagugcuauggcguuuccccgacgaagcucaaugaucugugcuuuacuaacguuuacgcagacucuuucg
ucaucagaggcgaugaagucaggcaaauagcuccuggucaaaccggcaagaucgccgacuacaacuauaaacugcccgau
gauuucacugggugugugaucgcguggaauuccaauaauuuggacucuaagguagguggcaacuauaacuaccucuauc
gacucuuccgaaaauccaaccuuaagccguuugaacgcgauauuaguaccgaaauauaccaagccgggucuacacccugu
aacggcguugaagguuucaauuguuacuuuccacugcagaguuauggcuuucaacccaccaacgggguuggcuaucagc
ccuauaggguugugguccucaguuuugagcuucugcaugcaccagcaaccgugugcggaccuaagaagucaacaaaucu
cgugaagaacaaguguguuaauuucaauuucaauggccuuacagggaccggagugcuuacagaaagcaauaagaaguuc
uugcccuuucaacaguucggcagggacauagcggacacgacagaugcaguucgagacccgcaaacucucgaaauucugga
uaucacaccuugcaguuuugguggcgugucuguuaucacaccaggcaccaacacuuccaaccagguggcaguuuuguac
caggauguuaauuguacagaggucccaguggcaauacacgcugaccaacugacuccaacuuggagagucuacucuacagg
cucaaacgucuuccaaacacgggcggggugucugaucggagcagaacacguuaauaacaguuacgagugugauaucccg
auaggagcugguauuugcgcuucauaccagacgcaaacgaacucaccacgaagacgccggucaguugcaucacaauccau
uauugcauacaccaugucacucggagcggagaauucuguagcauacaguaacaauaguaucgcaauaccuacgaacuuua
ccauuuccgucacaacugaaaucuugcccgucucaaugacaaagacaagcguagauuguacaauguauauuugcggagau
ucaacagagugcuccaaccugcugcuccaguacgguaguuucuguacccagcucaauagggcccucaccggaauugcagu
ugaacaagacaagaacacccaagaaguguuugcacaagucaaacaaaucuauaaaacacccccaaucaaagauuucggugg
cuucaacuuuucacaaauucucccugauccuagccgccgccgcagaucauucaucgaagacuugcucuucaauaagguua
cccuggcagacgccgguuuuauuaaacaauacggagauugccucggugacaucgccgcuagagaccuuaucugugccca
aaaguucaacggacucaccgugcugcccccauugcugaccgaugaaaugauugcucaauauacaucugcgcuccucgcag
ggaccauuacuucaggguggacuuuuggggcuggcgccgcauugcagauucccuucgccaugcagauggcauauaggu
uuaacggcauuggaguuacccaaaauguacucuacgagaaccaaaagcugauugcaaaucaguucaacagugcaauaggc
aaaauacaagacucucugucuucaaccgccagcgcucuuggaaagcuccaagauguuguuaaucaaaaugcccaagcguu
gaauacccucgugaagcaacucuccagcaauuuuggugccaucucuagcgugcugaacgacauucugucacggcucgau
cccccggaagccgagguacaaauugaccgauugauaaccgggcgacuccaaagccuucagaccuacguuacacaacagcu
cauucgcgcugcagaaauuagagccucugcaaaucuugcagcuacaaagaugucagagugcguucucggucaaagcaaaa
gaguggauuucugcggaaagggguaccaccucaugaguuucccacagagugccccucauggcguagucuuucuucaugu
uacuuauguaccagcccaagaaaagaauuucacuacagcacccgcgauuugucaugauggcaaagcgcacuucccucggg
aaggcguguucgugucuaauggaacacauugguucgugacgcaacggaauuucuacgagccccaaauuaucacuacuga
uaacaccuucgucuccggaaacugcgauguuguuauuggcauugucaacaauaccguuuacgacccgcuccaaccugagc
uggauucauuuaaagaggaauuggacaaauauuuuaagaaucauaccucuccagacguggauuugggugacauuagcgg
aauaaaugcaucuguggucaauauccaaaaggaaauugauaggcugaacgaggucgccaagaauuugaacgaaucuuug
auugaucuucaagaacuuggcaaguaugaacaauacauaaaauggcccuggugauag (SEQ ID NO: 126)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSPRR [symbol] RSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPS [symbol] RSFIEDLLFNKVTLADAGFIKQYGDCLGDIAARDLIC
AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ
LSSNFGAISSVLNDILSRLD [symbol] EAEVQIDRLITGRLQSLQTYVTQQLIRAAEIRASANLA
ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPW** (SEQ ID NO: 127)
WuS_3F_D2P_Gly_pVax
ggatccgccaccatggattggacatggatattgttcttggttgcagcagctacccgggtacattccatgttcgtcttcctcgtactgctccc
acttgtcagtagtcaatgtgtgaacttgactacccggacgcagttgcccccggcctacactaatagctttacgcgtggagtctattacccc
gacaaggtgttcaggtcatccgtcctgcatagcactcaagatctcttcttgcccttctttagtaacgtcacttggttccatgcaatccacgta
agtggcactaatggcaccaagcgattcgacaatcccgtactcccttttaacgatggggtgtatttcgcgagcacagagaagtccaacat
catccgtggttggatcttcggcaccacactggattctaaaacccaaagcctgcttatagtaaataatgcaacaaacgtggtcattaaagtt
tgcgaatttcagttttgtaacgaccccttccttggcgtatattaccacaagaacaataaatcctggatggagagcgaatttagggtttacag
ttcagccaataattgtacattcgaatacgtaagccaacccttcttgatggacctggaaggaaagcaaggaaatttcaagaatctccgtga
attcgtgttcaagaacatagacggctattttaaaatatattcaaaacacacaccgattaacctggtacgagatcttccgcaaggattctctg
cactggaaccgctggtcgatcttcctatcggcattaatatcactcggtttcaaacattgcttgctttgcatcggagttatcttacacccgggg
atagctcaagtggatggactgccggggctgctgcctattacgtaggctatctccaaccacggacattcctgctgaaatataacgagaat
gggacaatcacagatgctgttgactgcgctttggaccctttgagcgaaacaaagtgcacactcaaatccttcaccgtggaaaagggaat
ctaccaaacgtctaattttcgcgtccaaccaaccgagagcatcgtcagattcccaaacattactaatctttgcccctttggcgaagtcttca
atgctacgcgatttgcgtccgtctacgcgtggaatcggaagcgcattagcaattgcgtcgcagactattccgtgctctataactctgcatc
cttttctacctttaaatgttatggagtcaacgggacaaagctcaatgacctttgctttacaaatgtctatgcagactcttttgtcatccgtggtg
atgaggtacgacagatcgcgccaggacaaaccgggaagatcgccgattacaactacaaactgcccgacgatttcaccgggtgcgtta
ttgcttggaactccaataatcttgatagtaaagttggcggcaactacaactacctgtatcgacttttccgtaaaagtaatctcaagccatttg
aaagagacatcaacacaacaatttatcaggctggatctaccccatgcaacggcgttgaaggatttaactgctacttccctctccaaagtta
cggtttccaaccaacaaacggcgttggctatcaaccttatagagtcgttgtcctctcttttgagcttaaccatgccccagcgacagtgtgt
gggccgaagaaaagcactaatttggttaagaataaatgtgttaactttaattttaatggattgacggggacaggggttctgacagagtcta
acaagaaatttctgccgttccaacagtttgggcgagatattgcagataccacggacgccgttcgagacccccaaacacttgaaattctc
gatataactccctgcagctttggcggtgtatccgttatcacgcccgggacaaataccagtaaccaagtcgcagtcctgtatcaagacgta
aattgtacggaagtgcccgttgctatacacgctgaccaactgactcccacatggagagtctatagtactggttctaatgtgttccaaacac
gagccggttgcctgatcggagccgaacatgttaacaactcatacgaatgtgacataccgattggcgccggcatttgcgccagctatca
aacgcagaccaactcaccaagaaggcgtcgcagtgtagcaagtcaatctattatagcgtataccatgtctttgggagcagaaaactcc
gttgcttactctaataattctattgctatcccaaccaattttacaatctcagttactaccgaaatactgccggtaagcatgactaagacatccg
tggattgcactatgtacatctgtggggactcaacagagtgtagtaatttgctgcttcaatatggctccttctgcactcaactgaatcgtgctc
tcacgggaattgctgttgagcaagataagaatacccaggaagtgtttgcccaagtcaaacaaatttataagacaccaccaattaaagatt
ttggtggatttaatttcagccaaatacttccagatccctcacgcagacgacggtctttcatcgaggaccttctgttcaacaaagttactctg
gctgatgcaggcttcattaagcagtacggtgattgtcttggagacatcgctgcgcgcgacctcatatgcgcccagaaatttaatgggctg
accgtacttccccctttgctgactgatgagatgattgcacaatacacttccgcactccttgcgggtactatcacatccgggtggacttttgg
agctggcgccgctcttcaaattcccttcgccatgcaaatggcgtacaggtttaatggcatcggtgtgacacagaatgtgctctatgagaa
ccagaaacttatcgcaaaccagttcaattcagccatcgggaaaatccaagatagtctcagtagtactgcctcagctctcggcaagctcc
aggatgtagtgaatcagaatgcacaagccttgaacactctcgttaaacaactttcttccaactttggtgccatcagcagtgggcctaacg
atatattgagccgcttgcccaaagtggaagcggaagtccaaatagatagacttattaccggccggctgcaatctctgcaaacctatgtg
actcaacaattgatccgagctgccgaaatccgtgccagtgcaaatctcgccgcgaccaagatgagcgaatgtgtcttgggacagagc
aaaagagtcgatttctgcggaaaaggctaccacctgatgtctttccctcaatctgccccgcacggagtggtctttctccatgtgacttatgt
gccagcccaagaaaagaactttacaaccgcaccggcaatttgccatgacggaaaggcgcatttcccccgtgagggagtctttgtgag
caacgggacccattggttcgtgacacaacgcaatttctatgagcctcagatcattaccacggacaatactttcgtgtctggcaactgtga
cgtggtcataggcatcgtgaataataccgtctacgatcccttgcaacccgaactggactcattcaaagaagagctggataagtattttaa
gaaccatacaagccctgatgtcgatcttggggatatatcaggcataaacgcatctgttgtgaatatccaaaaggaaattgatagattgaa
cgaagttgccaagaacctcaatgaaagtcttatcgacctgcaagaactgggaaaatatgagcaatatataaaatggccatggagcggg
cgccggagacggagaaggggtagcggcggtagtggtagcgggtacatcccagaggcacccagagatggacaagcttacgtaagg
aaggacggggaatgggtgctgctcagtacatttcttggatgataa (SEQ ID NO: 128)
ggauccgccaccauggauuggacauggauauuguucuugguugcagcagcuacccggguacauuccauguucgucuucc
ucguacugcucccacuugucaguagucaaugugugaacuugacuacccggacgcaguugcccccggccuacacuaauagc
uuuacgcguggagucuauuaccccgacaagguguucaggucauccguccugcauagcacucaagaucucuucuugcccu
ucuuuaguaacgucacuugguuccaugcaauccacguaaguggcacuaauggcaccaagcgauucgacaaucccguacuc
ccuuuuaacgaugggguguauuucgcgagcacagagaaguccaacaucauccgugguuggaucuucggcaccacacugg
auucuaaaacccaaagccugcuuauaguaaauaaugcaacaaacguggucauuaaaguuugcgaauuucaguuuuguaac
gaccccuuccuuggcguauauuaccacaagaacaauaaauccuggauggagagcgaauuuaggguuuacaguucagccaa
uaauuguacauucgaauacguaagccaacccuucuugauggaccuggaaggaaagcaaggaaauuucaagaaucuccgug
aauucguguucaagaacauagacggcuauuuuaaaauauauucaaaacacacaccgauuaaccugguacgagaucuuccg
caaggauucucugcacuggaaccgcuggucgaucuuccuaucggcauuaauaucacucgguuucaaacauugcuugcuu
ugcaucggaguuaucuuacacccggggauagcucaaguggauggacugccggggcugcugccuauuacguaggcuaucu
ccaaccacggacauuccugcugaaauauaacgagaaugggacaaucacagaugcuguugacugcgcuuuggacccuuuga
gcgaaacaaagugcacacucaaauccuucaccguggaaaagggaaucuaccaaacgucuaauuuucgcguccaaccaaccg
agagcaucgucagauucccaaacauuacuaaucuuugccccuuuggcgaagucuucaaugcuacgcgauuugcguccgu
cuacgcguggaaucggaagcgcauuagcaauugcgucgcagacuauuccgugcucuauaacucugcauccuuuucuacc
uuuaaauguuauggagucaacgggacaaagcucaaugaccuuugcuuuacaaaugucuaugcagacucuuuugucaucc
guggugaugagguacgacagaucgcgccaggacaaaccgggaagaucgccgauuacaacuacaaacugcccgacgauuuc
accgggugcguuauugcuuggaacuccaauaaucuugauaguaaaguuggcggcaacuacaacuaccuguaucgacuuu
uccguaaaaguaaucucaagccauuugaaagagacaucaacacaacaauuuaucaggcuggaucuaccccaugcaacggc
guugaaggauuuaacugcuacuucccucuccaaaguuacgguuuccaaccaacaaacggcguuggcuaucaaccuuauag
agucguuguccucucuuuugagcuuaaccaugccccagcgacagugugugggccgaagaaaagcacuaauuugguuaag
aauaaauguguuaacuuuaauuuuaauggauugacggggacagggguucugacagagucuaacaagaaauuucugccgu
uccaacaguuugggcgagauauugcagauaccacggacgccguucgagacccccaaacacuugaaauucucgauauaacu
cccugcagcuuuggcgguguauccguuaucacgcccgggacaaauaccaguaaccaagucgcaguccuguaucaagacgu
aaauuguacggaagugcccguugcuauacacgcugaccaacugacucccacauggagagucuauaguacugguucuaau
guguuccaaacacgagccgguugccugaucggagccgaacauguuaacaacucauacgaaugugacauaccgauuggcgc
cggcauuugcgccagcuaucaaacgcagaccaacucaccaagaaggcgucgcaguguagcaagucaaucuauuauagcgu
auaccaugucuuugggagcagaaaacuccguugcuuacucuaauaauucuauugcuaucccaaccaauuuuacaaucuca
guuacuaccgaaauacugccgguaagcaugacuaagacauccguggauugcacuauguacaucuguggggacucaacag
aguguaguaauuugcugcuucaauauggcuccuucugcacucaacugaaucgugcucucacgggaauugcuguugagca
agauaagaauacccaggaaguguuugcccaagucaaacaaauuuauaagacaccaccaauuaaagauuuugguggauuua
auuucagccaaauacuuccagaucccucacgcagacgacggucuuucaucgaggaccuucuguucaacaaaguuacucug
gcugaugcaggcuucauuaagcaguacggugauugucuuggagacaucgcugcgcgcgaccucauaugcgcccagaaau
uuaaugggcugaccguacuucccccuuugcugacugaugagaugauugcacaauacacuuccgcacuccuugcggguac
uaucacauccggguggacuuuuggagcuggcgccgcucuucaaauucccuucgccaugcaaauggcguacagguuuaau
ggcaucggugugacacagaaugugcucuaugagaaccagaaacuuaucgcaaaccaguucaauucagccaucgggaaaau
ccaagauagucucaguaguacugccucagcucucggcaagcuccaggauguagugaaucagaaugcacaagccuugaaca
cucucguuaaacaacuuucuuccaacuuuggugccaucagcagugggccuaacgauauauugagccgcuugcccaaagu
ggaagcggaaguccaaauagauagacuuauuaccggccggcugcaaucucugcaaaccuaugugacucaacaauugaucc
gagcugccgaaauccgugccagugcaaaucucgccgcgaccaagaugagcgaaugugucuugggacagagcaaaagaguc
gauuucugcggaaaaggcuaccaccugaugucuuucccucaaucugccccgcacggaguggucuuucuccaugugacuu
augugccagcccaagaaaagaacuuuacaaccgcaccggcaauuugccaugacggaaaggcgcauuucccccgugaggga
gucuuugugagcaacgggacccauugguucgugacacaacgcaauuucuaugagccucagaucauuaccacggacaauac
uuucgugucuggcaacugugacguggucauaggcaucgugaauaauaccgucuacgaucccuugcaacccgaacuggac
ucauucaaagaagagcuggauaaguauuuuaagaaccauacaagcccugaugucgaucuuggggauauaucaggcauaa
acgcaucuguugugaauauccaaaaggaaauugauagauugaacgaaguugccaagaaccucaaugaaagucuuaucgac
cugcaagaacugggaaaauaugagcaauauauaaaauggccauggagcgggcgccggagacggagaagggguagcggcg
guagugguagcggguacaucccagaggcacccagagauggacaagcuuacguaaggaaggacggggaaugggugcugcu
caguacauuucuuggaugauaa (SEQ ID NO: 129)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGV [symbol] T
KLNDLCFTNVYADSFVIRGDEVRQIAPGQTGKIADYNYKLPDDFTGCVIAWNSNNLD
SKVGGNYNYLYRLFRKSNLKPFERDI [symbol] T [symbol] IYQAGSTPCNGVEGFNCYFPLQSYGFQPT
NGVGYQPYRVVVLSFEL [symbol] HAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRR [symbol] RSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIY
KTPPIKDFGGFNFSQILPDPS [symbol] RSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISS [symbol] NDILSRL [symbol] KVEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 130)
WuS_3F_D2P_rB_pVax
ggatccgccaccatggattggacatggattctgtttctcgttgccgccgctacgcgcgtgcacagcatgttcgttttcctcgtactgttgcc
tctggtatcatctcaatgtgtgaatcttacgacgaggacgcaactgccaccagcttacaccaactcttttactagaggggtctattacccc
gacaaggttttcagatcctcagtgctgcatagtacacaagatttgtttcttcccttcttctccaatgtcacttggtttcacgctatccacgtgtc
cggcactaatggaacgaagcggtttgataacccggtacttccatttaatgacggggtatactttgcaagcaccgagaaaagtaatatcat
tcgtgggtggatctttggcactacactcgactccaagacacaatcccttttgatagtaaataatgctacaaatgtggttataaaggtgtgcg
agtttcaattctgcaatgaccccttcctcggtgtctattatcacaagaacaataaatcttggatggaaagtgagttcagggtatacagctct
gcaaacaactgcacatttgagtatgtgagccaaccgtttcttatggatctggaaggtaagcagggtaactttaagaaccttcgggagttc
gtctttaagaatatagatggctattttaaaatctatagtaaacacactcctattaaccttgtgcgcgatctccctcaagggttctctgcattgg
aaccgcttgttgatttgcctataggaatcaatattacacgatttcaaacactcctcgctctccataggagctaccttaccccaggcgactca
agctctggttggacggcaggagctgcagcatactatgtgggttatcttcagccgcggacattcttgcttaagtataatgagaatggaact
atcactgacgctgttgactgtgccctggaccctctttcagaaacaaaatgtactctcaaatcattcaccgtggagaagggaatatatcaaa
caagtaactttagggtccaacctaccgagagcatcgtgcgattccctaatattaccaatttgtgtcccttcggtgaagtcttcaacgcgac
ccgctttgctagcgtctatgcgtggaacaggaagcgaattagcaactgtgttgcagattacagtgtgctgtacaatagtgcgagcttttcc
acgttcaaatgctatggcgtgaaacctaccaaacttaatgatttgtgcttcactaatgtttatgctgacagcttcgttatccggggtgatgag
gtgaggcagattgcccccggtcaaactggcaaaattgccgactacaattacaagctccctgatgattttactggatgtgtcatagcgtgg
aattccaataatctggactctaaggttggtggtaattataactatctctaccgcctgtttcgtaagagcaatctgaaaccctttgaaagagat
atttggactgagatatatcaagctggctcaactccttgcaacggggtcgaaggtttcaattgttactttccacttcaatcatacgggtttcaa
ccaactaacggtgtaggttatcaaccctatcgggtggttgtcctgagctttgagctgaaccatgccccggctacagtatgcggcccaaa
gaaatccactaacttggtcaagaacaaatgcgtcaactttaactttaacggactcacggggacaggagtccttaccgaatccaacaaga
aattcttgcctttccaacaatttggacgagacattgcggataccacagacgcagtacgcgacccacagactcttgaaatcctcgacataa
caccctgcagtttcggcggtgtaagtgtcattaccccaggcactaatacgagcaaccaagtggcggtgctctaccaagacgttaattgc
actgaggtcccagtggctattcacgctgaccaacttacacccacatggagagtgtatagtacaggctcaaacgtcttccagacacggg
cggggtgccttattggagcagaacatgttaacaattcctatgaatgcgatatcccgattggagccgggatctgtgctagctatcaaaccc
aaacaaatagccccagacgtcgacgttccgtggctagtcaaagcatcatcgcctacactatgagtcttggggccgaaaattccgttgctt
acagtaacaacagtatcgctatccccaccaattttactattagtgtaactacagagattctgccggtttccatgacaaagacttccgtggat
tgtacgatgtatatttgcggcgacagcacagagtgcagcaatctgctgctgcaatacggtagtttctgcacccaattgaaccgtgctctg
acgggaattgcagttgagcaggacaagaatactcaagaagtatttgcacaagtcaaacagatatacaagacgcccccgattaaagattt
cggcgggtttaactttagccaaattcttccggaccccagcagacgccgccgaagctttattgaggacctgctgtttaataaagttaccctt
gctgatgctggttttatcaagcaatacggagattgcctgggagatatcgccgccagggatttgatctgtgcgcaaaagtttaacggcctt
accgttctcccgccccttctgaccgatgaaatgatagcccaatacacttccgcactcctggcaggcacaattacttccggctggacgttt
ggggccggggcagccttgcaaattccgtttgctatgcaaatggcatatcgtttcaatggtatcggcgtaacacaaaatgtcctttatgaga
accagaaactcattgctaatcagtttaattccgctatcggcaagattcaagacagtctcagcagcacggcgagcgcacttggtaaacttc
aagacgttgtcaaccagaatgctcaagccctgaacactctggtaaaacaacttagctctaatttcggtgcaattagctccggtccgaacg
atattctgtcacggctcccgaaagtcgaagccgaagtccagatcgataggctgatcacagggcgcttgcagagtctccaaacctacgt
gacgcaacaactcattcgggcggctgaaattcgtgcaagcgctaatctggccgctaccaaaatgagtgagtgtgttctcggtcaatcaa
agagggttgacttttgcggcaaaggatatcatttgatgagttttccgcaatctgcccctcatggggtagtatttctgcacgtaacttatgtac
cagcacaagaaaagaacttcaccacggccccagcaatatgccacgatggcaaagctcatttccctcgcgaaggggtctttgtaagca
atggaacccactggtttgtcacacaacgcaacttttatgagcctcaaatcattacaaccgataacacttttgtctccgggaactgcgacgt
ggtgattggaatcgtcaacaacactgtctatgatcccctgcaacctgaactggattcctttaaagaagagcttgataagtatttcaagaac
cataccagccccgacgtcgatttgggagatattagtgggattaatgctagcgttgttaatatacaaaaggaaatagatcgattgaatgaa
gtggccaagaatctgaatgagtctctgattgacctgcaggagctcggaaagtatgagcaatatataaaatggccctggtcaggccgca
ggcgtcggcggcgcggtagcggcggttcaggatctgggtatatacctgaggccccacgagatgggcaggcttatgtacggaaagat
ggagaatgggtgttgctgagtactttcctcgggtaataa (SEQ ID NO: 131)
ggauccgccaccauggauuggacauggauucuguuucucguugccgccgcuacgcgcgugcacagcauguucguuuucc
ucguacuguugccucugguaucaucucaaugugugaaucuuacgacgaggacgcaacugccaccagcuuacaccaacucu
uuuacuagaggggucuauuaccccgacaagguuuucagauccucagugcugcauaguacacaagauuuguuucuucccu
ucuucuccaaugucacuugguuucacgcuauccacguguccggcacuaauggaacgaagcgguuugauaacccgguacu
uccauuuaaugacgggguauacuuugcaagcaccgagaaaaguaauaucauucguggguggaucuuuggcacuacacuc
gacuccaagacacaaucccuuuugauaguaaauaaugcuacaaaugugguuauaaaggugugcgaguuucaauucugca
augaccccuuccucggugucuauuaucacaagaacaauaaaucuuggauggaaagugaguucaggguauacagcucugc
aaacaacugcacauuugaguaugugagccaaccguuucuuauggaucuggaagguaagcaggguaacuuuaagaaccuu
cgggaguucgucuuuaagaauauagauggcuauuuuaaaaucuauaguaaacacacuccuauuaaccuugugcgcgauc
ucccucaaggguucucugcauuggaaccgcuuguugauuugccuauaggaaucaauauuacacgauuucaaacacuccu
cgcucuccauaggagcuaccuuaccccaggcgacucaagcucugguuggacggcaggagcugcagcauacuaugugggu
uaucuucagccgcggacauucuugcuuaaguauaaugagaauggaacuaucacugacgcuguugacugugcccuggacc
cucuuucagaaacaaaauguacucucaaaucauucaccguggagaagggaauauaucaaacaaguaacuuuaggguccaa
ccuaccgagagcaucgugcgauucccuaauauuaccaauuugugucccuucggugaagucuucaacgcgacccgcuuug
cuagcgucuaugcguggaacaggaagcgaauuagcaacuguguugcagauuacagugugcuguacaauagugcgagcuu
uuccacguucaaaugcuauggcgugaaaccuaccaaacuuaaugauuugugcuucacuaauguuuaugcugacagcuuc
guuauccggggugaugaggugaggcagauugcccccggucaaacuggcaaaauugccgacuacaauuacaagcucccug
augauuuuacuggaugugucauagcguggaauuccaauaaucuggacucuaagguuggugguaauuauaacuaucucu
accgccuguuucguaagagcaaucugaaacccuuugaaagagauauuuggacugagauauaucaagcuggcucaacucc
uugcaacggggucgaagguuucaauuguuacuuuccacuucaaucauacggguuucaaccaacuaacgguguagguuau
caacccuaucgggugguuguccugagcuuugagcugaaccaugccccggcuacaguaugcggcccaaagaaauccacuaa
cuuggucaagaacaaaugcgucaacuuuaacuuuaacggacucacggggacaggaguccuuaccgaauccaacaagaaau
ucuugccuuuccaacaauuuggacgagacauugcggauaccacagacgcaguacgcgacccacagacucuugaaauccuc
gacauaacacccugcaguuucggcgguguaagugucauuaccccaggcacuaauacgagcaaccaaguggcggugcucua
ccaagacguuaauugcacugaggucccaguggcuauucacgcugaccaacuuacacccacauggagaguguauaguacag
gcucaaacgucuuccagacacgggcggggugccuuauuggagcagaacauguuaacaauuccuaugaaugcgauauccc
gauuggagccgggaucugugcuagcuaucaaacccaaacaaauagccccagacgucgacguuccguggcuagucaaagca
ucaucgccuacacuaugagucuuggggccgaaaauuccguugcuuacaguaacaacaguaucgcuauccccaccaauuuu
acuauuaguguaacuacagagauucugccgguuuccaugacaaagacuuccguggauuguacgauguauauuugcggcg
acagcacagagugcagcaaucugcugcugcaauacgguaguuucugcacccaauugaaccgugcucugacgggaauugc
aguugagcaggacaagaauacucaagaaguauuugcacaagucaaacagauauacaagacgcccccgauuaaagauuucg
gcggguuuaacuuuagccaaauucuuccggaccccagcagacgccgccgaagcuuuauugaggaccugcuguuuaauaa
aguuacccuugcugaugcugguuuuaucaagcaauacggagauugccugggagauaucgccgccagggauuugaucug
ugcgcaaaaguuuaacggccuuaccguucucccgccccuucugaccgaugaaaugauagcccaauacacuuccgcacucc
uggcaggcacaauuacuuccggcuggacguuuggggccggggcagccuugcaaauuccguuugcuaugcaaauggcaua
ucguuucaaugguaucggcguaacacaaaauguccuuuaugagaaccagaaacucauugcuaaucaguuuaauuccgcu
aucggcaagauucaagacagucucagcagcacggcgagcgcacuugguaaacuucaagacguugucaaccagaaugcuca
agcccugaacacucugguaaaacaacuuagcucuaauuucggugcaauuagcuccgguccgaacgauauucugucacggc
ucccgaaagucgaagccgaaguccagaucgauaggcugaucacagggcgcuugcagagucuccaaaccuacgugacgcaa
caacucauucgggcggcugaaauucgugcaagcgcuaaucuggccgcuaccaaaaugagugaguguguucucggucaau
caaagaggguugacuuuugcggcaaaggauaucauuugaugaguuuuccgcaaucugccccucaugggguaguauuuc
ugcacguaacuuauguaccagcacaagaaaagaacuucaccacggccccagcaauaugccacgauggcaaagcucauuucc
cucgcgaaggggucuuuguaagcaauggaacccacugguuugucacacaacgcaacuuuuaugagccucaaaucauuaca
accgauaacacuuuugucuccgggaacugcgacguggugauuggaaucgucaacaacacugucuaugauccccugcaacc
ugaacuggauuccuuuaaagaagagcuugauaaguauuucaagaaccauaccagccccgacgucgauuugggagauauu
agugggauuaaugcuagcguuguuaauauacaaaaggaaauagaucgauugaaugaaguggccaagaaucugaaugagu
cucugauugaccugcaggagcucggaaaguaugagcaauauauaaaauggcccuggucaggccgcaggcgucggcggcg
cgguagcggcgguucaggaucuggguauauaccugaggccccacgagaugggcaggcuuauguacggaaagauggagaa
uggguguugcugaguacuuuccucggguaauaa (SEQ ID NO: 132)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGV [symbol] PT
KLNDLCFTNVYADSFVIRGDEVRQIAPGQTGKIADYNYKLPDDFTGCVIAWNSNNLD
SKVGGNYNYLYRLFRKSNLKPFERDI [symbol] TEIYQAGSTPCNGVEGFNCYFPLQSYGFQP
TNGVGYQPYRVVVLSFEL [symbol] HAPATVCGPKKSTNLVKNKCVNFNFNGLTGTGVLTES
NKKFLPFQQFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECDIPIGAGIC
ASYQTQTNSPRR [symbol] RSVASQSIIAYTMSLGAENSVAYSNNSIAIPTNFTISVTTEILPVSM
TKTSVDCTMYICGDSTECSNLLLQYGSFCTQLNRALTGIAVEQDKNTQEVFAQVKQI
YKTPPIKDFGGFNFSQILPDPS [symbol] RSFIEDLLFNKVTLADAGFIKQYGDCLGDIAARD
LICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTL
VKQLSSNFGAISS [symbol] NDILSRL [symbol] KVEAEVQIDRLITGRLQSLQTYVTQQLIRAAEIRASA
NLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKNL
NESLIDLQELGKYEQYIKWPWSGRRRRRRGSGGSGSGYIPEAPRDGQAYVRKDGEW
VLLSTFLG** (SEQ ID NO: 133)
WuS_3F_D2P_pVax
ggatccgccaccatggactggacctggatactctttctcgtagcagcagccacacgagtgcattcaatgtttgtcttcttggtcctcttgcc
actggttagctcccagtgtgtgaatcttactacaaggacacaactgcccccagcttacacaaactcctttactaggggtgtatattaccca
gacaaagtgtttcgcagttctgtcctgcatagcacccaagaccttttccttccgttcttcagcaacgtcacctggttccatgctatccatgtc
agtggaacgaatggcacaaagcggttcgataaccctgtcctgccctttaacgacggcgtctatttcgcttcaacagagaagagtaacat
tatcagaggatggatatttggtacaactcttgatagcaagacacaaagcctgctgattgtaaacaacgcgacaaatgtcgtcatcaaggt
ttgcgagtttcaattttgcaacgatcccttcttgggcgtgtactatcataagaacaataaaagctggatggagagcgaatttagggtgtata
gctcagctaataactgtacatttgaatatgtctctcaacccttcctcatggaccttgagggaaagcaaggaaatttcaagaatctcagaga
atttgtcttcaagaacatcgacgggtatttcaagatctactccaagcatacacccatcaacttggttagggaccttccgcaaggtttctcag
cactggagcctctggtagatctccctattgggattaatattacaagatttcaaacactcctggccttgcatagatcctatcttacccctggg
gattccagctcaggttggaccgcgggtgccgcggcgtactatgtcggatatctccaacctcggacattcctgctgaaatacaatgaaaa
tgggaccatcactgatgccgttgattgtgctctcgatcctctgagtgagaccaaatgcactcttaagagttttacagtggagaaaggtatc
tatcaaactagtaatttcagagttcaaccaaccgagtcaatagtgcgttttccaaatatcactaatctgtgtccatttggggaagtcttcaat
gctacccgattcgcaagtgtgtacgcctggaaccggaaacggatttctaactgcgttgccgattatagtgtcctctataattctgcttctttc
tctacttttaagtgctatggggtgtcccccaccaagctgaacgatctgtgtttcactaacgtctacgccgatagttttgtcattagagggga
cgaggtacggcaaatcgcgcccggccaaacggggaaaattgccgattacaactacaagcttccagacgacttcacaggttgcgtgat
tgcatggaattctaataatctggacagtaaagtgggcggcaactataactatctttaccggctgtttcggaagagcaacttgaagcccttc
gaacgcgacatatccaccgagatctatcaagccggaagtaccccgtgcaacggggtagaaggatttaattgttattttccattgcagtctt
atggatttcagcccaccaatggtgtgggataccaaccttatagggttgttgttctctccttcgaactcctgcacgctccagctactgtatgt
gggcctaagaaaagtactaatctcgttaagaataaatgcgtcaatttcaatttcaacggcttgaccgggactggagtgctcaccgaaag
caacaagaagtttctcccgtttcagcaattcggtagggatattgccgatacgacagatgcagtacgagatccccaaacactcgaaatcc
tggacattacgccatgtagctttggcggagtaagtgtcatcaccccagggactaacaccagtaaccaagttgcggtactctatcaggat
gtgaactgcactgaggtacctgtagcaattcacgcagaccaattgacgccgacgtggcgcgtctatagtacaggaagtaacgtctttca
gacaagagcgggttgtttgattggcgctgaacacgttaacaattcttacgagtgtgatatccccatcggtgcggggatctgcgccagct
atcagacacaaaccaattccccacgaaggagacgttccgtggccagccagtcaataatcgcgtatactatgtctctgggtgcggagaa
ttcagtggcctattccaataattctatagccattccaaccaattttactataagcgtcactacagagatcttgccagttagcatgacgaaaac
cagcgtcgattgtaccatgtatatatgcggcgacagtaccgaatgctcaaatctgctgctccaatatggctcattttgcactcaacttaata
gagctctgacagggatcgctgtcgaacaagataagaacactcaggaagttttcgcccaagttaagcagatatacaagaccccgcccat
caaggattttggcggatttaatttctctcagatcctgccggaccctagccgccgacgccggagctttatcgaagacttgctgtttaataag
gttactctcgcagatgcaggcttcatcaagcaatacggtgactgccttggggatatcgctgctcgggacctgatctgtgctcagaaattc
aacggtctcacggtgctgcccccactcctgaccgacgaaatgattgcccagtatacgtccgcattgctcgctggcaccatcactagcg
gctggacctttggggccggagccgcgctccaaataccttttgctatgcaaatggcttatcgcttcaatggtattggggttacgcaaaatgt
cctctacgaaaatcaaaagctcatagctaaccaattcaatagcgctatagggaaaattcaagacagcctgagttccacagcaagcgcc
ctcggcaaacttcaagatgtagtgaaccaaaatgctcaagcactcaatacactggtcaaacaactctcaagcaatttcggggcaatctc
atctggtcctaatgacatattgagcaggctccccaaagtggaagcagaagtacaaatcgacaggctgattaccggacgactccaaagc
ttgcaaacttatgtaacccaacaacttatcagggctgcagaaatccgtgcaagcgctaacctcgccgctacgaagatgtcagaatgtgt
acttgggcagtctaagagggttgatttctgtggaaaagggtaccatctgatgagttttccacagagcgctccacatggggtggtgtttctg
catgtaacctatgttcccgctcaagaaaagaattttactactgcccccgcaatttgccatgacgggaaagcccatttcccccgagaggg
agttttcgtgagtaacggaacgcactggtttgtcactcagagaaatttctacgagccccaaatcattacgaccgataatacattcgtaagc
ggtaactgcgatgtcgtcattggcatcgttaacaacactgtttatgatccccttcaacccgagcttgactcatttaaagaggaactggataa
gtactttaagaatcacacctctcccgatgtcgacctgggcgacatctctggaattaatgcctctgtcgtaaacatccaaaaggaaattgac
cgactgaatgaggtggcaaagaatcttaatgaatccctgatcgatctgcaggagcttgggaagtatgagcaatacatcaaatggccatg
gtctggcagacggcgccggagaaggggctctggcggctctggaagcgggtatattccagaggcgcccagggatgggcaagcatat
gttcggaaggatggggagtgggtgttgttgtccacgttccttggctagtga (SEQ ID NO: 134)
ggauccgccaccauggacuggaccuggauacucuuucucguagcagcagccacacgagugcauucaauguuugucuucu
ugguccucuugccacugguuagcucccagugugugaaucuuacuacaaggacacaacugcccccagcuuacacaaacucc
uuuacuagggguguauauuacccagacaaaguguuucgcaguucuguccugcauagcacccaagaccuuuuccuuccgu
ucuucagcaacgucaccugguuccaugcuauccaugucaguggaacgaauggcacaaagcgguucgauaacccuguccu
gcccuuuaacgacggcgucuauuucgcuucaacagagaagaguaacauuaucagaggauggauauuugguacaacucuu
gauagcaagacacaaagccugcugauuguaaacaacgcgacaaaugucgucaucaagguuugcgaguuucaauuuugcaa
cgaucccuucuugggcguguacuaucauaagaacaauaaaagcuggauggagagcgaauuuaggguguauagcucagcu
aauaacuguacauuugaauaugucucucaacccuuccucauggaccuugagggaaagcaaggaaauuucaagaaucucag
agaauuugucuucaagaacaucgacggguauuucaagaucuacuccaagcauacacccaucaacuugguuagggaccuuc
cgcaagguuucucagcacuggagccucugguagaucucccuauugggauuaauauuacaagauuucaaacacuccuggc
cuugcauagauccuaucuuaccccuggggauuccagcucagguuggaccgcgggugccgcggcguacuaugucggauau
cuccaaccucggacauuccugcugaaauacaaugaaaaugggaccaucacugaugccguugauugugcucucgauccucu
gagugagaccaaaugcacucuuaagaguuuuacaguggagaaagguaucuaucaaacuaguaauuucagaguucaaccaa
ccgagucaauagugcguuuuccaaauaucacuaaucuguguccauuuggggaagucuucaaugcuacccgauucgcaag
uguguacgccuggaaccggaaacggauuucuaacugcguugccgauuauaguguccucuauaauucugcuucuuucucu
acuuuuaagugcuauggggugucccccaccaagcugaacgaucuguguuucacuaacgucuacgccgauaguuuuguca
uuagaggggacgagguacggcaaaucgcgcccggccaaacggggaaaauugccgauuacaacuacaagcuuccagacgac
uucacagguugcgugauugcauggaauucuaauaaucuggacaguaaagugggcggcaacuauaacuaucuuuaccggc
uguuucggaagagcaacuugaagcccuucgaacgcgacauauccaccgagaucuaucaagccggaaguaccccgugcaac
gggguagaaggauuuaauuguuauuuuccauugcagucuuauggauuucagcccaccaauggugugggauaccaaccu
uauaggguuguuguucucuccuucgaacuccugcacgcuccagcuacuguaugugggccuaagaaaaguacuaaucucg
uuaagaauaaaugcgucaauuucaauuucaacggcuugaccgggacuggagugcucaccgaaagcaacaagaaguuucuc
ccguuucagcaauucgguagggauauugccgauacgacagaugcaguacgagauccccaaacacucgaaauccuggacau
uacgccauguagcuuuggcggaguaagugucaucaccccagggacuaacaccaguaaccaaguugcgguacucuaucag
gaugugaacugcacugagguaccuguagcaauucacgcagaccaauugacgccgacguggcgcgucuauaguacaggaa
guaacgucuuucagacaagagcggguuguuugauuggcgcugaacacguuaacaauucuuacgagugugauauccccau
cggugcggggaucugcgccagcuaucagacacaaaccaauuccccacgaaggagacguuccguggccagccagucaauaa
ucgcguauacuaugucucugggugcggagaauucaguggccuauuccaauaauucuauagccauuccaaccaauuuuac
uauaagcgucacuacagagaucuugccaguuagcaugacgaaaaccagcgucgauuguaccauguauauaugcggcgaca
guaccgaaugcucaaaucugcugcuccaauauggcucauuuugcacucaacuuaauagagcucugacagggaucgcugu
cgaacaagauaagaacacucaggaaguuuucgcccaaguuaagcagauauacaagaccccgcccaucaaggauuuuggcg
gauuuaauuucucucagauccugccggacccuagccgccgacgccggagcuuuaucgaagacuugcuguuuaauaaggu
uacucucgcagaugcaggcuucaucaagcaauacggugacugccuuggggauaucgcugcucgggaccugaucugugcu
cagaaauucaacggucucacggugcugcccccacuccugaccgacgaaaugauugcccaguauacguccgcauugcucgc
uggcaccaucacuagcggcuggaccuuuggggccggagccgcgcuccaaauaccuuuugcuaugcaaauggcuuaucgc
uucaaugguauugggguuacgcaaaauguccucuacgaaaaucaaaagcucauagcuaaccaauucaauagcgcuauagg
gaaaauucaagacagccugaguuccacagcaagcgcccucggcaaacuucaagauguagugaaccaaaaugcucaagcac
ucaauacacuggucaaacaacucucaagcaauuucggggcaaucucaucugguccuaaugacauauugagcaggcucccc
aaaguggaagcagaaguacaaaucgacaggcugauuaccggacgacuccaaagcuugcaaacuuauguaacccaacaacu
uaucagggcugcagaaauccgugcaagcgcuaaccucgccgcuacgaagaugucagaauguguacuugggcagucuaag
aggguugauuucuguggaaaaggguaccaucugaugaguuuuccacagagcgcuccacauggggugguguuucugcau
guaaccuauguucccgcucaagaaaagaauuuuacuacugcccccgcaauuugccaugacgggaaagcccauuucccccg
agagggaguuuucgugaguaacggaacgcacugguuugucacucagagaaauuucuacgagccccaaaucauuacgacc
gauaauacauucguaagcgguaacugcgaugucgucauuggcaucguuaacaacacuguuuaugauccccuucaacccg
agcuugacucauuuaaagaggaacuggauaaguacuuuaagaaucacaccucucccgaugucgaccugggcgacaucucu
ggaauuaaugccucugucguaaacauccaaaaggaaauugaccgacugaaugagguggcaaagaaucuuaaugaaucccu
gaucgaucugcaggagcuugggaaguaugagcaauacaucaaauggccauggucuggcagacggcgccggagaaggggc
ucuggcggcucuggaagcggguauauuccagaggcgcccagggaugggcaagcauauguucggaaggauggggagugg
guguuguuguccacguuccuuggcuaguga (SEQ ID NO: 135)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSPRRRRSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSRRRRSFIEDLLFNKVTLADAGFIKQYGDCLGDIAARDLIC
AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ
LSSNFGAISSGPNDILSRLPKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 136)
WuS_3F_2P_GlyDSol_pVax
ggatccgccaccatggactggacatggattttgtttcttgtggcggctgcaacgcgagttcattccatgtttgttttcctggttctcttgccg
ctcgtctctagtcaatgcgtcaatctgacgacaagaacgcaacttccccctgcctacaccaatagttttacccgtggcgtctattatccag
ataaagtgtttcgaagttcctgccttcattccacgcaagatctcttccttccattcttctctaatgtcacctggtttcatgcgatccacgtgtct
ggaaccaacgggactaaacgattcgacaatcccgtcctgccatttaacgatggagtatacttcgcatccaccgagaaatctggcattat
aagagggtggatattcgggactacactcgacagcaagacacaaagtctcctgattgttaacaacgcgacaaacgtcgtaattaaagttt
gcgaatttcaattttgtaatgacccgttcttgggcgtgtattatcacaagaataacaaaagttggatggaatccgaattccgggtttattcca
gcgcgaataattgcacatttgaatacgttagccaacctttcctgatggatctcgaaggcaaacaaggaaactttaagaatcttcgggaatt
cgttttcaagaacatcgacgggtactttaagatatactcaaaacacaccccaattaatttggtccgagatctgccgcaaggctttagtgcg
cttgagcccctcgtagatctcccaattggtattaatatcacacgctttcaaaccctgctggcattgcatcggagctatctgactcccggcg
atagttcttcagggtggacggcgggtgccgctgcttactatgtaggctatctgcagcctcgtacatttctcctcaaatacaatgaaaatgg
cactattaccgacgccgttgattgcgctctggacccactgagcgagactaaatgcaccctcaaatcattcactgttgagaagggaattta
ccaaacatcaaacttcagggtccaacctacggaaagcatcgtgcggttccccaacatcactaacctctgcccctttggagaagtatttaa
cgctacaaggttcgcttccgtctacgcctggaacaggaaaagaatcagtaattgcgtggctgattactccgtgctgtacaattccgcctc
attttctacatttaagtgttatggcgttaacgggactaagcttaacgacctctgcttcacaaacgtctatgccgacagctttgtcattcgcgg
ggatgaagtaagacagatagcacccggtcaaactggcaaaattgctgattacaattacaagttgccagatgatttcactggatgcgttat
agcatggaactctaacaaccttgactcaaaggttggtggcaactataattatttgtatcgcctgtttcgcaaatctaatctcaagcctttcga
gcgcgacataaatacgaccatataccaagcggggtccaccccttgtaatggagtcgaggggtttaattgctattttccgttgcaatcctac
gggttccaaccaacaaacggcgtcggctatcaaccctatcgggttgtcgtactctcattcgagctcaaccatgcaccagcaacagtttgt
ggccccaagaagagcacaaatttggtcaagaataaatgcgttaattttaatttcaatggtctgactggcacaggggttcttaccgaatcaa
ataagaagtttctgccatttcagcagttcggaagggactgtgcagggaccacagatgccgttagagacccccaaacactcgaaattctg
gacatcacgccatgcagtttcggtggtgttagcgtgattactccgggtactaatacgtccaaccaagtggctgtgttgtatcaagacgtta
actgtaccgaagttcctgtagcaatccatgccggtcaactgacccccacgtggcgagtttatagcaccggttccaacgtctttcaaacaa
gagccggatgtctcataggcgctgaacatgtgaataattcatacgaatgtgacattccaatcggcgcagggatttgcgcctcatatcaga
cacaaactaactccccgagaagacgtcgctcagtggcgtcacaaagcatcatcgcttatacgatgagcctctgcgccgagaactctgt
cgcatattctaacaactctattgcaattcctacaaattttacaatttcttgcactactgagatcctgcccgtaagcatgacgaaaacatcctg
cgactgcacaatgtatatctgtggcgactcaactgagtgctccaatctcctcttgcaatacggatctttctgtactcaactcaacagagcac
ttacaggaatagccgtcgaacaagacaagaacacacaagaggtcttcgcccaagtaaagcaatgttacaaaaccccacctattaaaga
ctttggtgggtttaatttctcacagattcttccagatccttcccgtagaaggagaagctttattgaagacctcttgtttaataaagtcactcttg
cagacgctgggtttattaaacaatatggagactgcttgggagacatagcggcaagagacctgatctgcgctcaaaagtttaatgggtgc
actgtgttgccaccccttctgaccgacgagatgatcgctcagtataccagtgccttgctggcagggaccataactagcggatggactttc
ggtgcaggagctgctctgcaaatcccttttgcgatgcaaatggcctacaggtttaatggtataggagttactcagaatgtcctgtacgaaa
atcaaaagctgatcgccaatcaattcaacagtgctattgggaaaatacaggacagtttgagttcaacagcgagcgctctcggcaaactg
caggatgttgtgaatcaaaacgcgcaagctttgaacactcttgtgaagcagctttcatccaacttcggagcgatctcatccgtcctgaac
gacatattgtcaagacttgacccacctgaagcggaagttcagatagaccgactcataacgggccgacttcagtccttgcagacatacgt
gacccaacaacttatccgcgcagccgaaataagggcttcagctaaccttgcagcaaccaaaatgtcagagtgcgtgctcggtcaaag
caagcgggtagacttttgtggcaaggggtatcatcttatgtcctttcctcaatccgcccctcacggggtggtcttcttgcactgcacttatgt
acctgctcaagagaagaattttacgaccgcccctgcgatctgtcacgacgggaaagcacatttcccccgcgagggagtctttgtgtcta
atggtactcattggtttgttacgcagcggaacttttacgaacctcaaataattacaacggataatacagatgttagtgggaattgcgacgtg
gtgatcggtatagtcaacaatacggtgtatgatccacttcaaccagaacttgattcctttaaggaagagctggacaaatatttcaagaacc
atacatcccctgacgtggaccttggcgatataagcggcattaatgcttcagtggtcaatatacaaaaggaaatcgatcgcctgaatgag
gtcgcaaagaatttgaatgagtccctgatcgacctgcaagagctcgggaaatatgagcagtacatcaagtggccctggtcaggtagac
gtaggcggcgccggggcagtggcggctcagggagcggttatatacccgaagcccctagagatgggcaagcttatgtccgaaagga
cggcgaatgggtgctcctttccactttcttgggataatag (SEQ ID NO: 137)
ggauccgccaccauggacuggacauggauuuuguuucuuguggcggcugcaacgcgaguucauuccauguuuguuuuc
cugguucucuugccgcucgucucuagucaaugcgucaaucugacgacaagaacgcaacuucccccugccuacaccaauag
uuuuacccguggcgucuauuauccagauaaaguguuucgaaguuccugccuucauuccacgcaagaucucuuccuucca
uucuucucuaaugucaccugguuucaugcgauccacgugucuggaaccaacgggacuaaacgauucgacaaucccgucc
ugccauuuaacgauggaguauacuucgcauccaccgagaaaucuggcauuauaagaggguggauauucgggacuacacu
cgacagcaagacacaaagucuccugauuguuaacaacgcgacaaacgucguaauuaaaguuugcgaauuucaauuuugua
augacccguucuugggcguguauuaucacaagaauaacaaaaguuggauggaauccgaauuccggguuuauuccagcgc
gaauaauugcacauuugaauacguuagccaaccuuuccugauggaucucgaaggcaaacaaggaaacuuuaagaaucuuc
gggaauucguuuucaagaacaucgacggguacuuuaagauauacucaaaacacaccccaauuaauuugguccgagaucug
ccgcaaggcuuuagugcgcuugagccccucguagaucucccaauugguauuaauaucacacgcuuucaaacccugcugg
cauugcaucggagcuaucugacucccggcgauaguucuucaggguggacggcgggugccgcugcuuacuauguaggcu
aucugcagccucguacauuucuccucaaauacaaugaaaauggcacuauuaccgacgccguugauugcgcucuggaccca
cugagcgagacuaaaugcacccucaaaucauucacuguugagaagggaauuuaccaaacaucaaacuucaggguccaacc
uacggaaagcaucgugcgguuccccaacaucacuaaccucugccccuuuggagaaguauuuaacgcuacaagguucgcuu
ccgucuacgccuggaacaggaaaagaaucaguaauugcguggcugauuacuccgugcuguacaauuccgccucauuuuc
uacauuuaaguguuauggcguuaacgggacuaagcuuaacgaccucugcuucacaaacgucuaugccgacagcuuuguc
auucgcggggaugaaguaagacagauagcacccggucaaacuggcaaaauugcugauuacaauuacaaguugccagauga
uuucacuggaugcguuauagcauggaacucuaacaaccuugacucaaagguugguggcaacuauaauuauuuguaucgc
cuguuucgcaaaucuaaucucaagccuuucgagcgcgacauaaauacgaccauauaccaagcgggguccaccccuuguaa
uggagucgagggguuuaauugcuauuuuccguugcaauccuacggguuccaaccaacaaacggcgucggcuaucaaccc
uaucggguugucguacucucauucgagcucaaccaugcaccagcaacaguuuguggccccaagaagagcacaaauuugg
ucaagaauaaaugcguuaauuuuaauuucaauggucugacuggcacagggguucuuaccgaaucaaauaagaaguuucu
gccauuucagcaguucggaagggacugugcagggaccacagaugccguuagagacccccaaacacucgaaauucuggaca
ucacgccaugcaguuucggugguguuagcgugauuacuccggguacuaauacguccaaccaaguggcuguguuguauca
agacguuaacuguaccgaaguuccuguagcaauccaugccggucaacugacccccacguggcgaguuuauagcaccggu
uccaacgucuuucaaacaagagccggaugucucauaggcgcugaacaugugaauaauucauacgaaugugacauuccaau
cggcgcagggauuugcgccucauaucagacacaaacuaacuccccgagaagacgucgcucaguggcgucacaaagcauca
ucgcuuauacgaugagccucugcgccgagaacucugucgcauauucuaacaacucuauugcaauuccuacaaauuuuaca
auuucuugcacuacugagauccugcccguaagcaugacgaaaacauccugcgacugcacaauguauaucuguggcgacuc
aacugagugcuccaaucuccucuugcaauacggaucuuucuguacucaacucaacagagcacuuacaggaauagccgucg
aacaagacaagaacacacaagaggucuucgcccaaguaaagcaauguuacaaaaccccaccuauuaaagacuuugguggg
uuuaauuucucacagauucuuccagauccuucccguagaaggagaagcuuuauugaagaccucuuguuuaauaaaguca
cucuugcagacgcuggguuuauuaaacaauauggagacugcuugggagacauagcggcaagagaccugaucugcgcuca
aaaguuuaaugggugcacuguguugccaccccuucugaccgacgagaugaucgcucaguauaccagugccuugcuggca
gggaccauaacuagcggauggacuuucggugcaggagcugcucugcaaaucccuuuugcgaugcaaauggccuacaggu
uuaaugguauaggaguuacucagaauguccuguacgaaaaucaaaagcugaucgccaaucaauucaacagugcuauugg
gaaaauacaggacaguuugaguucaacagcgagcgcucucggcaaacugcaggauguugugaaucaaaacgcgcaagcuu
ugaacacucuugugaagcagcuuucauccaacuucggagcgaucucauccguccugaacgacauauugucaagacuugac
ccaccugaagcggaaguucagauagaccgacucauaacgggccgacuucaguccuugcagacauacgugacccaacaacu
uauccgcgcagccgaaauaagggcuucagcuaaccuugcagcaaccaaaaugucagagugcgugcucggucaaagcaagc
ggguagacuuuuguggcaagggguaucaucuuauguccuuuccucaauccgccccucacgggguggucuucuugcacu
gcacuuauguaccugcucaagagaagaauuuuacgaccgccccugcgaucugucacgacgggaaagcacauuucccccgc
gagggagucuuugugucuaaugguacucauugguuuguuacgcagcggaacuuuuacgaaccucaaauaauuacaacgg
auaauacagauguuagugggaauugcgacguggugaucgguauagucaacaauacgguguaugauccacuucaaccaga
acuugauuccuuuaaggaagagcuggacaaauauuucaagaaccauacauccccugacguggaccuuggcgauauaagcg
gcauuaaugcuucaguggucaauauacaaaaggaaaucgaucgccugaaugaggucgcaaagaauuugaaugagucccu
gaucgaccugcaagagcucgggaaauaugagcaguacaucaaguggcccuggucagguagacguaggcggcgccggggc
aguggcggcucagggagcgguuauauacccgaagccccuagagaugggcaagcuuauguccgaaaggacggcgaauggg
ugcuccuuuccacuuucuugggauaauag (SEQ ID NO: 138)
MDWTWILFLVAAATRVHSMFVFLVLLPLVSSQCVNLTTRTQLPPAYTNSFTRGVYY
PDKVFRSSCLHSTQDLFLPFFSNVTWFHAIHVSGTNGTKRFDNPVLPFNDGVYFASTE
KSGIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDCAGTTDAVRDPQTLEILDITPCSFGGVSVITPGTNTSNQVAVLYQD
VNCTEVPVAIHAGQLTPTWRVYSTGSNVFQTRAGCLIGAEHVNNSYECDIPIGAGICA
SYQTQTNSPRRRRSVASQSIIAYTMSLCAENSVAYSNNSIAIPTNFTISCTTEILPVSMT
KTSCDCTMYICGDSTECSNLLLQYGSFCTQLNRALTGIAVEQDKNTQEVFAQVKQCY
KTPPIKDFGGFNFSQILPDPSRRRRSFIEDLLFNKVTLADAGFIKQYGDCLGDIAARDLI
CAQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTDVSGNCDVVIGIVNNTV
YDPLQPELDSFKEELDKYFKNHTSPDVDLGDISGINASVVNIQKEIDRLNEVAKNLNE
SLIDLQELGKYEQYIKWPWSGRRRRRRGSGGSGSGYIPEAPRDGQAYVRKDGEWVL
LSTFLG** (SEQ ID NO: 139)
WuS_3F_2P_Gly_pVax
ggatccgccaccatggattggacctggatacttttcctcgtggccgcagcaacaagagtccactccatgttcgtcttcttggtacttttgcc
actcgtcagttctcagtgcgttaacctgactactagaacccaattgcccccggcatacacaaactctttcacccggggtgtctactatccc
gacaaagtgtttagaagtagcgtgctgcacagcacccaagatctctttctgccattcttctcaaacgtcacctggtttcacgccatccatgt
aagcgggaccaacggcacaaagcgttttgataaccctgttttgccattcaatgatggcgtgtattttgcttccactgagaaaagcaacatc
attagagggtggatatttggcacaacgcttgactccaagacgcagagtcttttgatagtaaacaacgcaactaatgtggtcattaaagtct
gtgaatttcaattttgcaatgaccccttccttggagtctattaccacaagaacaacaaaagctggatggaaagcgaatttagggtctacag
ctctgccaataactgcacattcgaatacgtcagccaaccattcttgatggacctggaaggcaagcaaggaaactttaagaatctgaggg
aatttgtgtttaagaatatcgacggatattttaagatctattccaagcatactcccattaatctcgttcgtgaccttcctcagggtttctctgcat
tggaacccctcgtagatttgcccattgggattaatatcactagattccagacgctgcttgcactccatcgatcttatctgacccctggtgac
tcctcttccgggtggacggcgggtgctgcagcctactacgttggctatttgcaacctaggacctttctgttgaagtataatgagaatggga
ctattactgatgccgttgattgcgccctcgatccgctgtcagaaacaaagtgcaccctgaagagcttcacagtagaaaagggaatctatc
aaacctcaaatttccgcgttcaaccaactgaatcaatcgtgcgttttcctaacatcacaaatctgtgtccgtttggagaagtatttaatgcga
cgcgtttcgcaagcgtctacgcgtggaatcgcaaacgtatctctaattgcgtagcagattattctgtgctgtacaatagcgcatctttctca
acgtttaagtgctacggcgttaatgggaccaagctgaatgatctctgtttcactaatgtgtacgcagacagttttgtaattagaggagacg
aggttaggcaaatagcaccgggtcaaactggcaaaatcgccgactataactacaagctccctgatgacttcacgggctgcgtaattgct
tggaactctaataacctggactctaaagtcggcgggaattataattatctctatcggttgtttcgaaaatccaatctcaaaccctttgagcg
ggacatcaatactacaatttatcaagctggtagtactccttgcaatggggtagaaggcttcaattgttatttcccccttcaatcttacggattt
caacccacgaacggcgtagggtaccagccctatcgagtggtggtactgtcattcgaacttaatcacgccccagcaacagtctgcggg
cctaagaaaagcacgaatcttgtcaagaataagtgtgtaaatttcaacttcaatggtcttacaggcacgggagtgctcactgagtctaata
agaaatttcttcctttccaacaattcggtcgtgatattgccgatactactgatgcagtccgagatccacaaactctcgaaatcctcgatatta
ctccttgtagttttggcggcgtctccgtgatcaccccagggaccaacactagtaaccaagtggcggtgctctaccaagatgttaactgca
cagaagtcccggtagcgatccatgccgaccagctcactcccacatggcgtgtttacagcacagggtcaaacgttttccagacccgtgc
cggatgtcttataggagccgaacacgtaaataacagttatgaatgcgatatcccaattggtgcaggtatctgtgcgtcatatcaaaccca
aactaattctccgagacgacgacggagcgttgcctcacaatcaataatcgcctacacaatgtccctcggtgccgaaaattcagtcgctta
ctctaacaatagcattgctatccctaccaacttcactatttctgttaccacggaaattttgcctgtatccatgaccaaaacatctgttgattgc
acgatgtacatctgcggggattctaccgaatgttctaacctgcttctgcaatacggctccttctgcacccaattgaaccgcgcactgactg
ggattgctgtggaacaagacaagaatactcaagaagtatttgcccaggtcaaacagatttacaaaactcccccaattaaagatttcggc
ggtttcaattttagtcaaattctgccagatccaagtcgacgccgcaggagctttattgaggacctgctctttaataaagtcacgctggccg
acgccggcttcataaaacagtatggcgattgtcttggagacatcgccgcccgcgacctcatttgcgcacaaaagttcaatgggctcacc
gtgttgccaccactgctcacagatgagatgatcgcacagtacacgagcgcccttcttgccggcactatcacgtctggttggacgttcgg
tgccggagccgctctgcaaattccctttgcaatgcaaatggcctatagatttaatggaattggcgtaacacagaacgtgttgtacgagaa
ccagaagctcattgccaaccagttcaattccgctattggcaaaatacaagactctctcagctcaactgctagcgcactgggaaaattgca
agacgtagtcaatcaaaatgcccaagccctcaatactctcgtcaaacagttgtcttccaactttggggctatcagtagtgtactcaatgac
attctttcaagactggacccgcccgaggcggaagtccaaattgatcgtctgataactggaaggttgcaaagccttcagacctacgttac
gcaacaacttattagggctgccgaaataagggcatccgctaatctggcagctacaaagatgtctgaatgtgttttgggacagagcaaac
gggttgacttctgcggtaaaggttaccatctcatgtcttttccacaaagcgcaccgcacggagtcgtcttcctgcatgtaacatacgtccc
agcccaagaaaagaattttaccacagccccagccatctgccacgacggcaaggcgcatttcccaagggaaggcgtgtttgtatccaa
cgggacgcattggtttgtcactcaaaggaacttttacgaaccccaaattattaccactgataacaccttcgtttctgggaactgtgatgtcg
tgattgggatagtaaacaacacggtatatgatccactgcaaccagaactggattccttcaaagaagagctggacaaatacttcaagaat
catactagtcctgacgtcgacctgggcgatatcagtggaatcaacgctagcgtcgtaaacattcaaaaggagatcgatagacttaacga
ggtcgccaagaatctcaatgaaagcctcatcgatttgcaagaactcggaaaatatgagcaatacataaaatggccatggtctggcagg
agaagacgcaggagaggtagcggcggcagcggatcagggtacattccggaagcccccagggacggacaggcatatgtccgcaa
ggacggagaatgggttcttcttagcacttttctggggtaataa (SEQ ID NO: 140)
ggauccgccaccauggauuggaccuggauacuuuuccucguggccgcagcaacaagaguccacuccauguucgucuucu
ugguacuuuugccacucgucaguucucagugcguuaaccugacuacuagaacccaauugcccccggcauacacaaacucu
uucacccggggugucuacuaucccgacaaaguguuuagaaguagcgugcugcacagcacccaagaucucuuucugccau
ucuucucaaacgucaccugguuucacgccauccauguaagcgggaccaacggcacaaagcguuuugauaacccuguuuu
gccauucaaugauggcguguauuuugcuuccacugagaaaagcaacaucauuagaggguggauauuuggcacaacgcuu
gacuccaagacgcagagucuuuugauaguaaacaacgcaacuaauguggucauuaaagucugugaauuucaauuuugca
augaccccuuccuuggagucuauuaccacaagaacaacaaaagcuggauggaaagcgaauuuagggucuacagcucugcc
aauaacugcacauucgaauacgucagccaaccauucuugauggaccuggaaggcaagcaaggaaacuuuaagaaucugag
ggaauuuguguuuaagaauaucgacggauauuuuaagaucuauuccaagcauacucccauuaaucucguucgugaccuu
ccucaggguuucucugcauuggaaccccucguagauuugcccauugggauuaauaucacuagauuccagacgcugcuug
cacuccaucgaucuuaucugaccccuggugacuccucuuccggguggacggcgggugcugcagccuacuacguuggcua
uuugcaaccuaggaccuuucuguugaaguauaaugagaaugggacuauuacugaugccguugauugcgcccucgauccg
cugucagaaacaaagugcacccugaagagcuucacaguagaaaagggaaucuaucaaaccucaaauuuccgcguucaacc
aacugaaucaaucgugcguuuuccuaacaucacaaaucuguguccguuuggagaaguauuuaaugcgacgcguuucgca
agcgucuacgcguggaaucgcaaacguaucucuaauugcguagcagauuauucugugcuguacaauagcgcaucuuucu
caacguuuaagugcuacggcguuaaugggaccaagcugaaugaucucuguuucacuaauguguacgcagacaguuuugu
aauuagaggagacgagguuaggcaaauagcaccgggucaaacuggcaaaaucgccgacuauaacuacaagcucccugaug
acuucacgggcugcguaauugcuuggaacucuaauaaccuggacucuaaagucggcgggaauuauaauuaucucuaucg
guuguuucgaaaauccaaucucaaacccuuugagcgggacaucaauacuacaauuuaucaagcugguaguacuccuugca
augggguagaaggcuucaauuguuauuucccccuucaaucuuacggauuucaacccacgaacggcguaggguaccagcc
cuaucgaguggugguacugucauucgaacuuaaucacgccccagcaacagucugcgggccuaagaaaagcacgaaucuug
ucaagaauaaguguguaaauuucaacuucaauggucuuacaggcacgggagugcucacugagucuaauaagaaauuucu
uccuuuccaacaauucggucgugauauugccgauacuacugaugcaguccgagauccacaaacucucgaaauccucgaua
uuacuccuuguaguuuuggcggcgucuccgugaucaccccagggaccaacacuaguaaccaaguggcggugcucuacca
agauguuaacugcacagaagucccgguagcgauccaugccgaccagcucacucccacauggcguguuuacagcacagggu
caaacguuuuccagacccgugccggaugucuuauaggagccgaacacguaaauaacaguuaugaaugcgauaucccaauu
ggugcagguaucugugcgucauaucaaacccaaacuaauucuccgagacgacgacggagcguugccucacaaucaauaau
cgccuacacaaugucccucggugccgaaaauucagucgcuuacucuaacaauagcauugcuaucccuaccaacuucacua
uuucuguuaccacggaaauuuugccuguauccaugaccaaaacaucuguugauugcacgauguacaucugcggggauuc
uaccgaauguucuaaccugcuucugcaauacggcuccuucugcacccaauugaaccgcgcacugacugggauugcugug
gaacaagacaagaauacucaagaaguauuugcccaggucaaacagauuuacaaaacucccccaauuaaagauuucggcgg
uuucaauuuuagucaaauucugccagauccaagucgacgccgcaggagcuuuauugaggaccugcucuuuaauaaaguc
acgcuggccgacgccggcuucauaaaacaguauggcgauugucuuggagacaucgccgcccgcgaccucauuugcgcaca
aaaguucaaugggcucaccguguugccaccacugcucacagaugagaugaucgcacaguacacgagcgcccuucuugccg
gcacuaucacgucugguuggacguucggugccggagccgcucugcaaauucccuuugcaaugcaaauggccuauagauu
uaauggaauuggcguaacacagaacguguuguacgagaaccagaagcucauugccaaccaguucaauuccgcuauuggca
aaauacaagacucucucagcucaacugcuagcgcacugggaaaauugcaagacguagucaaucaaaaugcccaagcccuca
auacucucgucaaacaguugucuuccaacuuuggggcuaucaguaguguacucaaugacauucuuucaagacuggaccc
gcccgaggcggaaguccaaauugaucgucugauaacuggaagguugcaaagccuucagaccuacguuacgcaacaacuua
uuagggcugccgaaauaagggcauccgcuaaucuggcagcuacaaagaugucugaauguguuuugggacagagcaaacg
gguugacuucugcgguaaagguuaccaucucaugucuuuuccacaaagcgcaccgcacggagucgucuuccugcaugua
acauacgucccagcccaagaaaagaauuuuaccacagccccagccaucugccacgacggcaaggcgcauuucccaagggaa
ggcguguuuguauccaacgggacgcauugguuugucacucaaaggaacuuuuacgaaccccaaauuauuaccacugaua
acaccuucguuucugggaacugugaugucgugauugggauaguaaacaacacgguauaugauccacugcaaccagaacu
ggauuccuucaaagaagagcuggacaaauacuucaagaaucauacuaguccugacgucgaccugggcgauaucaguggaa
ucaacgcuagcgucguaaacauucaaaaggagaucgauagacuuaacgaggucgccaagaaucucaaugaaagccucauc
gauuugcaagaacucggaaaauaugagcaauacauaaaauggccauggucuggcaggagaagacgcaggagagguagcg
gcggcagcggaucaggguacauuccggaagcccccagggacggacaggcauauguccgcaaggacggagaauggguucu
ucuuagcacuuuucugggguaauaa (SEQ ID NO: 141)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIY
KTPPIKDFGGFNFSQILPDPSRRRRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 142)
WuS_3F_2P_NoClev_pVax
ggatccgccaccatggattggacgtggattctgtttctggtggccgcagcgacaagggtgcattcaatgtttgtgttcctggtcctgctgc
cactggtctcatcacagtgtgtaaacctgactacaagaacgcagcttccgcctgcctacacgaacagcttcaccaggggagtgtattat
cctgacaaagtctttaggagctctgttctccactccactcaagacctgtttctgcccttcttcagtaacgtgacttggtttcacgcaatacat
gtctccggcacaaatggaaccaaaagattcgataaccctgttctcccattcaatgatggagtatattttgctagcactgaaaagtctaacat
tattagaggctggatatttggcacgacattggactccaagacgcaaagtctcttgattgtgaacaacgcaacaaacgtggtgataaaagt
ttgcgaattccaattttgcaatgacccattcctgggagtttactaccacaagaataacaaaagctggatggaatccgagttccgggtttact
cctctgctaacaactgtacctttgagtatgtgagtcaaccattccttatggatctcgaaggaaaacaaggtaacttcaagaacctgaggg
agtttgtgtttaagaatatcgatggctattttaagatttatagcaaacacactccgattaatctggtgagagatctcccgcaaggattttctgc
tttggagccattggttgacttgcctattggaatcaacatcacccgttttcaaactctgcttgcgctgcatagatcctaccttacgcctggcga
ttcaagcagtggctggaccgcgggagcggccgcctattatgtaggctacttgcagcctcgcacctttctcctcaagtacaatgaaaacg
gcacaattacagacgcagtggattgtgctctggaccccctcagtgaaactaaatgtaccctgaaaagcttcactgttgagaaaggcatat
atcaaacctcaaactttagagtgcaacctactgaaagcattgtaagattccctaacattacaaacctgtgcccctttggcgaagtctttaat
gcaacccggtttgctagcgtgtatgcttggaaccgcaagaggatatccaattgcgtcgcagattattccgtcctgtataactctgccagct
ttagtaccttcaaatgttatggggtatctcccacaaaactcaatgatctttgtttcacaaatgtctatgctgactcctttgttatcagagggga
cgaagttcgccaaattgctccaggtcaaacaggaaagattgcagattataactacaagcttcccgacgattttacaggttgtgtgatagct
tggaactccaataatctggattccaaggtaggcgggaactacaattatctctacaggctcttccggaaatccaatctcaagccgttcgaa
agggatataagcactgagatctatcaagcaggcagtacaccctgtaacggagtagagggcttcaactgctactttccactgcaatcctat
gggtttcaaccgactaacggtgtcgggtaccaaccctatcgtgtcgtggtcctgtcctttgagcttctgcacgctcctgctaccgtttgcg
gccccaagaaaagcacgaatttggtcaagaacaaatgtgtcaactttaacttcaacggattgacagggaccggagtattgaccgaatct
aataagaaatttctgcccttccaacaattcggacgggacatagcagacacaaccgatgctgtcagggacccacagacacttgaaatac
tcgatatcaccccatgcagctttggcggagtctcagtcattacgcctggcaccaatacttccaatcaagttgcagtgctctatcaggatgt
caattgtactgaggtccccgtcgccatccacgcggatcaacttacccccacatggcgagtatatagtaccgggagcaacgtctttcaaa
cccgagcaggatgtctgataggtgccgaacacgtaaacaacagctacgaatgtgatatcccgatcggcgcagggatttgcgctagct
accaaacccaaactaattctccgcgccgccgcaggtccgtagcaagtcaatcaataatagcatacaccatgtcattgggagctgaaaa
cagcgtggcatatagcaacaattccatagctatccctacaaatttcacgatttctgttaccaccgaaattctgccagtgagcatgaccaaa
acctcagtggattgtacgatgtacatatgcggcgattccacggaatgttccaatctccttttgcaatacggcagcttttgtacccaactgaa
tagagctctgacgggtatagcagtagagcaggataagaacactcaagaggtgtttgcccaggtcaaacaaatttacaagactccccca
ataaaagactttggcggcttcaatttcagccaaatcttgccagacccttccaggcggcggcgctcatttatcgaagatttgcttttcaataa
agtcaccctggccgacgccggatttattaaacaatacggcgattgtctgggcgacatcgccgcaagggacctcatctgtgcgcaaaag
ttcaatggcctgacggtgcttccaccactcctgactgatgagatgattgcccaatacacatctgccctgctggctggtacaataacgagt
gggtggacctttggggctggagcagcattgcaaattccattcgccatgcaaatggcatatcgttttaacggcattggagtgactcaaaat
gtgctgtatgaaaaccaaaagcttattgcaaatcagtttaattccgccattggcaaaatccaggatagcctcagtagtacagcaagcgcc
ttggggaaactgcaagatgtggttaatcaaaatgcacaagctctcaataccctggtcaagcaacttagtagtaactttggtgccatcagc
agcgttctcaacgacatcctgagtcgtcttgatcccccagaggcagaggttcaaattgaccggcttatcactggaaggcttcaatccctg
caaacttacgtgactcagcaactgatacgcgctgcagaaattcgggcctcagcaaaccttgccgcgacaaagatgagcgaatgcgtg
ctgggacaatccaagcgggtcgacttttgtggtaaaggctatcatctgatgagcttcccacagtccgctccacacggcgtcgttttcctg
cacgtgacctatgtgccagcacaggagaagaactttacaacagccccggctatctgccacgatggcaaagctcactttcctagagagg
gagtgtttgtaagcaatggaacccattggttcgttacacaaagaaacttttatgagccgcaaattatcacaacagataatacattcgtctcc
gggaactgtgacgttgtgatagggattgtcaacaacacagtgtacgaccccctgcaacccgagctggattcatttaaagaagaactcg
acaagtacttcaagaatcatactagtccagatgtggatctgggcgatatatcaggaatcaatgccagcgtggtcaatattcaaaaggag
attgatagactgaacgaggttgccaagaatctgaatgaaagcctgatcgatctgcaagaattgggcaagtatgagcagtacattaaatg
gccctggtctggcgggagcggcggatctgggtctggatatattcccgaagctcctagagatggacaagcttacgtccgtaaagacgg
cgagtgggttcttctctccacattcctcggctgatga (SEQ ID NO: 143)
ggauccgccaccauggauuggacguggauucuguuucugguggccgcagcgacaagggugcauucaauguuuguguuc
cugguccugcugccacuggucucaucacaguguguaaaccugacuacaagaacgcagcuuccgccugccuacacgaacag
cuucaccaggggaguguauuauccugacaaagucuuuaggagcucuguucuccacuccacucaagaccuguuucugccc
uucuucaguaacgugacuugguuucacgcaauacaugucuccggcacaaauggaaccaaaagauucgauaacccuguucu
cccauucaaugauggaguauauuuugcuagcacugaaaagucuaacauuauuagaggcuggauauuuggcacgacauug
gacuccaagacgcaaagucucuugauugugaacaacgcaacaaacguggugauaaaaguuugcgaauuccaauuuugcaa
ugacccauuccugggaguuuacuaccacaagaauaacaaaagcuggauggaauccgaguuccggguuuacuccucugcu
aacaacuguaccuuugaguaugugagucaaccauuccuuauggaucucgaaggaaaacaagguaacuucaagaaccugag
ggaguuuguguuuaagaauaucgauggcuauuuuaagauuuauagcaaacacacuccgauuaaucuggugagagaucuc
ccgcaaggauuuucugcuuuggagccauugguugacuugccuauuggaaucaacaucacccguuuucaaacucugcuug
cgcugcauagauccuaccuuacgccuggcgauucaagcaguggcuggaccgcgggagcggccgccuauuauguaggcua
cuugcagccucgcaccuuucuccucaaguacaaugaaaacggcacaauuacagacgcaguggauugugcucuggaccccc
ucagugaaacuaaauguacccugaaaagcuucacuguugagaaaggcauauaucaaaccucaaacuuuagagugcaaccu
acugaaagcauuguaagauucccuaacauuacaaaccugugccccuuuggcgaagucuuuaaugcaacccgguuugcua
gcguguaugcuuggaaccgcaagaggauauccaauugcgucgcagauuauuccguccuguauaacucugccagcuuuag
uaccuucaaauguuaugggguaucucccacaaaacucaaugaucuuuguuucacaaaugucuaugcugacuccuuuguu
aucagaggggacgaaguucgccaaauugcuccaggucaaacaggaaagauugcagauuauaacuacaagcuucccgacga
uuuuacagguugugugauagcuuggaacuccaauaaucuggauuccaagguaggcgggaacuacaauuaucucuacagg
cucuuccggaaauccaaucucaagccguucgaaagggauauaagcacugagaucuaucaagcaggcaguacacccuguaa
cggaguagagggcuucaacugcuacuuuccacugcaauccuauggguuucaaccgacuaacggugucggguaccaaccc
uaucgugucgugguccuguccuuugagcuucugcacgcuccugcuaccguuugcggccccaagaaaagcacgaauuugg
ucaagaacaaaugugucaacuuuaacuucaacggauugacagggaccggaguauugaccgaaucuaauaagaaauuucug
cccuuccaacaauucggacgggacauagcagacacaaccgaugcugucagggacccacagacacuugaaauacucgauau
caccccaugcagcuuuggcggagucucagucauuacgccuggcaccaauacuuccaaucaaguugcagugcucuaucagg
augucaauuguacugagguccccgucgccauccacgcggaucaacuuacccccacauggcgaguauauaguaccgggagc
aacgucuuucaaacccgagcaggaugucugauaggugccgaacacguaaacaacagcuacgaaugugauaucccgaucgg
cgcagggauuugcgcuagcuaccaaacccaaacuaauucuccgcgccgccgcagguccguagcaagucaaucaauaauag
cauacaccaugucauugggagcugaaaacagcguggcauauagcaacaauuccauagcuaucccuacaaauuucacgauu
ucuguuaccaccgaaauucugccagugagcaugaccaaaaccucaguggauuguacgauguacauaugcggcgauuccac
ggaauguuccaaucuccuuuugcaauacggcagcuuuuguacccaacugaauagagcucugacggguauagcaguagag
caggauaagaacacucaagagguguuugcccaggucaaacaaauuuacaagacucccccaauaaaagacuuuggcggcuu
caauuucagccaaaucuugccagacccuuccaggcggcggcgcucauuuaucgaagauuugcuuuucaauaaagucaccc
uggccgacgccggauuuauuaaacaauacggcgauugucugggcgacaucgccgcaagggaccucaucugugcgcaaaa
guucaauggccugacggugcuuccaccacuccugacugaugagaugauugcccaauacacaucugcccugcuggcuggu
acaauaacgaguggguggaccuuuggggcuggagcagcauugcaaauuccauucgccaugcaaauggcauaucguuuua
acggcauuggagugacucaaaaugugcuguaugaaaaccaaaagcuuauugcaaaucaguuuaauuccgccauuggcaaa
auccaggauagccucaguaguacagcaagcgccuuggggaaacugcaagaugugguuaaucaaaaugcacaagcucucaa
uacccuggucaagcaacuuaguaguaacuuuggugccaucagcagcguucucaacgacauccugagucgucuugauccc
ccagaggcagagguucaaauugaccggcuuaucacuggaaggcuucaaucccugcaaacuuacgugacucagcaacugau
acgcgcugcagaaauucgggccucagcaaaccuugccgcgacaaagaugagcgaaugcgugcugggacaauccaagcggg
ucgacuuuugugguaaaggcuaucaucugaugagcuucccacaguccgcuccacacggcgucguuuuccugcacgugac
cuaugugccagcacaggagaagaacuuuacaacagccccggcuaucugccacgauggcaaagcucacuuuccuagagagg
gaguguuuguaagcaauggaacccauugguucguuacacaaagaaacuuuuaugagccgcaaauuaucacaacagauaau
acauucgucuccgggaacugugacguugugauagggauugucaacaacacaguguacgacccccugcaacccgagcugg
auucauuuaaagaagaacucgacaaguacuucaagaaucauacuaguccagauguggaucugggcgauauaucaggaauc
aaugccagcguggucaauauucaaaaggagauugauagacugaacgagguugccaagaaucugaaugaaagccugaucg
aucugcaagaauugggcaaguaugagcaguacauuaaauggcccuggucuggcgggagcggcggaucugggucuggau
auauucccgaagcuccuagagauggacaagcuuacguccguaaagacggcgaguggguucuucucuccacauuccucgg
cugauga (SEQ ID NO: 144)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSPRRRRSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSRRRRSFIEDLLFNKVTLADAGFIKQYGDCLGDIAARDLIC
AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ
LSSNFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGGSGGSGSGYIPEAPRDGQAYVRKDGEWVLLSTFLG**
(SEQ ID NO: 145)
WuS_3F_2P_pVax
ggatccgccaccatggattggacctggattctctttcttgtggcggcggcaacacgcgtccacagcatgttcgtctttctggtattgctgc
cgcttgtgagtagccagtgtgtgaacttgactaccaggacccagctcccaccggcttataccaattccttcacaagaggtgtctactatc
cagataaagttttccgcagctcagtgttgcatagcacacaggatctctttctgccattcttcagcaacgtcacgtggtttcatgcgatacac
gttagtggaacaaacggaacaaaacgcttcgacaaccctgttctgccattcaatgacggagtttactttgcgagtaccgagaaatctaac
atcattagagggtggatctttgggactacattggattctaaaacccagtcactcctcatagtcaataacgctacaaatgtggtgattaaggt
atgcgaatttcagttttgcaacgacccatttctcggtgtatattatcacaagaataataaaagttggatggagtccgagttccgcgtctattc
atcagccaataattgtactttcgaatatgtttctcaaccgtttctcatggatctcgaaggaaagcaagggaattttaagaatctccgggagtt
tgtcttcaagaacatagatggctattttaaaatttactcaaagcatactcctattaacctcgttcgggatctcccccagggttttagcgccct
ggagccactggttgatctgccaattgggattaatatcacacgctttcaaactctcctggcccttcacagatcttacttgaccccaggtgatt
caagtagtggttggacagctggagctgctgcatattatgtaggttatctccaaccccgcacctttctcctcaaatataacgagaacggca
ccattacagatgcggtggactgtgccttggaccctctttctgagaccaagtgcacactcaaaagcttcactgtcgaaaaggggatttacc
agacatcaaattttagagttcaacccaccgaaagcattgtgagatttcctaacattacaaacttgtgcccatttggggaagtctttaacgct
acacgctttgctagcgtctatgcttggaaccgaaaacggattagtaactgcgtagctgattattccgtcttgtacaacagcgcatcttttag
cactttcaagtgttatggagtaagcccaacaaagctcaacgacctttgttttactaacgtctatgctgattcattcgtgattcgtggggatga
ggttcgtcagatcgccccaggccaaaccgggaaaatcgctgattataattataaattgcctgacgattttaccggctgtgtaatcgcctg
gaattccaacaatcttgattccaaggttggcggcaactacaactatctctaccgtctgtttcgcaaatccaatctcaagcccttcgaacgc
gatatttcaactgaaatctatcaggcagggtccactccgtgtaacggcgtagaaggatttaattgttacttcccattgcaaagttatggcttt
caacccaccaacggagtcgggtaccaaccatacagagtcgtcgtgctctcatttgagctccttcatgcacctgccacggtgtgcggcc
caaagaaatcaacgaaccttgtgaagaataaatgtgtcaattttaactttaatggcctgacagggactggcgtcctcacagaatctaataa
gaagtttctccctttccagcaatttggtcgcgatatagctgataccacagatgcagttagagacccacagacacttgagattctcgatatta
ccccgtgctcctttggcggcgtgtccgtcattactcccggtaccaatacgtctaaccaggtagcagtgctctaccaagatgtaaattgtac
tgaggtacccgtggcaatccatgccgaccaactgactccaacgtggcgggtttattcaaccggaagcaacgtgtttcaaacacgggct
ggctgccttataggcgctgagcacgtgaataatagttacgagtgtgatatcccgatcggagccggcatctgtgcatcttatcaaacacaa
acaaactccccgcgccggcggagaagcgtggctagccaaagtataatcgcttatacaatgtccttgggcgcggaaaattcagtggctt
attccaataattcaattgccattcctaccaactttacaattagcgtgaccacagaaatcttgcctgtgtctatgaccaagaccagcgtcgatt
gcaccatgtatatctgtggagatagcaccgagtgttcaaatttgctcctgcaatacggttccttttgtacacagcttaaccgcgccctcaca
ggtattgctgttgaacaagacaagaatactcaagaggtatttgctcaggtaaaacaaatttataagaccccaccgataaaagattttggc
ggtttcaatttctcccaaatattgccagatcctagtaggcgtcgtagatcatttatcgaggatctcctgttcaataaagtaaccctcgccgac
gctggtttcatcaaacaatatggcgactgcctgggagatattgcagctagggatttgatttgtgcacagaagttcaatggactcaccgttc
tcccgcctctcctgacagatgagatgattgcacaatacacctctgctcttttggccgggaccattacgagcggttggacttttggcgcgg
gtgcggctctccaaattcctttcgcgatgcaaatggcgtatagatttaatggaattggcgttactcaaaacgtcttgtacgagaatcagaa
actgatcgccaaccaatttaacagtgcaattggcaaaatccaagatagccttagttctactgcttcagcattgggtaagttgcaagatgtg
gtcaaccaaaacgcacaagcactcaataccctcgtgaagcaattgtccagcaattttggagctatctcaagtgtgctcaacgacatccttt
ctaggcttgatccacccgaggcagaggttcaaatcgacagactgataactggcaggctccaatctctgcaaacgtacgtgacacagca
actgattagggctgctgagatcagggcgtccgcgaatttggcagcaaccaaaatgagcgaatgcgtgctgggacaatcaaagagagt
tgatttctgtggaaagggttaccatctcatgtccttccctcaatcagctccccatggagttgtgtttctgcacgttacttacgtgccggcaca
agaaaagaatttcaccactgcaccggctatatgtcatgatgggaaagcccacttcccgcgggaaggcgttttcgtgtccaacgggact
cattggttcgtcacacaaaggaacttctatgagccacaaataattacaacagacaacacctttgtctctgggaactgcgatgtcgtgattg
gaatcgtgaacaacactgtctacgatccgctgcaacccgaactcgactcattcaaagaggaactggataagtatttcaagaaccatacc
agccccgatgtcgatctgggcgatatctccgggataaatgcttcagtagtaaacattcaaaaggaaatcgaccggctgaacgaggttg
cgaagaatcttaatgagtcattgatcgacctgcaagaacttggtaagtatgagcagtacatcaagtggccttggtcaggccgcaggcgt
cggcgtcgtgggagcggcggcagtgggagcggatatattccagaagcgccccgagacggacaagcttacgtacgaaaagacgga
gaatgggtactgctttccacttttcttggctaatga (SEQ ID NO: 146)
ggauccgccaccauggauuggaccuggauucucuuucuuguggcggcggcaacacgcguccacagcauguucgucuuuc
ugguauugcugccgcuugugaguagccagugugugaacuugacuaccaggacccagcucccaccggcuuauaccaauuc
cuucacaagaggugucuacuauccagauaaaguuuuccgcagcucaguguugcauagcacacaggaucucuuucugcca
uucuucagcaacgucacgugguuucaugcgauacacguuaguggaacaaacggaacaaaacgcuucgacaacccuguucu
gccauucaaugacggaguuuacuuugcgaguaccgagaaaucuaacaucauuagaggguggaucuuugggacuacauug
gauucuaaaacccagucacuccucauagucaauaacgcuacaaauguggugauuaagguaugcgaauuucaguuuugca
acgacccauuucucgguguauauuaucacaagaauaauaaaaguuggauggaguccgaguuccgcgucuauucaucagc
caauaauuguacuuucgaauauguuucucaaccguuucucauggaucucgaaggaaagcaagggaauuuuaagaaucuc
cgggaguuugucuucaagaacauagauggcuauuuuaaaauuuacucaaagcauacuccuauuaaccucguucgggauc
ucccccaggguuuuagcgcccuggagccacugguugaucugccaauugggauuaauaucacacgcuuucaaacucuccu
ggcccuucacagaucuuacuugaccccaggugauucaaguagugguuggacagcuggagcugcugcauauuauguaggu
uaucuccaaccccgcaccuuucuccucaaauauaacgagaacggcaccauuacagaugcgguggacugugccuuggaccc
ucuuucugagaccaagugcacacucaaaagcuucacugucgaaaaggggauuuaccagacaucaaauuuuagaguucaac
ccaccgaaagcauugugagauuuccuaacauuacaaacuugugcccauuuggggaagucuuuaacgcuacacgcuuugc
uagcgucuaugcuuggaaccgaaaacggauuaguaacugcguagcugauuauuccgucuuguacaacagcgcaucuuuu
agcacuuucaaguguuauggaguaagcccaacaaagcucaacgaccuuuguuuuacuaacgucuaugcugauucauucg
ugauucguggggaugagguucgucagaucgccccaggccaaaccgggaaaaucgcugauuauaauuauaaauugccuga
cgauuuuaccggcuguguaaucgccuggaauuccaacaaucuugauuccaagguuggcggcaacuacaacuaucucuac
cgucuguuucgcaaauccaaucucaagcccuucgaacgcgauauuucaacugaaaucuaucaggcaggguccacuccgug
uaacggcguagaaggauuuaauuguuacuucccauugcaaaguuauggcuuucaacccaccaacggagucggguaccaa
ccauacagagucgucgugcucucauuugagcuccuucaugcaccugccacggugugcggcccaaagaaaucaacgaaccu
ugugaagaauaaaugugucaauuuuaacuuuaauggccugacagggacuggcguccucacagaaucuaauaagaaguuu
cucccuuuccagcaauuuggucgcgauauagcugauaccacagaugcaguuagagacccacagacacuugagauucucga
uauuaccccgugcuccuuuggcggcguguccgucauuacucccgguaccaauacgucuaaccagguagcagugcucuac
caagauguaaauuguacugagguacccguggcaauccaugccgaccaacugacuccaacguggcggguuuauucaaccg
gaagcaacguguuucaaacacgggcuggcugccuuauaggcgcugagcacgugaauaauaguuacgagugugauauccc
gaucggagccggcaucugugcaucuuaucaaacacaaacaaacuccccgcgccggcggagaagcguggcuagccaaagua
uaaucgcuuauacaauguccuugggcgcggaaaauucaguggcuuauuccaauaauucaauugccauuccuaccaacuu
uacaauuagcgugaccacagaaaucuugccugugucuaugaccaagaccagcgucgauugcaccauguauaucugugga
gauagcaccgaguguucaaauuugcuccugcaauacgguuccuuuuguacacagcuuaaccgcgcccucacagguauug
cuguugaacaagacaagaauacucaagagguauuugcucagguaaaacaaauuuauaagaccccaccgauaaaagauuuu
ggcgguuucaauuucucccaaauauugccagauccuaguaggcgucguagaucauuuaucgaggaucuccuguucaaua
aaguaacccucgccgacgcugguuucaucaaacaauauggcgacugccugggagauauugcagcuagggauuugauuug
ugcacagaaguucaauggacucaccguucucccgccucuccugacagaugagaugauugcacaauacaccucugcucuuu
uggccgggaccauuacgagcgguuggacuuuuggcgcgggugcggcucuccaaauuccuuucgcgaugcaaauggcgu
auagauuuaauggaauuggcguuacucaaaacgucuuguacgagaaucagaaacugaucgccaaccaauuuaacagugca
auuggcaaaauccaagauagccuuaguucuacugcuucagcauuggguaaguugcaagauguggucaaccaaaacgcaca
agcacucaauacccucgugaagcaauuguccagcaauuuuggagcuaucucaagugugcucaacgacauccuuucuaggc
uugauccacccgaggcagagguucaaaucgacagacugauaacuggcaggcuccaaucucugcaaacguacgugacacag
caacugauuagggcugcugagaucagggcguccgcgaauuuggcagcaaccaaaaugagcgaaugcgugcugggacaau
caaagagaguugauuucuguggaaaggguuaccaucucauguccuucccucaaucagcuccccauggaguuguguuucu
gcacguuacuuacgugccggcacaagaaaagaauuucaccacugcaccggcuauaugucaugaugggaaagcccacuucc
cgcgggaaggcguuuucguguccaacgggacucauugguucgucacacaaaggaacuucuaugagccacaaauaauuac
aacagacaacaccuuugucucugggaacugcgaugucgugauuggaaucgugaacaacacugucuacgauccgcugcaac
ccgaacucgacucauucaaagaggaacuggauaaguauuucaagaaccauaccagccccgaugucgaucugggcgauauc
uccgggauaaaugcuucaguaguaaacauucaaaaggaaaucgaccggcugaacgagguugcgaagaaucuuaaugagu
cauugaucgaccugcaagaacuugguaaguaugagcaguacaucaaguggccuuggucaggccgcaggcgucggcgucg
ugggagcggcggcagugggagcggauauauuccagaagcgccccgagacggacaagcuuacguacgaaaagacggagaa
uggguacugcuuuccacuuuucuuggcuaauga (SEQ ID NO: 147)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSPRRRRSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSRRRRSFIEDLLFNKVTLADAGFIKQYGDCLGDIAARDLIC
AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ
LSSNFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 148)
WuS_3F_D2P_GlyDSol_pVax
ggatccgccaccatggattggacctggatcttgtttctcgtcgcagctgccacaagagtccacagtatgtttgtatttctcgttctcctgcct
cttgtgtcctctcagtgtgtgaatctgacgaccagaactcagctcccacccgcatacactaacagtatcacgaggggcgtttattaccca
gacaaggttttccgaagcagttgtctgtatagtacacaggatttgttcctgccattctttagtaacgtgacttggtttcatgcgatccacgttt
ctggcacaaacggtaccaaaagattcgataaccccgtgctgcctttcaatgacggcgtgtatttcgcttctactgaaaagtccggcatca
tccggggatggatcttcggcactactttggatagcaaaacccaatccctgctgattgtgaataatgcaaccaacgtggtgatcaaagtct
gtgagtttcaattctgtaatgacccatttctgggcgtttactaccacaagaacaacaaaagttggatggagtccgaatttcgcgtctactca
tccgcgaacaattgcacatttgagtatgtgagtcaaccattcttgatggatcttgaagggaagcaaggcaatttcaagaacctgcggga
gtttgtatttaagaacattgacggctatttcaagatatattctaaacatactcccattaacctcgtgcgcgacttgccccagggattcagtgc
tctggaaccactggtcgatctccccatcggcattaatattacacgctttcaaactctcctcgctttgcatcggtcctatcttactccgggaga
tagctcaagcggatggacggcaggcgcggcggcatattatgttggatatctccaaccacgcacgttccttctgaaatataatgaaaatg
gcactattactgatgcagtcgactgcgccctggaccctctgtctgagaccaagtgtaccctgaagtcattcaccgttgagaagggaatct
accaaacctctaactttagggtacaacctactgagagcattgtccgcttcccgaatatcaccaatctttgtccgtttggtgaggtgtttaac
gcgacacgatttgcttccgtatacgcctggaatcgcaaacgcatcagcaactgtgtggctgattactcatttctctataattccgcttccttc
tctactttcaagtgttatggggtgaatgggactaaacttaatgacttgtgctttactaacgtgtatgctgatagcttcgtcattcgtggagatg
aggtcaggcaaatagctcccggacaaacagggaagatagcggactataactacaaactgcctgatgatttcaccgggtgcgtcatcg
cgtggaacagcaataacctcgatagcaaggttggcggaaactataattatctctatcgtttgtttaggaaaagcaatctcaagcccttcga
gcgggatattaatacaacgatatatcaagctggctctaccccgtgcaacggagtagagggcttcaattgctactttcctttgcagtcctac
ggattccaacccaccaacggagtgggctaccaaccataccgtgtcgtggttttgagtttcgaactgaaccacgcaccagcaacagtct
gcggaccgaagaagagtacaaaccttgtgaagaataagtgcgtgaactttaatttcaatggcctgactggaaccggagttctgacgga
atccaataagaaatttctgccgtttcagcaatttggacgggattgtgctggaacgactgatgccgtacgtgatcctcaaacactggaaatc
ctggacataaccccttgttcctttggtggcgtaagcgttattactccaggcacaaacacatcaaatcaagtcgccgtactgtatcaaggtg
tcaactgtactgaagtacctgtagccattcatgcaggacaactgacccctacatggcgagtgtattcaacgggaagcaacgtatttcaaa
ccagggccggctgtctcatcggagcagagcatgtcaataatagttatgaatgcgacatcccaataggtgctgggatctgcgcgagcta
ccaaacccaaactaatagcccacgaagacggagatctgtcgcgtcccaaagcattattgcgtacacgatgagcctctgtgcagaaaat
tcagttgcctacagcaacaatagcatcgctattccaaccaatttcactatcagctgtacaacagaaattctcccagtctccatgacgaaga
catcctgcgattgtacaatgtatatatgcggcgactcaacagaatgttcaaatttgttgctgcaatacgggtccttctgcacccaactcaat
cgagctcttacaggcatagcggtcgaacaagacaagaacacacaagaagtgtttgcccaagttaaacagtgttacaagacaccaccta
tcaaagatttcggcggttttaacttttctcagatcttgccagacccatctaggcggcggcgatcctttatcgaggaccttctcttcaataagg
taactcttgcagacgctggatttattaagcaatacggcgactgtctcggggatatcgccgctagggatctgatctgtgcccagaaatttaa
cggctgcacggtgctgccccctctgctgactgatgaaatgatagcacaatatacttctgcattgctggccggtaccattacatcaggatg
gacatttggtgccggggcggcgctccaaattcccttcgccatgcaaatggcctataggtttaacggcatcggggtgacccaaaacgtc
ctctatgagaatcaaaagctgattgctaaccagtttaactcagcaataggaaagattcaagactctctgtcaagtaccgcatccgcccttg
gaaagctccaagacgttgttaaccagaatgcacaagctctcaacacgctcgtgaaacaactctcttcaaattttggtgcgatctcttctgg
cccaaatgacattttgagccggcttcccaaggtagaagctgaagtacaaattgatcgcctgatcaccggacggctccaaagtctgcag
acgtacgtcacccagcaactgatacgggcagcggagatccgggcttctgccaacctggccgccacgaagatgagcgaatgcgtgct
cggacagtccaaaagagtagatttctgtggcaagggctatcatctcatgtcctttccccaatccgcccctcacggagttgtcttccttcatt
gcacttacgtccccgctcaagaaaagaattttactacggcacctgctatctgtcacgacgggaaagcccattttcctagagaaggtgtgt
ttgtatctaacggcacgcactggttcgtcacgcaacgtaacttttacgagccccagatcatcaccacagacaatacggatgtatcaggta
attgtgatgtcctgattggtatcgtcaataacactgtatacgatcctttgcaaccggaactggactcctttaaagaggaacttgataagtatt
tcaagaatcacacttccccagatgtcgatctcggggacatctcaggaattaatgcatcagtggtcaatattcaaaaggaaattgatcgctt
gaatgaggttgcaaagaatttgaatgaaagccttatcgaccttcaagagctgggcaaatatgagcagtacattaaatggccttggagcg
gtcgccggcgccgaaggcggggttccggcggtagcggtagcggttatattccagaagctcctcgcgatgggcaggcttatgtgagg
aaagatggtgaatgggtccttttgtccacgttcctcgggtagtaa (SEQ ID NO: 149)
ggauccgccaccauggauuggaccuggaucuuguuucucgucgcagcugccacaagaguccacaguauguuuguauuuc
ucguucuccugccucuuguguccucucagugugugaaucugacgaccagaacucagcucccacccgcauacacuaacagu
aucacgaggggcguuuauuacccagacaagguuuuccgaagcaguugucuguauaguacacaggauuuguuccugccau
ucuuuaguaacgugacuugguuucaugcgauccacguuucuggcacaaacgguaccaaaagauucgauaaccccgugcu
gccuuucaaugacggcguguauuucgcuucuacugaaaaguccggcaucauccggggauggaucuucggcacuacuuug
gauagcaaaacccaaucccugcugauugugaauaaugcaaccaacguggugaucaaagucugugaguuucaauucugua
augacccauuucugggcguuuacuaccacaagaacaacaaaaguuggauggaguccgaauuucgcgucuacucauccgcg
aacaauugcacauuugaguaugugagucaaccauucuugauggaucuugaagggaagcaaggcaauuucaagaaccugc
gggaguuuguauuuaagaacauugacggcuauuucaagauauauucuaaacauacucccauuaaccucgugcgcgacuu
gccccagggauucagugcucuggaaccacuggucgaucuccccaucggcauuaauauuacacgcuuucaaacucuccucg
cuuugcaucgguccuaucuuacuccgggagauagcucaagcggauggacggcaggcgcggcggcauauuauguuggaua
ucuccaaccacgcacguuccuucugaaauauaaugaaaauggcacuauuacugaugcagucgacugcgcccuggacccuc
ugucugagaccaaguguacccugaagucauucaccguugagaagggaaucuaccaaaccucuaacuuuaggguacaaccu
acugagagcauuguccgcuucccgaauaucaccaaucuuuguccguuuggugagguguuuaacgcgacacgauuugcuu
ccguauacgccuggaaucgcaaacgcaucagcaacuguguggcugauuacucauuucucuauaauuccgcuuccuucuc
uacuuucaaguguuauggggugaaugggacuaaacuuaaugacuugugcuuuacuaacguguaugcugauagcuucgu
cauucguggagaugaggucaggcaaauagcucccggacaaacagggaagauagcggacuauaacuacaaacugccugaug
auuucaccgggugcgucaucgcguggaacagcaauaaccucgauagcaagguuggcggaaacuauaauuaucucuaucg
uuuguuuaggaaaagcaaucucaagcccuucgagcgggauauuaauacaacgauauaucaagcuggcucuaccccgugca
acggaguagagggcuucaauugcuacuuuccuuugcaguccuacggauuccaacccaccaacggagugggcuaccaacca
uaccgugucgugguuuugaguuucgaacugaaccacgcaccagcaacagucugcggaccgaagaagaguacaaaccuug
ugaagaauaagugcgugaacuuuaauuucaauggccugacuggaaccggaguucugacggaauccaauaagaaauuucu
gccguuucagcaauuuggacgggauugugcuggaacgacugaugccguacgugauccucaaacacuggaaauccuggac
auaaccccuuguuccuuugguggcguaagcguuauuacuccaggcacaaacacaucaaaucaagucgccguacuguauca
aggugucaacuguacugaaguaccuguagccauucaugcaggacaacugaccccuacauggcgaguguauucaacggga
agcaacguauuucaaaccagggccggcugucucaucggagcagagcaugucaauaauaguuaugaaugcgacaucccaau
aggugcugggaucugcgcgagcuaccaaacccaaacuaauagcccacgaagacggagaucugucgcgucccaaagcauua
uugcguacacgaugagccucugugcagaaaauucaguugccuacagcaacaauagcaucgcuauuccaaccaauuucacu
aucagcuguacaacagaaauucucccagucuccaugacgaagacauccugcgauuguacaauguauauaugcggcgacuc
aacagaauguucaaauuuguugcugcaauacggguccuucugcacccaacucaaucgagcucuuacaggcauagcgguc
gaacaagacaagaacacacaagaaguguuugcccaaguuaaacaguguuacaagacaccaccuaucaaagauuucggcgg
uuuuaacuuuucucagaucuugccagacccaucuaggcggcggcgauccuuuaucgaggaccuucucuucaauaaggua
acucuugcagacgcuggauuuauuaagcaauacggcgacugucucggggauaucgccgcuagggaucugaucugugccc
agaaauuuaacggcugcacggugcugcccccucugcugacugaugaaaugauagcacaauauacuucugcauugcuggc
cgguaccauuacaucaggauggacauuuggugccggggcggcgcuccaaauucccuucgccaugcaaauggccuauagg
uuuaacggcaucggggugacccaaaacguccucuaugagaaucaaaagcugauugcuaaccaguuuaacucagcaauagg
aaagauucaagacucucugucaaguaccgcauccgcccuuggaaagcuccaagacguuguuaaccagaaugcacaagcuc
ucaacacgcucgugaaacaacucucuucaaauuuuggugcgaucucuucuggcccaaaugacauuuugagccggcuucc
caagguagaagcugaaguacaaauugaucgccugaucaccggacggcuccaaagucugcagacguacgucacccagcaac
ugauacgggcagcggagauccgggcuucugccaaccuggccgccacgaagaugagcgaaugcgugcucggacaguccaa
aagaguagauuucuguggcaagggcuaucaucucauguccuuuccccaauccgccccucacggaguugucuuccuucau
ugcacuuacguccccgcucaagaaaagaauuuuacuacggcaccugcuaucugucacgacgggaaagcccauuuuccuag
agaagguguguuuguaucuaacggcacgcacugguucgucacgcaacguaacuuuuacgagccccagaucaucaccacag
acaauacggauguaucagguaauugugauguccugauugguaucgucaauaacacuguauacgauccuuugcaaccgga
acuggacuccuuuaaagaggaacuugauaaguauuucaagaaucacacuuccccagaugucgaucucggggacaucucag
gaauuaaugcaucaguggucaauauucaaaaggaaauugaucgcuugaaugagguugcaaagaauuugaaugaaagccu
uaucgaccuucaagagcugggcaaauaugagcaguacauuaaauggccuuggagcggucgccggcgccgaaggcggggu
uccggcgguagcgguagcgguuauauuccagaagcuccucgcgaugggcaggcuuaugugaggaaagauggugaaugg
guccuuuuguccacguuccucggguaguaa (SEQ ID NO: 150)
MDWTWILFLVAAATRVHSMFVFLVLLPLVSSQCVNLTTRTQLPPAYTNSITRGVYYP
DKVFRSSCLYSTQDLFLPFFSNVTWFHAIHVSGTNGTKRFDNPVLPFNDGVYFASTEK
SGIIRGWIFGTTLDSKTQSLLIVNNATNVVIKVCEFQFCNDPFLGVYYHKNNKSWMES
EFRVYSSANNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAAAYYVGYLQ
PRTFLLKYNENGTITDAVDCALDPLSETKCTLKSFTVEKGIYQTSNFRVQPTESIVRFP
NITNLCPFGEVFNATRFASVYAWNRKRISNCVADYSFLYNSASFSTFKCYGVNGTKL
NDLCFTNVYADSFVIRGDEVRQIAPGQTGKIADYNYKLPDDFTGCVIAWNSNNLDSK
VGGNYNYLYRLFRKSNLKPFERDINTTIYQAGSTPCNGVEGFNCYFPLQSYGFQPTN
GVGYQPYRVVVLSFELNHAPATVCGPKKSTNLVKNKCVNFNFNGLTGTGVLTESNK
KFLPFQQFGRDCAGTTDAVRDPQTLEILDITPCSFGGVSVITPGTNTSNQVAVLYQGV
NCTEVPVAIHAGQLTPTWRVYSTGSNVFQTRAGCLIGAEHVNNSYECDIPIGAGICAS
YQTQTNSPRRRRSVASQSIIAYTMSLCAENSVAYSNNSIAIPTNFTISCTTEILPVSMTK
TSCDCTMYICGDSTECSNLLLQYGSFCTQLNRALTGIAVEQDKNTQEVFAQVKQCYK
TPPIKDFGGFNFSQILPDPSRRRRSFIEDLLFNKVTLADAGFIKQYGDCLGDIAARDLIC
AQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTDVSGNCDVLIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 151)
WuS_3F_D2P_GlyD3_pVax
ggatccgccaccatggactggacatggatacttttcttggtagcggcggcgacacgcgtgcactccatgtttgttttcctcgtcctgctcc
cacttgtctcaagtcaatgcgttaacctgactacgaggacgcagctcccgcccgcctacacaaactcttttacccggggtgtgtactacc
ccgacaaagttttccgcagttcatgtctccactcaacacaggacctctttctgccattcttctcaaatgtcacatggtttcacgccatccacg
tttccggcactaacggtaccaaacggttcgacaaccctgttctgccattcaatgatggggtgtattttgcgagcacagagaagtccaatat
aatcagaggttggatcttcggtacaacgctggacagtaaaactcaatctctgctgatagtgaataacgctacgaacgtcgtcattaaggt
gtgcgagtttcaattttgcaacgatccattcttgggagtgtactatcataagaacaacaaatcatggatggagagcgagtttagggtgtatt
cctctgcaaacaactgtacatttgaatacgtgagccagccttttcttatggacctcgaaggtaagcaaggtaacttcaagaacttgcggg
aatttgttttcaagaacattgatggatacttcaaaatttactccaaacatacccctatcaatctggtccgcgaccttccacaaggattttccgc
acttgaacccttggtcgacctgcctattggaatcaatatcacgcggtttcagacgcttctcgctctccatagatcctacctcacgcccggc
gacagttcaagtgggtggaccgcaggcgcggcggcctattatgtgggatacttgcaaccccgcacttttctcctgaaatataatgagaa
tgggaccataaccgatgcagttgattgtgccttggaccccctgtccgagaccaaatgcacgctgaagtctttcacagtagagaaggga
atttaccaaacttccaacttcagagttcaacccacagaatctatcgttcgctttcccaatattacaaatttgtgtccgtttggagaggtgttca
atgctacaaggtttgcttccgtatatgcctggaatcgtaaacgcatctctaattgcgtagcggactactcagttttgtataacagtgctagct
tctccactttcaagtgttacggcgttaatgggaccaagctgaatgacctgtgttttaccaacgtgtatgctgactccttcgtaataagaggg
gatgaggttaggcaaatcgcccctggccagacagggaaaatcgctgattacaattacaagttgccagatgactttaccgggtgtgtcat
cgcttggaactccaataatctggattccaaagttggtgggaactataattacctctatcggctgttcagaaaatccaaccttaagcccttcg
aaagagatatcaacactacaatttatcaggctggttcaactccgtgtaatggggtcgagggtttcaactgctacttcccgttgcagagttat
gggttccagccgacgaatggggtcgggtaccaaccgtacagagtagtagttctgtcctttgagttgaatcatgccccagcaacagtgtg
cggcccaaagaaatcaacaaaccttgttaagaataaatgcgtgaacttcaactttaacgggcttactgggactggggtgctcacagaat
ccaacaagaaattcttgccattccaacaatttggccgcgattgtgcagatacaaccgacgccgtgagagatccccaaacattggagata
cttgatatcactccctgctcttttggtggcgtcagcgtcatcaccccaggaaccaatacaagcaatcaagtggctgtcctttatcaagatgt
caattgtaccgaagtcccagtcgcaatacatgcggatcaactgaccccaacatggagagtttactcaacgggatctaacgtgtttcaaa
ctcgtgctggctgcctgataggagcggagcatgtgaataattcctatgaatgcgacattcccattggggctggaatctgtgcatcctatc
aaacacaaactaactctccccgccggcggcggagcgtcgccagccaaagcattattgcatatacgatgtccctgtgcgcagaaaattc
tgttgcatacagcaataactccatcgctatccctacaaactttaccatcagctgtacaaccgaaatcttgcccgtttctatgactaaaacaa
gttgtgactgcactatgtacatctgtggcgactcaacagagtgttctaaccttctgcttcaatatggatctttctgtacacaacttaatcgcgc
tctcaccggtatagctgttgagcaagataagaacactcaggaagttttcgcccaagtcaaacaatgttataaaacaccacccataaaag
acttcggcggatttaatttctctcaaatactgccggacccatccaggagacgaagaagcttcatagaagatcttctcttcaacaaggtgac
cctggccgatgcggggtttatcaagcaatatggcgactgtctcggcgatattgctgcacgcgatctgatatgtgcacagaaattcaatgg
gtgtaccgtgctcccacctctgctgacagatgaaatgatcgctcaatataccagtgcgctcttggctggaacaattactagtggttggact
tttggggctggagccgcactccaaatcccttttgccatgcaaatggcctatcgctttaatgggataggggtcactcagaatgtcttgtatg
aaaaccagaagttgattgctaaccaatttaattcagctatagggaaaattcaagacagcctcagtagtactgccagtgccctgggcaaa
ctgcaagatgtcgtgaaccaaaatgctcaagccctgaataccctcgttaagcaacttagctcaaactttggtgcgatttcctcaggcccta
atgacatcctctcaaggctgcctaaagtggaagctgaggtccaaatcgatcgcctgattacgggtcgcctgcaatcactccaaacatat
gtcacccagcagttgatcagagcggccgagatacgggcatcagcaaatttggcggccacgaaaatgtcagagtgcgtacttggtcaa
agtaaaagagttgatttctgcggaaaaggttaccaccttatgtctttcccccagtccgctccacatggagtggtctttctgcattgtacttat
gtgccagcccaagaaaagaattttactaccgcccccgctatttgtcatgatggtaaggcgcacttccccagagaaggagtgtttgtgtcc
aacgggactcactggtttgtgactcaaaggaacttttatgaacctcaaattatcaccacagataacacatttgtgtccgggaattgcgatgt
ggttatcggcattgttaataataccgtttacgatcccttgcaacctgagttggatagtttcaaggaagaacttgacaaatactttaagaatca
cacttccccggatgtagacctcggggacatttccggaattaatgcgagtgttgtgaatatacagaaagagatagaccgactcaacgag
gttgctaagaacctcaacgagagccttatcgatcttcaagaactcggcaaatacgagcaatacattaaatggccttggtccggcagaag
gagacggcgaaggggaagtggcggcagcggctctggatacatcccggaagctccacgggatgggcaagcatatgttcgcaaggat
ggagaatgggtccttcttagcaccttcttgggataatga (SEQ ID NO: 152)
ggauccgccaccauggacuggacauggauacuuuucuugguagcggcggcgacacgcgugcacuccauguuuguuuucc
ucguccugcucccacuugucucaagucaaugcguuaaccugacuacgaggacgcagcucccgcccgccuacacaaacucu
uuuacccgggguguguacuaccccgacaaaguuuuccgcaguucaugucuccacucaacacaggaccucuuucugccau
ucuucucaaaugucacaugguuucacgccauccacguuuccggcacuaacgguaccaaacgguucgacaacccuguucug
ccauucaaugaugggguguauuuugcgagcacagagaaguccaauauaaucagagguuggaucuucgguacaacgcugg
acaguaaaacucaaucucugcugauagugaauaacgcuacgaacgucgucauuaaggugugcgaguuucaauuuugcaa
cgauccauucuugggaguguacuaucauaagaacaacaaaucauggauggagagcgaguuuaggguguauuccucugca
aacaacuguacauuugaauacgugagccagccuuuucuuauggaccucgaagguaagcaagguaacuucaagaacuugc
gggaauuuguuuucaagaacauugauggauacuucaaaauuuacuccaaacauaccccuaucaaucugguccgcgaccuu
ccacaaggauuuuccgcacuugaacccuuggucgaccugccuauuggaaucaauaucacgcgguuucagacgcuucucg
cucuccauagauccuaccucacgcccggcgacaguucaaguggguggaccgcaggcgcggcggccuauuaugugggaua
cuugcaaccccgcacuuuucuccugaaauauaaugagaaugggaccauaaccgaugcaguugauugugccuuggaccccc
uguccgagaccaaaugcacgcugaagucuuucacaguagagaagggaauuuaccaaacuuccaacuucagaguucaaccc
acagaaucuaucguucgcuuucccaauauuacaaauuuguguccguuuggagagguguucaaugcuacaagguuugcuu
ccguauaugccuggaaucguaaacgcaucucuaauugcguagcggacuacucaguuuuguauaacagugcuagcuucuc
cacuuucaaguguuacggcguuaaugggaccaagcugaaugaccuguguuuuaccaacguguaugcugacuccuucgua
auaagaggggaugagguuaggcaaaucgccccuggccagacagggaaaaucgcugauuacaauuacaaguugccagaug
acuuuaccgggugugucaucgcuuggaacuccaauaaucuggauuccaaaguuggugggaacuauaauuaccucuaucg
gcuguucagaaaauccaaccuuaagcccuucgaaagagauaucaacacuacaauuuaucaggcugguucaacuccgugua
auggggucgaggguuucaacugcuacuucccguugcagaguuauggguuccagccgacgaauggggucggguaccaacc
guacagaguaguaguucuguccuuugaguugaaucaugccccagcaacagugugcggcccaaagaaaucaacaaaccuug
uuaagaauaaaugcgugaacuucaacuuuaacgggcuuacugggacuggggugcucacagaauccaacaagaaauucuu
gccauuccaacaauuuggccgcgauugugcagauacaaccgacgccgugagagauccccaaacauuggagauacuugaua
ucacucccugcucuuuugguggcgucagcgucaucaccccaggaaccaauacaagcaaucaaguggcuguccuuuaucaa
gaugucaauuguaccgaagucccagucgcaauacaugcggaucaacugaccccaacauggagaguuuacucaacgggauc
uaacguguuucaaacucgugcuggcugccugauaggagcggagcaugugaauaauuccuaugaaugcgacauucccauu
ggggcuggaaucugugcauccuaucaaacacaaacuaacucuccccgccggcggcggagcgucgccagccaaagcauuau
ugcauauacgaugucccugugcgcagaaaauucuguugcauacagcaauaacuccaucgcuaucccuacaaacuuuacca
ucagcuguacaaccgaaaucuugcccguuucuaugacuaaaacaaguugugacugcacuauguacaucuguggcgacuc
aacagaguguucuaaccuucugcuucaauauggaucuuucuguacacaacuuaaucgcgcucucaccgguauagcuguu
gagcaagauaagaacacucaggaaguuuucgcccaagucaaacaauguuauaaaacaccacccauaaaagacuucggcgga
uuuaauuucucucaaauacugccggacccauccaggagacgaagaagcuucauagaagaucuucucuucaacaaggugac
ccuggccgaugcgggguuuaucaagcaauauggcgacugucucggcgauauugcugcacgcgaucugauaugugcacag
aaauucaauggguguaccgugcucccaccucugcugacagaugaaaugaucgcucaauauaccagugcgcucuuggcug
gaacaauuacuagugguuggacuuuuggggcuggagccgcacuccaaaucccuuuugccaugcaaauggccuaucgcuu
uaaugggauaggggucacucagaaugucuuguaugaaaaccagaaguugauugcuaaccaauuuaauucagcuauaggg
aaaauucaagacagccucaguaguacugccagugcccugggcaaacugcaagaugucgugaaccaaaaugcucaagcccu
gaauacccucguuaagcaacuuagcucaaacuuuggugcgauuuccucaggcccuaaugacauccucucaaggcugccua
aaguggaagcugagguccaaaucgaucgccugauuacgggucgccugcaaucacuccaaacauaugucacccagcaguug
aucagagcggccgagauacgggcaucagcaaauuuggcggccacgaaaaugucagagugcguacuuggucaaaguaaaa
gaguugauuucugcggaaaagguuaccaccuuaugucuuucccccaguccgcuccacauggaguggucuuucugcauug
uacuuaugugccagcccaagaaaagaauuuuacuaccgcccccgcuauuugucaugaugguaaggcgcacuuccccagag
aaggaguguuuguguccaacgggacucacugguuugugacucaaaggaacuuuuaugaaccucaaauuaucaccacaga
uaacacauuuguguccgggaauugcgaugugguuaucggcauuguuaauaauaccguuuacgaucccuugcaaccugag
uuggauaguuucaaggaagaacuugacaaauacuuuaagaaucacacuuccccggauguagaccucggggacauuuccg
gaauuaaugcgaguguugugaauauacagaaagagauagaccgacucaacgagguugcuaagaaccucaacgagagccuu
aucgaucuucaagaacucggcaaauacgagcaauacauuaaauggccuugguccggcagaaggagacggcgaaggggaag
uggcggcagcggcucuggauacaucccggaagcuccacgggaugggcaagcauauguucgcaaggauggagaauggguc
cuucuuagcaccuucuugggauaauga (SEQ ID NO: 153)
MDWTWILFLVAAATRVHSMFVFLVLLPLVSSQCVNLTTRTQLPPAYTNSFTRGVYY
PDKVFRSSCLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDCADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLCAENSVAYSNNSIAIPTNFTISCTTEILPVSMT
KTSCDCTMYICGDSTECSNLLLQYGSFCTQLNRALTGIAVEQDKNTQEVFAQVKQCY
KTPPIKDFGGFNFSQILPDPSRRRRSFIEDLLFNKVTLADAGFIKQYGDCLGDIAARDLI
CAQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 154)
WuS_3F_D2P_GlyD2_pVax
ggatccgccaccatggattggacatggatactgtttctggtcgctgctgccacacgtgtccacagcatgtttgtcttcttggtgctcttgcc
tcttgtgagctcccaatgtgtgaatctgactacacgtacgcaacttccgcctgcctacaccaactctttcaccagaggcgtgtattatccg
gataaggtgttcaggagctcctgccttcattcaacacaggatttgtttctgcctttcttttcaaacgttacttggttccatgccatccacgtgtc
aggaacaaatggtaccaagagattcgataacccagttctcccttttaatgatggagtctattttgcaagcactgagaaaagtaatattatac
gaggttggattttcggaacgacactcgacagtaaaacacaatccctgttgatagtcaacaatgccacgaacgtagttataaaagtttgcg
aatttcaattttgcaacgatcctttcctgggtgtgtactatcacaagaacaacaaatcttggatggaaagcgagtttcgagtgtattcttcag
caaacaactgtactttcgaatatgtttctcaaccattcctgatggatctcgaaggtaaacagggcaactttaagaatctgagagagtttgtg
tttaagaacattgacggctattttaagatttacagcaaacatacgcctataaaccttgtgagagacctgcctcaagggtttagcgccctgg
aaccactcgtggacctgcctatcggcatcaatattaccagatttcaaacgctccttgccctgcataggagctatttgacacctggggactc
ttctagcggctggactgcaggcgctgccgcttattacgtgggatatctccagcctagaactttcctcttgaaatacaacgagaatggaac
cataacagacgcagttgattgtgctctcgaccccttgtccgagaccaaatgcacactgaaaagttttaccgtggagaaagggatctatca
aactagtaatttccgcgttcaacccactgagagcatagtgaggtttcctaacattacaaatctttgcccgtttggggaagtgtttaatgcca
ctcgttttgctagtgtatacgcctggaatcgaaagcggatttccaattgcgttgctgactacagtgtactctataatagcgcttcatttagca
ccttcaagtgctacggggttaacgggaccaaactcaatgacctctgcttcacgaacgtttacgccgactcctttgtcattcgaggtgacg
aagtaagacaaatcgccccaggccagactggaaagatcgcggactacaactataagctgccagacgacttcactggatgtgtgatcg
cctggaatagtaacaacctcgactccaaggtgggtggcaattacaattatctctataggctgttcaggaagagtaatttgaaaccattcga
gcgcgacataaatacaacaatctaccaagcgggttctaccccttgcaacggcgtggaaggttttaattgttacttccctctccaaagctac
gggtttcaaccaacaaacggcgtgggataccaaccatacagggtggttgtgttgagcttcgaattgaatcatgcacctgcaacagtgtg
tgggcccaagaagtccaccaatctcgttaagaataaatgcgtgaacttcaactttaacgggttgacagggaccggcgtgcttacggaa
agtaataagaaattccttcccttccagcaatttggtcgcgactgtgcggatacaacggacgcagtgcgagacccacagacattggagat
cctggacataacaccttgctcttttggcggggtctccgtaataacacctggaacaaataccagcaatcaagtagcggtcttgtatcaaga
tgtaaactgtactgaagtcccagttgctatacatgcagaccaacttacaccgacgtggcgcgtgtattctacgggctccaacgtattcca
aaccagagcagggtgcttgataggggcagagcacgtcaacaatagctatgagtgtgatatcccgataggtgctggaatctgcgcaag
ttaccagacccaaaccaatagcccccgccggagacgatcagtggcaagccagtctataatagcctacacgatgtcactgtgtgccga
aaatagcgttgcctatagtaacaatagcatcgccattccaaccaatttcacaatatcagtcactactgagattctgcctgtgtcaatgacta
aaactagtgtggactgcacaatgtatatttgcggcgattccacagaatgtagcaatcttctgctgcaatatgggagtttctgtacacaattg
aatcgggcccttactggaatcgccgtagagcaggacaagaacacccaagaagtctttgcgcaagtcaaacaatgttataagactcccc
caattaaagattttggcggctttaattttagccaaatacttcccgaccccagccgccgacgacgctcctttatcgaagatctgttgtttaata
aagtcacattggctgatgctggctttatcaaacaatacggtgattgtctgggtgatattgcagcccgagatctgatctgcgcccaaaagtt
taacggcttgaccgttctcccgccactcctgacagatgagatgatcgcgcaatatacctctgcactcctggcgggaacaatcactagtg
gttggactttcggcgccggcgctgcactgcaaattcccttcgccatgcaaatggcctatcggtttaacggaattggtgtgactcagaatg
tgctttacgaaaatcagaaactcatagctaatcagtttaacagcgcaatcgggaaaattcaggattccctcagcagcaccgctagcgcct
tgggcaagctgcaggacgttgtaaaccagaacgctcaggccctcaacactctcgttaaacaattgagctctaactttggggccataagc
agtggtcctaacgacatcctgagtcgtctgccaaaggtagaggccgaagtgcaaatcgaccggctcatcactggaagactgcaaagc
ctgcaaacctatgtcacacagcaacttatacgggccgccgaaatcagggcctcagcaaacctcgcagcaacaaagatgagcgagtgt
gtgctgggccaatccaagcgcgtggacttctgtggtaagggataccatctgatgtcctttccccaatccgcgcctcatggagtagttttcc
tgcacgttacgtatgtgcctgcccaagagaagaactttacaacagcaccagccatttgtcatgacggaaaagcccattttcctagagaa
ggagtctttgtttccaatgggacacattggtttgttacccagcgtaacttttatgagccacaaatcatcaccacggacaatactttcgtgag
cggtaattgtgatgtggtcattggcatagtgaataacactgtttacgaccccctgcaaccggaattggacagcttcaaagaagaactgga
caagtacttcaagaaccacacatccccagacgtagacctcggagatatttccggaattaacgcatcagtagttaacatccagaaagaaa
tagatcgactgaatgaggtcgctaagaacttgaacgaatcacttatagatctccaggaactcggcaaatatgagcaatatattaaatggc
cctggtcaggtcgcagaagacgccgccggggttccggcggatctggatctggatatattcccgaagctccacgggatgggcaagcc
tacgtaagaaaggatggagaatgggtacttttgtccacgttcttgggctagtag (SEQ ID NO: 155)
ggauccgccaccauggauuggacauggauacuguuucuggucgcugcugccacacguguccacagcauguuugucuucu
uggugcucuugccucuugugagcucccaaugugugaaucugacuacacguacgcaacuuccgccugccuacaccaacuc
uuucaccagaggcguguauuauccggauaagguguucaggagcuccugccuucauucaacacaggauuuguuucugccu
uucuuuucaaacguuacuugguuccaugccauccacgugucaggaacaaaugguaccaagagauucgauaacccaguuc
ucccuuuuaaugauggagucuauuuugcaagcacugagaaaaguaauauuauacgagguuggauuuucggaacgacacu
cgacaguaaaacacaaucccuguugauagucaacaaugccacgaacguaguuauaaaaguuugcgaauuucaauuuugca
acgauccuuuccuggguguguacuaucacaagaacaacaaaucuuggauggaaagcgaguuucgaguguauucuucagc
aaacaacuguacuuucgaauauguuucucaaccauuccugauggaucucgaagguaaacagggcaacuuuaagaaucuga
gagaguuuguguuuaagaacauugacggcuauuuuaagauuuacagcaaacauacgccuauaaaccuugugagagaccu
gccucaaggguuuagcgcccuggaaccacucguggaccugccuaucggcaucaauauuaccagauuucaaacgcuccuug
cccugcauaggagcuauuugacaccuggggacucuucuagcggcuggacugcaggcgcugccgcuuauuacgugggaua
ucuccagccuagaacuuuccucuugaaauacaacgagaauggaaccauaacagacgcaguugauugugcucucgaccccu
uguccgagaccaaaugcacacugaaaaguuuuaccguggagaaagggaucuaucaaacuaguaauuuccgcguucaaccc
acugagagcauagugagguuuccuaacauuacaaaucuuugcccguuuggggaaguguuuaaugccacucguuuugcua
guguauacgccuggaaucgaaagcggauuuccaauugcguugcugacuacaguguacucuauaauagcgcuucauuuag
caccuucaagugcuacgggguuaacgggaccaaacucaaugaccucugcuucacgaacguuuacgccgacuccuuuguca
uucgaggugacgaaguaagacaaaucgccccaggccagacuggaaagaucgcggacuacaacuauaagcugccagacgac
uucacuggaugugugaucgccuggaauaguaacaaccucgacuccaagguggguggcaauuacaauuaucucuauaggc
uguucaggaagaguaauuugaaaccauucgagcgcgacauaaauacaacaaucuaccaagcggguucuaccccuugcaac
ggcguggaagguuuuaauuguuacuucccucuccaaagcuacggguuucaaccaacaaacggcgugggauaccaaccau
acagggugguuguguugagcuucgaauugaaucaugcaccugcaacagugugugggcccaagaaguccaccaaucucgu
uaagaauaaaugcgugaacuucaacuuuaacggguugacagggaccggcgugcuuacggaaaguaauaagaaauuccuu
cccuuccagcaauuuggucgcgacugugcggauacaacggacgcagugcgagacccacagacauuggagauccuggacau
aacaccuugcucuuuuggcggggucuccguaauaacaccuggaacaaauaccagcaaucaaguagcggucuuguaucaag
auguaaacuguacugaagucccaguugcuauacaugcagaccaacuuacaccgacguggcgcguguauucuacgggcuc
caacguauuccaaaccagagcagggugcuugauaggggcagagcacgucaacaauagcuaugagugugauaucccgaua
ggugcuggaaucugcgcaaguuaccagacccaaaccaauagcccccgccggagacgaucaguggcaagccagucuauaau
agccuacacgaugucacugugugccgaaaauagcguugccuauaguaacaauagcaucgccauuccaaccaauuucacaa
uaucagucacuacugagauucugccugugucaaugacuaaaacuaguguggacugcacaauguauauuugcggcgauuc
cacagaauguagcaaucuucugcugcaauaugggaguuucuguacacaauugaaucgggcccuuacuggaaucgccgua
gagcaggacaagaacacccaagaagucuuugcgcaagucaaacaauguuauaagacucccccaauuaaagauuuuggcgg
cuuuaauuuuagccaaauacuucccgaccccagccgccgacgacgcuccuuuaucgaagaucuguuguuuaauaaaguca
cauuggcugaugcuggcuuuaucaaacaauacggugauugucugggugauauugcagcccgagaucugaucugcgccca
aaaguuuaacggcuugaccguucucccgccacuccugacagaugagaugaucgcgcaauauaccucugcacuccuggcgg
gaacaaucacuagugguuggacuuucggcgccggcgcugcacugcaaauucccuucgccaugcaaauggccuaucgguu
uaacggaauuggugugacucagaaugugcuuuacgaaaaucagaaacucauagcuaaucaguuuaacagcgcaaucggg
aaaauucaggauucccucagcagcaccgcuagcgccuugggcaagcugcaggacguuguaaaccagaacgcucaggcccu
caacacucucguuaaacaauugagcucuaacuuuggggccauaagcagugguccuaacgacauccugagucgucugccaa
agguagaggccgaagugcaaaucgaccggcucaucacuggaagacugcaaagccugcaaaccuaugucacacagcaacuu
auacgggccgccgaaaucagggccucagcaaaccucgcagcaacaaagaugagcgagugugugcugggccaauccaagcg
cguggacuucugugguaagggauaccaucugauguccuuuccccaauccgcgccucauggaguaguuuuccugcacguu
acguaugugccugcccaagagaagaacuuuacaacagcaccagccauuugucaugacggaaaagcccauuuuccuagaga
aggagucuuuguuuccaaugggacacauugguuuguuacccagcguaacuuuuaugagccacaaaucaucaccacggac
aauacuuucgugagcgguaauugugauguggucauuggcauagugaauaacacuguuuacgacccccugcaaccggaau
uggacagcuucaaagaagaacuggacaaguacuucaagaaccacacauccccagacguagaccucggagauauuuccgga
auuaacgcaucaguaguuaacauccagaaagaaauagaucgacugaaugaggucgcuaagaacuugaacgaaucacuuau
agaucuccaggaacucggcaaauaugagcaauauauuaaauggcccuggucaggucgcagaagacgccgccgggguucc
ggcggaucuggaucuggauauauucccgaagcuccacgggaugggcaagccuacguaagaaaggauggagaauggguac
uuuuguccacguucuugggcuaguag (SEQ ID NO: 156)
MDWTWILFLVAAATRVHSMFVFLVLLPLVSSQCVNLTTRTQLPPAYTNSFTRGVYY
PDKVFRSSCLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDCADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLCAENSVAYSNNSIAIPTNFTISVTTEILPVSMT
KTSVDCTMYICGDSTECSNLLLQYGSFCTQLNRALTGIAVEQDKNTQEVFAQVKQC
YKTPPIKDFGGFNFSQILPDPSRRRRSFIEDLLFNKVTLADAGFIKQYGDCLGDIAARD
LICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTL
VKQLSSNFGAISSGPNDILSRLPKVEAEVQIDRLITGRLQSLQTYVTQQLIRAAEIRASA
NLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKNL
NESLIDLQELGKYEQYIKWPWSGRRRRRRGSGGSGSGYIPEAPRDGQAYVRKDGEW
VLLSTFLG** (SEQ ID NO: 157)
WuS_3F_D2P_GlyD1_pVax
ggatccgccaccatggactggacatggatacttttcttggtggcagctgctacacgcgtccactcaatgttcgtctttctggtgctcttgcc
actggtgagcagccaatgcgttaacctcaccacacgcacgcagcttccacccgcatacactaactcctttacgcgcggcgtgtactatc
cagataaagtgttccgaagtagcgtcttgcatagcacccaggatctgtttctcccattctttagcaatgtcacatggttccacgctatccac
gtgtctgggacgaatggaactaaacgttttgacaatcctgttcttccttttaacgacggcgtatactttgctagtactgagaagtctaacatt
atccgcggctggattttcgggacaaccctggactccaaaacccagtctctgctgatagtaaacaatgccaccaacgtcgtcattaaagt
gtgcgagtttcaattctgcaacgacccctttctgggtgtctattaccacaagaacaataagtcttggatggagtcagaatttcgtgtctattc
ttctgccaataattgtacatttgagtatgtttctcaaccctttctcatggacctcgaaggcaagcaggggaattttaagaacctgcgggaatt
cgtctttaagaatatcgacggctatttcaaaatttacagcaaacacacgcctataaacctcgtgcgagatctcccccaaggcttctcagca
ttggagccattggtcgacttgccaatcggaattaatatcacaaggtttcagactctgctggccctgcatcgctcctatcttacccctggcg
attcctcaagtggctggacggccggcgcagcagcctattacgtcggctatctccagccaaggacgtttcttttgaagtataatgaaaatg
ggactattactgacgccgtcgactgcgctttggaccccctgagcgagacaaagtgcacattgaaaagcttcacggtggagaagggtat
ttatcaaacttccaactttagggtgcaaccaacagagagcatcgtgaggttccctaatatcactaatctctgtccatttggcgaggtgttta
acgcgaccagatttgcaagcgtatatgcctggaataggaagagaataagcaattgtgttgccgattactctgtcttgtataacagcgcat
ctttcagcacttttaagtgctatggtgtcaacgggacaaaacttaacgatctttgcttcaccaacgtttacgcagactcttttgtcatacgcg
gagatgaggtccgacaaatagctcccggccagactgggaaaatcgctgattataactataagcttccagatgacttcacaggatgcgta
attgcatggaactctaacaacctggactcaaaagttggtggcaactataactatctctatcgtttgttccgaaaatcaaaccttaaacccttt
gaacgggatattaatacgacaatttaccaagcagggagcactccttgtaacggtgtagaaggtttcaattgttattttcctctgcaatcata
cggattccaaccaacaaacggtgtgggttatcaaccttatcgggttgtagttttgagcttcgagcttaaccatgcacccgccacagtatgc
ggaccgaagaagagtacaaacctggttaagaataaatgtgtaaacttcaactttaatggactgacggggacgggagtactcactgaaa
gcaataagaaattcttgccttttcagcaattcgggcgggacatagcggacactacagacgccgtgcgcgacccccagactctcgaaat
cctggacataaccccgtgctcatttggcggagtttcagtcatcactccagggaccaatacctcaaaccaagtagctgtgctgtatcaaga
tgtgaattgcaccgaagtaccagtggccattcacgccgatcagctgaccccgacatggcgggtgtactcaaccggttcaaatgtgtttc
aaacaagagcaggttgtcttattggcgctgaacacgtgaataactcctatgaatgcgacatcccaattggtgccggaatctgtgcctctta
tcaaacacaaactaattcaccaaggcgtaggcgcagcgtcgcctctcaatcaattatagcctacaccatgtcactgggtgccgaaaact
ccgtcgcgtacagcaacaatagcattgccatccctaccaacttcaccatcagctgtacaactgagatcctgcctgtatccatgacaaaga
catcctgcgattgcactatgtacatctgtggagactctactgagtgtagcaacctcttgctccaatacgggagtttctgtacgcaactcaac
cgtgccctcaccggcatagccgtagagcaagataagaatacccaggaagtatttgcccaagtaaagcaaatttataagacgccaccca
ttaaagactttggcggtttcaacttcagtcaaatactgccagacccgtctcgcaggagaaggagttttattgaagacctgctctttaacaa
ggtgactcttgccgatgctggatttattaaacaatatggggattgtctcggagatatcgctgctcgggatcttatctgcgcgcagaaattca
acgggtgtaccgtgctcccacccttgctcactgacgaaatgatcgcgcaatatacctcagcacttctggcgggaactattacatctggtt
ggacattcggcgcaggggcagctctccaaattcccttcgcaatgcaaatggcttacaggttcaatggcataggtgtcacacaaaacgt
gctgtacgagaatcaaaagcttatagccaatcagtttaatagcgccataggcaagatccaagattccctgagctccacggcaagcgctc
tgggaaaattgcaagacgtagtcaatcaaaacgctcaagcgctgaatacccttgtgaaacaactttcttcaaactttggagctatctcatct
gggcccaacgatattctgagtcgactgccaaaggttgaagctgaagtccaaattgatcggttgatcacaggaaggctgcaatccctgc
agacttacgtgacccagcaactgatcagggcagccgaaataagggcttccgccaatctggcagccacaaagatgtctgaatgtgtctt
gggtcaaagcaaacgcgtcgatttctgtggcaaggggtaccatctgatgtcattccctcaatctgcccctcacggtgtggtatttctccatt
gcacttatgttcccgcacaggagaagaacttcacaacagctcccgccatttgccacgacggaaaggcgcattttccccgcgaaggtgt
cttcgtgtccaatgggactcattggtttgtgactcagaggaatttctatgagccgcagattatcaccaccgacaacactttcgtctccggta
actgcgacgtcgttatcggaatcgtcaataacacagtgtatgatcctctgcagccggagctggactcattcaaagaggagttggataaat
attttaagaatcatacaagccccgacgtcgatctgggcgatattagtggtatcaatgcgtccgtggttaacattcagaaagagattgaca
gactcaatgaggtcgccaagaacttgaacgaatccttgattgatctccaggagttgggcaagtatgagcaatatatcaagtggccatgg
tctgggcgaaggcgccgtcgcagagggtccggcggtagtggttccgggtacataccagaagctccacgagatggtcaagcttatgta
aggaaagacggagagtgggtcctgcttagcacattcttgggttgataa (SEQ ID NO: 158)
ggauccgccaccauggacuggacauggauacuuuucuugguggcagcugcuacacgcguccacucaauguucgucuuuc
uggugcucuugccacuggugagcagccaaugcguuaaccucaccacacgcacgcagcuuccacccgcauacacuaacucc
uuuacgcgcggcguguacuauccagauaaaguguuccgaaguagcgucuugcauagcacccaggaucuguuucucccau
ucuuuagcaaugucacaugguuccacgcuauccacgugucugggacgaauggaacuaaacguuuugacaauccuguucu
uccuuuuaacgacggcguauacuuugcuaguacugagaagucuaacauuauccgcggcuggauuuucgggacaacccug
gacuccaaaacccagucucugcugauaguaaacaaugccaccaacgucgucauuaaagugugcgaguuucaauucugcaa
cgaccccuuucugggugucuauuaccacaagaacaauaagucuuggauggagucagaauuucgugucuauucuucugcc
aauaauuguacauuugaguauguuucucaacccuuucucauggaccucgaaggcaagcaggggaauuuuaagaaccugc
gggaauucgucuuuaagaauaucgacggcuauuucaaaauuuacagcaaacacacgccuauaaaccucgugcgagaucuc
ccccaaggcuucucagcauuggagccauuggucgacuugccaaucggaauuaauaucacaagguuucagacucugcugg
cccugcaucgcuccuaucuuaccccuggcgauuccucaaguggcuggacggccggcgcagcagccuauuacgucggcua
ucuccagccaaggacguuucuuuugaaguauaaugaaaaugggacuauuacugacgccgucgacugcgcuuuggacccc
cugagcgagacaaagugcacauugaaaagcuucacgguggagaaggguauuuaucaaacuuccaacuuuagggugcaac
caacagagagcaucgugagguucccuaauaucacuaaucucuguccauuuggcgagguguuuaacgcgaccagauuugc
aagcguauaugccuggaauaggaagagaauaagcaauuguguugccgauuacucugucuuguauaacagcgcaucuuuc
agcacuuuuaagugcuauggugucaacgggacaaaacuuaacgaucuuugcuucaccaacguuuacgcagacucuuuug
ucauacgcggagaugagguccgacaaauagcucccggccagacugggaaaaucgcugauuauaacuauaagcuuccagau
gacuucacaggaugcguaauugcauggaacucuaacaaccuggacucaaaaguugguggcaacuauaacuaucucuaucg
uuuguuccgaaaaucaaaccuuaaacccuuugaacgggauauuaauacgacaauuuaccaagcagggagcacuccuugua
acgguguagaagguuucaauuguuauuuuccucugcaaucauacggauuccaaccaacaaacgguguggguuaucaacc
uuaucggguuguaguuuugagcuucgagcuuaaccaugcacccgccacaguaugcggaccgaagaagaguacaaaccug
guuaagaauaaauguguaaacuucaacuuuaauggacugacggggacgggaguacucacugaaagcaauaagaaauucu
ugccuuuucagcaauucgggcgggacauagcggacacuacagacgccgugcgcgacccccagacucucgaaauccuggac
auaaccccgugcucauuuggcggaguuucagucaucacuccagggaccaauaccucaaaccaaguagcugugcuguauca
agaugugaauugcaccgaaguaccaguggccauucacgccgaucagcugaccccgacauggcggguguacucaaccggu
ucaaauguguuucaaacaagagcagguugucuuauuggcgcugaacacgugaauaacuccuaugaaugcgacaucccaa
uuggugccggaaucugugccucuuaucaaacacaaacuaauucaccaaggcguaggcgcagcgucgccucucaaucaauu
auagccuacaccaugucacugggugccgaaaacuccgucgcguacagcaacaauagcauugccaucccuaccaacuucacc
aucagcuguacaacugagauccugccuguauccaugacaaagacauccugcgauugcacuauguacaucuguggagacuc
uacugaguguagcaaccucuugcuccaauacgggaguuucuguacgcaacucaaccgugcccucaccggcauagccguag
agcaagauaagaauacccaggaaguauuugcccaaguaaagcaaauuuauaagacgccacccauuaaagacuuuggcggu
uucaacuucagucaaauacugccagacccgucucgcaggagaaggaguuuuauugaagaccugcucuuuaacaagguga
cucuugccgaugcuggauuuauuaaacaauauggggauugucucggagauaucgcugcucgggaucuuaucugcgcgc
agaaauucaacggguguaccgugcucccacccuugcucacugacgaaaugaucgcgcaauauaccucagcacuucuggcg
ggaacuauuacaucugguuggacauucggcgcaggggcagcucuccaaauucccuucgcaaugcaaauggcuuacaggu
ucaauggcauaggugucacacaaaacgugcuguacgagaaucaaaagcuuauagccaaucaguuuaauagcgccauaggc
aagauccaagauucccugagcuccacggcaagcgcucugggaaaauugcaagacguagucaaucaaaacgcucaagcgcu
gaauacccuugugaaacaacuuucuucaaacuuuggagcuaucucaucugggcccaacgauauucugagucgacugccaa
agguugaagcugaaguccaaauugaucgguugaucacaggaaggcugcaaucccugcagacuuacgugacccagcaacu
gaucagggcagccgaaauaagggcuuccgccaaucuggcagccacaaagaugucugaaugugucuugggucaaagcaaac
gcgucgauuucuguggcaagggguaccaucugaugucauucccucaaucugccccucacggugugguauuucuccauug
cacuuauguucccgcacaggagaagaacuucacaacagcucccgccauuugccacgacggaaaggcgcauuuuccccgcg
aaggugucuucguguccaaugggacucauugguuugugacucagaggaauuucuaugagccgcagauuaucaccaccga
caacacuuucgucuccgguaacugcgacgucguuaucggaaucgucaauaacacaguguaugauccucugcagccggag
cuggacucauucaaagaggaguuggauaaauauuuuaagaaucauacaagccccgacgucgaucugggcgauauuagug
guaucaaugcguccgugguuaacauucagaaagagauugacagacucaaugaggucgccaagaacuugaacgaauccuu
gauugaucuccaggaguugggcaaguaugagcaauauaucaaguggccauggucugggcgaaggcgccgucgcagaggg
uccggcgguagugguuccggguacauaccagaagcuccacgagauggucaagcuuauguaaggaaagacggagaguggg
uccugcuuagcacauucuuggguugauaa (SEQ ID NO: 159)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLGAENSVAYSNNSIAIPTNFTISCTTEILPVSMT
KTSCDCTMYICGDSTECSNLLLQYGSFCTQLNRALTGIAVEQDKNTQEVFAQVKQIY
KTPPIKDFGGFNFSQILPDPSRRRRSFIEDLLFNKVTLADAGFIKQYGDCLGDIAARDLI
CAQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 160)

In some embodiments therefore, the expressible nucleic acid sequence comprised in the composition of the disclosure comprises at least about 70%, 75%, 80%, 85%, 86%, 87%, 88%, 89%, 90%, 91%, 92%, 93%, 94%, 95%, 96%, 97%, 98% or 99% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the expressible nucleic acid sequence comprises the nucleic acid sequence of SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the expressible nucleic acid sequence comprised in the composition of the disclosure encodes a polypeptide comprising at least about 70%, 75%, 80%, 85%, 86%, 87%, 88%, 89%, 90%, 91%, 92%, 93%, 94%, 95%, 96%, 97%, 98% or 99% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof. In some embodiments, the expressible nucleic acid sequence encodes a polypeptide comprising the amino acid sequence of SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

### B. Nucleic Acid Molecule

In one aspect, the present disclosure also relates to a nucleic acid molecule that comprises any of the disclosed expressible nucleic acid sequences. In some embodiments, the nucleic acid molecule comprising one or a plurality of the disclosed expressible nucleic acid sequences is in form of a messanger RNA. In some embodiments, the nucleic acid molecule comprising one or a plurality of the disclosed expressible nucleic acid sequences is in form of a DNA. In some embodiments, the expressible nucleic acid sequence disclosed herein can be part of a plasmid and thus the nucleic acid molecule is a plasmid comprising such an expressible nucleic acid sequence. In some embodiments, provided herein is a vector or plasmid that is capable of expressing at least a monomer of a self-assembling nanoparticle and a viral antigen construct or constructs in the cell of a mammal in a quantity effective to elicit an immune response in the mammal. The vector or plasmid may comprise heterologous nucleic acid encoding the one or more viral antigens (such as SARS-CoV-2 antigens). In some embodiments, provided herein is a vector or plasmid that is capable of expressing at least one soluble trimer of a coronavirus or SARS-CoV-2 envelope polypeptide or constructs in the cell of a mammal in a quantity effective to elicit an immune response in the mammal. In some embodiments, the nucleic acid expresses a trimer of the spike protein of SARS-CoV-2 or a functional fragment or variant thereof. The vector may be a plasmid. The plasmid may be useful for transfecting cells with nucleic acid encoding a viral antigen, which the transformed host cell is cultured and maintained under conditions wherein expression of the viral antigen takes place and wherein the structure of the nanoparticle with the antigen or trimer elicits an immune response of a magnitude greater than and/or more therapeutically effective than the immune repsonse elicited by the antigen alone. The plasmid may further comprise an initiation codon, which may be upstream of the expressible sequence, and a stop codon, which may be downstream of the coding sequence. The initiation and termination codon may be in frame with the expressible sequence.

The plasmid may also comprise a promoter that is operably linked to the coding sequence. The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication No. US20040175727, the contents of which are incorporated herein in its entirety. The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhancers are described in U.S. Pat. Nos. 5,593,972, 5,962,428, and WO94/016737, the contents of each are fully incorporated by reference. The plasmid may also comprise a mammalian origin of replication in order to maintain the plasmid extrachromosomally and produce multiple copies of the plasmid in a cell. The plasmid may be pVAX1, pCEP4 or pREP4 from ThermoFisher Scientific (San Diego, Calif.), which may comprise the Epstein Barr virus origin of replication and nuclear antigen EBNA-1 coding region, which may produce high copy episomal replication without integration.

In some embodiments, the vector can be pVAX1 or a pVax1 variant with changes such as the variant plasmid described herein. The variant pVax1 plasmid is a 2998 basepair variant of the backbone vector plasmid pVAX1 (Invitrogen, Carlsbad Calif.). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993. The vaccine may comprise the consensus antigens and plasmids at quantities of from about 1 nanogram to 100 milligrams; about 1 microgram to about 10 milligrams; or preferably about 0.1 microgram to about 10 milligrams; or more preferably about 1 milligram to about 2 milligram. In some embodiments, pharmaceutical compositions according to the present disclosure comprise from about 1 nanogram to about 1000 micrograms of DNA. The nucleic acid sequence for the pVAX1 backbone sequence is as follows:

(SEQ ID NO: 161)
gactcttcgcgatgtacgggccagatatacgcgttgacat
tgattattgactagttattaatagtaatcaattacggggt
cattagttcatagcccatatatggagttccgcgttacata
acttacggtaaatggcccgcctggctgaccgcccaacgac
ccccgcccattgacgtcaataatgacgtatgttcccatag
taacgccaatagggactttccattgacgtcaatgggtgga
ctatttacggtaaactgcccacttggcagtacatcaagtg
tatcatatgccaagtacgccccctattgacgtcaatgacg
gtaaatggcccgcctggcattatgcccagtacatgacctt
atgggactttcctacttggcagtacatctacgtattagtc
atcgctattaccatggtgatgcggttttggcagtacatca
atgggcgtggatagcggtttgactcacggggatttccaag
tctccaccccattgacgtcaatgggagtttgttttggcac
caaaatcaacgggactttccaaaatgtcgtaacaactccg
ccccattgacgcaaatgggcggtaggcgtgtacggtggga
ggtctatataagcagagctctctggctaactagagaaccc
actgcttactggcttatcgaaattaatacgactcactata
gggagacccaagctggctagcgtttaaacttaagcttggt
accgagctcggatccactagtccagtgtggtggaattctg
cagatatccagcacagtggcggccgctcgagtctagaggg
cccgtttaaacccgctgatcagcctcgactgtgccttcta
gttgccagccatctgttgtttgcccctcccccgtgccttc
cttgaccctggaaggtgccactcccactgtcctttcctaa
taaaatgaggaaattgcatcgcattgtctgagtaggtgtc
attctattctggggggtggggtggggcaggacagcaaggg
ggaggattgggaagacaatagcaggcatgctggggatgcg
gtgggctctatggcttctactgggcggttttatggacagc
aagcgaaccggaattgccagctggggcgccctctggtaag
gttgggaagccctgcaaagtaaactggatggctttctcgc
cgccaaggatctgatggcgcaggggatcaagctctgatca
agagacaggatgaggatcgtttcgcatgattgaacaagat
ggattgcacgcaggttctccggccgcttgggtggagaggc
tattcggctatgactgggcacaacagacaatcggctgctc
tgatgccgccgtgttccggctgtcagcgcaggggcgcccg
gttctttttgtcaagaccgacctgtccggtgccctgaatg
aactgcaagacgaggcagcgcggctatcgtggctggccac
gacgggcgttccttgcgcagctgtgctcgacgttgtcact
gaagcgggaagggactggctgctattgggcgaagtgccgg
ggcaggatctcctgtcatctcaccttgctcctgccgagaa
agtatccatcatggctgatgcaatgcggcggctgcatacg
cttgatccggctacctgcccattcgaccaccaagcgaaac
atcgcatcgagcgagcacgtactcggatggaagccggtct
tgtcgatcaggatgatctggacgaagagcatcaggggctc
gcgccagccgaactgttcgccaggctcaaggcgagcatgc
ccgacggcgaggatctcgtcgtgacccatggcgatgcctg
cttgccgaatatcatggtggaaaatggccgcttttctgga
ttcatcgactgtggccggctgggtgtggcggaccgctatc
aggacatagcgttggctacccgtgatattgctgaagagct
tggcggcgaatgggctgaccgcttcctcgtgctttacggt
atcgccgctcccgattcgcagcgcatcgccttctatcgcc
ttcttgacgagttcttctgaattattaacgcttacaattt
cctgatgcggtattttctccttacgcatctgtgcggtatt
tcacaccgcatacaggtggcacttttcggggaaatgtgcg
cggaacccctatttgtttatttttctaaatacattcaaat
atgtatccgctcatgagacaataaccctgataaatgcttc
aataatagcacgtgctaaaacttcatttttaatttaaaag
gatctaggtgaagatcctttttgataatctcatgaccaaa
atcccttaacgtgagttttcgttccactgagcgtcagacc
ccgtagaaaagatcaaaggatcttcttgagatcctttttt
tctgcgcgtaatctgctgcttgcaaacaaaaaaaccaccg
ctaccagcggtggtttgtttgccggatcaagagctaccaa
ctctttttccgaaggtaactggcttcagcagagcgcagat
accaaatactgtccttctagtgtagccgtagttaggccac
cacttcaagaactctgtagcaccgcctacatacctcgctc
tgctaatcctgttaccagtggctgctgccagtggcgataa
gtcgtgtcttaccgggttggactcaagacgatagttaccg
gataaggcgcagcggtcgggctgaacggggggttcgtgca
cacagcccagcttggagcgaacgacctacaccgaactgag
atacctacagcgtgagctatgagaaagcgccacgcttccc
gaagggagaaaggcggacaggtatccggtaagcggcaggg
tcggaacaggagagcgcacgagggagcttccagggggaaa
cgcctggtatctttatagtcctgtcgggtttcgccacctc
tgacttgagcgtcgatttttgtgatgctcgtcaggggggc
ggagcctatggaaaaacgccagcaacgcggcctttttacg
gttcctgggcttttgctggccttttgctcacatgttctt

Other vectors or plasmids that can be used herein to produce the vaccine of the present disclosure include, but not limited to, pcDNA3.1(+), pCI mammalian expression vector, pSI vector, pZeoSV2(+), phCMV1, pTCP and pIRES with their respective backbone sequence as follows.

The pcDNA3.1(+) backbone sequence (SEQ ID NO: 162):

gacggatcgggagatctcccgatcccctatggtgcactct
cagtacaatctgctctgatgccgcatagttaagccagtat
ctgctccctgcttgtgtgttggaggtcgctgagtagtgcg
cgagcaaaatttaagctacaacaaggcaaggcttgaccga
caattgcatgaagaatctgcttagggttaggcgttttgcg
ctgcttcgcgatgtacgggccagatatacgcgttgacatt
gattattgactagttattaatagtaatcaattacggggtc
attagttcatagcccatatatggagttccgcgttacataa
cttacggtaaatggcccgcctggctgaccgcccaacgacc
cccgcccattgacgtcaataatgacgtatgttcccatagt
aacgccaatagggactttccattgacgtcaatgggtggag
tatttacggtaaactgcccacttggcagtacatcaagtgt
atcatatgccaagtacgccccctattgacgtcaatgacgg
taaatggcccgcctggcattatgcccagtacatgacctta
tgggactttcctacttggcagtacatctacgtattagtca
tcgctattaccatggtgatgcggttttggcagtacatcaa
tgggcgtggatagcggtttgactcacggggatttccaagt
ctccaccccattgacgtcaatgggagtttgttttggcacc
aaaatcaacgggactttccaaaatgtcgtaacaactccgc
cccattgacgcaaatgggcggtaggcgtgtacggtgggag
gtctatataagcagagctctctggctaactagagaaccca
ctgcttactggcttatcgaaattaatacgactcactatag
ggagacccaagctggctagcgtttaaacttaagcttggta
ccgagctcggatccactagtccagtgtggtggaattctgc
agatatccagcacagtggcggccgctcgagtctagagggc
ccgtttaaacccgctgatcagcctcgactgtgccttctag
ttgccagccatctgttgtttgcccctcccccgtgccttcc
ttgaccctggaaggtgccactcccactgtcctttcctaat
aaaatgaggaaattgcatcgcattgtctgagtaggtgtca
ttctattctggggggtggggtggggcaggacagcaagggg
gaggattgggaagacaatagcaggcatgctggggatgcgg
tgggctctatggcttctgaggcggaaagaaccagctgggg
ctctagggggtatccccacgcgccctgtagcggcgcatta
agcgcggcgggtgtggtggttacgcgcagcgtgaccgcta
cacttgccagcgccctagcgcccgctcctttcgctttctt
cccttcctttctcgccacgttcgccggctttccccgtcaa
gctctaaatcgggggctccctttagggttccgatttagtg
ctttacggcacctcgaccccaaaaaacttgattagggtga
tggttcacgtagtgggccatcgccctgatagacggttttt
cgccctttgacgttggagtccacgttctttaatagtggac
tcttgttccaaactggaacaacactcaaccctatctcggt
ctattcttttgatttataagggattttgccgatttcggcc
tattggttaaaaaatgagctgatttaacaaaaatttaacg
cgaattaattctgtggaatgtgtgtcagttagggtgtgga
aagtccccaggctccccagcaggcagaagtatgcaaagca
tgcatctcaattagtcagcaaccaggtgtggaaagtcccc
aggctccccagcaggcagaagtatgcaaagcatgcatctc
aattagtcagcaaccatagtcccgcccctaactccgccca
tcccgcccctaactccgcccagttccgcccattctccgcc
ccatggctgactaattttttttatttatgcagaggccgag
gccgcctctgcctctgagctattccagaagtagtgaggag
gcttttttggaggcctaggcttttgcaaaaagctcccggg
agcttgtatatccattttcggatctgatcaagagacagga
tgaggatcgtttcgcatgattgaacaagatggattgcacg
caggttctccggccgcttgggtggagaggctattcggcta
tgactgggcacaacagacaatcggctgctctgatgccgcc
gtgttccggctgtcagcgcaggggcgcccggttctttttg
tcaagaccgacctgtccggtgccctgaatgaactgcagga
cgaggcagcgcggctatcgtggctggccacgacgggcgtt
ccttgcgcagctgtgctcgacgttgtcactgaagcgggaa
gggactggctgctattgggcgaagtgccggggcaggatct
cctgtcatctcaccttgctcctgccgagaaagtatccatc
atggctgatgcaatgcggcggctgcatacgcttgatccgg
ctacctgcccattcgaccaccaagcgaaacatcgcatcga
gcgagcacgtactcggatggaagccggtcttgtcgatcag
gatgatctggacgaagagcatcaggggctcgcgccagccg
aactgttcgccaggctcaaggcgcgcatgcccgacggcga
ggatctcgtcgtgacccatggcgatgcctgcttgccgaat
atcatggtggaaaatggccgcttttctggattcatcgact
gtggccggctgggtgtggcggaccgctatcaggacatagc
gttggctacccgtgatattgctgaagagcttggcggcgaa
tgggctgaccgcttcctcgtgctttacggtatcgccgctc
ccgattcgcagcgcatcgccttctatcgccttcttgacga
gttcttctgagcgggactctggggttcgaaatgaccgacc
aagcgacgcccaacctgccatcacgagatttcgattccac
cgccgccttctatgaaaggttgggcttcggaatcgttttc
cgggacgccggctggatgatcctccagcgcggggatctca
tgctggagttcttcgcccaccccaacttgtttattgcagc
ttataatggttacaaataaagcaatagcatcacaaatttc
acaaataaagcatttttttcactgcattctagttgtggtt
tgtccaaactcatcaatgtatcttatcatgtctgtatacc
gtcgacctctagctagagcttggcgtaatcatggtcatag
ctgtttcctgtgtgaaattgttatccgctcacaattccac
acaacatacgagccggaagcataaagtgtaaagcctgggg
tgcctaatgagtgagctaactcacattaattgcgttgcgc
tcactgcccgctttccagtcgggaaacctgtcgtgccagc
tgcattaatgaatcggccaacgcgcggggagaggcggttt
gcgtattgggcgctcttccgcttcctcgctcactgactcg
ctgcgctcggtcgttcggctgcggcgagcggtatcagctc
actcaaaggcggtaatacggttatccacagaatcagggga
taacgcaggaaagaacatgtgagcaaaaggccagcaaaag
gccaggaaccgtaaaaaggccgcgttgctggcgtttttcc
ataggctccgcccccctgacgagcatcacaaaaatcgacg
ctcaagtcagaggtggcgaaacccgacaggactataaaga
taccaggcgtttccccctggaagctccctcgtgcgctctc
ctgttccgaccctgccgcttaccggatacctgtccgcctt
tctcccttcgggaagcgtggcgctttctcatagctcacgc
tgtaggtatctcagttcggtgtaggtcgttcgctccaagc
tgggctgtgtgcacgaaccccccgttcagcccgaccgctg
cgccttatccggtaactatcgtcttgagtccaacccggta
agacacgacttatcgccactggcagcagccactggtaaca
ggattagcagagcgaggtatgtaggcggtgctacagagtt
cttgaagtggtggcctaactacggctacactagaagaaca
gtatttggtatctgcgctctgctgaagccagttaccttcg
gaaaaagagttggtagctcttgatccggcaaacaaaccac
cgctggtagcggtttttttgtttgcaagcagcagattacg
cgcagaaaaaaaggatctcaagaagatcctttgatctttt
ctacggggtctgacgctcagtggaacgaaaactcacgtta
agggattttggtcatgagattatcaaaaaggatcttcacc
tagatccttttaaattaaaaatgaagttttaaatcaatct
aaagtatatatgagtaaacttggtctgacagttaccaatg
cttaatcagtgaggcacctatctcagcgatctgtctattt
cgttcatccatagttgcctgactccccgtcgtgtagataa
ctacgatacgggagggcttaccatctggccccagtgctgc
aatgataccgcgagacccacgctcaccggctccagattta
tcagcaataaaccagccagccggaagggccgagcgcagaa
gtggtcctgcaactttatccgcctccatccagtctattaa
ttgttgccgggaagctagagtaagtagttcgccagttaat
agtttgcgcaacgttgttgccattgctacaggcatcgtgg
tgtcacgctcgtcgtttggtatggcttcattcagctccgg
ttcccaacgatcaaggcgagttacatgatcccccatgttg
tgcaaaaaagcggttagctccttcggtcctccgatcgttg
tcagaagtaagttggccgcagtgttatcactcatggttat
ggcagcactgcataattctcttactgtcatgccatccgta
agatgcttttctgtgactggtgagtactcaaccaagtcat
tctgagaatagtgtatgcggcgaccgagttgctcttgccc
ggcgtcaatacgggataataccgcgccacatagcagaact
ttaaaagtgctcatcattggaaaacgttcttcggggcgaa
aactctcaaggatcttaccgctgttgagatccagttcgat
gtaacccactcgtgcacccaactgatcttcagcatctttt
actttcaccagcgtttctgggtgagcaaaaacaggaaggc
aaaatgccgcaaaaaagggaataagggcgacacggaaatg
ttgaatactcatactcttcctttttcaatattattgaagc
atttatcagggttattgtctcatgagcggatacatatttg
aatgtatttagaaaaataaacaaataggggttccgcgcac
atttccccgaaaagtgccacctgacgtc

The pCI mammalian expression vector backbone sequence (SEQ ID NO: 163):

tcaatattggccattagccatattattcattggttatata
gcataaatcaatattggctattggccattgcatacgttgt
atctatatcataatatgtacatttatattggctcatgtcc
aatatgaccgccatgttggcattgattattgactagttat
taatagtaatcaattacggggtcattagttcatagcccat
atatggagttccgcgttacataacttacggtaaatggccc
gcctggctgaccgcccaacgacccccgcccattgacgtca
ataatgacgtatgttcccatagtaacgccaatagggactt
tccattgacgtcaatgggtggagtatttacggtaaactgc
ccacttggcagtacatcaagtgtatcatatgccaagtccg
ccccctattgacgtcaatgacggtaaatggcccgcctggc
attatgcccagtacatgaccttacgggactttcctacttg
gcagtacatctacgtattagtcatcgctattaccatggtg
atgcggttttggcagtacaccaatgggcgtggatagcggt
ttgactcacggggatttccaagtctccaccccattgacgt
caatgggagtttgttttggcaccaaaatcaacgggacttt
ccaaaatgtcgtaataaccccgccccgttgacgcaaatgg
gcggtaggcgtgtacggtgggaggtctatataagcagagc
tcgtttagtgaaccgtcagatcactagaagctttattgcg
gtagtttatcacagttaaattgctaacgcagtcagtgctt
ctgacacaacagtctcgaacttaagctgcagaagttggtc
gtgaggcactgggcaggtaagtatcaaggttacaagacag
gtttaaggagaccaatagaaactgggcttgtcgagacaga
gaagactcttgcgtttctgataggcacctattggtcttac
tgacatccactttgcctttctctccacaggtgtccactcc
cagttcaattacagctcttaaggctagagtacttaatacg
actcactataggctagcctcgagaattcacgcgtggtacc
tctagagtcgacccgggcggccgcttcgagcagacatgat
aagatacattgatgagtttggacaaaccacaactagaatg
cagtgaaaaaaatgctttatttgtgaaatttgtgatgcta
ttgctttatttgtaaccattataagctgcaataaacaagt
taacaacaacaattgcattcattttatgtttcaggttcag
ggggagatgtgggaggttttttaaagcaagtaaaacctct
acaaatgtggtaaaatcgataaggatccgggctggcgtaa
tagcgaagaggcccgcaccgatcgcccttcccaacagttg
cgcagcctgaatggcgaatggacgcgccctgtagcggcgc
attaagcgcggcgggtgtggtggttacgcgcagcgtgacc
gctacacttgccagcgccctagcgcccgctcctttcgctt
tcttcccttcctttctcgccacgttcgccggctttccccg
tcaagctctaaatcgggggctccctttagggttccgattt
agtgctttacggcacctcgaccccaaaaaacttgattagg
gtgatggttcacgtagtgggccatcgccctgatagacggt
ttttcgccctttgacgttggagtccacgttctttaatagt
ggactcttgttccaaactggaacaacactcaaccctatct
cggtctattcttttgatttataagggattttgccgatttc
ggcctattggttaaaaaatgagctgatttaacaaaaattt
aacgcgaattttaacaaaatattaacgcttacaatttcct
gatgcggtattttctccttacgcatctgtgcggtatttca
caccgcatatggtgcactctcagtacaatctgctctgatg
ccgcatagttaagccagccccgacacccgccaacacccgc
tgacgcgccctgacgggcttgtctgctcccggcatccgct
tacagacaagctgtgaccgtctccgggagctgcatgtgtc
agaggttttcaccgtcatcaccgaaacgcgcgagacgaaa
gggcctcgtgatacgcctatttttataggttaatgtcatg
ataataatggtttcttagacgtcaggtggcacttttcggg
gaaatgtgcgcggaacccctatttgtttatttttctaaat
acattcaaatatgtatccgctcatgagacaataaccctga
taaatgcttcaataatattgaaaaaggaagagtatgagta
ttcaacatttccgtgtcgcccttattcccttttttgcggc
attttgccttcctgtttttgctcacccagaaacgctggtg
aaagtaaaagatgctgaagatcagttgggtgcacgagtgg
gttacatcgaactggatctcaacagcggtaagatccttga
gagttttcgccccgaagaacgttttccaatgatgagcact
tttaaagttctgctatgtggcgcggtattatcccgtattg
acgccgggcaagagcaactcggtcgccgcatacactattc
tcagaatgacttggttgagtactcaccagtcacagaaaag
catcttacggatggcatgacagtaagagaattatgcagtg
ctgccataaccatgagtgataacactgcggccaacttact
tctgacaacgatcggaggaccgaaggagctaaccgctttt
ttgcacaacatgggggatcatgtaactcgccttgatcgtt
gggaaccggagctgaatgaagccataccaaacgacgagcg
tgacaccacgatgcctgtagcaatggcaacaacgttgcgc
aaactattaactggcgaactacttactctagcttcccggc
aacaattaatagactggatggaggcggataaagttgcagg
accacttctgcgctcggcccttccggctggctggtttatt
gctgataaatctggagccggtgagcgtgggtctcgcggta
tcattgcagcactggggccagatggtaagccctcccgtat
cgtagttatctacacgacggggagtcaggcaactatggat
gaacgaaatagacagatcgctgagataggtgcctcactga
ttaagcattggtaactgtcagaccaagtttactcatatat
actttagattgatttaaaacttcatttttaatttaaaagg
atctaggtgaagatcctttttgataatctcatgaccaaaa
tcccttaacgtgagttttcgttccactgagcgtcagaccc
cgtagaaaagatcaaaggatcttcttgagatccttttttt
ctgcgcgtaatctgctgcttgcaaacaaaaaaaccaccgc
taccagcggtggtttgtttgccggatcaagagctaccaac
tctttttccgaaggtaactggcttcagcagagcgcagata
ccaaatactgttcttctagtgtagccgtagttaggccacc
acttcaagaactctgtagcaccgcctacatacctcgctct
gctaatcctgttaccagtggctgctgccagtggcgataag
tcgtgtcttaccgggttggactcaagacgatagttaccgg
ataaggcgcagcggtcgggctgaacggggggttcgtgcac
acagcccagcttggagcgaacgacctacaccgaactgaga
tacctacagcgtgagctatgagaaagcgccacgcttcccg
aagggagaaaggcggacaggtatccggtaagcggcagggt
cggaacaggagagcgcacgagggagcttccagggggaaac
gcctggtatctttatagtcctgtcgggtttcgccacctct
gacttgagcgtcgatttttgtgatgctcgtcaggggggcg
gagcctatggaaaaacgccagcaacgcggcctttttacgg
ttcctggccttttgctggccttttgctcacatggctcgac
agatct

The pSI vector backbone sequence (SEQ ID NO: 164):

gcgcagcaccatggcctgaaataacctctgaaagaggaac
ttggttaggtaccttctgaggcggaaagaaccagctgtgg
aatgtgtgtcagttagggtgtggaaagtccccaggctccc
cagcaggcagaagtatgcaaagcatgcatctcaattagtc
agcaaccaggtgtggaaagtccccaggctccccagcaggc
agaagtatgcaaagcatgcatctcaattagtcagcaacca
tagtcccgcccctaactccgcccatcccgcccctaactcc
gcccagttccgcccattctccgccccatggctgactaatt
ttttttatttatgcagaggccgaggccgcctcggcctctg
agctattccagaagtagtgaggaggcttttttggaggcct
aggcttttgcaaaaagcttgattcttctgacacaacagtc
tcgaacttaagctgcagaagttggtcgtgaggcactgggc
aggtaagtatcaaggttacaagacaggtttaaggagacca
atagaaactgggcttgtcgagacagagaagactcttgcgt
ttctgataggcacctattggtcttactgacatccactttg
cctttctctccacaggtgtccactcccagttcaattacag
ctcttaaggctagagtacttaatacgactcactataggct
agcctcgagaattcacgcgtggtacctctagagtcgaccc
gggcggccgcttcgagcagacatgataagatacattgatg
agtttggacaaaccacaactagaatgcagtgaaaaaaatg
ctttatttgtgaaatttgtgatgctattgctttatttgta
accattataagctgcaataaacaagttaacaacaacaatt
gcattcattttatgtttcaggttcagggggaggtgtggga
ggttttttaaagcaagtaaaacctctacaaatgtggtaaa
atcgataaggatccgggctggcgtaatagcgaagaggccc
gcaccgatcgcccttcccaacagttgcgcagcctgaatgg
cgaatggacgcgccctgtagcggcgcattaagcgcggcgg
gtgtggtggttacgcgcagcgtgaccgctacacttgccag
cgccctagcgcccgctcctttcgctttcttcccttccttt
ctcgccacgttcgccggctttccccgtcaagctctaaatc
gggggctccctttagggttccgatttagtgctttacggca
cctcgaccccaaaaaacttgattagggtgatggttcacgt
agtgggccatcgccctgatagacggtttttcgccctttga
cgttggagtccacgttctttaatagtggactcttgttcca
aactggaacaacactcaaccctatctcggtctattctttt
gatttataagggattttgccgatttcggcctattggttaa
aaaatgagctgatttaacaaaaatttaacgcgaattttaa
caaaatattaacgcttacaatttcctgatgcggtattttc
tccttacgcatctgtgcggtatttcacaccgcatatggtg
cactctcagtacaatctgctctgatgccgcatagttaagc
cagccccgacacccgccaacacccgctgacgcgccctgac
gggcttgtctgctcccggcatccgcttacagacaagctgt
gaccgtctccgggagctgcatgtgtcagaggttttcaccg
tcatcaccgaaacgcgcgagacgaaagggcctcgtgatac
gcctatttttataggttaatgtcatgataataatggtttc
ttagacgtcaggtggcacttttcggggaaatgtgcgcgga
acccctatttgtttatttttctaaatacattcaaatatgt
atccgctcatgagacaataaccctgataaatgcttcaata
atattgaaaaaggaagagtatgagtattcaacatttccgt
gtcgcccttattcccttttttgcggcattttgccttcctg
tttttgctcacccagaaacgctggtgaaagtaaaagatgc
tgaagatcagttgggtgcacgagtgggttacatcgaactg
gatctcaacagcggtaagatccttgagagttttcgccccg
aagaacgttttccaatgatgagcacttttaaagttctgct
atgtggcgcggtattatcccgtattgacgccgggcaagag
caactcggtcgccgcatacactattctcagaatgacttgg
ttgagtactcaccagtcacagaaaagcatcttacggatgg
catgacagtaagagaattatgcagtgctgccataaccatg
agtgataacactgcggccaacttacttctgacaacgatcg
gaggaccgaaggagctaaccgcttttttgcacaacatggg
ggatcatgtaactcgccttgatcgttgggaaccggagctg
aatgaagccataccaaacgacgagcgtgacaccacgatgc
ctgtagcaatggcaacaacgttgcgcaaactattaactgg
cgaactacttactctagcttcccggcaacaattaatagac
tggatggaggcggataaagttgcaggaccacttctgcgct
cggcccttccggctggctggtttattgctgataaatctgg
agccggtgagcgtgggtctcgcggtatcattgcagcactg
gggccagatggtaagccctcccgtatcgtagttatctaca
cgacggggagtcaggcaactatggatgaacgaaatagaca
gatcgctgagataggtgcctcactgattaagcattggtaa
ctgtcagaccaagtttactcatatatactttagattgatt
taaaacttcatttttaatttaaaaggatctaggtgaagat
cctttttgataatctcatgaccaaaatcccttaacgtgag
ttttcgttccactgagcgtcagaccccgtagaaaagatca
aaggatcttcttgagatcctttttttctgcgcgtaatctg
ctgcttgcaaacaaaaaaaccaccgctaccagcggtggtt
tgtttgccggatcaagagctaccaactctttttccgaagg
taactggcttcagcagagcgcagataccaaatactgttct
tctagtgtagccgtagttaggccaccacttcaagaactct
gtagcaccgcctacatacctcgctctgctaatcctgttac
cagtggctgctgccagtggcgataagtcgtgtcttaccgg
gttggactcaagacgatagttaccggataaggcgcagcgg
tcgggctgaacggggggttcgtgcacacagcccagcttgg
agcgaacgacctacaccgaactgagatacctacagcgtga
gctatgagaaagcgccacgcttcccgaagggagaaaggcg
gacaggtatccggtaagcggcagggtcggaacaggagagc
gcacgagggagcttccagggggaaacgcctggtatcttta
tagtcctgtcgggtttcgccacctctgacttgagcgtcga
tttttgtgatgctcgtcaggggggcggagcctatggaaaa
acgccagcaacgcggcctttttacggttcctggccttttg
ctggccttttgctcacatggctcgacagatct

The pZeoSV2(+) backbone sequence (SEQ ID NO: 165):

ggatcgatccggctgtggaatgtgtgtcagttagggtgtg
gaaagtccccaggctccccagcaggcagaagtatgcaaag
catgcatctcaattagtcagcaaccaggtgtggaaagtcc
ccaggctccccagcaggcagaagtatgcaaagcatgcatc
tcaattagtcagcaaccatagtcccgcccctaactccgcc
catcccgcccctaactccgcccagttccgcccattctccg
ccccatggctgactaattttttttatttatgcagaggccg
aggccgcctcggcctctgagctattccagaagtagtgagg
aggcttttttggaggcctaggcttttgcaaaaagctctct
ggctaactagagaacccactgcttactggcttatcgaaat
taatacgactcactatagggagacccaagctggctagcgt
ttaaacttaagcttggtaccgagctcggatccactagtcc
agtgtggtggaattctgcagatatccagcacagtggcggc
cgctcgagtctagagggcccgtttaaacccgctgatcagc
ctcgactgtgccttctagttgccagccatctgttgtttgc
ccctcccccgtgccttccttgaccctggaaggtgccactc
ccactgtcctttcctaataaaatgaggaaattgcatcgca
ttgtctgagtaggtgtcattctattctggggggtggggtg
gggcaggacagcaagggggaggattgggaagacaatagca
ggcatgctggggatgcggtgggctctatggcttctgaggc
ggaaagaaccagcatgtgagcaaaaggccagcaaaaggcc
aggaaccgtaaaaaggccgcgttgctggcgtttttccata
ggctccgcccccctgacgagcatcacaaaaatcgacgctc
aagtcagaggtggcgaaacccgacaggactataaagatac
caggcgtttccccctggaagctccctcgtgcgctctcctg
ttccgaccctgccgcttaccggatacctgtccgcctttct
cccttcgggaagcgtggcgctttctcatagctcacgctgt
aggtatctcagttcggtgtaggtcgttcgctccaagctgg
gctgtgtgcacgaaccccccgttcagcccgaccgctgcgc
cttatccggtaactatcgtcttgagtccaacccggtaaga
cacgacttatcgccactggcagcagccactggtaacagga
ttagcagagcgaggtatgtaggcggtgctacagagttctt
gaagtggtggcctaactacggctacactagaagaacagta
tttggtatctgcgctctgctgaagccagttaccttcggaa
aaagagttggtagctcttgatccggcaaacaaaccaccgc
tggtagcggtggtttttttgtttgcaagcagcagattacg
cgcagaaaaaaaggatctcaagaagatcctttgatctttt
ctacggggtctgacgctcagtggaacgaaaactcacgtta
agggattttggtcatgacattaacctataaaaataggcgt
atcacgaggccctttcgtctcgcgcgtttcggtgatgacg
gtgaaaacctctgacacatgcagctcccggagacggtcac
agcttgtctgtaagcggatgccgggagcagacaagcccgt
cagggcgcgtcagcgggtgttggcgggtgtcggggctggc
ttaactatgcggcatcagagcagattgtactgagagtgca
ccatatgcggtgtgaaataccgcacagatgcgtaaggaga
aaataccgcatcaggacgcgccctgtagcggcgcattaag
cgcggcgggtgtggtggttacgcgcagcgtgaccgctaca
cttgccagcgccctagcgcccgctcctttcgctttcttcc
cttcctttctcgccacgttcgccggctttccccgtcaagc
tctaaatcgggggctccctttagggttccgatttagtgct
ttacggcacctcgaccccaaaaaacttgattagggtgatg
gttcacgtagtgggccatcgccctgatagacggtttttcg
ccctttgacgttggagtccacgttctttaatagtggactc
ttgttccaaactggaacaacactcaaccctatctcggtct
attcttttgatttataagggattttgccgatttcggccta
ttggttaaaaaatgagctgatttaacaaaaatttaacgcg
aattttaacaaaatattaacgcttacaatttccattcgcc
attcaggctgaactagatctagagtccgttacataactta
cggtaaatggcccgcctggctgaccgcccaacgacccccg
cccattgacgtcaataatgacgtatgttcccatagtaacg
ccaatagggactttccattgacgtcaatgggtggagtatt
tacggtaaactgcccacttggcagtacatcaagtgtatca
tatgccaagtacgccccctattgacgtcaatgacggtaaa
tggcccgcctggcattatgcccagtacatgaccttatggg
actttcctacttggcagtacatctacgtattagtcatcgc
tattaccatggtgatgcggttttggcagtacatcaatggg
cgtggatagcggtttgactcacggggatttccaagtctcc
accccattgacgtcaatgggagtttgttttggcaccaaaa
tcaacgggactttccaaaatgtcgtaacaactccgcccca
ttgacgcaaatgggcggtaggcgtgtacggtgggaggtct
atataagcagagctcgtttagtgaaccgtcagatcgcctg
gagacgccatccacgctgttttgacctccatagaagacac
cgggaccgatccagcctccgcggccgggaacggtgcattg
gaacggaccgtgttgacaattaatcatcggcatagtatat
cggcatagtataatacgacaaggtgaggaactaaaccatg
gccaagttgaccagtgccgttccggtgctcaccgcgcgcg
acgtcgccggagcggtcgagttctggaccgaccggctcgg
gttctcccgggacttcgtggaggacgacttcgccggtgtg
gtccgggacgacgtgaccctgttcatcagcgcggtccagg
accaggtggtgccggacaacaccctggcctgggtgtgggt
gcgcggcctggacgagctgtacgccgagtggtcggaggtc
gtgtccacgaacttccgggacgcctccgggccggccatga
ccgagatcggcgagcagccgtgggggcgggagttcgccct
gcgcgacccggccggcaactgcgtgcacttcgtggccgag
gagcaggactgacactcgacctcgaaacttgtttattgca
gcttataatggttacaaataaagcaatagcatcacaaatt
tcacaaataaagcatttttttcactgcattctagttgtgg
tttgtccaaactcatcaatgtatcttatcatgtct

The phCMV1 backbone sequence (SEQ ID NO: 166):

tagttattaatagtaatcaattacggggtcattagttcat
agcccatatatggagttccgcgttacataacttacggtaa
atggcccgcctggctgaccgcccaacgacccccgcccatt
gacgtcaataatgacgtatgttcccatagtaacgccaata
gggactttccattgacgtcaatgggtggagtatttacggt
aaactgcccacttggcagtacatcaagtgtatcatatgcc
aagtacgccccctattgacgtcaatgacggtaaatggccc
gcctggcattatgcccagtacatgaccttatgggactttc
ctacttggcagtacatctacgtattagtcatcgctattac
catggtgatgcggttttggcagtacatcaatgggcgtgga
tagcggtttgactcacggggatttccaagtctccacccca
ttgacgtcaatgggagtttgttttggcaccaaaatcaacg
ggactttccaaaatgtcgtaacaactccgccccattgacg
caaatgggcggtaggcgtgtacggtgggaggtctatataa
gcagagctcgtttagtgaaccgtcagatcgcctggagacg
ccatccacgctgttttgacctccatagaagacaccgggac
cgatccagcctccgcggccgggaacggtgcattggaacgc
ggattccccgtgccaagagtgacgtaagtaccgcctatag
actctataggcacacccctttggctcttatgcatgaatta
atacgactcactatagggagacagactgttcctttcctgg
gtcttttctgcaggcaccgtcgtcgacttaacagatctcg
agctcaagcttcgaattctgcagtcgacggtaccgcgggc
ccgggatccaccgggtacaagtaaagcggccgcgactcta
gatcataatcagccataccacatttgtagaggttttactt
gctttaaaaaacctcccacacctccccctgaacctgaaac
ataaaatgaatgcaattgttgttgttaacttgtttattgc
agcttataatggttacaaataaagcaatagcatcacaaat
ttcacaaataaagcatttttttcactgcattctagttgtg
gtttgtccaaactcatcaatgtatcttaaggcgtaaattg
taagcgttaatattttgttaaaattcgcgttaaatttttg
ttaaatcagctcattttttaaccaataggccgaaatcggc
aaaatcccttataaatcaaaagaatagaccgagatagggt
tgagtgttgttccagtttggaacaagagtccactattaaa
gaacgtggactccaacgtcaaagggcgaaaaaccgtctat
cagggcgatggcccactacgtgaaccatcaccctaatcaa
gttttttggggtcgaggtgccgtaaagcactaaatcggaa
ccctaaagggagcccccgatttagagcttgacggggaaag
ccggcgaacgtggcgagaaaggaagggaagaaagcgaaag
gagcgggcgctagggcgctggcaagtgtagcggtcacgct
gcgcgtaaccaccacacccgccgcgcttaatgcgccgcta
cagggcgcgtcaggtggcacttttcggggaaatgtgcgcg
gaacccctatttgtttatttttctaaatacattcaaatat
gtatccgctcatgagacaataaccctgataaatgcttcaa
taatattgaaaaaggaagagtcctgaggcggaaagaacca
gctgtggaatgtgtgtcagttagggtgtggaaagtcccca
ggctccccagcaggcagaagtatgcaaagcatgcatctca
attagtcagcaaccaggtgtggaaagtccccaggctcccc
agcaggcagaagtatgcaaagcatgcatctcaattagtca
gcaaccatagtcccgcccctaactccgcccatcccgcccc
taactccgcccagttccgcccattctccgccccatggctg
actaattttttttatttatgcagaggccgaggccgcctcg
gcctctgagctattccagaagtagtgaggaggcttttttg
gaggcctaggcttttgcaaagatcgatcaagagacaggat
gaggatcgtttcgcatgattgaacaagatggattgcacgc
aggttctccggccgcttgggtggagaggctattcggctat
gactgggcacaacagacaatcggctgctctgatgccgccg
tgttccggctgtcagcgcaggggcgcccggttctttttgt
caagaccgacctgtccggtgccctgaatgaactgcaagac
gaggcagcgcggctatcgtggctggccacgacgggcgttc
cttgcgcagctgtgctcgacgttgtcactgaagcgggaag
ggactggctgctattgggcgaagtgccggggcaggatctc
ctgtcatctcaccttgctcctgccgagaaagtatccatca
tggctgatgcaatgcggcggctgcatacgcttgatccggc
tacctgcccattcgaccaccaagcgaaacatcgcatcgag
cgagcacgtactcggatggaagccggtcttgtcgatcagg
atgatctggacgaagagcatcaggggctcgcgccagccga
actgttcgccaggctcaaggcgagcatgcccgacggcgag
gatctcgtcgtgacccatggcgatgcctgcttgccgaata
tcatggtggaaaatggccgcttttctggattcatcgactg
tggccggctgggtgtggcggaccgctatcaggacatagcg
ttggctacccgtgatattgctgaagagcttggcggcgaat
gggctgaccgcttcctcgtgctttacggtatcgccgctcc
cgattcgcagcgcatcgccttctatcgccttcttgacgag
ttcttctgagcgggactctggggttcgaaatgaccgacca
agcgacgcccaacctgccatcacgagatttcgattccacc
gccgccttctatgaaaggttgggcttcggaatcgttttcc
gggacgccggctggatgatcctccagcgcggggatctcat
gctggagttcttcgcccaccctagggggaggctaactgaa
acacggaaggagacaataccggaaggaacccgcgctatga
cggcaataaaaagacagaataaaacgcacggtgttgggtc
gtttgttcataaacgcggggttcggtcccagggctggcac
tctgtcgataccccaccgagaccccattggggccaatacg
cccgcgtttcttccttttccccaccccaccccccaagttc
gggtgaaggcccagggctcgcagccaacgtcggggcggca
ggccctgccatagcctcaggttactcatatatactttaga
ttgatttaaaacttcatttttaatttaaaaggatctaggt
gaagatcctttttgataatctcatgaccaaaatcccttaa
cgtgagttttcgttccactgagcgtcagaccccgtagaaa
agatcaaaggatcttcttgagatcctttttttctgcgcgt
aatctgctgcttgcaaacaaaaaaaccaccgctaccagcg
gtggtttgtttgccggatcaagagctaccaactctttttc
cgaaggtaactggcttcagcagagcgcagataccaaatac
tgtccttctagtgtagccgtagttaggccaccacttcaag
aactctgtagcaccgcctacatacctcgctctgctaatcc
tgttaccagtggctgctgccagtggcgataagtcgtgtct
taccgggttggactcaagacgatagttaccggataaggcg
cagcggtcgggctgaacggggggttcgtgcacacagccca
gcttggagcgaacgacctacaccgaactgagatacctaca
gcgtgagctatgagaaagcgccacgcttcccgaagggaga
aaggcggacaggtatccggtaagcggcagggtcggaacag
gagagcgcacgagggagcttccagggggaaacgcctggta
tctttatagtcctgtcgggtttcgccacctctgacttgag
cgtcgatttttgtgatgctcgtcaggggggcggagcctat
ggaaaaacgccagcaacgcggcctttttacggttcctggc
cttttgctggccttttgctcacatgttctttcctgcgtta
tcccctgattctgtggataaccgtattaccgccatgcat

The pTCP backbone sequence (SEQ ID NO: 167):

tagttattaatagtaatcaattacggggtcattagttcat
agcccatatatggagttccgcgttacataacttacggtaa
atggcccgcctggctgaccgcccaacgacccccgcccatt
gacgtcaataatgacgtatgttcccatagtaacgccaata
gggactttccattgacgtcaatgggtggagtatttacggt
aaactgcccacttggcagtacatcaagtgtatcatatgcc
aagtacgccccctattgacgtcaatgacggtaaatggccc
gcctggcattatgcccagtacatgaccttatgggactttc
ctacttggcagtacatctacgtattagtcatcgctattac
catggtgatgcggttttggcagtacatcaatgggcgtgga
tagcggtttgactcacggggatttccaagtctccacccca
ttgacgtcaatgggagtttgttttggcaccaaaatcaacg
ggactttccaaaatgtcgtaacaactccgccccattgacg
caaatgggcggtaggcgtgtacggtgggaggtctatataa
gcagagctggtttagtgaaccgtggatcccgtcgcttacc
gattcagaatggttgatatccgccattctgaatcggtaag
cgacgaagcttaataaaggatcttttattttcattggatc
tgtgtgttggttttttgtgtgcggccgccctcgactgtgc
cttctagaagacaatagcaggcatgctggggatgcggtgg
gctctatggcttctgaggcggaaagaaccagctggggctc
tagggggtatccccacgcgccctgtagcggcgcattaagc
gcggcgggtgtggtggttacgcgcagcgtgaccgctacac
ttgccagcgccctagcgcccgctcctttcgctttcttccc
ttcctttctcgccacgttcgccggctttccccgtcaagct
ctaaatcgggggctccctttagggttccgatttagtgctt
tacggcacctcgaccccaaaaaacttgattagggtgatgg
ttcacgtagtgggccatcgccctgatagacggtttttcgc
cctttgacgttggagtccacgttctttaatagtggactct
tgttccaaactggaacaacactcaaccctatctcggtcta
ttcttttgatttataagggattttgccgatttcggcctat
tggttaaaaaatgagctgatttaacaaaaatttaacgcga
attaattctgtggaatgtgtgtcagttagggtgtggaaag
tccccaggctccccagcaggcagaagtatgcaaagcatgc
atctcaattagtcagcaaccaggtgtggaaagtccccagg
ctccccagcaggcagaagtatgcaaagcatgcatctcaat
tagtcagcaaccatagtcccgcccctaactccgcccatcc
cgcccctaactccgcccagttccgcccattctccgcccca
tggctgactaattttttttatttatgcagaggccgaggcc
gcctctgcctctgagctattccagaagtagtgaggaggct
tttttggaggcctaggcttttgcaaaaagctcccgggatg
accgagtacaagcccacggtgcgcctcgccacccgcgacg
acgtcccgcgggccgtacgcaccctcgccgccgcgttcgc
cgactaccccgccacgcgccacaccgtcgacccggaccgc
cacatcgagcgggtcaccgagctgcaagaactcttcctca
cgcgcgtcgggctcgacatcggcaaggtgtgggtcgcgga
cgacggcgccgcggtggcggtctggaccacgccggagagc
gtcgaagcgggggcggtgttcgccgagatcggcccgcgca
tggccgagttgagcggttcccggctggccgcgcagcaaca
gatggaaggcctcctggcgccgcaccggcccaaggagccc
gcgtggttcctggccaccgtcggcgtctcgcccgaccacc
agggcaagggtctgggcagcgccgtcgtgctccccggagt
ggaggcggccgagcgcgccggggtgcccgccttcctggag
acctccgcgccccgcaacctccccttctacgagcggctcg
gcttcaccgtcaccgccgacgtcgaggtgcccgaaggacc
gcgcacctggtgcatgacccgcaagcccggtgcctgattc
gaatgaccgaccaagcgacgcccaacctgccatcacgaga
tttcgattccaccgccgccttctatgaaaggttgggcttc
ggaatcgttttccgggacgccggctggatgatcctccagc
gcggggatctcatgctggagttcttcgcccaccccaactt
gtttattgcagcttataatggttacaaataaagcaatagc
atcacaaatttcacaaataaagcatttttttcactgcatt
ctagttgtggtttgtccaaactcatcaatgtatcttatca
tgtctgtataccgtcgacctctagctagagcttggcgtaa
tcatggtcatagctgtttcctgtgtgaaattgttatccgc
tcacaattccacacaacatacgagccggaagcataaagtg
taaagcctggggtgcctaatgagtgagctaactcacatta
attgcgttgcgctcactgcccgctttccagtcgggaaacc
tgtcgtgccagctgcattaatgaatcggccaacgcgcggg
gagaggcggtttgcgtattgggcgctcttccgcttcctcg
ctcactgactcgctgcgctcggtcgttcggctgcggcgag
cggtatcagctcactcaaaggcggtaatacggttatccac
agaatcaggggataacgcaggaaagaacatgtgagcaaaa
ggccagcaaaaggccaggaaccgtaaaaaggccgcgttgc
tggcgtttttccataggctccgcccccctgacgagcatca
caaaaatcgacgctcaagtcagaggtggcgaaacccgaca
ggactataaagataccaggcgtttccccctggaagctccc
tcgtgcgctctcctgttccgaccctgccgcttaccggata
cctgtccgcctttctcccttcgggaagcgtggcgctttct
catagctcacgctgtaggtatctcagttcggtgtaggtcg
ttcgctccaagctgggctgtgtgcacgaaccccccgttca
gcccgaccgctgcgccttatccggtaactatcgtcttgag
tccaacccggtaagacacgacttatcgccactggcagcag
ccactggtaacaggattagcagagcgaggtatgtaggcgg
tgctacagagttcttgaagtggtggcctaactacggctac
actagaagaacagtatttggtatctgcgctctgctgaagc
cagttaccttcggaaaaagagttggtagctcttgatccgg
caaacaaaccaccgctggtagcggtttttttgtttgcaag
cagcagattacgcgcagaaaaaaaggatctcaagaagatc
ctttgatcttttctacggggtctgacgctcagtggaacga
aaactcacgttaagggattttggtcatgagattatcaaaa
aggatcttcacctagatccttttaaattaaaaatgaagtt
ttaaatcaatctaaagtatatatgagtaaacttggtctga
cagttaccaatgcttaatcagtgaggcacctatctcagcg
atctgtctatttcgttcatccatagttgcctgactccccg
tcgtgtagataactacgatacgggagggcttaccatctgg
ccccagtgctgcaatgataccgcgagacccacgctcaccg
gctccagatttatcagcaataaaccagccagccggaaggg
ccgagcgcagaagtggtcctgcaactttatccgcctccat
ccagtctattaattgttgccgggaagctagagtaagtagt
tcgccagttaatagtttgcgcaacgttgttgccattgcta
caggcatcgtggtgtcacgctcgtcgtttggtatggcttc
attcagctccggttcccaacgatcaaggcgagttacatga
tcccccatgttgtgcaaaaaagcggttagctccttcggtc
ctccgatcgttgtcagaagtaagttggccgcagtgttatc
actcatggttatggcagcactgcataattctcttactgtc
atgccatccgtaagatgcttttctgtgactggtgagtact
caaccaagtcattctgagaatagtgtatgcggcgaccgag
ttgctcttgcccggcgtcaatacgggataataccgcgcca
catagcagaactttaaaagtgctcatcattggaaaacgtt
cttcggggcgaaaactctcaaggatcttaccgctgttgag
atccagttcgatgtaacccactcgtgcacccaactgatct
tcagcatcttttactttcaccagcgtttctgggtgagcaa
aaacaggaaggcaaaatgccgcaaaaaagggaataagggc
gacacggaaatgttgaatactcatactcttcctttttcaa
tattattgaagcatttatcagggttattgtctcatgagcg
gatacatatttgaatgtatttagaaaaataaacaaatagg
ggttccgcgcacatttccccgaaaagtgccacctgacgtc
gacggatcgggagatctcccgatcccctatggtgcactct
cagtacaatctgctctgatgccgcatagttaagccagtat
ctgctccctgcttgtgtgttggaggtcgctgagtagtgcg
cgagcaaaatttaagctacaacaaggcaaggcttgaccga
caattgcatgaagaatctgcttagggttaggcgttttgcg
ctgcttcgcgatgtacgggccagatatacgcgttgacatt
gattattgac

The pIRES backbone sequence (SEQ ID NO: 168):

tcaatattggccattagccatattattcattggttatata
gcataaatcaatattggctattggccattgcatacgttgt
atctatatcataatatgtacatttatattggctcatgtcc
aatatgaccgccatgttggcattgattattgactagttat
taatagtaatcaattacggggtcattagttcatagcccat
atatggagttccgcgttacataacttacggtaaatggccc
gcctggctgaccgcccaacgacccccgcccattgacgtca
ataatgacgtatgttcccatagtaacgccaatagggactt
tccattgacgtcaatgggtggagtatttacggtaaactgc
ccacttggcagtacatcaagtgtatcatatgccaagtccg
ccccctattgacgtcaatgacggtaaatggcccgcctggc
attatgcccagtacatgaccttacgggactttcctacttg
gcagtacatctacgtattagtcatcgctattaccatggtg
atgcggttttggcagtacaccaatgggcgtggatagcggt
ttgactcacggggatttccaagtctccaccccattgacgt
caatgggagtttgttttggcaccaaaatcaacgggacttt
ccaaaatgtcgtaacaactgcgatcgcccgccccgttgac
gcaaatgggcggtaggcgtgtacggtgggaggtctatata
agcagagctcgtttagtgaaccgtcagatcactagaagct
ttattgcggtagtttatcacagttaaattgctaacgcagt
cagtgcttctgacacaacagtctcgaacttaagctgcagt
gactctcttaaggtagccttgcagaagttggtcgtgaggc
actgggcaggtaagtatcaaggttacaagacaggtttaag
gagaccaatagaaactgggcttgtcgagacagagaagact
cttgcgtttctgataggcacctattggtcttactgacatc
cactttgcctttctctccacaggtgtccactcccagttca
attacagctcttaaggctagagtacttaatacgactcact
ataggctagcctcgagaattcacgcgtcgagcatgcatct
agggcggccaattccgcccctctcccccccccccctctcc
ctcccccccccctaacgttactggccgaagccgcttggaa
taaggccggtgtgcgtttgtctatatgttattttccacca
tattgccgtcttttggcaatgtgagggcccggaaacctgg
ccctgtcttcttgacgagcattcctaggggtctttcccct
ctcgccaaaggaatgcaaggtctgttgaatgtcgtgaagg
aagcagttcctctggaagcttcttgaagacaaacaacgtc
tgtagcgaccctttgcaggcagcggaaccccccacctggc
gacaggtgcctctgcggccaaaagccacgtgtataagata
cacctgcaaaggcggcacaaccccagtgccacgttgtgag
ttggatagttgtggaaagagtcaaatggctctcctcaagc
gtattcaacaaggggctgaaggatgcccagaaggtacccc
attgtatgggatctgatctggggcctcggtgcacatgctt
tacatgtgtttagtcgaggttaaaaaaacgtctaggcccc
ccgaaccacggggacgtggttttcctttgaaaaacacgat
gataagcttgccacaacccgggatcctctagagtcgaccc
gggcggccgcttccctttagtgagggttaatgcttcgagc
agacatgataagatacattgatgagtttggacaaaccaca
actagaatgcagtgaaaaaaatgctttatttgtgaaattt
gtgatgctattgctttatttgtaaccattataagctgcaa
taaacaagttaacaacaacaattgcattcattttatgttt
caggttcagggggagatgtgggaggttttttaaagcaagt
aaaacctctacaaatgtggtaaaatccgataaggatcgat
ccgggctggcgtaatagcgaagaggcccgcaccgatcgcc
cttcccaacagttgcgcagcctgaatggcgaatggacgcg
ccctgtagcggcgcattaagcgcggcgggtgtggtggtta
cgcgcagcgtgaccgctacacttgccagcgccctagcgcc
cgctcctttcgctttcttcccttcctttctcgccacgttc
gccggctttccccgtcaagctctaaatcgggggctccctt
tagggttccgatttagtgctttacggcacctcgaccccaa
aaaacttgattagggtgatggttcacgtagtgggccatcg
ccctgatagacggtttttcgccctttgacgttggagtcca
cgttctttaatagtggactcttgttccaaactggaacaac
actcaaccctatctcggtctattcttttgatttataaggg
attttgccgatttcggcctattggttaaaaaatgagctga
tttaacaaaaatttaacgcgaattttaacaaaatattaac
gcttacaatttcctgatgcggtattttctccttacgcatc
tgtgcggtatttcacaccgcatacgcggatctgcgcagca
ccatggcctgaaataacctctgaaagaggaacttggttag
gtaccttctgaggcggaaagaaccagctgtggaatgtgtg
tcagttagggtgtggaaagtccccaggctccccagcaggc
agaagtatgcaaagcatgcatctcaattagtcagcaacca
ggtgtggaaagtccccaggctccccagcaggcagaagtat
gcaaagcatgcatctcaattagtcagcaaccatagtcccg
cccctaactccgcccatcccgcccctaactccgcccagtt
ccgcccattctccgccccatggctgactaattttttttat
ttatgcagaggccgaggccgcctcggcctctgagctattc
cagaagtagtgaggaggcttttttggaggcctaggctttt
gcaaaaagcttgattcttctgacacaacagtctcgaactt
aaggctagagccaccatgattgaacaagatggattgcacg
caggttctccggccgcttgggtggagaggctattcggcta
tgactgggcacaacagacaatcggctgctctgatgccgcc
gtgttccggctgtcagcgcaggggcgcccggttctttttg
tcaagaccgacctgtccggtgccctgaatgaactgcagga
cgaggcagcgcggctatcgtggctggccacgacgggcgtt
ccttgcgcagctgtgctcgacgttgtcactgaagcgggaa
gggactggctgctattgggcgaagtgccggggcaggatct
cctgtcatctcaccttgctcctgccgagaaagtatccatc
atggctgatgcaatgcggcggctgcatacgcttgatccgg
ctacctgcccattcgaccaccaagcgaaacatcgcatcga
gcgagcacgtactcggatggaagccggtcttgtcgatcag
gatgatctggacgaagagcatcaggggctcgcgccagccg
aactgttcgccaggctcaaggcgcgcatgcccgacggcga
ggatctcgtcgtgacccatggcgatgcctgcttgccgaat
atcatggtggaaaatggccgcttttctggattcatcgact
gtggccggctgggtgtggcggaccgctatcaggacatagc
gttggctacccgtgatattgctgaagagcttggcggcgaa
tgggctgaccgcttcctcgtgctttacggtatcgccgctc
ccgattcgcagcgcatcgccttctatcgccttcttgacga
gttcttctgagcgggactctggggttcgaaatgaccgacc
aagcgacgcccaacctgccatcacgatggccgcaataaaa
tatctttattttcattacatctgtgtgttggttttttgtg
tgaatcgatagcgataaggatccgcgtatggtgcactctc
agtacaatctgctctgatgccgcatagttaagccagcccc
gacacccgccaacacccgctgacgcgccctgacgggcttg
tctgctcccggcatccgcttacagacaagctgtgaccgtc
tccgggagctgcatgtgtcagaggttttcaccgtcatcac
cgaaacgcgcgagacgaaagggcctcgtgatacgcctatt
tttataggttaatgtcatgataataatggtttcttagacg
tcaggtggcacttttcggggaaatgtgcgcggaaccccta
tttgtttatttttctaaatacattcaaatatgtatccgct
catgagacaataaccctgataaatgcttcaataatattga
aaaaggaagagtatgagtattcaacatttccgtgtcgccc
ttattcccttttttgcggcattttgccttcctgtttttgc
tcacccagaaacgctggtgaaagtaaaagatgctgaagat
cagttgggtgcacgagtgggttacatcgaactggatctca
acagcggtaagatccttgagagttttcgccccgaagaacg
ttttccaatgatgagcacttttaaagttctgctatgtggc
gcggtattatcccgtattgacgccgggcaagagcaactcg
gtcgccgcatacactattctcagaatgacttggttgagta
ctcaccagtcacagaaaagcatcttacggatggcatgaca
gtaagagaattatgcagtgctgccataaccatgagtgata
acactgcggccaacttacttctgacaacgatcggaggacc
gaaggagctaaccgcttttttgcacaacatgggggatcat
gtaactcgccttgatcgttgggaaccggagctgaatgaag
ccataccaaacgacgagcgtgacaccacgatgcctgtagc
aatggcaacaacgttgcgcaaactattaactggcgaacta
cttactctagcttcccggcaacaattaatagactggatgg
aggcggataaagttgcaggaccacttctgcgctcggccct
tccggctggctggtttattgctgataaatctggagccggt
gagcgtgggtctcgcggtatcattgcagcactggggccag
atggtaagccctcccgtatcgtagttatctacacgacggg
gagtcaggcaactatggatgaacgaaatagacagatcgct
gagataggtgcctcactgattaagcattggtaactgtcag
accaagtttactcatatatactttagattgatttaaaact
tcatttttaatttaaaaggatctaggtgaagatccttttt
gataatctcatgaccaaaatcccttaacgtgagttttcgt
tccactgagcgtcagaccccgtagaaaagatcaaaggatc
ttcttgagatcctttttttctgcgcgtaatctgctgcttg
caaacaaaaaaaccaccgctaccagcggtggtttgtttgc
cggatcaagagctaccaactctttttccgaaggtaactgg
cttcagcagagcgcagataccaaatactgttcttctagtg
tagccgtagttaggccaccacttcaagaactctgtagcac
cgcctacatacctcgctctgctaatcctgttaccagtggc
tgctgccagtggcgataagtcgtgtcttaccgggttggac
tcaagacgatagttaccggataaggcgcagcggtcgggct
gaacggggggttcgtgcacacagcccagcttggagcgaac
gacctacaccgaactgagatacctacagcgtgagctatga
gaaagcgccacgcttcccgaagggagaaaggcggacaggt
atccggtaagcggcagggtcggaacaggagagcgcacgag
ggagcttccagggggaaacgcctggtatctttatagtcct
gtcgggtttcgccacctctgacttgagcgtcgatttttgt
gatgctcgtcaggggggcggagcctatggaaaaacgccag
caacgcggcctttttacggttcctggccttttgctggcct
tttgctcacatggctcgacagatct

In some embodiments therefore, the composition of the disclosure comprises a nucleic acid molecule comprising at least about 70%, 75%, 80%, 85%, 86%, 87%, 88%, 89%, 90%, 91%, 92%, 93%, 94%, 95%, 96%, 97%, 98% or 99% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof. In some embodiments, the composition of the disclosure comprises a nucleic acid molecule comprising the nucleotide sequence of SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof. In some embodiments, the composition of the disclosure comprises a nucleic acid molecule that is a pVax variant.

In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%. 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising a first nucleic acid sequence encoding a scaffold domain comprising any of the self-assembling polypeptides disclosed herein, or a functional fragment or variant thereof, and a second nucleic acid sequence encoding an antigen domain comprising any of the viral antigens disclosed herein, or a functional fragment or variant thereof. In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising a first nucleic acid sequence encoding a scaffold domain comprising a self-assembling polypeptide comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 8, SEQ ID NO: 10, SEQ ID NO: 12, SEQ ID NO: 14, SEQ ID NO: 16, SEQ ID NO: 18 or SEQ ID NO: 20, or a functional fragment or variant thereof, and a second nucleic acid sequence encoding an antigen domain comprising a viral antigen comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising a first nucleic acid sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment or variant thereof, and a second nucleic acid sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 59, SEQ ID NO: 61, SEQ ID NO: 63 or SEQ ID NO: 65, or a functional fragment or variant thereof. In some embodiments, such nucleic acid molecules or plasmids may further comprise a third nucleic acid sequence encoding a leader sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 or SEQ ID NO: 5, or a functional fragment or variant thereof. In such embodiments, the third nucleic acid sequence encoding a leader sequence may comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 2, SEQ ID NO: 3, SEQ ID NO: 4 or SEQ ID NO: 6, or a functional fragment or variant thereof.

In some embodiments, the nucleic acid molecules or plasmids of the disclosure may additionally comprise another nucleic acid sequence encoding a linker comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the nucleic acid sequence encoding a linker may comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof.

In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising a first nucleic acid sequence encoding a leader sequence comprising any of the leader sequences disclosed herein, or a functional fragment or variant thereof, and a second nucleic acid sequence encoding a viral trimer (or three viral monomers) comprising any of the viral antigens disclosed herein, or a functional fragment or variant thereof. In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising a first nucleic acid sequence encoding a leader sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 or SEQ ID NO: 5, or a functional fragment or variant thereof, and a second nucleic acid sequence encoding three viral monomers, each viral monomer independently comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising a first nucleic acid sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 2, SEQ ID NO: 3, SEQ ID NO: 4 or SEQ ID NO: 6, or a functional fragment or variant thereof, and a second nucleic acid sequence encoding three viral monomers, each viral monomer independently being encoded by a nucleic acid sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 59, SEQ ID NO: 61, SEQ ID NO: 63 or SEQ ID NO: 65, or a functional fragment or variant thereof. In some embodiments, each of the viral monomers is linked by one or more linker peptides comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, each of the viral monomers is linked by one or more linker peptides encoded by a nucleic acid sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof.

In some embodiments, any of the nucleic acid molecules or plasmids of the disclosure additionally comprises a nucleic acid sequence encoding a furin cleavage site comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 67, or a functional fragment or variant thereof.

In some embodiments, the nucleic acid molecule or plasmid may further comprises a nucleic acid encoding a transmembrane domain and a foldon domain. A non-limiting example of the transmembrane domain is the transmembrane domain of a platelet derived growth factor receptor comprising the sequence of AVGQDTQEVIVVPHSL PFKVVVISAILALVVLTIISLIILIMLWQKKPR (SEQ ID NO: 169). A non-limiting example of the foldon domain may comprise the sequence of YIPEAPRDGQAYVRKD GEWVLLSTFL (SEQ ID NO: 170). Thus, in some embodiments, the composition of the disclosure comprises a nucleic acid molecule or a plasmid comprising a nucleic acid sequence encoding a transmembrane domain comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 169, or a functional fragment or variant thereof. In some embodiments, the composition of the disclosure comprises a nucleic acid molecule or a plasmid comprising a nucleic acid sequence encoding a foldon domain comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 170, or a functional fragment or variant thereof.

In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the composition of the disclosure comprises a nucleic acid molecule or a plasmid comprising the nucleotide sequence of SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence comprising the nucleotide sequence of SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the composition of the disclosure comprises a nucleic acid molecule or a plasmid comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence encoding a polypeptide comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof. In some embodiments, the composition of the disclosure comprises a nucleic acid molecule or a plasmid comprising the nucleotide sequence of SEQ ID NO: 161, SEQ ID NO: 162, SEQ ID NO: 163, SEQ ID NO: 164, SEQ ID NO: 165, SEQ ID NO: 166, SEQ ID NO: 1676 or SEQ ID NO: 168, or a functional fragment or variant thereof, and an expressible nucleic acid sequence encoding a polypeptide comprising the amino acid sequence of SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

In some embodiments, the disclosure relates to a vector or a plasmid comprising one or a plurality of regulatory sequences operably linked to one or more of any of the disclosed expressible nucleic acid sequences. In some embodiments, the disclosure relates to a composition comprising a nucleic acid molecule comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 161, or a functional fragment or variant thereof, and positioned within the multiple cloning site thereof is one or more expressible nucleic acid sequences according to the present disclosure. In some embodiments, the disclosure relates to a composition comprising one or a plurality of RNA molecules, each individually comprising the RNA sequences disclosed herein, including but not limited to SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, SEQ ID NO: 156 or SEQ ID NO: 159, or a functional fragment or variant thereof.

### C. Polypeptide Sequences

Disclosed are the polypeptide sequences encoded by the disclosed nucleic acid sequences. In some embodiments, the disclosure relates to compositions comprising polypeptide sequences encoded by the expressible nucleic acid molecules of the present disclosure comprising a scaffold domain comprising a self-assembling polypeptide and an antigen domain comprising a viral antigen, and optionally comprising a leader domain comprising a leader sequence and/or a linker domain comprising a linker peptide. In some embodiments, the disclosure relates to compositions comprising polypeptide sequences encoded by the expressible nucleic acid molecules of the present disclosure comprising a leader domain comprising a leader sequence and an antigen domain comprising three viral monomers (trimer), and optionally comprising one or plurality of linker domains each comprising a linker peptide. The disclosure also relates to cells expressing one or more such polypeptides disclosed herein.

In some embodiments, the antigen domain comprised in the polypeptides of the disclosure comprises a soluble viral trimer or a soluble monomer thereof, wherein the soluble viral trimer or the soluble monomer thereof is from a virus of the family Coronaviridae. A “viral trimer” refers to a protein complex formed by three covalently or non-convalently bound viral proteins. Each of these viral proteins, when present by itself, is called as a “monomer” of the viral trimer.

Solubility of the disclosed viral trimer or monomer thereof can be determined using any methods known in the art. Protein solubility is a thermodynamic parameter defined as the concentration of protein in a saturated solution that is in equilibrium with a solid phase, either crystalline or amorphous, under a given set of conditions. Solubility can be influenced by a number of extrinsic and intrinsic factors. Extrinsic factors that influence protein solubility include pH, ionic strength, temperature, and the presence of various solvent additives. Varying these extrinsic factors can lead to increased solubility; however, altering the solution conditions is not always appropriate or sufficient to increase protein solubility to the extent required. The intrinsic factors that influence protein solubility are defined primarily by the amino acids on the protein surface, but a detailed understanding of how one can alter the intrinsic properties of a protein to increase its solubility is lacking.

There are generally two methods used to measure protein solubility in aqueous solution: (1) adding lyophilized protein to solvent; and (2) concentrating a protein solution by ultrafiltration. Both of these methods require that the concentration of protein in solution be increased until saturation is reached; however, this is often difficult to do, especially with very soluble proteins, because gel-like or supersaturated solutions may form, making it difficult to determine the solubility values accurately. When lyophilized protein is added to solvent, the variable water and salt content of the lyophilized powder is difficult to control and can have a significant effect on solubility measurements.

One way to avoid the difficulties of measuring protein solubility is to make use of an extraneous agent that lowers the solubility of a protein called a precipitant. Protein precipitants can be divided into three classes: salts, organic solvents, and long-chain polymers. These precipitants are used by crystallographers to achieve slow precipitation and crystal formation; however, they can also be used to induce amorphous precipitation by direct mixing with protein solutions. Common examples from each of these three classes of precipitants, respectively, are ammonium sulfate, isopropanol, and polyethylene glycol (PEG). The relationship between precipitant concentration to protein solubility is described by the following general expression:

Log S=constant−β[Precipitant], Equation (1)

where S is the measured solubility at a given concentration of precipitant, and 13 is the dependence of solubility on precipitant concentration for a given protein. The constant is the y-intercept of the solubility plot, and for PEG precipitations is equal to the logarithm of the protein activity. For dilute protein solutions, as the activity constant approaches one, Equation (1) becomes

Log S=Log Sₒ−β[Precipitant], Equation (2)

where S₀ is the solubility in the absence of precipitant. It is showed that for PEG precipitations, the linearity of Equation (1) extends to zero precipitant for proteins whose solubility can be accurately measured in buffer alone. In this case, the constant portion of Equation (2), Log S₀, can be used as an estimate of solubility in the absence of precipitant. For salts, Equation (1) only describes the salting-out region of the solubility plot. At low salt concentrations, salting-in is observed and the solubility is higher than in the absence of salt. Therefore, the constant obtained from salt precipitations represents a projection of the salting-out region onto the y axis. In some embodiments, the nucleic acid sequences of certain compositions encode viral monomers that, upon expression, preferentially form into viral trimers. In some embodiments, the viral antigens form into viral trimer proteins displayed upon a scaffold polypeptide, such as a self-assembling polypeptide polyhedron. In some embodiments, the viral anigens are at a concentration within the disclosed compositions that does not exceed a concentration that causes precipitation out of solution. In some embodiments, the scaffold peptide and the displayed viral antigens are soluble in aqueous solution, such as PBS, sterile water or buffered Ringer's solution.

In some embodiments, the polypeptide encoded by the expressible nucleic acid molecule of the present disclosure comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof. In some embodiments, the polypeptide is encoded by a nucleic acid sequence comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof.

In some embodiments, the leader sequence encoded by the expressible nucleic acid sequence of the disclosure comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 or SEQ ID NO: 5, or a functional fragment or variant thereof. In some embodiments, the leader sequence is encoded by a nucleic acid sequence comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 2, SEQ ID NO: 3, SEQ ID NO: 4 or SEQ ID NO: 6, or a functional fragment or variant thereof.

In some embodiments, the self-assembling polypeptide encoded by the expressible nucleic acid sequence of the disclosure comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 8, SEQ ID NO: 10, SEQ ID NO: 12, SEQ ID NO: 14, SEQ ID NO: 16, SEQ ID NO: 18 or SEQ ID NO: 20, or a functional fragment or variant thereof. In some embodiments, the self-assembling polypeptide is encoded by a nucleic acid sequence comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment or variant thereof.

In some embodiments, the linker peptide encoded by the expressible nucleic acid sequence of the disclosure comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the linker peptide is encoded by a nucleic acid sequence comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof.

In some embodiments, the viral antigen or monomer encoded by the expressible nucleic acid sequence of the disclosure comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the viral antigen or monomer is encoded by a nucleic acid sequence comprises at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 59, SEQ ID NO: 61, SEQ ID NO: 63 or SEQ ID NO: 65, or a functional fragment or variant thereof.

In some embodiments, the polypeptides encoded by the expressible nucleic acid molecule of the present disclosure comprises a furin cleavage site comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 67. In some embodiments, the polypeptides encoded by the expressible nucleic acid molecule of the present disclosure comprises a transmembrane domain comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 169. In some embodiments, the polypeptides encoded by the expressible nucleic acid molecule of the present disclosure comprises a foldon domain comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 170.

### D. Pharmaceutical Compositions

Disclosed are pharmaceutical compositions comprising any one or more of the disclosed compositions and a pharmaceutically acceptable carrier. Disclosed are pharmaceutical compositions comprising therapeutically effective amounts of any one or more of the disclosed compositions and a pharmaceutically acceptable carrier.

In some embodiments, any of the disclosed compositions is from about 1 to about 30 micrograms of the disclosed DNA and/or RNA vaccine. For example, any of the disclosed compositions can be from about 1 to about 5 micrograms the disclosed DNA and/or RNA vaccine. In some preferred embodiments, the pharmaceutical compositions contain from about 5 nanograms to about 800 micrograms of the disclosed DNA and/or RNA vaccine. In some preferred embodiments, the pharmaceutical compositions contain about 25 to about 250 micrograms, from about 100 to about 200 micrograms, from about 1 nanogram to 100 milligrams; from about 1 microgram to about 10 milligrams; from about 0.1 microgram to about 10 milligrams; from about 1 milligram to about 2 milligrams, from about 5 nanograms to about 1000 micrograms, from about 10 nanograms to about 800 micrograms, from about 0.1 to about 500 micrograms, from about 1 to about 350 micrograms, from about 25 to about 250 micrograms, from about 100 to about 200 micrograms of the DNA and/or RNA vaccine or plasmid thereof. The pharmaceutical compositions can comprise from about 5 nanograms to about 10 mg of the disclosed DNA and/or RNA vaccine. In some embodiments, pharmaceutical compositions according to the present disclosure comprise from about 25 nanograms to about 5 mg of the disclosed DNA and/or RNA vaccine. In some embodiments, the pharmaceutical compositions contain from about 50 nanograms to about 1 mg of the disclosed DNA and/or RNA vaccine. In some embodiments, the pharmaceutical compositions contain about from about 0.1 to about 500 micrograms of the disclosed DNA and/or RNA vaccine. In some embodiments, the pharmaceutical compositions contain from about 1 to about 350 micrograms of the disclosed DNA and/or RNA vaccine. In some embodiments, the pharmaceutical compositions contain from about 5 to about 250 micrograms of the disclosed DNA and/or RNA vaccine. In some embodiments, the pharmaceutical compositions contain from about 10 to about 200 micrograms of the disclosed DNA and/or RNA vaccine. In some embodiments, the pharmaceutical compositions contain from about 15 to about 150 micrograms of the disclosed DNA and/or RNA vaccine. In some embodiments, the pharmaceutical compositions contain about 20 to about 100 micrograms of the disclosed DNA and/or RNA vaccine. In some embodiments, the pharmaceutical compositions contain about 25 to about 75 micrograms of the disclosed DNA and/or RNA vaccine. In some embodiments, the pharmaceutical compositions contain about 30 to about 50 micrograms of the disclosed DNA and/or RNA vaccine. In some embodiments, the pharmaceutical compositions contain about 35 to about 40 micrograms of the disclosed DNA and/or RNA vaccine. In some embodiments, the pharmaceutical compositions contain about 100 to about 200 micrograms the disclosed DNA and/or RNA vaccine. In some embodiments, the pharmaceutical compositions comprise about 10 micrograms to about 100 micrograms of the disclosed DNA and/or RNA vaccine. In some embodiments, the pharmaceutical compositions comprise about 20 micrograms to about 80 micrograms of the disclosed DNA and/or RNA vaccine. In some embodiments, the pharmaceutical compositions comprise about 25 micrograms to about 60 micrograms of the disclosed DNA and/or RNA vaccine. In some embodiments, the pharmaceutical compositions comprise about 30 nanograms to about 50 micrograms of the disclosed DNA and/or RNA vaccine. In some embodiments, the pharmaceutical compositions comprise about 35 nanograms to about 45 micrograms of the disclosed DNA and/or RNA vaccine. In some preferred embodiments, the pharmaceutical compositions contain about 0.1 to about 500 micrograms of the disclosed DNA and/or RNA vaccine. In some preferred embodiments, the pharmaceutical compositions contain about 1 to about 350 micrograms of the disclosed DNA and/or RNA vaccine. In some preferred embodiments, the pharmaceutical compositions contain about 1 to about 250 micrograms of the disclosed DNA and/or RNA vaccine. In some preferred embodiments, the pharmaceutical compositions contain about 2 to about 200 micrograms the disclosed DNA and/or RNA vaccine.

In some embodiments, pharmaceutical compositions according to the present disclosure comprise at least about 10, 15, 20, 25, 30, 35, 40, 45, 50, 55, 60, 65, 70, 75, 80, 85, 90, 95 or 100 nanograms of the disclosed DNA and/or RNA vaccine. In some embodiments, the pharmaceutical compositions can comprise at least about 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995 or 1000 micrograms of the disclosed DNA and/or RNA vaccine. In some embodiments, the pharmaceutical composition can comprise at least 1.5, 2, 2.5, 3, 3.5, 4, 4.5, 5, 5.5, 6, 6.5, 7, 7.5, 8, 8.5, 9, 9.5 or 10 mg or more of the disclosed DNA and/or RNA vaccine.

In other embodiments, the pharmaceutical composition can comprise up to and including about 10, 15, 20, 25, 30, 35, 40, 45, 50, 55, 60, 65, 70, 75, 80, 85, 90, 95 or 100 nanograms of the disclosed DNA and/or RNA vaccine. In some embodiments, the pharmaceutical composition can comprise up to and including about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 micrograms of the disclosed DNA and/or RNA vaccine. In some embodiments, the pharmaceutical composition can comprise up to and including about 1.5, 2, 2.5, 3, 3.5, 4, 4.5, 5, 5.5, 6, 6.5, 7, 7.5, 8, 8.5, 9, 9.5 or about 10 mg of the disclosed DNA and/or RNA vaccine. The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or other known transfection facilitating agents. In some embodiments, the vaccine is a composition comprising a plasmid DNA molecule, RNA molecule or DNA/RNA hybrid molecule encoding an expressible nucleic acid sequence, the expressible nucleic acid sequence comprising a first nucleic acid encoding a self-assembling nanoparticle comprising a viral antigen, optionally encoding a leader sequence disclosed herein.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA vector vaccines can also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid. Concentration of the transfection agent in the vaccine is less than about 4 mg/ml, less than about 2 mg/ml, less than about 1 mg/ml, less than about 0.750 mg/ml, less than about 0.500 mg/ml, less than about 0.250 mg/ml, less than about 0.100 mg/ml, less than about 0.050 mg/ml, or less than about 0.010 mg/ml.

The pharmaceutically acceptable excipient can be an adjuvant. The adjuvant can be other genes that are expressed in alternative plasmid or are deneurological systemed as proteins in combination with the plasmid above in the vaccine. The adjuvant can be selected from: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. In an exemplary embodiment, the adjuvant is IL-12.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRCS, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof.

In some embodiments adjuvant may be one or more proteins and/or nucleic acid molecules that encode proteins selected from the group consisting of: CCL-20, IL-12, IL-15, IL-28, CTACK, TECK, MEC or RANTES. Examples of IL-12 constructs and sequences are disclosed in PCT application No. PCT/US1997/019502 (published as WO98/017799) and corresponding U.S. application Ser. No. 08/956,865, and U.S. Provisional Application No. 61/569,600 filed Dec. 12, 2011, which are each incorporated herein by reference in their entireties. Examples of IL-15 constructs and sequences are disclosed in PCT application No. PCT/US04/18962 (published as WO2005/000235) and corresponding U.S. application Ser. No. 10/560,650, and in PCT application No. PCT/US07/00886 (published as WO2007/087178) and corresponding U.S. application Ser. No. 12/160,766, and in PCT Application Serial No. PCT/US10/048827 (published as WO2011/032179), which are each incorporated herein by reference in their entireties. Examples of IL-28 constructs and sequences are disclosed in PCT application no. PCT/US09/039648 (published as WO2009/124309) and corresponding U.S. application Ser. No. 12/936,192, which are each incorporated herein by reference in their entireties. Examples of RANTES and other constructs and sequences are disclosed in PCT application No. PCT/US 1999/004332 (published as WO99/043839) and corresponding U.S. application Ser. No. 09/622,452, which are each incorporated herein by reference in their entieties. Other examples of RANTES constructs and sequences are disclosed in PCT Application No. PCT/US Ser. No. 11/024,098 (published as WO2011/097640), which is incorporated herein by reference. Examples of RANTES and other constructs and sequences are disclosed in PCT Application No. PCT/US 1999/004332 and corresponding U.S. application Ser. No. 09/622,452, which are each incorporated herein by reference. Other examples of RANTES constructs and sequences are disclosed in PCT application No. PCT/US11/024098 (published as WO2011/097640), which is incorporated herein by reference in its entirety. Examples of chemokines CTACK, TECK and MEC constructs and sequences are disclosed in PCT Application No. PCT/US2005/042231 (published as WO2007/050095) and corresponding U.S. application Ser. No. 11/719,646, which are each incorporated herein by reference in their entireties. Examples of OX40 and other immunomodulators are disclosed in U.S. application Ser. No. 10/560,653, which is incorporated herein by reference in its entirety. Examples of DR5 and other immunomodulators are disclosed in U.S. application Ser. No. 09/622,452, which is incorporated herein by reference in its entirety.

The pharmaceutial compoistion may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

The vaccine can be a DNA or RNA vaccine. In some embodiments, the vaccine is a DNA vaccine. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome. Examples of attenuated live vaccines, those using recombinant vectors to foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference in their entireties. In other embodiments, the vaccine is a RNA vaccine. RNA vaccines are disclosed in, for example, U.S. Pat. Nos. 10,064,934; 10,064,935; 10,124,055; 10,238,731; 10,272,150; 10,383,937; 10,449,244; 10,517,940; 10,543,269; 10,675,342; 10,702,597; 10,702,599; and 10,716,846; and U.S. Application Publication Nos. 2011/0033416; 2014/0134129; 2017/0136121; 2018/0344838; 2020/0016274; 2020/0030432; 2020/0405844; and 2021/0046173, which are each incorporated herein by reference in their entireties.

In some embodiments, the vaccine is a lipid nanoparticles (LNP) comprising one or a modified RNA molecule. In some embodiments, the vaccine comprises a modified mRNA. Modified polynucleotides (such as, but not limited to, primary constructs), formulations and compositions comprising modified polynucleotides, and methods of making, using and administering modified polynucleotides are described in U.S. Provisional Patent Application Nos. 61/618,862; 61/681,645; 61/737,130; 61/618,866; 61/681,647; 61/737,134; 61/618,868; 61/681,648; 61/737,135; 61/618,873; 61/681,650; 61/737,147; 61/618,878; 61/681,654; 61/737,152; 61/618,885; 61/681,658; 61/737,155; 61/618,896; 61/668,157; 61/681,661; 61/737,160; 61/618,911; 61/681,667; 61/737,168; 61/618,922; 61/681,675; 61/737,174; 61/618,935; U61/681,687; 61/737,184; 61/618,945; 61/681,696; 61/737,191; 61/618,953; 61/681,704; 61/737,203; 61/681,720; 61/737,213; 61/681,742; 61/618,870; 61/681,649; and 61/737,139; and International Application Nos. PCT/US2013/030062; PCT/US2013/030064; PCT/US2013/030059; PCT/US2013/030066; PCT/US2013/030067; PCT/US2013/030060; PCT/US2013/030061; PCT/US2013/030070; PCT/US2013/030068; PCT/US2013/030063; and PCT/US2013/031821, the contents of each of which are herein incorporated by reference in their entireties. Any of the recited polypeptides of the modified polynucleotides of the foregoing are considered useful as a polypeptide of interest or antigen of the LNPs of the present disclosure.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells.

In some embodiments, the disclosure relates to a DNA vector pVAX1 comprising any one or more of the expressible nucliec acid sequences disclosed herein or an RNA transcript thereof. In some embodiments, the disclosure relates to a pharmaceuical composition comprising a nucleic acid sequencethat includes one or a plurality of the expressible nucleic acid sequences discloed herein or an RNA transcript thereof, and a pharmaceutically acceptable carrier.

### E. Methods

Provided herein are compositions (e.g., pharmaceutical compositions), methods, kits and reagents for prevention and/or treatment of an infection of a virus from the family of Coronaviridae, particularly a coronavirus infection, in humans and other mammals. Virus vaccines can be used as therapeutic or prophylactic agents. They may be used in medicine to prevent and/or treat infectious disease. In exemplary aspects, the vaccines of the present disclosure are used to provide prophylactic protection from coronavirus. Prophylactic protection from coronavirus can be achieved following administration of a prophylactically effective dose of a coronavirus DNA or RNA vaccine of the present disclosure. A prophylactically effective dose is a therapeutically effective dose that prevents infection with the virus at a clinically acceptable level. In some embodiments, the therapeutically effective dose is a dose listed in a package insert for the vaccine. Vaccines can be administered once, twice, three times, four times or more. It is possible, although less desirable, to administer the vaccine to an infected individual to achieve a therapeutic response. Dosing may need to be adjusted accordingly.

In some embodiments, disclosed are methods of vaccinating a subject comprising administering a therapeutically effective amount of any of the disclosed nucleic acid molecules, compositions, pharmaceutical compositions, cells or vaccines to the subject. In some embodiments, the vaccination is against viral infection. In some embodiments, the viral infection is an infection of a virus from the family of Coronaviridae. In some embodiments, the viral infection is an infection of a coronavirus. In some embodiments, the viral infection is an infection of SARS-CoV. In some embodiments, the viral infection is an infection of HCoV NL63. In some embodiments, the viral infection is an infection of HKU1. In some embodiments, the viral infection is an infection of MERS-CoV. In some embodiments, the viral infection is an infection of SARS-CoV-2.

In some embodiments, disclosed are methods of inducing an immune response in a subject comprising administering to the subject any of the disclosed pharmaceutical compositions. In some embodiments, the methods are for inducing an immune response against a viral antigen in the subject. In some embodiments, the immune response induced by the disclosed methods is against a viral antigen from a virus from the family of Coronaviridae. In some embodiments, the immune response induced by the disclosed methods is against a viral antigen from a coronavirus. In some embodiments, the immune response induced by the disclosed methods is against a viral antigen from SARS-CoV. In some embodiments, the immune response induced by the disclosed methods is against a viral antigen from HCoV NL63. In some embodiments, the immune response induced by the disclosed methods is against a viral antigen from HKU1. In some embodiments, the immune response induced by the disclosed methods is against a viral antigen from MERS-CoV. In some embodiments, the immune response induced by the disclosed methods is against a viral antigen from SARS-CoV-2.

In some embodiments, disclosed are methods of neutralizing one or a plurality of viruses in a subject comprising administering to the subject any of the disclosed pharmaceutical compositions. In some embodiments, the virus being neutralized by the disclosed method is a virus from the family of Coronaviridae. In some embodiments, the virus being neutralized by the disclosed method is a coronavirus. In some embodiments, the virus being neutralized by the disclosed method is SARS-CoV. In some embodiments, the virus being neutralized by the disclosed method is HCoV NL63. In some embodiments, the virus being neutralized by the disclosed method is HKU1. In some embodiments, the virus being neutralized by the disclosed method is MERS-CoV. In some embodiments, the virus being neutralized by the disclosed method is SARS-CoV-2.

In some embodiments, disclosed are methods of neutralizing infection of one or a plurality of viruses in a subject comprising administering to the subject any of the disclosed pharmaceutical compositions. In some embodiments, the viral infection being neutralized by the disclosed method is an infection of a virus from the family of Coronaviridae. In some embodiments, the viral infection being neutralized by the disclosed method is an infection of coronavirus. In some embodiments, the viral infection being neutralized by the disclosed method is an infection of SARS-CoV. In some embodiments, the viral infection being neutralized by the disclosed method is an infection of HCoV NL63. In some embodiments, the viral infection being neutralized by the disclosed method is an infection of HKU1. In some embodiments, the viral infection being neutralized by the disclosed method is an infection of MERS-CoV. In some embodiments, the viral infection being neutralized by the disclosed method is an infection of SARS-CoV-2.

In some embodiments, disclosed are methods of stimulating a therapeutically effective antigen-specific immune response against a virus in a mammal infected with the virus comprising administering any of the disclosed pharmaceutical compositions. In some embodiments, the disclosed method is a method of stimulating a therapeutically effective antigen-specific immune response against a virus from the family of Coronaviridae. In some embodiments, the disclosed method is a method of stimulating a therapeutically effective antigen-specific immune response against a coronavirus. In some embodiments, the disclosed method is a method of stimulating a therapeutically effective antigen-specific immune response against SARS-CoV. In some embodiments, the disclosed method is a method of stimulating a therapeutically effective antigen-specific immune response against HCoV NL63. In some embodiments, the disclosed method is a method of stimulating a therapeutically effective antigen-specific immune response against HKU1. In some embodiments, the disclosed method is a method of stimulating a therapeutically effective antigen-specific immune response against MERS-CoV. In some embodiments, the disclosed method is a method of stimulating a therapeutically effective antigen-specific immune response against SARS-CoV-2.

In some embodiments, disclosed are methods of inducing expression of a self-assembling vaccine in a subject comprising administering any of the disclosed pharmaceutical compositions. Also disclosed are methods of treating a subject having a viral infection or susceptible to becoming infected with a virus comprising administering to the subject a therapeutically effective amount of any of the disclosed pharmaceutical compositions. In some embodiments, the viral infection is an infection of a virus from the family of Coronaviridae. In some embodiments, the viral infection is an infection of coronavirus. In some embodiments, the viral infection is an infection of SARS-CoV. In some embodiments, the viral infection is an infection of HCoV NL63. In some embodiments, the viral infection is an infection of HKU1. In some embodiments, the viral infection is an infection of MERS-CoV. In some embodiments, the viral infection is an infection of SARS-CoV-2.

The disclosed pharmaceutical compositions may be administered by any route of administration. Accordingly, in some embodiments, the administration is accomplished by oral administration. In some embodiments, the administration is accomplished by parenteral administration. In some embodiments, the administration is accomplished by sublingual administration. In some embodiments, the administration is accomplished by transdermal administration. In some embodiments, the administration is accomplished by rectal administration. In some embodiments, the administration is accomplished by transmucosal administration. In some embodiments, the administration is accomplished by topical administration. In some embodiments, the administration is accomplished by inhalation. In some embodiments, the administration is accomplished by buccal administration. In some embodiments, the administration is accomplished by intrapleural administration. In some embodiments, the administration is accomplished by intravenous administration. In some embodiments, the administration is accomplished by intraarterial administration. In some embodiments, the administration is accomplished by intraperitoneal administration. In some embodiments, the administration is accomplished by subcutaneous administration. In some embodiments, the administration is accomplished by intramuscular administration. In some embodiments, the administration is accomplished by intranasal administration. In some embodiments, the administration is accomplished by intrathecal administration. In some embodiments, the administration is accomplished by intraarticular administration. In some embodiments, the administration is accomplished by intradermal administration. In some embodiments, the above modes of action are accomplished by injection of the pharmaceutical compositions disclosed herein.

In some embodiments, the therapeutically effective dose can be from about 1 to about 30 micrograms of expressible nucleic acid sequence. In some embodiments, the therapeutically effective dose can be from about 0.001 micrograms of the composition per kilogram of subject to about 0.050 micrograms per kilogram of subject.

In some embodiments, any of the disclosed methods can be free of activating any mannose-binding lectin or complement process. In some embodiments, any of the disclosed methods is performed without inducing the MBL-complement pathway.

In some embodiments, the subject can be a human. In some embodiments, the subject is diagnosed with or suspected of having a viral infection. In some embodiments, the subject is diagnosed with or suspected of having an infection of a virus from the family of Coronaviridae. In some embodiments, the subject is diagnosed with or suspected of having an infection of coronavirus. In some embodiments, the subject is diagnosed with or suspected of having an infection of SARS-CoV. In some embodiments, the subject is diagnosed with or suspected of having an infection of HCoV NL63. In some embodiments, the subject is diagnosed with or suspected of having an infection of HKU1. In some embodiments, the subject is diagnosed with or suspected of having an infection of MERS-CoV. In some embodiments, the subject is diagnosed with or suspected of having an infection of SARS-CoV-2.

In some embodiments of the methods of inducing an immune response, the immune response can be an antigen-specific imune response. In some embodiments, the antigen-specific immune response can be an antigen-specific to SARS-CoV-2 antigen immune response. In some embodiments, the antigen-specific immune response can be a therapeutically effective CD-4+ antigen-specific SARS-CoV-2 immune response. In some embodiments, the antigen-specific immune response can be a therapeutically effective CD-8+ antigen-specific SARS-CoV-2 immune response. In some embodiments, the antigen-specific immune response can be a therapeutically effective CD-4+ and CD-8+ antigen-specific SARS-CoV-2 immune response.

In some embodiments, the methods are free of administering any polypeptide directly to the subject.

In some embodiments, any of the disclosed methods can further comprise administering to the subject a pharmaceutical composition comprising one or more pharmaceutically active agents, such as antiviral drugs, among many others. In some embodiments, the one or more pharmaceutically active agents include other anticoronarival medications used to inhibit coronavirus, for example nucleoside analog reverse transcriptase inhibitors, non-nucleoside reverse transcriptase inhibitors, and protease inhibitors. Among the available drugs that may be used as a pharmaceutically active agent are zidovudine or AZT (or Retrovir®), didanosine or DDI (or Videx®), stavudine or D4T (or Zerit®), lamivudine or 3TC (or Epivir®), zalcitabine or DDC (or Hivid®), abacavir succinate (or Ziagen″), tenofovir disoproxil fumarate salt (or Viread®), emtricitabine (or Emtriva®), Combivir® (contains 3TC and AZT), Trizivir® (contains abacavir, 3TC and AZT); three non-nucleoside reverse transcriptase inhibitors: nevirapine (or Viramune®), delavirdine (or Rescriptor®) and efavirenz (or Sustiva®), eight peptidomimetic protease inhibitors or approved formulations: saquinavir (or Invirase® or Fortovase″), indinavir (or Crixivan®), ritonavir (or Norvir®), nelfinavir (or Viracept″), amprenavir (or Agenerase®), atazanavir (Reyataz), fosamprenavir (or Lexiva), Kaletra®(contains lopinavir and ritonavir), and one fusion inhibitor enfuvirtide (or T-20 or Fuzeon®).

In some embodiments, the methods of inducing an immune response can include inducing a humoral or cellular immune response. A humoral immune response mainly refers to antibody production. A cellular immune response can include activation of CD4+ T-cells and activation CD8+ cells and associated cytotoxic activity. In one aspect, the present disclosure features a method of inducing an immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein. In one aspect, the present disclosure features a method of inducing a CD8+ T cell immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein.

In one aspect, the present disclosure features a method of enhancing an immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein.

In one aspect, the present disclosure features a method of enhancing a CD8+ T cell immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein.

In some embodiments, the subject has a viral infection and is in need of therapy for the viral infection. In some embodiments, the viral infection is an infection of a virus from the family of Coronaviridae. In some embodiments, the viral infection is an infection of coronavirus. In some embodiments, the viral infection is an infection of SARS-CoV. In some embodiments, the viral infection is an infection of HCoV NL63. In some embodiments, the viral infection is an infection of HKU1. In some embodiments, the viral infection is an infection of MERS-CoV. In some embodiments, the viral infection is an infection of SARS-CoV-2.

In some embodiments, the subject has previously been treated, and not responded to anti-viral therapy. In some embodiments, the nucleic acid molecule and/or the expressible nucleic acid sequence of the disclosure is administered to the subject by electroporation.

The vaccine may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns,” or other physical methods such as electroporation (“EP”), “hydrodynamic method,” or ultrasound.

The plasmid of the vaccine may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The antigen may be delivered via DNA injection and along with in vivo electroporation.

The vaccine or pharmaceutical composition can be administered by electroporation. Administration of the vaccine via electroporation of the plasmids of the vaccine may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including controller, current waveform generator, impedance tester, waveform logger, input element, status reporting element, communication port, memory component, power source, and power switch. The electroporation can be accomplished using an in vivo electroporation device, for example CELLECTRA® EP system (Inovio Pharmaceuticals, Inc., Blue Bell, Pa.) or Elgen electroporator (Inovio Pharmaceuticals, Inc.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers the same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present disclosure, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference in its entirety.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference in its entirety. The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes. The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in U.S. Pat. Nos. 5,273,525; 6,110,161; 6,261,281; 6,958,060; and 6,939,862. Furthermore, patents covering subject matter provided in U.S. Pat. No. 6,697,669, which concerns delivery of DNA using any of a variety of devices, and U.S. Pat. No. 7,328,064, drawn to a method of injecting DNA, are contemplated herein. The above patents are incorporated by reference in their entireties.

Methods of preparing the nucleic acid molecules are disclosed. In some embodiments, plasmids with one or more multiple cloning sites may be purchased from commercially available vendors and the expressible nucleic acids disclosed herein may be ligated into the plasmids after a digestion with a known restriction enzyme needed to cut the plasmid DNA. In another alternative embodiment, membrane-based purification methods disclosed herein offer reduced cost, high binding capacity, and high flow rates, resulting in a superior purification process. The purification process is further demonstrated to produce plasmid products substantially free of genomic DNA, RNA, protein, and endotoxin.

In some embodiments, all of the described aspects of the present disclosure are advantageously combined to provide an integrated process for preparing substantially purified cellular components of interest from cells in bioreactors. Again, the cells are most preferably plasmid-containing cells, and the cellular components of interest are most preferably plasmids. The substantially purified plasmids are suitable for various uses, including, but not limited to, gene therapy, plasmid-mediated therapy, as DNA vaccines for human, veterinary, or agricultural use, or for any other application that requires large quantities of purified plasmid. In this aspect, all of the advantages described for individual aspects of the present disclosure accrue to the complete, integrated process, providing a highly advantageous method that is rapid, scalable, and inexpensive. Enzymes and other animal-derived or biologically sourced products are avoided, as are carcinogenic, mutagenic, or otherwise toxic substances. Potentially flammable, explosive, or toxic organic solvents are similarly avoided.

One aspect of the present disclosure is an apparatus for isolating plasmid DNA from a suspension of cells having both plasmid DNA and genomic DNA. An embodiment of the apparatus comprises a first tank and second tank in fluid communication with a mixer. The first tank is used for holding the suspension cells and the second tank is used for holding a lysis solution. The suspension of cells from the first tank and the lysis solution from the second tank are both allowed to flow into the mixer forming a lysate mixture or lysate fluid. The mixer comprises a high shear, low residence-time mixing device with a residence time of equal to or less than about 1 second. In a preferred embodiment, the mixing device comprises a flow through, rotor/stator mixer or emulsifier having linear flow rates from about 0.1 L/min to about 20 L/min. The lysate-mixture flows from the mixer into a holding coil for a period of time sufficient to lyse the cells and forming a cell lysate suspension, wherein the lysate-mixture has resident time in the holding coil in a range of about 2-8 minutes with a continuous linear flow rate.

The cell lysate suspension is then allowed to flow into a bubble-mixer chamber for precipitation of cellular components from the plasmid DNA. In the bubble mixer chamber, the cell lysate suspension and a precipitation solution or a neutralization solution from a third tank are mixed together using gas bubbles, which forms a mixed gas suspension comprising a precipitate and an unclarified lysate or plasmid containing fluid. The precipitate of the mixed gas suspension is less dense than the plasmid containing fluid, which facilitates the separation of the precipitate from the plasmid containing fluid. The precipitate is removed from the mixed gas suspension to give a clarified lysate having the plasmid DNA, and the precipitate having cellular debris and genomic DNA.

In some embodiments, the bubble mixer-chamber comprises a closed vertical column with a top, a bottom, a first, and a second side with a vent proximal to the top of the column. A first inlet port of the bubble mixer-chamber is on the first side proximal to the bottom of the column and in fluid communication with the holding coil. A second inlet port of the bubble mixer-chamber is proximal to the bottom on a second side opposite of the first inlet port and in fluid communication with a third tank, wherein the third tank is used for holding a precipitation or a neutralization solution. A third inlet port of the bubble mixer-chamber is proximal to the bottom of the column and about in the middle of the first and second inlets and is in fluid communication with a gas source the third inlet entering the bubble-mixer-chamber. A preferred embodiment utilizes a sintered sparger inside the closed vertical column of the third inlet port. The outlet port exiting the bubble mixing chamber is proximal to the top of the closed vertical column. The outlet port is in fluid communication with a fourth tank, wherein the mixed gas suspension containing the plasmid DNA is allowed to flow from the bubble-mixer-chamber into the fourth tank. The fourth tank is used for separating the precipitate of the mixed gas suspension having a plasmid containing fluid, and can also include an impeller mixer sufficient to provide uniform mixing of fluid without disturbing the precipitate. A fifth tank is used for a holding the clarified lysate or clarified plasmid containing fluid. The clarified lysate is then filtered at least once. A first filter has a particle size limit of about 5-10 μm and the second filter has a cut of about 0.2 μm. Although gravity, pressure, vacuum, or a mixture thereof can be used for transporting: suspension of cells; lysis solutions; precipitation solutions; neutralization solutions; or mixed gas suspensions from any of the tanks to mixers, holding coils or different tanks, pumps are utilized in a preferred embodiments. In a more preferred embodiment, at least one pump having a linear flow rate from about 0.1 to about 1 ft/second is used.

In another specific embodiment, a Y-connector having a having a first bifurcated branch, a second bifurcated branch and an exit branch is used to contact the cell suspension and the lysis solutions before they enter the high shear, low residence-time mixing device. The first tank holding the cell suspension is in fluid communication with the first bifurcated branch of the Y-connector through the first pump and the second tank holding the lysis solution is in fluid communication with the second bifurcated branch of the Y-connector through the second pump. The high shear, low residence-time mixing device is in fluid communication with an exit branch of the Y-connector, wherein the first and second pumps provide a linear flow rate of about 0.1 to about 2 ft/second for a contacted fluid exiting the Y-connector.

Another specific aspect of the present disclosure is a method of substantially separating plasmid DNA and genomic DNA from a bacterial cell lysate. The method comprises: delivering a cell lysate into a chamber; delivering a precipitation fluid or a neutralization fluid into the chamber; mixing the cell lysate and the precipitation fluid or a neutralization fluid in the chamber with gas bubbles forming a gas mixed suspension, wherein the gas mixed suspension comprises the plasmid DNA in a fluid portion (i.e. an unclarified lysate) and the genomic DNA is in a precipitate that is less dense than the fluid portion; floating the precipitate on top of the fluid portion; removing the fluid portion from the precipitate forming a clarified lysate, whereby the plasmid DNA in the clarified lysate is substantially separated from genomic DNA in the precipitate. In some embodiments, the chamber is the bubble mixing chamber as described above; the lysing solution comprises an alkali, an acid, a detergent, an organic solvent, an enzyme, a chaotrope, or a denaturant; the precipitation fluid or the neutralization fluid comprises potassium acetate, ammonium acetate, or a mixture thereof; and the gas bubbles comprise compressed air or an inert gas. Additionally, the decanted-fluid portion containing the plasmid DNA is preferably further purified with one or more purification steps selected from a group consisting of: ion exchange, hydrophobic interaction, size exclusion, reverse phase purification, endotoxin depletion, affinity purification, adsorption to silica, glass, or polymeric materials, expanded bed chromatography, mixed mode chromatography, displacement chromatography, hydroxyapatite purification, selective precipitation, aqueous two-phase purification, DNA condensation, thiophilic purification, ion-pair purification, metal chelate purification, filtration through nitrocellulose, or ultrafiltration.

In some embodiments, a method for isolating a plasmid DNA from cells comprising: mixing a suspension of cells having the plasmid DNA and genomic DNA with a lysis solution in a high-shear-low-residence-time-mixing-device for a first period of time forming a cell lysate fluid; incubating the cell lysate fluid for a second period of time in a holding coil forming a cell lysate suspension; delivering the cell lysate suspension into a chamber; delivering a precipitation/neutralization fluid into the chamber; mixing the cell lysate suspension and the a precipitation/neutralization fluid in the chamber with gas bubbles forming a gas mixed suspension, wherein the gas mixed suspension comprises an unclarified lysate containing the plasmid DNA and a precipitate containing the genomic DNA, wherein the precipitate is less dense than the unclarified lysate; floating the precipitate on top of the unclarified lysate; removing the precipitate from the unclarified lysate forming a clarified lysate, whereby the plasmid DNA is substantially separated from genomic DNA; precipitating the plasmid DNA from the clarified lysate forming a precipitated plasmid DNA; and resuspending the precipitated plasmid DNA in an aqueous solution.

The disclosure also relates to a method of producing a polypeptide of interest in a mammalian cell, the method comprising contacting the cell with a composition comprising one or a plurality of the RNA molecules disclosed herein. In some embodiments, the therapeutic and/or prophylactic agent is an mRNA, and wherein the mRNA encodes the polypeptide of interest, whereby the mRNA is capable of being translated in the cell to produce the polypeptide of interest (e.g., nanoparticle or trimer of the disclosure). Compositions comprising RNA nucleic acid sequences of the disclosure can be delivered via lipid-containing nanoparticles and/or modification of the RNA nucleic acid sequence encoding the one or more viral polypeptides.

In some embodiments, the composition includes at least one RNA polynucleotide having an open reading frame encoding at least one SARS-CoV-2 antigenic polypeptide having at least one modification, at least one 5′ terminal cap, and is formulated within a lipid nanoparticle.

In some embodiments, a 5′ terminal cap is 7mG(5′)ppp(5′)NlmpNp. In some embodiments, at least one chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments, a lipid nanoparticle comprises a cationic lipid, a PEG-modified lipid, a sterol, and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino) butanoyl)oxy)heptadecanedioate (L319), (12Z,15Z)-N,N-dimethyl-2-nonylhenicosa-12,15-dien-1-amine (L608), and N,N-dimethyl-1-[(1S,2R)-2-octylcyclopropyl]heptadecan-8-amine (L530).

In some embodiments, SARS-CoV-2 RNA (e.g., mRNA) vaccines are formulated in a lipid nanoparticle. In some embodiments, SARS-CoV-2 RNA (e.g., mRNA) vaccines are formulated in a lipid-polycation complex, referred to as a cationic lipid nanoparticle. The formation of the lipid nanoparticle may be accomplished by methods known in the art and/or as described in U.S. Publication No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Publication No. WO2012013326 or U.S. Publication No. US20130142818; each of which is herein incorporated by reference in its entirety. In some embodiments, SARS-CoV-2 RNA (e.g., mRNA) vaccines are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components, and biophysical parameters such as size. In one example by Semple et al. (Nature Biotech. 2010 28:172-176; herein incorporated by reference in its entirety), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid was shown to more effectively deliver siRNA to various antigen presenting cells (Basha et al. Mol Ther. 2011 19:2186-2200; herein incorporated by reference in its entirety).

In some embodiments, lipid nanoparticle formulations may comprise about 35% to about 45% cationic lipid, about 40% to about 50% cationic lipid, about 50% to about 60% cationic lipid and/or about 55% to about 65% cationic lipid. In some embodiments, the ratio of lipid to RNA (e.g., mRNA) in lipid nanoparticles may be about 5:1 to about 20:1, about 10:1 to about 25:1, about 15:1 to about 30:1, and/or at least about 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain about 0.5% to about 3.0%, about 1.0% to about 3.5%, about 1.5% to about 4.0%, about 2.0% to about 4.5%, about 2.5% to about 5.0%, and/or about 3.0% to about 6.0% of the lipid molar ratio of PEG-c-DOMG (R-3-[(co-methoxy-poly(ethyleneglycol)2000) carb-amoyl)]-1,2-dimyristyloxypropyl-3-amine) (also referred to herein as PEG-DOMG) as compared to the cationic lipid, DSPC, and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200, and DLin-KC2-DMA.

In some embodiments, a SARS-CoV-2 RNA (e.g., mRNA) vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, (12Z,15Z)—N,N-dimethyl-2-nonylhenicosa-12,15-dien-1-amine (L608), N,N-dimethyl-1-[(1S,2R)-2-octylcyclopropyl]heptadecan-8-amine (L530), PEGylated lipids, and amino alcohol lipids.

In some embodiments, a lipid nanoparticle formulation includes about 25% to about 75% on a molar basis of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., about 35% to about 65%, about 45% to about 65%, about 60%, 57.5%, 50% or 40% on a molar basis.

In some embodiments, a lipid nanoparticle formulation includes about 0.5% to about 15% on a molar basis of the neutral lipid, e.g., about 3% to about 12%, about 5% to about 10% or about 15%, 10%, or 7.5% on a molar basis. Examples of neutral lipids include, without limitation, DSPC, POPC, DPPC, DOPE, and SM. In some embodiments, the formulation includes about 5% to about 50% on a molar basis of the sterol (e.g., about 15% to about 45%, about 20% to about 40%, about 40%, 38.5%, 35%, or 31% on a molar basis. A non-limiting example of a sterol is cholesterol. In some embodiments, a lipid nanoparticle formulation includes about 0.5% to about 20% on a molar basis of the PEG or PEG-modified lipid (e.g., about 0.5% to about 10%, about 0.5% to about 5%, about 1.5%, 0.5%, 1.5%, 3.5%, or 5% on a molar basis. In some embodiments, a PEG or PEG modified lipid comprises a PEG molecule of an average molecular weight of about 2,000 Da. In some embodiments, a PEG or PEG modified lipid comprises a PEG molecule of an average molecular weight of less than about 2,000, for example about 1,500 Da, about 1,000 Da, or about 500 Da. Non-limiting examples of PEG-modified lipids include PEG-distearoyl glycerol (PEG-DMG) (also referred herein as PEG-C14 or C14-PEG), and PEG-cDMA (further discussed in Reyes et al. J. Controlled Release, 107, 276-287 (2005) the content of which is herein incorporated by reference in its entirety).

In some embodiments, lipid nanoparticle formulations include about 25-75% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 0.5-15% of the neutral lipid, about 5-50% of the sterol, and about 0.5-20% of the PEG or PEG-modified lipid on a molar basis.

In some embodiments, lipid nanoparticle formulations include about 35-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 3-12% of the neutral lipid, about 15-45% of the sterol, and about 0.5-10% of the PEG or PEG-modified lipid on a molar basis.

In some embodiments, lipid nanoparticle formulations include about 45-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 5-10% of the neutral lipid, about 25-40% of the sterol, and about 0.5-10% of the PEG or PEG-modified lipid on a molar basis.

In some embodiments, lipid nanoparticle formulations include about 60% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

Some embodiments of the present disclosure provide a SARS-CoV-2 vaccine that includes at least one ribonucleic acid (RNA) polynucleotide having an open reading frame encoding at least one SARS-CoV-2 antigenic polypeptide, wherein at least about 80% of the uracil in the open reading frame have a chemical modification, optionally wherein the SARS-CoV-2 vaccine is formulated in a lipid nanoparticle. In some embodiments, the RNA vaccine pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel). In some embodiments, the RNA vaccines may be formulated in a lyophilized gel-phase liposomal composition as described in U.S. Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present disclosure may be made by the methods described in International Publication No. WO2013033438 or U.S. Publication No. US20130196948, the content of each of which is herein incorporated by reference in its entirety. As a non-limiting example, the phosphate conjugates may include a compound of any one of the formulas described in International Publication No. WO2013033438, herein incorporated by reference in its entirety. In particular, the present disclosure relates to a pharmaceutical composition comprising nanoparticles which comprise RNA encoding at least one antigen, wherein: (i) the number of positive charges in the nanoparticles does not exceed the number of negative charges in the nanoparticles and/or (ii) the nanoparticles have a neutral or net negative charge and/or (iii) the charge ratio of positive charges to negative charges in the nanoparticles is about 1.4:1 or less and/or (iv) the zeta potential of the nanoparticles is about 0 or less.

In some embodiments, the nanoparticles described herein are colloidally stable for at least about 2 hours in the sense that no aggregation, precipitation or increase of size and polydispersity index by more than about 30% as measured by dynamic light scattering takes place. In some embodiments, the charge ratio of positive charges to negative charges in the nanoparticles is between about 1.4:1 and about 1:8, preferably between about 1.2:1 and about 1:4, e.g. between about 1:1 and about 1:3 such as between about 1:1.2 and about 1:2, about 1:1.2 and about 1:1.8, about 1:1.3 and about 1:1.7, in particular between about 1:1.4 and about 1:1.6, such as about 1:1.5. In some embodiments, the zeta potential of the nanoparticles is about −5 or less, about −10 or less, about −15 or less, about −20 or less or about −25 or less. In various embodiments, the zeta potential of the nanoparticles is about −35 or higher, about −30 or higher or about −25 or higher. In some embodiments, the nanoparticles have a zeta potential from about 0 mV to about −50 mV, preferably about 0 mV to about −40 mV or about −10 mV to about −30 mV.

In some embodiments pharamceutical compositions of the disclosure comprise a nanoparticle or a liposome that encapsulates a DNA, RNA or DNA/RNA hydbrid comprising at least one expressible nucleic acid sequence. Liposomes are microscopic lipidic vesicles often having one or more bilayers of a vesicle-forming lipid, such as a phospholipid, and are capable of encapsulating a drug. Different types of liposomes may be employed in the context of the present disclosure, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation and the selection of the type of vesicles to be used will depend on the preferred mode of administration. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. The described phases may be present in the nanoparticulate RNA formulations of the present disclosure.

For formation of RNA lipoplexes from RNA and liposomes, any suitable method of forming liposomes can be used so long as it provides the envisaged RNA lipoplexes. Liposomes may be formed using standard methods such as the reverse evaporation method (REV), the ethanol injection method, the dehydration-rehydration method (DRV), sonication or other suitable methods.

After liposome formation, the liposomes can be sized to obtain a population of liposomes having a substantially homogeneous size range.

Bilayer-forming lipids have typically two hydrocarbon chains, particularly acyl chains, and a head group, either polar or nonpolar. Bilayer-forming lipids are either composed of naturally-occurring lipids or of synthetic origin, including the phospholipids, such as phosphatidylcholine, phosphatidylethanolamine, phosphatide acid, phosphatidylinositol, and sphingomyelin, where the two hydrocarbon chains are typically between about 14-22 carbon atoms in length, and have varying degrees of unsaturation. Other suitable lipids for use in the composition of the present disclosure include glycolipids and sterols such as cholesterol and its various analogs which can also be used in the liposomes.

Cationic lipids typically have a lipophilic moiety, such as a sterol, an acyl or diacyl chain, and have an overall net positive charge. The head group of the lipid typically carries the positive charge. The cationic lipid preferably has a positive charge of about 1 to about 10 valences, more preferably a positive charge of about 1 to about 3 valences, and more preferably a positive charge of about 1 valence. Examples of cationic lipids include, but are not limited to 1,2-di-O-octadecenyl-3-trimethylammonium propane (DOTMA); dimethyl-dioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. Most preferred is DOTMA.

In addition, the nanoparticles described herein preferably further include a neutral lipid in view of structural stability and the like. The neutral lipid can be appropriately selected in view of the delivery efficiency of the RNA-lipid complex. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, sterol, and cerebroside. Preferred is DOPE and/or DOPC. Most preferred is DOPE. In the case where a cationic liposome includes both a cationic lipid and a neutral lipid, the molar ratio of the cationic lipid to the neutral lipid can be appropriately determined in view of stability of the liposome and the like.

According to one embodiment, the nanoparticles described herein may comprise phospholipids. The phospholipids may be a glycerophospholipid. Examples of glycerophospholipid include, without being limited thereto, three types of lipids: (i) zwitterionic phospholipids, which include, for example, phosphatidylcholine (PC), egg yolk phosphatidylcholine, soybean-derived PC in natural, partially hydrogenated or fully hydrogenated form, dimyristoyl phosphatidylcholine (DMPC) sphingomyelin (SM); (ii) negatively charged phospholipids: which include, for example, phosphatidylserine (PS), phosphatidylinositol (PI), phosphatidic acid (PA), phosphatidylglycerol (PG) dipalmipoyl PG, dimyristoyl phosphatidylglycerol (DMPG); synthetic derivatives in which the conjugate renders a zwitterionic phospholipid negatively charged such is the case of methoxypolyethylene, glycol-distearoyl phosphatidylethanolamine (mPEG-DSPE); and (iii) cationic phospholipids, which include, for example, phosphatidylcholine or sphingomyelin of which the phosphomonoester was O-methylated to form the cationic lipids.

Association of RNA to the lipid carrier can occur, for example, by the RNA filling interstitial spaces of the carrier, such that the carrier physically entraps the RNA, or by covalent, ionic, or hydrogen bonding, or by means of adsorption by non-specific bonds. Whatever the mode of association, the RNA must retain its therapeutic, i.e. antigen-encoding, properties.

In some embodiments, the nanoparticles comprise at least one lipid. In some embodiments, the nanoparticles comprise at least one cationic lipid. The cationic lipid can be monocationic or polycationic. Any cationic amphiphilic molecule, eg, a molecule which comprises at least one hydrophilic and lipophilic moiety is a cationic lipid within the meaning of the present disclosure. In some embodiments, the positive charges are contributed by the at least one cationic lipid and the negative charges are contributed by the RNA. In some embodiments, the nanoparticles comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In some embodiments, the cationic lipid and/or the helper lipid is a bilayer forming lipid.

In some embodiments, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof. In some embodiments, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof. In some embodiments, the molar ratio of the at least one cationic lipid to the at least one helper lipid is from about 10:0 to about 3:7, preferably about 9:1 to about 3:7, about 4:1 to about 1:2, about 4:1 to about 2:3, about 7:3 to about 1:1, or about 2:1 to about 1:1, preferably about 1:1. In some embodiments, in this ratio, the molar amount of the cationic lipid results from the molar amount of the cationic lipid multiplied by the number of positive charges in the cationic lipid. In various embodiments, the lipids are not functionalized such as functionalized by mannose, histidine and/or imidazole, the nanoparticles do not comprise a targeting ligand such as mannose functionalized lipids and/or the nanoparticles do not comprise one or more of the following: pH dependent compounds, cationic polymers such as polymers containing histidine and/or polylysine, wherein the polymers may optionally be PEGylated and/or histidylated, or divalent ions such as Ca 2+.

In various embodiments, the RNA nanoparticles may comprise peptides, preferentially with a molecular weight of up to about 2500 Da.

In the nanoparticles described herein the lipid may form a complex with and/or may encapsulate the RNA. In some embodiments, the nanoparticles comprise a lipoplex or liposome. In some embodiments, the lipid is comprised in a vesicle encapsulating said RNA. The vesicle may be a multilamellar vesicle, an unilamellar vesicle, or a mixture thereof. The vesicle may be a liposome. In some embodiments, the nanoparticles are lipoplexes comprising DOTMA and DOPE in a molar ratio of about 10:0 to about 1:9, preferably about 8:2 to about 3:7, and more preferably of about 7:3 to about 5:5 and wherein the charge ratio of positive charges in DOTMA to negative charges in the RNA is about 1.8:2 to about 0.8:2, more preferably about 1.6:2 to about 1:2, even more preferably about 1.4:2 to about 1.1:2 and even more preferably about 1.2:2.

In some embodiments, the nanoparticles are lipoplexes comprising DOTMA and Cholesterol in a molar ratio of about 10:0 to about 1:9, preferably about 8:2 to about 3:7, and more preferably of about 7:3 to about 5:5 and wherein the charge ratio of positive charges in DOTMA to negative charges in the RNA is about 1.8:2 to about 0.8:2, more preferably about 1.6:2 to about 1:2, even more preferably about 1.4:2 to about 1.1:2 and even more preferably about 1.2:2. In some embodiments, the nanoparticles are lipoplexes comprising DOTAP and DOPE in a molar ratio of about 10:0 to about 1:9, preferably about 8:2 to about 3:7, and more preferably of about 7:3 to about 5:5 and wherein the charge ratio of positive charges in DOTMA to negative charges in the RNA is about 1.8:2 to about 0.8:2, more preferably about 1.6:2 to about 1:2, even more preferably about 1.4:2 to about 1.1:2 and even more preferably about 1.2:2. In some embodiments, the nanoparticles are lipoplexes comprising DOTMA and DOPE in a molar ratio of about 2:1 to about 1:2, preferably about 2:1 to about 1:1, and wherein the charge ratio of positive charges in DOTMA to negative charges in the RNA is about 1.4:1 or less. In some embodiments, the nanoparticles are lipoplexes comprising DOTMA and cholesterol in a molar ratio of about 2:1 to about 1:2, preferably about 2:1 to about 1:1, and wherein the charge ratio of positive charges in DOTMA to negative charges in the RNA is about 1.4:1 or less. In some embodiments, the nanoparticles are lipoplexes comprising DOTAP and DOPE in a molar ratio of about 2:1 to about 1:2, preferably about 2:1 to about 1:1, and wherein the charge ratio of positive charges in DOTAP to negative charges in the RNA is about 1.4:1 or less. In some embodiments, the nanoparticles have an avarage diameter in the range of from about 50 nm to about 1000 nm, preferably from about 50 nm to about 400 nm, preferably about 100 nm to about 300 nm such as about 150 nm to about 200 nm. In some embodiments, the nanoparticles have a diameter in the range of about 200 to about 700 nm, about 200 to about 600 nm, preferably about 250 to about 550 nm, in particular about 300 to about 500 nm or about 200 to about 400 nm.

In some embodiments, the polydispersity index of the nanoparticles described herein as measured by dynamic light scattering is about 0.5 or less, preferably about 0.4 or less or even more preferably about 0.3 or less. In some embodiments, the nanoparticles described herein are obtainable by one or more of the following: (i) incubation of liposomes in an aqueous phase with the RNA in an aqueous phase, (ii) incubation of the lipid dissolved in an organic, water miscible solvent, such as ethanol, with the RNA in aqueous solution, (iii) reverse phase evaporation technique, (iv) freezing and thawing of the product, (v) dehydration and rehydration of the product, (vi) lyophilization and rehydration of the of the product, or (vii) spray drying and rehydration of the product.

The nanoparticle formulation may comprise a polymer conjugate. The polymer conjugate may be a water-soluble conjugate. The polymer conjugate may have a structure as described in U.S. Publication No. 20130059360, the content of which is herein incorporated by reference in its entirety. In some aspects, polymer conjugates with the polynucleotides of the present disclosure may be made using the methods and/or segmented polymeric reagents described in U.S. Publication No. 20130072709, herein incorporated by reference in its entirety. In other aspects, the polymer conjugate may have pendant side groups comprising ring moieties such as, but not limited to, the polymer conjugates described in U.S. Publication No. US20130196948, the contents of which is herein incorporated by reference in its entirety.

The nanoparticle formulations may comprise a conjugate to enhance the delivery of nanoparticles of the present disclosure in a subject. Further, the conjugate may inhibit phagocytic clearance of the nanoparticles in a subject. In some aspects, the conjugate may be a “self” peptide designed from the human membrane protein CD47 (e.g., the “self” particles described by Rodriguez et al. (Science 2013, 339, 971-975), herein incorporated by reference in its entirety). As shown by Rodriguez et al., the self peptides delayed macrophage-mediated clearance of nanoparticles which enhanced delivery of the nanoparticles. In other aspects, the conjugate may be the membrane protein CD47 (e.g., see Rodriguez et al. Science 2013, 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, about 100% of the uracil in the open reading frame have a chemical modification. In some embodiments, a chemical modification is in the 5-position of the uracil. In some embodiments, a chemical modification is a N₁-methyl pseudouridine. In some embodiments, about 100% of the uracil in the open reading frame have a N₁-methyl pseudouridine in the 5-position of the uracil.

In some embodiments, efficacy of RNA vaccines RNA (e.g., mRNA) can be significantly enhanced when combined with a flagellin adjuvant, in particular, when one or more antigen-encoding mRNAs is combined with an mRNA encoding flagellin.

RNA (e.g., mRNA) vaccines combined with the flagellin adjuvant (e.g., mRNA-encoded flagellin adjuvant) have superior properties in that they may produce much larger antibody titers and produce responses earlier than commercially available vaccine formulations. While not wishing to be bound by theory, it is believed that the RNA vaccines, for example, as mRNA polynucleotides, are better designed to produce the appropriate protein conformation upon translation, for both the antigen and the adjuvant, as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Some embodiments of the present disclosure provide RNA (e.g., mRNA) vaccines that include at least one RNA (e.g., mRNA) polynucleotide having an open reading frame encoding at least one antigenic polypeptide or an immunogenic fragment thereof (e.g., an immunogenic fragment capable of inducing an immune response to the antigenic polypeptide) and at least one RNA (e.g., mRNA polynucleotide) having an open reading frame encoding a flagellin adjuvant.

In some embodiments, at least one flagellin polypeptide (e.g., encoded flagellin polypeptide) is a flagellin protein. In some embodiments, at least one flagellin polypeptide (e.g., encoded flagellin polypeptide) is an immunogenic flagellin fragment. In some embodiments, at least one flagellin polypeptide and at least one antigenic polypeptide are encoded by a single RNA (e.g., mRNA) polynucleotide. In other embodiments, at least one flagellin polypeptide and at least one antigenic polypeptide are each encoded by a different RNA polynucleotide.

Some embodiments of the present disclosure provide methods of inducing an antigen specific immune response in a subject, comprising administering to the subject a SARS-CoV-2 vaccine in an amount effective to produce an antigen specific immune response.

In some aspects, vaccines of the disclosure (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as about 1:40, 1:100, etc.

In exemplary embodiments of the disclosure, an efficacious vaccine produces an antibody titer of greater than about 1:40, greater that about 1:100, greater than about 1:400, greater than about 1:1000, greater than about 1:2000, greater than about 1:3000, greater than about 1:4000, greater than about 1:500, greater than about 1:6000, greater than about 1:7500, greater than about 1:10000. In exemplary embodiments, the antibody titer is produced or reached by about 10 days following vaccination, by about 20 days following vaccination, by about 30 days following vaccination, by about 40 days following vaccination, or by about 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In exemplary aspects of the disclosure, antigen-specific antibodies are measured in units of g/ml or are measured in units of IU/L (International Units per liter) or mIU/ml (milli International Units per ml). In exemplary embodiments of the disclosure, an efficacious vaccine produces more than about 0.5 μg/ml, 0.1 μg/ml, 0.2 μg/ml, 0.35 μg/ml, 0.5 μg/ml, 1 μg/ml, 2 μg/ml, 5 μg/ml or 10 μg/ml of antigen-specific antibodies. In exemplary embodiments of the disclosure, an efficacious vaccine produces more than about 10 mIU/ml, 20 mIU/ml, 50 mIU/ml, 100 mIU/ml, 200 mIU/ml, 500 mIU/ml or 1000 mIU/ml of antigen-specific antibodies. In exemplary embodiments, the antibody level or concentration is produced or reached by about 10 days following vaccination, by about 20 days following vaccination, by about 30 days following vaccination, by about 40 days following vaccination, or by about 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose). In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

In some embodiments, the SARS-CoV-2 vaccine includes at least one RNA polynucleotide having an open reading frame encoding at least one SARS-CoV-2 antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

When transfected into mammalian cells, the modified mRNAs have a stability of from about 12 to about 18 hours or more than about 18 hours, e.g., about 24, 36, 48, 60, 72, or greater than about 72 hours.

In some embodiments, a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 1-ethyl-pseudouridine (e1ψ), 5-methoxyuridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethyl aminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQ0base, preQ1base, and combinations of two or more thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polyribonucleotide (e.g., RNA polyribonucleotide, such as mRNA polyribonucleotide) includes a combination of at least two (e.g., 2, 3, 4 or more) of the aforementioned modified nucleobases. In some embodiments, polynucleotides (e.g., RNA polynucleotides, such as mRNA polynucleotides) include a combination of at least two (e.g., 2, 3, 4 or more) of the aforementioned modified nucleobases.

The expressible nucleic acid sequence of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may be any one of nucleotides A, G, U, C, or any one of the combinations A+G, A+U, A+C, G+U, G+C, U+C, A+G+U, A+G+C, G+U+C, or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, and from about 95% to about 100%). It will be understood that any remaining percentage is accounted for by the presence of unmodified A, G, U, or C.

The nucleic acid sequences may contain at a minimum about 1% and at maximum about 100% modified nucleotides, or any intervening percentage, such as at least about 5% modified nucleotides, at least about 10% modified nucleotides, at least about 25% modified nucleotides, at least about 50% modified nucleotides, at least about 80% modified nucleotides, or at least about 90% modified nucleotides. For example, the polynucleotides may contain a modified pyrimidine such as a modified uracil or cytosine. In some embodiments, at least about 5%, at least about 10%, at least about 25%, at least about 50%, at least about 80%, at least about 90% or about 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., about 2, 3, 4, or more unique structures). In some embodiments, at least about 5%, at least about 10%, at least about 25%, at least about 50%, at least about 80%, at least about 90%, or about 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., about 2, 3, 4, or more unique structures).

Thus, in some embodiments, the RNA vaccines and/or RNA nucleic acid seqeunces comprise a 5′UTR element, an optionally codon optimized open reading frame, and a 3′UTR element, a poly(A) sequence and/or a polyadenylation signal wherein the RNA is not chemically modified.

Viral vaccines of the present disclosure comprise at least one RNA polynucleotide, such as a mRNA (e.g., modified mRNA). mRNA, for example, is transcribed in vitro from template DNA, referred to as an “in vitro transcription template.” In some embodiments, the at least one RNA polynucleotide has at least one chemical modification. The at least one chemical modification may include, but is expressly not limited to, any modification described herein.

In vitro transcription of RNA is known in the art and is described in WO/2014/152027, which is incorporated by reference herein in its entirety. For example, in some embodiments, the RNA transcript is generated using a non-amplified, linearized DNA template in an in vitro transcription reaction to generate the RNA transcript. In some embodiments, the RNA transcript is capped via enzymatic capping. In some embodiments, the RNA transcript is purified via chromatographic methods, e.g., use of an oligo dT substrate. Some embodiments exclude the use of DNase. In some embodiments, the RNA transcript is synthesized from a non-amplified, linear DNA template coding for the gene of interest via an enzymatic in vitro transcription reaction utilizing a T7 phage RNA polymerase and nucleotide triphosphates of the desired chemistry. Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNa polymerase, and/or mutant polymerases such as, but not limited to, polymerases able to incorporate modified nucleic acids and/or modified nucleotides, including chemically modified nucleic acids and/or nucleotides.

In some embodiments, a non-amplified, linearized plasmid DNA is utilized as the template DNA for in vitro transcription. In some embodiments, the template DNA is isolated DNA. In some embodiments, the template DNA is cDNA. In some embodiments, the cDNA is formed by reverse transcription of a RNA polynucleotide, for example, but not limited to SARS-CoV-2 RNA, e.g. SARS-CoV-2 mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

### F. Vaccines

Disclosed are DNA vaccines comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 68, SEQ ID NO: 71, SEQ ID NO: 74, SEQ ID NO: 77, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155 or SEQ ID NO: 158, or a functional fragment or variant thereof. Also disclosed are RNA vaccines comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, SEQ ID NO: 156 or SEQ ID NO: 159, or a functional fragment or variant thereof. In some embodiment, the DNA or RNA vaccine disclosed herein encodes a polypeptide comprising at least about 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

The vaccines of the disclosure can be formulated using one or more excipients to: increase stability; increase cell transfection; permit the sustained or delayed release (e.g., from a depot formulation); alter the biodistribution (e.g., target to specific tissues or cell types); increase the translation of encoded protein in vivo; and/or alter the release profile of encoded protein (antigen) in vivo. In some embodiments therefore, the disclosed vaccine further comprises a pharmaceutically acceptable excipient. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, hyaluronidase, nanoparticle mimics and combinations thereof. In some embodiments, the pharmaceutically acceptable excipient is an adjuvant. In some embodiments, the vaccines of the disclosure do not include an adjuvant (they are adjuvant free).

In some embodiments, the vaccines of the disclosure are formulated in a nanoparticle as described herein elsewhere. In some embodiments, the vaccines of the disclosure are formulated in a lipid nanoparticle as described herein elsewhere. In some embodiments, the vaccines of the disclosure are formulated in a lipid-polycation complex, referred to as a cationic lipid nanoparticle, as described herein elsewhere. As a non-limiting example, the polycation may include a cationic peptide or a polypeptide such as, but not limited to, polylysine, polyornithine and/or polyarginine. In some embodiments, the vaccines of the disclosure are formulated in a lipid nanoparticle that includes a non-cationic lipid such as, but not limited to, cholesterol or dioleoyl phosphatidylethanolamine (DOPE) as described herein elsewhere.

In some embodiments, the vaccines of the disclosure are administered prophylactically as part of an active immunization scheme to healthy individuals. In some embodiments, the vaccines of the disclosure are administered therapeutically early in infection during the incubation phase. In some embodiments, the vaccines of the disclosure are administered therapeutically during active infection after onset of symptoms. In some embodiments, the amount of DNA or RNA (e.g., mRNA) vaccine of the present disclosure provided to a cell, a tissue or a subject may be an amount effective for immune prophylaxis. In some embodiments, the vaccines of the disclosure are administrated with other prophylactic or therapeutic compounds. In some embodiments, the prophylactic or therapeutic compound is an adjuvant. In some embodiments, the prophylactic or therapeutic compound is a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, about 1, 2, 3, 4, 5, 6, 7, 8, 9, 10, 15, 20, 25, 30, 35, 40, 45, 50 or 55 minutes, or about 1, 2, 3, 4, 5, 6, 7, 8, 9, 10, 11, 12, 13, 14, 15, 16, 17, 18, 19, 20, 21, 22 or 23 hours, or about 1, 2, 3, 4, 5, 6, 7, 8, 9, 10, 15, 20, 25 or 30 days, or about 1, 2, 3, 4, 5, or 6 weeks, or about 1, 2, 3, 4, 5, 6, 7, 8, 9, 10, 11 or 12 months, or about 1, 2, 3, 4, 5, 6, 7, 8, 9, 10, 15, 20, 25, 30, 35, 40, 45 or 50 years. In some embodiments, the time of administration between the initial administration of the prophylactic composition and the booster is about 1 week. In some embodiments, the time of administration between the initial administration of the prophylactic composition and the booster is about 2 weeks. In some embodiments, the time of administration between the initial administration of the prophylactic composition and the booster is about 3 weeks. In some embodiments, the time of administration between the initial administration of the prophylactic composition and the booster is about 1 month. In some embodiments, the time of administration between the initial administration of the prophylactic composition and the booster is about 2 months. In some embodiments, the time of administration between the initial administration of the prophylactic composition and the booster is about 3 months. In some embodiments, the time of administration between the initial administration of the prophylactic composition and the booster is about 6 months. In some embodiments, the time of administration between the initial administration of the prophylactic composition and the booster is about 1 year.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any of the elements of the disclosed nucleic acid compositions. For example, disclosed are kits comprising nucleic acid sequences comprising a leader sequence, a linker sequence, a nucleic acid sequence encoding a self-assembling polypeptide, and/or a nucleic acid sequence encoding a viral antigen. In some embodiments, the kits can further comprise a plasmid backbone.

Other embodiments are described in the following non-limiting Examples. Various publications, including patents, published applications, technical articles and scholarly articles are cited throughout the specification. Each of these cited publications is incorporated by reference herein in its entirety.

### EXAMPLES

### Example 1. Experimental Validation of Prefusion CoV2 Trimers

The SARS-CoV-2 prefusion trimers would be tested by size-exclusion chromatography, multi-angle light scattering, ELISA binding to various receptors and anti-CoV2 antibodies, negative-stain EM to determine relative populations of prefusion states, SPR binding to various receptors and anti-CoV2 antibodies, native page western blots and thermal stability in buffer and serum using DSF. Collectively, these measurements will enable us to determine if the SARS-CoV-2 trimers have the relevant design characteristics. The trimers must demonstrate a homogenous, trimeric oligomerization state as determined by SEC-MALS, western blot and negative stain EM. The trimers must also have alterations in frequencies of relevant prefusion conformations as determined by negative stain EM. The trimers must bind to receptors (such as ACE2) or selected anti-CoV2 antibodies as determined by ELISA and SPR. The trimers must have an increase in thermal stability in PBS and serum as determined by DSF. Other equivalent biophysical methods to determine these parameters may be alternatively employed.

### Size-Exclusion Chromatography and Multi-Angle Light Scattering

Expi293F cells will be transfected with a plasmid vector carrying the trimer transgene with PEI/OPTI-MEM and harvested 6 days post-transfection. Transfection supernatant will be first purified with either affinity chromatography using the AKTA pure 25 system and an IMAC Nickel column OR gravity flow columns filled with GNL Lectin beads. The eluate fractions from the affinity purification will be pooled, concentrated and dialyzed into 1×PBS buffer before being loaded onto the SEC column and then purified with size exclusion chromatography, for which the Superdex 200 Increase 10/300 GL column or Superose 6 Increase 10/300 GL column or equivalent will be run at 0.5 mL/min. Identified eluate fractions will then collected and concentrated to 1 mg/mL in PBS. The oligomeric state of the trimers will then be confirmed by size exclusion chromatography-multi-angle light scattering (SECMALS) using the DAWN HELEOS II multi-angle light scattering system with Optilab T-rEX refractometer (Wyatt Technology).

### ELISA

The trimers will be assessed by ELISAs to determine binding against human ACE2 with a detectable tag. Nunc ELISA plates will be coated with 1 μg/ml rabbit anti-His6X in 1× PBS for 4-6 hours at room temperature and washed 4 times with washing buffer (1× PBS and 0.05% Tween 20). Plates will be blocked overnight at 4° C. with blocking buffer (1× PBS, 0.05% Tween 20, 5% evaporated milk and 1% FBS). Plates will be washed four times with washing buffer then incubated with full length (S1+S2) spike protein containing a C-terminal His tag (Sino Biologics, cat. 40589-V08B1) at 10 ug/ml for 1 hour at room temperature. Plates will be washed and then serial dilutions of recombinant human ACE2 with a human Fc tag (ACE2-IgHu) and incubated for 1-2 hours at room temperature. Plates will be again washed and then incubated with 1:10,000 dilution of horseradish peroxidase (HRP) conjugated anti-human IgG secondary antibody (Bethyl, cat. A80-304P) and incubated for 1 hour at room temperature. After final wash plates will be developed using 1-Step Ultra TMB-ELISA Substrate (ThermoFisher, cat. 34029) and the reaction stopped with 1 M Sulfuric Acid. Plates will be read at 450 nm wavelength within 30 minutes using a SpectraMax Plus 384 Microplate Reader (Molecular Devices, Sunnyvale, Calif.).

### Negative Stain EM of Purified SARS-CoV-2 Trimers

The trimers will be produced in Expi293 cells, purified using Agarose bound lectin beads (Agarose Galanthus Nivalis Lectin, Vector Laboratories) followed by size exclusion chromatography (GE Healthcare) using the Superose 6 Increase 10/300 GL column. The proteins will be further dialyzed into Tris-buffered saline (TBS). A total of 3 μL of purified proteins was adsorbed onto glow discharged carbon-coated Cu400 EM grids. The grids will be then stained with 3 μL of 2% uranyl acetate, blotted, and stained again with 3 μL of the stain followed by a final blot. Image collection and data processing will be performed on a FEI Tecnai T12 microscope equipped with a Oneview Gatan camera at 90,450× magnification at the camera and a pixel size of 1.66 Å.

### Surface Plasmon Resonance (SPR)

Kinetics and affinities of antibody-antigen interactions will be measured on a Biacore 8K (GE/Cytiva) using CAP (GE/Cytiva) or Protein A (GE/Cytiva) chips and 1× HBS-EP+ pH 7.4 running buffer (20× stock from Teknova, Cat. No H8022) supplemented with BSA at 1 mg/ml. Typically, 100-200 RUs of antigen or antibody will be captured onto each flow cell and analytes will be passed over the flow cell at 50 μL/min for 3 min followed by a 5 min dissociation time. Regeneration is accomplished using 6M Guanidine-HCL in 02.5M NaOH or 10 mM glycine-HCl at pH 1.5 with 180 seconds contact time. Raw sensograms will be analyzed using Biacore evaluation software (GE/Cytiva), including flow cell and blank double referencing, and either Equilibrium fits or Kinetic fits with Langmuir model, or both, will be employed when applicable. Analyte concentrations will be measured on a NanoDrop 2000c Spectrophotometer using Absorption signal at 280 nm.

### Differential Scanning Fluorimetry

The assay will employ a real-time PCR instrument to monitor changes in fluorescence of SYPRO Orange (or similar dye) and to determine melting temperatures. At least three temperature gradient scans will be run from room temperature to 120C at rate of 1-2C/min. Signals are normalized from 0 to 100. The SYPRO orange dye and the trimers added at appropriate concentration (2×-10× range and 100-1000 ug/ml, respectively) to optimize the experimental conditions. SYPRO orange has an excitation maximum near 500 nm and emission maximum near 600 nm. The real time system channel will be selected to optimize excitation and emission of SPYRO orange.

### Western Blot

Proteins will be separated on a 4-12% BIS-TRIS gel (ThermoFisher Scientific), then following transfer, blots will be incubated with ACE2 or an anti-SARS-CoV spike protein polyclonal antibody (Novus Biologicals) then visualized with horseradish peroxidase (HRP)-conjugated anti-mouse IgG (GE Amersham).

### Immunofluorescence of Transfected 293T Cells

For in vitro staining of trimer protein expression 293T cells will be cultured on 4-well glass slides (Lab-Tek) and transfected with 3 μg per well of pDNA using TurboFectin8.0 (OriGene) transfection reagent following the manufacturer's protocol. Cells will be fixed 48 hrs after transfection with 10% Neutral-buffered Formalin (BBC Biochemical, Wash. State) for 10 min at room temperature (RT) and then washed with PBS. Before staining, chamber slides will be blocked with 0.3% (v/v) Triton-X (Sigma), 2% (v/v) donkey serum in PBS for 1 hr at RT. Cells will be stained with a rabbit anti-SARS-CoV spike protein polyclonal antibody (Novus Biologicals) diluted in 1% (w/v) BSA (Sigma), 2% (v/v) donkey serum, 0.3% (v/v) Triton-X (Sigma) and 0.025% (v/v) 1 g ml⁻¹ Sodium Azide (Sigma) in PBS for 2 hrs at RT. Slides will be washed three times for 5 min in PBS and then stained with donkey anti-rabbit IgG AF488 (lifetechnologies) for 1 hr at RT. Slides will be washed again and mounted and covered with DAPI-Fluoromount (SouthernBiotech).

### Example 2. Vaccine Delivery Systems

### Recombinant Protein Vaccines

The SARS-CoV-2 Spike trimers described herein are amenable to delivery as a recombinant subunit vaccine. The trimers can be encoded into mammalian expression plasmids, such as pHLSEC. These plasmids can be employed to expressed and purified recombinant vaccines as described in this document. The recombinant proteins can be easily formulated with any number of adjuvant systems (such as aluminum phosphate) to enhance immunity.

### DNA Plasmid Vaccines

The SARS-CoV-2 Spike trimers described herein are amenable to delivery as a transgene in a DNA plasmid (such as Inovio Pharmaceutical's synDNA platform). DNA vaccines are non-infectious and have a long record of safety in humans. DNA vaccines can induce high seroconversion rates in people. DNA vaccines can induce both B and T cell immunities. DNA plasmids can be formulated with adjuvating material to improve take-up, immune trafficking and resulting immune responses.

### RNA Vaccines

The SARS-CoV-2 Spike trimers described herein are amenable to delivery as a transgene in an RNA vaccine (such as Moderna's mRNA platform). RNA vaccines are non-infectious, non-integrating and are naturally degrading. RNA vaccines have been shown to stimulate strong B cell responses to encoded transgenes. Naked RNA can be formulated with lipid nanoparticles (LNPs) to encapsulate RNA protecting the transgene from degradation.

### Self-Replicating RNA Vaccines

The SARS-CoV-2 Spike trimers described herein are amenable to delivery as a transgene in a self-replicating RNA vaccine formulated as DNA. RNA replicons can be delivered by a DNA or mRNA to self-amplify resulting in higher transgene expression.

### Adeno-Associated Virus Vaccines:

Modified versions of the SARS-CoV-2 Spike trimers described herein (such as ones which are capable of cell fusion by removal of key mutations impacting this function) are amenable to delivery as a transgene in AAV (or similar vector). AAV can be employed to obtain high expression of vaccine antigens in vivo. Numerous AAV variants exist to help target specific cell types and each can be explored with a SARS-CoV-2 Spike trimer.

### REFERENCES

[1] L. C. Lambert, A. S. Fauci, N Engl J Med 2010, 363, 2036.
[2] Y. Gao, P. F. McKay, J. F. S. Mann, Viruses 2018, 10.
[3] S. Plotkin, Proc Natl Acad Sci USA 2014, 111, 12283.
[4] C. L. Trimble, M. P. Morrow, K. A. Kraynyak, et al., Lancet 2015, 386, 2078.
[5] A. E. Gregory, R. Titball, D. Williamson, Front Cell Infect Microbiol 2013, 3, 13.
[6] L. Zhao, A. Seth, N. Wibowo, et al., Vaccine 2014, 32, 327.
[7] S. Al-Halifa, L. Gauthier, D. Arpin, et al., Front Immunol 2019, 10, 22.
[8] W. Szmuness, C. E. Stevens, E. J. Harley, et al., N Engl J Med 1980, 303, 833.
[9] D. M. Harper, L. R. DeMars, Gynecol Oncol 2017, 146, 196.
[10] J. W. Stone, N. J. Thornburg, D. L. Blum, et al., Nanotechnology 2013, 24, 295102.
[11] K. Cheng, K. El-Boubbou, C. C. Landry, ACS Appl Mater Interfaces 2012, 4, 235.
[12] A. K. Giddam, M. Zaman, et al., Nanomedicine (Lond) 2012, 7, 1877.
[13] J. Fuenmayor, F. Godia, L. Cervera, N Biotechnol 2017, 39, 174.
[14] S. A. Kaba, C. P. Karch, L. Seth, K. M. B. Ferlez, et al., Vaccine 2018, 36, 906.
[15] Z. Xu, D. W. Kulp, Curr Opin Immunol 2019, 59, 49.
[16] J. Marcandalli, B. Fiala, S. Ols, M. Perotti, et al., Cell 2019, 176, 1420.
[17] K. Sliepen, G. Ozorowski, J. A. Burger, et al., Retrovirology 2015, 12, 82.
[18] S. Chattopadhyay, J. Y. Chen, H. W. Chen, C. J. Hu, Nanotheranostics 2017, 1, 244.
[19] N. Desai, AAPS J 2012, 14, 282.
[20] J. Feng, et al., Journal of Translational Medicine 2019, 17, 200.
[21] L. H. Lua, N. K. Connors, F. Sainsbury, et al., Biotechnol Bioeng 2014, 111, 425.
[22] A. Urakami, M. M. Ngwe Tim, M. L. Moi, et al., J Virol 2017, 91.
[23] Z. Jiang, G. Tong, B. Cai, Y. Xu, J. Lou, Protein Expr Purif 2011, 80, 203.
[24] A. C. Eifler, C. S. Thaxton, Methods Mol Biol 2011, 726, 325.
[25] B. S. Graham, M. S. A. Gilman, J. S. McLellan, Annu Rev Med 2019, 70, 91.
[26] T. Tokatlian, B. J. Read, C. A. Jones, et al., Science 2019, 363, 649.
[27] J. G. Jardine, T. Ota, D. Sok, M. Pauthner, et al., Science 2015, 349, 156.
[28] D. Sok, B. Briney, J. G. Jardine, D. W. Kulp, et al., Science 2016, 353, 1557.
[29] J. G. Jardine, D. W. Kulp, C. Havenar-Daughton, et al., Science 2016, 351, 1458.
[30] K. A. Dowd, S. Y. Ko, K. M. Morabito, et al., Science 2016, 354, 237.
[31] B. S. Davis, G. J. Chang, B. Cropp, J. T. Roehrig, et al., J Virol 2001, 75, 4040.
[32] B. Wang, K. E. Ugen, et al., Proc Natl Acad Sci USA 1993, 90, 4156.
[33] N. Y. Sardesai, D. B. Weiner, Curr Opin Immunol 2011, 23, 421.
[34] Z. Xu, M. C. Wise, H. Choi, A. Perales-Puchalt, et al., EBioMedicine 2018, 35, 97.
[35] M. Khoshnejad, A. Patel, K. Wojtak, et al., Mol Ther 2019, 27, 188.
[36] M. C. Wise, Z. Xu, E. Tello-Ruiz, C. Beck, et al., J Clin Invest 2019.
[37] P. Tebas, C. C. Roberts, K. Muthumani, E. L. Reuschel, et al., N Engl J Med 2017.
[38] P. Tebas, K. A. Kraynyak, A. Patel, J. N. Maslow, et al., J Infect Dis 2019.
[39] S. A. Kalams, S. D. Parker, M. Elizaga, B. Metch, et al., J Infect Dis 2013, 208, 818.
[40] K. Modjarrad, C. C. Roberts, K. T. Mills, et al., Lancet Infect Dis 2019.
[41] F. Brod, K. Miura, I. Taylor, Y. Li, A. Marini, et al., Front Immunol 2018, 9, 2780.
[42] K. Tomusange, D. Wijesundara, J. Gummow, et al., Sci Rep 2016, 6, 29131.
[43] P. Abbink, R. A. Larocca, R. A. De La Barrera, C. A. Bricault, E. T. Moseley, M. Boyd, M. Kirilova, Z. Li, D. Ng'ang'a, O. Nanayakkara, et al., Science 2016, 353, 1129.
[44] C. Bohannon, R. Powers, L. Satyabhama, et al., Nat Commun 2016, 7, 11826.
[45] B. Selander, U. Martensson, A. Weintraub, et al., J Clin Invest 2006, 116, 1425.
[46] A. Benoit, J. Beran, J. M. Devaster, et al., Open Forum Infect Dis 2015, 2, ofv067.
[47] M. Kanekiyo, M. G. Joyce, R. A. Gillespie, et al., Nat Immunol 2019, 20, 362.
[48] M. A. Kutzler, D. B. Weiner, Nat Rev Genet 2008, 9, 776.
[49] S. Lu, S. Wang, J. M. Grimes-Serrano, Expert Rev Vaccines 2008, 7, 175.
[50] D. Hobernik, M. Bros, Int J Mol Sci 2018, 19.
[51] M. Kanekiyo, C. J. Wei, H. M. Yassine, et al., Nature 2013, 499, 102.
[52] R. B. Zinyama-Gutsire, C. Chasela, et al., PLoS One 2015, 10, e0122659.
[53] M. Dahl, A. Tybjaerg-Hansen, P. Schnohr, et al., J Exp Med 2004, 199, 1391.
[54] N. Zhang, M. J. Bevan, Immunity 2011, 35, 161.
[55] N. Van Braeckel-Budimir, J. T. Harty, Front Microbiol 2014, 5, 272.
[56] E. J. Grant, S. M. Quinones-Parra, et al., Curr Opin Virol 2016, 16, 132.
[57] M. Koutsakos, P. T. Illing, T. H. O. Nguyen, et al., Nat Immunol 2019, 20, 613.
[58] A. Durgeau, Y. Virk, S. Corgnac, F. Mami-Chouaib, Front Immunol 2018, 9, 14.
[59] S. Sasaki, R. R. Amara, A. E. Oran, et al., Nat Biotechnol 2001, 19, 543.
[60] M. A. Chattergoon, J. J. Kim, J. S. Yang, et al., Nat Biotechnol 2000, 18, 974.
[61] M. A. Chattergoon, T. M. Robinson, et al., J Immunol 1998, 160, 5707.
[62] O. Akbari, N. Panjwani, S. Garcia, R. Tascon, et al., J Exp Med 1999, 189, 169.
[63] J. Yan, M. P. Morrow, J. S. Chu, T. Racine, et al., Vaccine 2018, 36, 3079.
[64] P. M. Patel, C. H. Ottensmeier, et al., Oncoimmunology 2018, 7, e1433516.
[65] J. W. Labonte, J. Adolf-Bryfogle, et al., J Comput Chem 2017, 38, 276.
[66] D. W. Kulp, S. Subramaniam, J. E. Donald, et al., J Comput Chem 2012, 33, 1645.
[67] B. Briney, D. Sok, J. G. Jardine, D. W. Kulp, et al., Cell 2016, 166, 1459.
[68] E. K. Duperret, M. C. Wise, A. Trautz, et al., Mol Ther 2018, 26, 435.
[69] Torrents de la Pena A, et al. Cell Rep. 2017; 20(8):1805-17.
[70] Sanders R W, Moore J P. Immunol Rev. 2017; 275(1):161-82.
[71] Medina-Ramirez, M, et al., Journal of experimental medicine. 2017; 214(9):2573-90.
[72] F. Li, Annu. Rev. Virol., 2016, 3, 237-261.
[73] B. J. Bosch, R. van der Zee, et al., J. Virol., 2003, 77, 8801-8811.
[74] A. C. Walls, et al., Proc. Natl. Acad. Sci. U.S.A., 2017, 114, 11157-111629.
[75] M. Gui, et al., Cell Res., 2017, 27, 119-129.
[76] J. Pallesen, et al., Proc. Natl. Acad. Sci. U.S.A., 2017, 114, E7348-E7357.
[77] A. C. Walls, et al., Cell, 2019, 176, 1026-1039.e15.
[78] Y. Yuan et al., Nat. Commun., 2017, 8, 15092.

1. A composition comprising an expressible nucleic acid sequence comprising:
a) a first nucleic acid sequence encoding a scaffold domain comprising a self-assembling polypeptide; and
b) a second nucleic acid sequence encoding a viral antigen from a virus of the family Coronaviridae.

2. The composition of claim 1, wherein the self-assembling polypeptide is from Aquifex aeolicus, Helicobacter pylori, Pyrococcus furiosus or Thermotoga maritima.

3. The composition of claim 1, wherein the self-assembling polypeptide comprises at least 70% sequence identity to SEQ ID NO: 8, SEQ ID NO: 10, SEQ ID NO: 12, SEQ ID NO: 14, SEQ ID NO: 16, SEQ ID NO: 18, SEQ ID NO: 20, or a functional fragment or variant thereof.

4. (canceled)

5. The composition of claim 1, wherein the viral antigen is an antigen from SARS-CoV-2 comprising at least 70% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof.

6. (canceled)

7. The composition of claim 1, wherein the expressible nucleic acid sequence further comprises a third nucleic acid sequence encoding a leader sequence.

8. (canceled)

9. The composition of claim 1, wherein the expressible nucleic acid sequence further comprises a nucleic acid sequence encoding a linker, said nucleic acid sequence positioned between the first nucleic acid sequence and the second nucleic acid sequence in the 5′ to 3′ orientation.

10. (canceled)

11. The composition of claim 1, wherein the expressible nucleic acid sequence comprises at least 70% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89 or SEQ ID NO: 90, or a functional fragment or variant thereof; wherein the expressible nucleic acid sequence encodes a polypeptide comprising at least 70% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88 or SEQ ID NO: 91, or a functional fragment or variant thereof.

12. (canceled)

13. A composition comprising an expressible nucleic acid sequence comprising:
a) a first nucleic acid sequence encoding a leader sequence; and
b) a second nucleic acid sequence encoding a soluble viral trimer or a soluble monomer thereof, wherein the soluble viral trimer or the soluble monomer thereof is from a virus of the family Coronaviridae.

14. The composition of claim 13, wherein the leader sequence comprises at least 70% sequence identity to SEQ ID NO: 1 or SEQ ID NO: 5, or a functional fragment or variant thereof.

15. The composition of claim 13, wherein the soluble viral trimer or the soluble monomer thereof is from a coronavirus.

16. (canceled)

17. The composition of claim 13, wherein the soluble viral trimer or the soluble monomer thereof comprises at least 70% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof.

18. The composition of claim 13, wherein the expressible nucleic acid sequence further comprises a third nucleic acid sequence encoding a linker.

19. (canceled)

20. The composition of claim 13, wherein the soluble viral trimer or the soluble monomer thereof comprises at least a portion of SARS-CoV-2 spike protein.

21. The composition of claim 13, wherein the expressible nucleic acid sequence further comprises a nucleic acid sequence encoding a self-assembling polypeptide or a functional fragment or variant thereof.

22. (canceled)

23. The composition of claim 1, wherein the expressible nucleic acid sequence comprises at least 70% sequence identity to SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof or wherein the expressible nucleic acid sequence encodes a polypeptide comprising at least 70% sequence identity to SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

24.-27. (canceled)

28. A pharmaceutical composition comprising (i) the composition of claim 13, and (ii) a pharmaceutically acceptable carrier.

29. The pharmaceutical composition of claim 28, wherein the composition of claim 13 is in an amount from about 1 to about 100 micrograms.

30. (canceled)

31. A method of vaccinating a subject comprising administering a therapeutically effective amount of the pharmaceutical composition of claim 28 to the subject.

32.-34. (canceled)

35. The method of claim 31, wherein the method is free of activating any mannose-binding lectin or complement process.

36.-37. (canceled)

38. The method of claim 31, wherein the therapeutically effective dose is from about 0.001 microgram of composition per kilogram of the subject to about 0.05 microgram per kilogram of the subject.

39. A method of inducing an immune response in a subject comprising administering to the subject the pharmaceutical composition of claim 28.

40.-50. (canceled)

51. A method of neutralizing one or a plurality of viruses in a subject comprising administering to the subject the pharmaceutical composition of claim 28.

52.-67. (canceled)

68. A method of inducing expression of a self-assembling vaccine in a subject comprising administering the pharmaceutical composition of claim 28.

69. The method of claim 68, wherein the method is free of administering any polypeptide directly to the subject.

70.-126. (canceled)
